(19) United States
(12) Patent Application Publication
(10) Pub. No.: US 2026/0103703 A1
(43) Pub. Date: Apr. 16, 2026

(54) siRNA COMPOSITIONS AND METHODS TARGETING ALPHA-SYNUCLEIN NUCLEIC ACIDS

(71) Applicant: Atalanta Therapeutics, Inc., Boston, MA (US)

(72) Inventors: Corrie GALLANT-BEHM, Wellesley, MA (US); Matthew HASSLER, Boston, MA (US); Daniel CURTIS, Belmont, MA (US); Bruno Miguel DA CRUZ GODINHO, Reading, MA (US)

(21) Appl. No.: 19/115,966

(22) PCT Filed: Sep. 28, 2023
(86) PCT No.: PCT/US2023/075434

§ 371 Date: Mar. 27, 2025

Related U.S. Application Data
(60) Provisional application No. 63/377,699, filed on Sep. 29, 2022.

(51) Int. Cl. C12N 15/113

(52) U.S. Cl. CPC C12N 15/113; C12N 2310/14; C12N 2310/315; C12N 2310/321; C12N 2310/322

(57) ABSTRACT

The instant application is directed to single- or double-stranded interfering RNA molecules (e.g., siRNA) that target an alpha-synuclein (SNCA) gene. The interfering RNA molecules may contain specific patterns of nucleoside modifications and internucleoside linkage modifications, as pharmaceutical compositions including the same. The siRNA molecules may be branched siRNA molecules, such as di-branched, tri-branched, or tetra-branched siRNA molecules. The disclosed siRNA molecules may further feature a 5′ phosphorus stabilizing moiety and/or a hydrophobic moiety. Additionally, the disclosure provides methods for delivering the siRNA molecule of the disclosure to the central nervous system of a subject, such as a subject identified as having a synucleinopathy.

### 1. SEQUENCE LISTING

The instant application contains a Sequence Listing which has been submitted electronically in XML format and is hereby incorporated by reference in its entirety. Said XML copy, created on Sep. 27, 2023, is named “51436-042WO2_Sequence_Listing_9_27_23” and is 820,156 bytes in size.

### 2. BACKGROUND

Expression of the alpha-synuclein (SNCA) gene produces the protein alpha-synuclein. While the alpha-synuclein protein is a soluble monomer normally localized at the presynaptic region of axons, the protein can form filamentous aggregates that are the major component of intracellular inclusions in neurodegenerative synucleinopathies. For example, mutations in the SNCA gene and SNCA gene multiplications have been linked to the formation of the filamentous aggregates that are a hallmark of familial Parkinson's disease. Additional neurodegenerative synucleinopathies associated with alpha-synuclein aggregates include sporadic Parkinson's disease, Alzheimer's disease, multiple system atrophy, and Lewy body dementia. Currently there are limited treatment options available for neurodegenerative synucleinopathies. Accordingly, there remains a need for therapeutics that can selectively diminish SNCA activity in a manner that provides effective treatment for neurodegenerative synucleinopathies.

### 3. SUMMARY

The instant application is directed to single- or double-stranded interfering RNA molecules (e.g., siRNA molecules) that target an SNCA gene. The interfering RNA molecules may contain specific patterns of nucleoside modifications and internucleoside linkage modifications. The disclosure also features pharmaceutical compositions including the same. The siRNA molecules may be branched siRNA molecules, such as di-branched, tri-branched, or tetra-branched siRNA molecules. The disclosed siRNA molecules may further feature a 5′ phosphorus stabilizing moiety and/or a hydrophobic moiety. Additionally, the disclosure provides methods for delivering the siRNA molecule of the disclosure to the central nervous system of a subject, such as a subject identified as having a synucleinopathy. In certain embodiments, an interfering RNA molecule of the present disclosure is a di-branched siRNA molecule. In certain embodiments, the di-branched siRNA molecules of the present disclosure comprise:

a) a sense strand comprising the sequence
(SEQ ID NO: 841)
(mU)#(mA)#(mC)(fC)(mA)(fC)(mU)(fU)(mA)(fU)(mU)
(mU)(mC)(fU)#(mA)#(mA)-DIO
and an antisense strand comprising the sequence
(SEQ ID NO: 842)
V(mU)#(fU)#(mA)(fG)(fA)(fA)(mA)(fU)(mA)(fA)(mG)
(fU)(mG)(fG)(mU)(fA)#(mG)#(mU)#(mC)#(mA)#(mC);
b) a sense strand comprising the sequence
(SEQ ID NO: 843)
(mC)#(mA)#(mA)(fG)(mU)(fG)(mC)(fU)
(mC)(fA)(mG)(mU)(mU)(fC)#(mC)#(mA)-DIO
and an antisense strand comprising the sequence
(SEQ ID NO: 844)
V(mU)#(fG)#(mG)(fA)(fA)(fC)(mU)(fG)(mA)(fG)(mC)
(fA)(mC)(fU)(mU)(fG)#(mU)#(mA)#(mC)#(mA)#(mG);
c) a sense strand comprising the sequence
(SEQ ID NO: 845)
(mA)#(mG)#(mU)(fG)(mG)(fU)(mG)(fC)(mA)(fU)(mG)
(mG)(mU)(fG)#(mU)#(mA)-DIO
and an antisense strand comprising the sequence
(SEQ ID NO: 846)
V(mU)#(fA)#(mC)(fA)(fC)(fC)(mA)(fU)(mG)(fC)
(mA)(fC)(mC)(fA)(mC)(fU)#(mC)#(mC)#(mC)#(mU)
#(mC).
d) a sense strand comprising the sequence
(SEQ ID NO: 847)
(mC)#(mA)#(mA)(fU)(mG)(fA)(mG)(fG)(mC)(fU)
(mU)(mA)(mU)(fG)#(mA)#(mA)-DIO
and an antisense strand comprising the sequence
(SEQ ID NO: 848)
V(mU)#(fU)#(mU)(fG)(fG)(fA)(mA)(fC)(mU)(fG)
(mA)(fG)(mC)(fA)(mC)(fU)#(mU)#(mG)#(mU)#(mA)
#(mC);
e) a sense strand comprising the sequence
(SEQ ID NO: 849)
(mU)#(mC)#(mA)(fG)(mU)(fU)(mC)(fC)(mA)(fA)
(mU)(mG)(mU)(fG)#(mC)#(mA)-DIO
and an antisense strand comprising the sequence
(SEQ ID NO: 850)
V(mU)#(fU)#(mU)(fG)(fG)(fA)(mA)(fC)(mU)(fG)
(mA)(fG)(mC)(fA)(mC)(fU)#(mU)#(mG)#(mU)#(mA)
#(mC).
f) a sense strand comprising the sequence
(SEQ ID NO: 851)
(mC)#(mU)#(mA)(fC)(mG)(fA)(mU)(fG)(mU)(fU)
(mA)(mA)(mA)(fA)#(mC)#(mA)-DIO
and an antisense strand comprising the sequence
(SEQ ID NO: 852)
V(mU)#(fG)#(mC)(fA)(fC)(fA)(mU)(fU)(mG)(fG)
(mA)(fA)(mC)(fU)(mG)(fA)#(mG)#(mC)#(mA)#(mC)
#(mU);
or
g) a sense strand comprising the sequence
(SEQ ID NO: 853)
(mC)#(mU)#(mA)(fC)(mG)(fA)(mU)(fG)(mU)(fU)
(mA)(mA)(mA)(fA)#(mC)#(mA)-DIO
and an antisense strand comprising the sequence
(SEQ ID NO: 854)
V(mU)#(fG)#(mU)(fU)(fU)(fU)(mA)(fA)(mC)(fA)
(mU)(fC)(mG)(fU)(mA)(fG)#(mA)#(mU)#(mU)#(mG)
#(mA);
wherein m represents a 2′-O-Me ribonucleoside,
f represents a 2′-F ribonucleoside,
# represents a phosphorothioate
internucleoside linkage,
-DIO represents a divalent
oligonucleotide (DIO) linker;
and V represents a vinyl phosphonate.

In some embodiments, the siRNA molecule comprises:

a) a sense strand comprising the sequence
(SEQ ID NO: 841)
(mU)#(mA)#(mC)(fC)(mA)(fC)(mU)(fU)(mA)
(fU)(mU)(mU)(mC)(fU)#(mA)#(mA)-DIO
and an antisense strand comprising the sequence
(SEQ ID NO: 842)
V(mU)#(fU)#(mA)(fG)(fA)(fA)(mA)(fU)(mA)
(fA)(mG)(fU)(mG)(fG)(mU)(fA)#(mG)#(mU)#
(mC)#(mA)#(mC);
b) a sense strand comprising the sequence
(SEQ ID NO: 843)
(mC)#(mA)#(mA)(fG)(mU)(fG)(mC)(fU)(mC)
(fA)(mG)(mU)(mU)(fC)#(mC)#(mA)-DIO
and an antisense strand comprising the sequence
(SEQ ID NO: 844)
V(mU)#(fG)#(mG)(fA)(fA)(fC)(mU)(fG)(mA)
(fG)(mC)(fA)(mC)(fU)(mU)(fG)#(mU)#(mA)
#(mC)#(mA)#(mG);
c) a sense strand comprising the sequence
(SEQ ID NO: 845)
(mA)#(mG)#(mU)(fG)(mG)(fU)(mG)(fC)(mA)
(fU)(mG)(mG)(mU)(fG)#(mU)#(mA)-DIO
and an antisense strand comprising the sequence
(SEQ ID NO: 846)
V(mU)#(fA)#(mC)(fA)(fC)(fC)(mA)(fU)(mG)
(fC)(mA)(fC)(mC)(fA)(mC)(fU)#(mC)#
(mC)#(mC)#(mU)#(mC).
d) a sense strand comprising the sequence
(SEQ ID NO: 847)
(mC)#(mA)#(mA)(fU)(mG)(fA)(mG)(fG)(mC)
(fU)(mU)(mA)(mU)(fG)#(mA)#(mA)-DIO
and an antisense strand comprising the sequence
(SEQ ID NO: 855)
V(mU)#(fU)#(mC)(fA)(fU)(fA)(mA)(fG)(mC)
(fC)(mU)(fC)(mA)(fU)(mU)(fG)#(mU)#
(mC)#(mA)#(mG)#(mG)
e) a sense strand comprising the sequence
(SEQ ID NO: 856)
(mA)#(mG)#(mU)(fG)(mC)(fU)(mC)(fA)(mG)
(fU)(mU)(mC)(mC)(fA)#(mA)#(mA)-DIO
and an antisense strand comprising the sequence
(SEQ ID NO: 850)
V(mU)#(fU)#(mU)(fG)(fG)(fA)(mA)(fC)(mU)
(fG)(mA)(fG)(mC)(fA)(mC)(fU)#(mU)#(mG)#
(mU)#(mA)#(mC).
f) a sense strand comprising the sequence
(SEQ ID NO: 857)
(mU)#(mC)#(mA)(fG)(mU)(fU)(mC)(fC)(mA)
(fA)(mU)(mG)(mU)(fG)#(mC)#(mA)-DIO
and an antisense strand comprising the sequence
(SEQ ID NO: 852)
V(mU)#(fG)#(mC)(fA)(fC)(fA)(mU)(fU)(mG)
(fG)(mA)(fA)(mC)(fU)(mG)(fA)#(mG)#(mC)
#(mA)#(mC)#(mU);
or
g) a sense strand comprising the sequence
(SEQ ID NO: 853)
(mC)#(mU)#(mA)(fC)(mG)(fA)(mU)(fG)(mU)
(fU)(mA)(mA)(mA)(fA)#(mC)#(mA)-DIO
and an antisense strand comprising the sequence
(SEQ ID NO: 854)
V(mU)#(fG)#(mU)(fU)(fU)(fU)(mA)(fA)(mC)
(fA)(mU)(fC)(mG)(fU)(mA)(fG)#(mA)#(mU)
#(mU)#(mG)#(mA);
wherein m represents a 2′-O-Me ribonucleoside,
f represents a 2′-F ribonucleoside,
# represents a phosphorothioate
internucleoside linkage,
-DIO represents a divalent oligonucleotide
(DIO) linker;
and V represents a vinyl phosphonate.

In another aspect, the disclosure features a small interfering RNA (siRNA) molecule comprising an antisense strand and sense strand having complementarity to the antisense strand. The antisense strand may be, e.g., from 10 to 30 nucleotides in length and may have complementarity sufficient to hybridize to a region within an alpha-synuclein (SNCA) mRNA transcript having the nucleic acid sequence of any one of SEQ ID NOs: 421-840.

In some embodiments, the antisense strand has at least 70% complementarity to a region of 21 contiguous nucleobases within the SNCA mRNA transcript having the nucleic acid sequence of any one of SEQ ID NOs: 421-840. In some embodiments, the antisense strand has at least 75% complementarity to a region of 21 contiguous nucleobases within the SNCA mRNA transcript having the nucleic acid sequence of any one of SEQ ID NOs: 421-840. In some embodiments, the antisense strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the SNCA mRNA transcript having the nucleic acid sequence of any one of SEQ ID Nos: 421-840.

In some embodiments, the antisense strand comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the SNCA mRNA transcript having the nucleic acid sequence of any one of SEQ ID NOs: 421-840.

In some embodiments, the antisense strand comprises from 10 to 30 contiguous nucleotides that are fully complementary to a contiguous polynucleotide segment of equal length within the region of the SNCA mRNA transcript having the nucleic acid sequence of any one of SEQ ID NOs: 421-840.

In some embodiments, the antisense strand comprises from 12 to 30 contiguous nucleotides that are fully complementary to a contiguous polynucleotide segment of equal length within the region of the SNCA mRNA transcript having the nucleic acid sequence of any one of SEQ ID NOs: 421-840.

In some embodiments, the antisense strand comprises from 15 to 30 contiguous nucleotides that are fully complementary to a contiguous polynucleotide segment of equal length within the region of the SNCA mRNA transcript having the nucleic acid sequence of any one of SEQ ID NOs: 421-840.

In some embodiments, the antisense strand comprises from 18 to 30 contiguous nucleotides that are fully complementary to a contiguous polynucleotide segment of equal length within the region of the SNCA mRNA transcript having the nucleic acid sequence of any one of SEQ ID NOs: 421-840.

In some embodiments, the antisense strand comprises from 21 to 30 contiguous nucleotides that are fully complementary to a contiguous polynucleotide segment of equal length within the region of the SNCA mRNA transcript having the nucleic acid sequence of any one of SEQ ID NOs: 421-840.

In some embodiments, the antisense strand comprises from 24 to 30 contiguous nucleotides that are fully complementary to a contiguous polynucleotide segment of equal length within the region of the SNCA mRNA transcript having the nucleic acid sequence of any one of SEQ ID NOs: 421-840.

In some embodiments, the antisense strand comprises 30 contiguous nucleotides that are fully complementary to a contiguous polynucleotide segment of equal length within the region of the SNCA mRNA transcript having the nucleic acid sequence of any one of SEQ ID NOs: 421-840.

In some embodiments, the antisense strand comprises 9 or fewer nucleotide mismatches relative to a region of 21 contiguous nucleobases of the SNCA mRNA transcript having the nucleic acid sequence of any one of SEQ ID NOs: 421-840. In some embodiments, the antisense strand comprises 8 or fewer, 7 or fewer, 6 or fewer, 5 or fewer, 4 or fewer, 3 or fewer, 2 or fewer, or only 1 mismatch relative to the region of the SNCA mRNA transcript having the nucleic acid sequence of any one of SEQ ID NOs: 421-840.

In some embodiments, the region of the SNCA mRNA transcript has the nucleic acid sequence of any one of SEQ ID NOs: 459, 535, 551, 599, 631, 663, 664, 666, 667, 671, 748, 750, 751, 752, 760, 762, and 777.

In some embodiments, the region of the SNCA mRNA transcript has the nucleic acid sequence of any one of SEQ ID NOs: 535, 599, 664, 666, 671, 750, 752, 760, and 762.

In some embodiments, the antisense strand has a nucleic acid sequence that is at least 85% identical to the nucleic acid sequence of any one of SEQ ID NOs: 1-420.

In some embodiments, the antisense strand has a nucleic acid sequence that is at least 90% identical to the nucleic acid sequence of any one of SEQ ID NOs: 1-420.

In some embodiments, the antisense strand has a nucleic acid sequence that is at least 95% identical to the nucleic acid sequence of SEQ ID NOs: 1-420. In some embodiments, the antisense strand has a nucleic acid sequence that is at least 96%, 97%, 98%, or 99% identical to the nucleic acid sequence of any one of SEQ ID NOs: 1-420.

In some embodiments, the antisense strand has the nucleic acid sequence of any one of SEQ ID NOs: 1-420.

In some embodiments, the nucleic acid sequence is any one of SEQ ID NOs: 39, 115, 131, 179, 211, 243, 244, 246, 247, 251, 328, 330, 331, 332, 340, 342, and 357.

In some embodiments, the nucleic acid sequence is any one of SEQ ID NOs: 115, 179, 244, 246, 251, 330, 332, 340, and 342.

In some embodiments, the sense strand has a nucleic acid sequence that is at least 85% identical to the nucleic acid sequence of any one of SEQ ID NOs: 421-840.

In some embodiments, the sense strand has a nucleic acid sequence that is at least 90% identical to the nucleic acid sequence of any one of SEQ ID NOs: 421-840.

In some embodiments, the sense strand has a nucleic acid sequence that is at least 95% identical to the nucleic acid sequence of SEQ ID NOs: 421-840. In some embodiments, the sense strand has a nucleic acid sequence that is at least 96%, 97%, 98%, or 99% identical to the nucleic acid sequence of any one of SEQ ID NOs: 421-840.

In some embodiments, the sense strand has the nucleic acid sequence of any one of SEQ ID NOs: 421-840.

In some embodiments, the nucleic acid sequence is any one of SEQ ID NOs: 459, 535, 551, 599, 631, 663, 664, 666, 667, 671, 748, 750, 751, 752, 760, 762, and 777.

In some embodiments, the nucleic acid sequence is any one of SEQ ID NOs: 535, 599, 664, 666, 671, 750, 752, 760, and 762.

In some embodiments, the antisense strand comprises a structure represented by Formula I, wherein Formula I is, in the 5′-to-3′ direction:

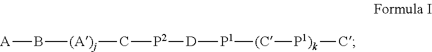

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

In some embodiments, the antisense strand comprises a structure represented by Formula A1, wherein Formula A1 is, in the 5′-to-3′ direction:

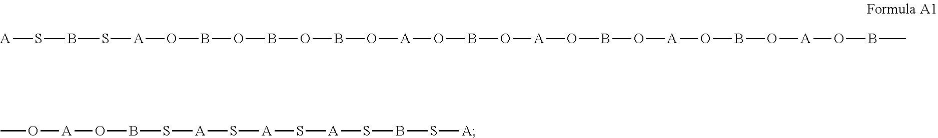

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand comprises a structure represented by Formula II, wherein Formula II is, in the 5′-to-3′ direction:

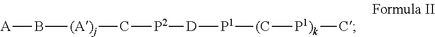

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

In some embodiments, the antisense strand comprises a structure represented by Formula A2, wherein Formula A2 is, in the 5′-to-3′ direction:

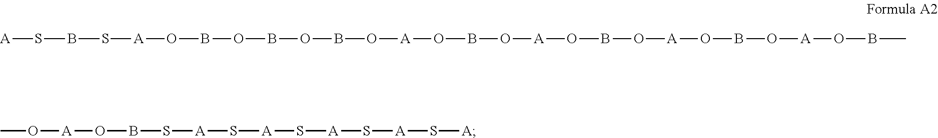

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand comprises a structure represented by Formula III, wherein Formula III is, in the 5′-to-3′ direction:

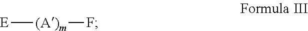

wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7.

In some embodiments, the sense strand comprises a structure represented by Formula S1, wherein Formula S1 is, in the 5′-to-3′ direction:

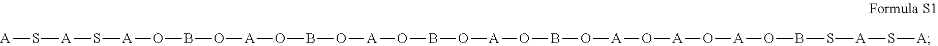

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand comprises a structure represented by Formula S2, wherein Formula S2 is, in the 5′-to-3′ direction:

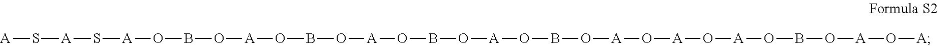

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand comprises a structure represented by Formula S3, wherein Formula S3 is, in the 5′-to-3′ direction:

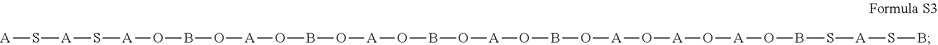

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand comprises a structure represented by Formula S4, wherein Formula S4 is, in the 5′-to-3′ direction:

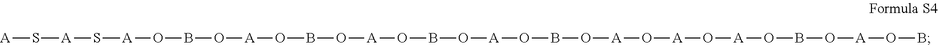

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand comprises a structure represented by Formula IV, wherein Formula IV is, in the 5′-to-3′ direction:

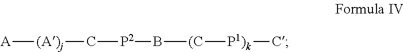

wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.

In some embodiments, the antisense strand comprises a structure represented by Formula A3, wherein Formula A3 is, in the 5′-to-3′ direction:

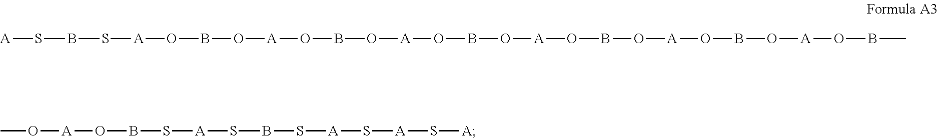

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand comprises a structure represented by Formula V, wherein Formula V is, in the 5′-to-3′ direction:

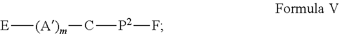

wherein E is represented by the formula (C—P¹)₂;
F is represented by the formula D-P¹—C—P¹—C, D-P²—C—P²—C, D-P¹—C—P¹-D, or D-P²—C—P²-D;
A′, C, D, P¹ and P² are as defined in Formula IV; and
m is an integer from 1 to 7.

In some embodiments, the sense strand comprises a structure represented by Formula S5, wherein Formula S5 is, in the 5′-to-3′ direction:

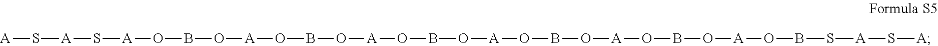

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand comprises a structure represented by Formula S6, wherein Formula S6 is, in the 5′-to-3′ direction:

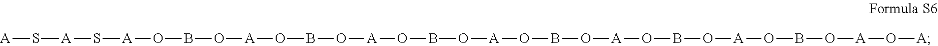

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand comprises a structure represented by Formula S7, wherein Formula S7 is, in the 5′-to-3′ direction:

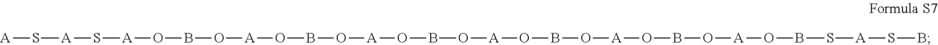

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand comprises a structure represented by Formula S8, wherein Formula S8 is, in the 5′-to-3′ direction:

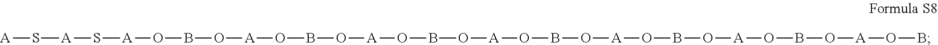

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand comprises a structure represented by Formula VI, wherein Formula VI is, in the 5′-to-3′ direction:

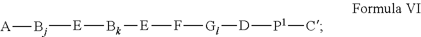

wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7.

In some embodiments, the antisense strand comprises a structure represented by Formula A4, wherein Formula A4 is, in the 5′-to-3′ direction:

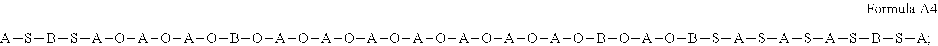

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the sense strand comprises a structure represented by Formula VII, wherein Formula VII is, in the 5′-to-3′ direction:

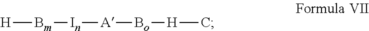

wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7;
n is an integer from 1 to 7; and
is an integer from 1 to 7.

In some embodiments, the sense strand comprises a structure represented by Formula S9, wherein Formula S9 is, in the 5′-to-3′ direction:

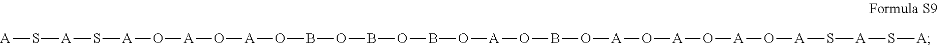

wherein A represents a 2′-O-Me ribonucleoside, B represents a 2′-F ribonucleoside, O represents a phosphodiester internucleoside linkage, and S represents a phosphorothioate internucleoside linkage.

In some embodiments, the antisense strand further comprises a 5′ phosphorus stabilizing moiety at the 5′ end of the antisense strand.

In some embodiments, the sense strand further comprises a 5′ phosphorus stabilizing moiety at the 5′ end of the sense strand.

In some embodiments, each 5′ phosphorus stabilizing moiety is, independently, represented by any one of Formulas IX-XVI:

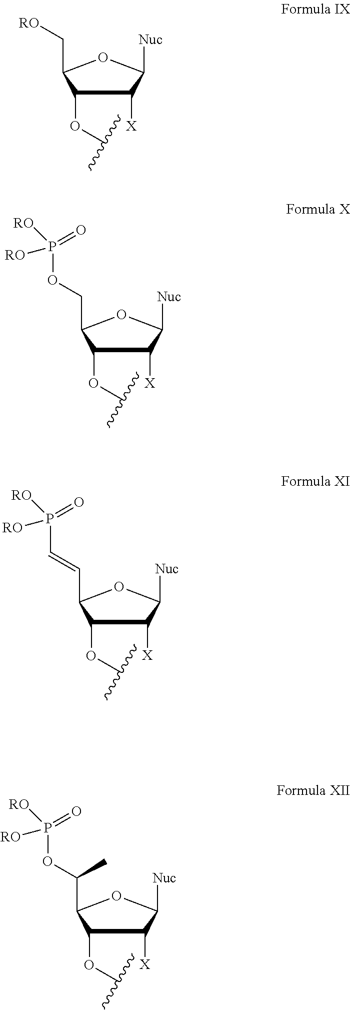

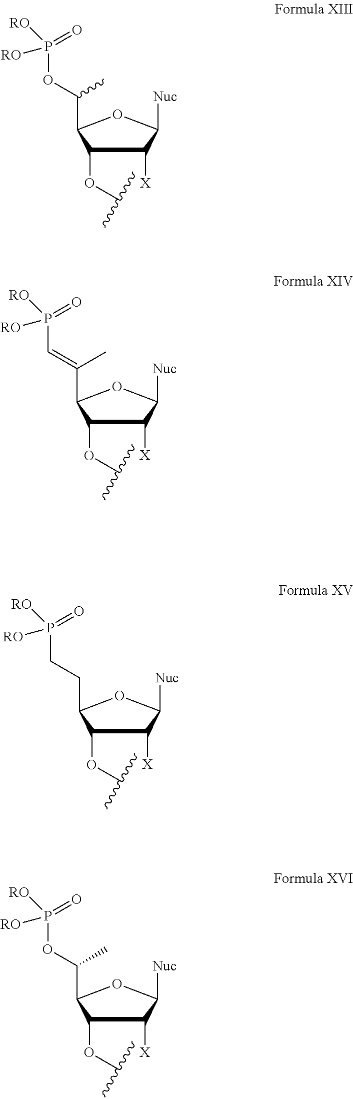

wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, hydroxy, or hydrogen.

In some embodiments, the nucleobase is an adenine, uracil, guanine, thymine, or cytosine.

In some embodiments, the 5′ phosphorus stabilizing moiety is (E)-vinylphosphonate represented by Formula XI.

In some embodiments, the siRNA molecule further comprises a hydrophobic moiety at the 5′ or the 3′ end of the siRNA molecule.

In some embodiments, the hydrophobic moiety is selected from a group consisting of cholesterol, vitamin D, or tocopherol.

In some embodiments, the length of the sense strand is between 12 and 30 nucleotides.

In some embodiments, the siRNA molecule is a branched siRNA molecule.

In some embodiments, the branched siRNA molecule is di-branched, tri-branched, or tetra-branched.

In some embodiments, the siRNA molecule is a di-branched siRNA molecule. In some embodiments, the di-branched siRNA molecule is represented by any one of Formulas XVII-XIX:

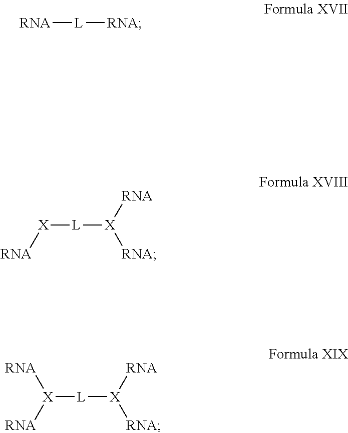

wherein each RNA is, independently, an siRNA molecule, L is a linker, and each X, independently, represents a branch point moiety.

In some embodiments, the siRNA molecule is a tri-branched siRNA molecule, optionally wherein the tri-branched siRNA molecule is represented by any one of Formulas XX-XXIII:

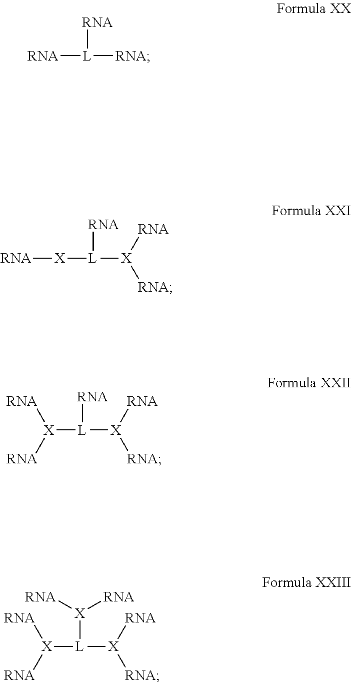

wherein each RNA is, independently, an siRNA molecule, L is a linker, and each X, independently, represents a branch point moiety.

In some embodiments, the siRNA molecule is a tetra-branched siRNA molecule, 15 optionally wherein the tetra-branched siRNA molecule is represented by any one of Formulas XXIV-XXVIII:

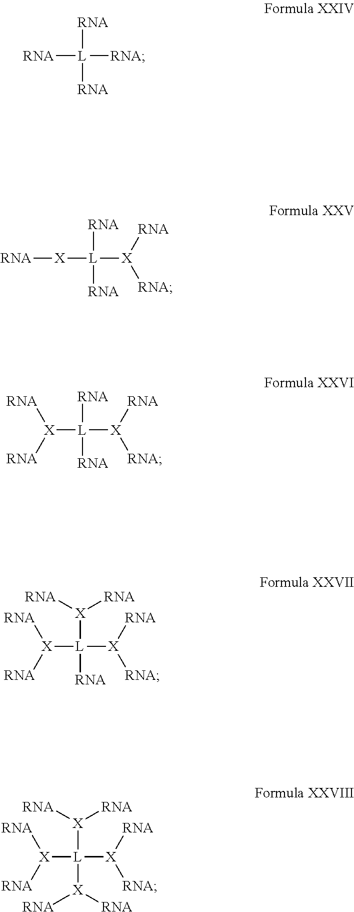

wherein each RNA is, independently, an siRNA molecule, L is a linker, and each X, independently, represents a branch point moiety.

In some embodiments, the linker is selected from a group consisting of one or more contiguous subunits of an ethylene glycol, alkyl, carbohydrate, block copolymer, peptide, RNA, and DNA.

In some embodiments, the one or more contiguous subunits is 2 to 20 contiguous subunits.

In another aspect, the disclosure features a pharmaceutical composition comprising the siRNA molecule of any of the foregoing aspects or embodiments of the disclosure, in combination with a pharmaceutically acceptable excipient, carrier, or diluent.

In another aspect, the disclosure features a method of delivering an siRNA molecule to a subject diagnosed as having a synucleinopathy, the method comprising administering to the subject a therapeutically effective amount of the siRNA molecule of any of the foregoing aspects or embodiments of the disclosure, optionally in combination with a pharmaceutically acceptable excipient, carrier, or diluent.

In another aspect, the disclosure features a method of treating a synucleinopathy in a subject in need thereof, the method comprising administering to the subject a therapeutically effective amount of the siRNA molecule of any of the foregoing aspects or embodiments of the disclosure, optionally in combination with a pharmaceutically acceptable excipient, carrier, or diluent.

In some embodiments, the synucleinopathy is Parkinson's disease. In some embodiments, the synucleinopathy is Alzheimer's disease. In some embodiments, the synucleinopathy is Lewy body dementia. In some embodiments, the synucleinopathy is a multiple symptom atrophy.

In another aspect, the disclosure features a method of reducing SNCA expression in a subject in need thereof, the method comprising administering to the subject a therapeutically effective amount of the siRNA molecule of any of the foregoing aspects or embodiments of the disclosure, optionally in combination with a pharmaceutically acceptable excipient, carrier, or diluent.

In some embodiments, the siRNA molecule or the pharmaceutical composition is administered to the subject by way of intracerebroventricular, intrastriatal, intraparenchymal, or intrathecal injection. In some embodiments, the siRNA molecule or the pharmaceutical composition is administered to the subject by way of intravenous, intramuscular, or subcutaneous injection.

In some embodiments, the subject is a human.

In a further aspect, the disclosure features a kit comprising (a) the siRNA molecule of any of the foregoing aspects or embodiments of the disclosure, optionally in combination with a pharmaceutically acceptable excipient, carrier, or diluent, and (b) a package insert. In some embodiments, the package insert instructs a user of the kit to perform the method of any of the above aspects or embodiments of the disclosure.

### 4. BRIEF DESCRIPTION OF THE FIGURES

FIG. 1 is a graph showing reduction in SNCA mRNA in hSNCA transgenic mice that were administered an siRNA molecule of the disclosure with a sense strand having SEQ ID No.: 664 and an antisense strand having SEQ ID No.: 244. mRNA was quantified in frontal cortex (fCTx), striatum (Cpu), hippocampus (Hp), temporal cortex (tCTx), midbrain, pons, medulla, and cerebellum (Cb), at a timepoint of 1 month, 2 months, 3 months, or 4 months following di-siRNA treatment at a dose level of 30 nmol.

FIG. 2 is a graph showing reduction in SNCA mRNA in hSNCA transgenic mice that were administered an siRNA molecule of the disclosure with a sense strand having SEQ ID No.: 664 and an antisense strand having SEQ ID No.: 244. mRNA was quantified in cervical spinal cord (SC-C), thoracic spinal cord (SC-T), and lumbar spinal cord (SC-L), at a timepoint of 1 month, 2 months, 3 months, or 4 months following di-siRNA treatment at a dose level of 30 nmol.

FIG. 3 is a graph showing reduction in human SNCA protein in hSNCA transgenic mice that were administered an siRNA molecule of the disclosure with a sense strand having SEQ ID No.: 664 and an antisense strand having SEQ ID No.: 244. Protein was quantified in frontal cortex (fCTx), striatum (Cpu), hippocampus (Hp), temporal cortex (tCTx), midbrain, and cerebellum (Cb), at a timepoint of 1 month, 2 months, 3 months, or 4 months following di-siRNA treatment at a dose level of 30 nmol.

FIG. 4 is a graph showing reduction in human SNCA protein in hSNCA transgenic mice that were administered an siRNA molecule of the disclosure with a sense strand having SEQ ID No.: 664 and an antisense strand having SEQ ID No.: 244. Protein was quantified in cervical spinal cord (SC-C), thoracic spinal cord (SC-T), and lumbar spinal cord (SC-L), at a timepoint of 1 month, 2 months, 3 months, or 4 months following di-siRNA treatment at a dose level of 30 nmol.

FIG. 5 is a graph showing reduction in mouse Snca mRNA in hSNCA transgenic mice that were administered an siRNA molecule of the disclosure with a sense strand having SEQ ID No.: 664 and an antisense strand having SEQ ID No.: 244. mRNA was quantified in frontal cortex, striatum, hippocampus, and temporal cortex, 3 months or 4 months following di-siRNA treatment at 2.5 nmol, 5 nmol, 15 nmol, or 20 nmol dose levels.

FIG. 6 is a graph showing reduction in human SNCA mRNA in hSNCA transgenic mice that were administered an siRNA molecule of the disclosure with a sense strand having SEQ ID No.: 664 and an antisense strand having SEQ ID No.: 244. mRNA was quantified in frontal cortex, striatum, hippocampus, and temporal cortex, 3 months or 4 months following di-siRNA treatment at 2.5 nmol, 5 nmol, 15 nmol, or 20 nmol dose levels.

FIG. 7 is a graph showing reduction in human SNCA protein in hSNCA transgenic mice that were administered an siRNA molecule of the disclosure with a sense strand having SEQ ID No.: 664 and an antisense strand having SEQ ID No.: 244. Protein was quantified in frontal cortex, striatum, hippocampus, and temporal cortex, 3 months or 4 months following di-siRNA treatment at 2.5 nmol, 5 nmol, 15 nmol, or 20 nmol dose levels.

### 5. DETAILED DESCRIPTION

The instant application is directed to single- or double-stranded interfering RNA molecules (e.g., siRNA) targeting an SNCA gene. The interfering RNA molecules may contain specific patterns of nucleoside modifications and internucleoside linkage modifications, as pharmaceutical compositions including the same. The siRNA molecules may be branched siRNA molecules, such as di-branched, tri-branched, or tetra-branched siRNA molecules. The disclosed siRNA molecules may further feature a 5′ phosphorus stabilizing moiety and/or a hydrophobic moiety. Additionally, the disclosure provides methods for delivering the siRNA molecule of the disclosure to the central nervous system of a subject, such as a subject identified as having a synucleinopathy.

### 5.1. Definitions

Unless otherwise defined herein, scientific, and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the term “nucleic acids” refers to RNA or DNA molecules consisting of a chain of ribonucleotides or deoxyribonucleotides, respectively. As used herein, the term “therapeutic nucleic acid” refers to a nucleic acid molecule (e.g., ribonucleic acid) that has partial or complete complementarity to, and interacts with, a disease-associated target mRNA and mediates silencing of expression of the mRNA.

As used herein, the term “carrier nucleic acid” refers to a nucleic acid molecule (e.g., ribonucleic acid) that has sequence complementarity with, and hybridizes with, a therapeutic nucleic acid. As used herein, the term “3′ end” refers to the end of the nucleic acid that contains an unmodified hydroxyl group at the 3′ carbon of the ribose ring.

As used herein, the term “nucleoside” refers to a molecule made up of a heterocyclic base and its sugar.

As used herein, the term “nucleotide” refers to a nucleoside having a phosphate group on its 3′ or 5′ sugar hydroxyl group.

As used herein, the term “siRNA” refers to small interfering RNA duplexes that induce the RNA interference (RNAi) pathway. siRNA molecules can vary in length (generally, between 18 and 30 base pairs) and contain varying degrees of complementarity to their target mRNA. The term “siRNA” includes duplexes of two separate strands, as well as single strands that optionally form hairpin structures including a duplex region.

As used herein, the term “antisense strand” refers to the strand of the siRNA duplex that contains some degree of complementarity to the target gene.

As used herein, the term “sense strand” refers to the strand of the siRNA duplex that contains complementarity to the antisense strand.

As used herein, the term “chemically modified nucleotide” refers to a non-standard nucleotide, including non-naturally occurring ribonucleotides or deoxyribonucleotides. Exemplary chemically modified nucleotides are modified at any position so as to alter certain chemical properties of the nucleotide yet retain the ability of the chemically modified nucleotide to perform its intended function.

As used herein, the term “metabolically stabilized” refers to RNA molecules that contain ribonucleotides that have been chemically modified from 2′-hydroxyl groups to 2′-O-methyl groups, 2′ fluoro groups, or other modifications known in the art to stabilized RNA to enzymatic and/or non-enzymatic degradation.

As used herein, the term “phosphorothioate” refers to a phosphate group of a nucleotide that is modified by substituting one or more of the oxygens of the phosphate group with sulfur.

As used herein, the term “ethylene glycol chain” refers to a carbon chain with the formula ((CH₂OH)₂).

As used herein, “alkyl” refers to a saturated hydrocarbon group. Alkyl groups may be acyclic or cyclic and contain only C and H when unsubstituted. When an alkyl residue having a specific number of carbons is named, all geometric isomers having that number of carbons are intended to be encompassed and described; thus, for example, “butyl” is meant to include n-butyl, sec-butyl, and iso-butyl. Examples of alkyl include ethyl, propyl, butyl, pentyl, hexyl, heptyl, octyl, cyclopropyl, cyclobutyl, cyclopentyl, cyclohexyl, cycloheptyl, cyclooctyl, and the like. In some embodiments, alkyl may be substituted. Suitable substituents that may be introduced into an alkyl group include, for example, hydroxy, alkoxy, amino, alkylamino, and halo, among others.

As used herein, “alkenyl” refers to an acyclic or cyclic unsaturated hydrocarbon group having at least one site of olefinic unsaturation (i.e., having at least one moiety of the formula C═C). Alkenyl groups contain only C and H when unsubstituted. When an alkenyl residue having a specific number of carbons is named, all geometric isomers having that number of carbons are intended to be encompassed and described; thus, for example, “butenyl” is meant to include n-butenyl, sec-butenyl, and iso-butenyl. Examples of alkenyl include —CH═CH₂, —CH₂—CH═CH₂, and —CH₂—CH═CH—CH═CH₂. In some embodiments, alkenyl may be substituted. Suitable substituents that may be introduced into an alkenyl group include, for example, hydroxy, alkoxy, amino, alkylamino, and halo, among others.

As used herein, “alkynyl” refers to an acyclic or cyclic unsaturated hydrocarbon group having at least one site of acetylenic unsaturation (i.e., having at least one moiety of the formula C≡C). Alkynyl groups contain only C and H when unsubstituted. When an alkynyl residue having a specific number of carbons is named, all geometric isomers having that number of carbons are intended to be encompassed and described; thus, for example, “pentynyl” is meant to include n-pentynyl, sec-pentynyl, iso-pentynyl, and tert-pentynyl. Examples of alkynyl include —C≡CH and —C≡C—CH₃. In some embodiments, alkynyl may be substituted. Suitable substituents that may be introduced into an alkynyl group include, for example, hydroxy, alkoxy, amino, alkylamino, and halo, among others.

As used herein the term “phenyl” denotes a monocyclic arene in which one hydrogen atom from a carbon atom of the ring has been removed. A phenyl group can be unsubstituted or substituted with one or more suitable substituents, wherein the substituent replaces an H of the phenyl group.

As used herein, the term “benzyl” refers to monovalent radical obtained when a hydrogen atom attached to the methyl group of toluene is removed. A benzyl generally has the formula of phenyl-CH₂—.

A benzyl group can be unsubstituted or substituted with one or more suitable substituents. For example, the substituent may replace an H of the phenyl component and/or an H of the methylene (—CH₂—) component.

As used herein, the term “amide” refers to an alkyl, alkenyl, alkynyl, or aromatic group that is attached to an amino-carbonyl functional group.

As used herein, the term “internucleoside” and “internucleotide” refer to the bonds between nucleosides and nucleotides, respectively.

As used herein, the term “triazol” refers to heterocyclic compounds with the formula (C₂H₃N₃), having a five-membered ring of two carbons and three nitrogens, the positions of which can change resulting in multiple isomers.

As used herein, the term “terminal group” refers to the group at which a carbon chain or nucleic acid ends.

As used herein, the term “lipophilic amino acid” refers to an amino acid including a hydrophobic moiety (e.g., an alkyl chain or an aromatic ring).

As used herein, the term “target of delivery” refers to the organ or part of the body that is desired to deliver the branched oligonucleotide compositions to.

As used herein, the term “branched siRNA” refers to a compound containing two or more double-stranded siRNA molecules covalently bound to one another. Branched siRNA molecules may be “di-branched,” also referred to herein as “di-siRNA,” wherein the siRNA molecule includes 2 siRNA molecules covalently bound to one another, e.g., by way of a linker. Branched siRNA molecules may be “tri-branched,” also referred to herein as “tri-siRNA,” wherein the siRNA molecule includes 3 siRNA molecules covalently bound to one another, e.g., by way of a linker. Branched siRNA molecules may be “tetra-branched,” also referred to herein as “tetra-siRNA,” wherein the siRNA molecule includes 4 siRNA molecules covalently bound to one another, e.g., by way of a linker.

As used herein, the term “branch point moiety” refers to a chemical moiety of a branched siRNA structure of the disclosure that may be covalently linked to a 5′ end or a 3′ end of an antisense strand or a sense strand of an siRNA molecule and which may support the attachment of additional single- or double-stranded siRNA molecules. Non-limiting examples of branch point moieties suitable for use in conjunction with the disclosed methods and compositions include, e.g., phosphoroamidite, tosylated solketal, 1,3-diaminopropanol, pentaerythritol, and any one of the branch point moieties described in U.S. Pat. No. 10,478,503.

As used herein, the term “5′ phosphorus stabilizing moiety” refers to a terminal phosphate group that includes phosphates as well as modified phosphates (e.g., phosphorothioates, phosphodiesters, phosphonates). The phosphate moiety can be located at either terminus, e.g., at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′), or alkyl where R′ is H, an amino protecting group, or unsubstituted or substituted alkyl. In some embodiments, the 5′ and or 3′ terminal group can include from 1 to 3 phosphate moieties that are each, independently, unmodified (di- or tri-phosphates) or modified.

As used herein, the term “between X and Y” is inclusive of the values of X and Y. For example, “between X and Y” refers to the range of values between the value of X and the value of Y, as well as the value of X and the value of Y.

As used herein, an “amino acid” refers to a molecule containing amine and carboxyl functional groups and a side chain specific to the amino acid.

In some embodiments the amino acid is chosen from the group of proteinogenic amino acids. In some embodiments, the amino acid is an L-amino acid or a D-amino acid. In some embodiments, the amino acid is a synthetic amino acid (e.g., a beta-amino acid).

It is understood that certain internucleoside linkages provided herein, including, e.g., phosphodiester and phosphorothioate, include a formal charge of −1 at physiological pH, and that said formal charge will be balanced by a cationic moiety, e.g., an alkali metal such as sodium or potassium, an alkali earth metal such as calcium or magnesium, or an ammonium or guanidinium ion.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 10:117-21, 2000; Rusckowski et al., Antisense Nucleic Acid Drug Dev. 10:333-45, 2000; Stein, Antisense Nucleic Acid Drug Dev. 11:317-25, 2001; Vorobjev et al., Antisense Nucleic Acid Drug Dev. 11:77-85, 2001; and U.S. Pat. No. 5,684,143. Certain of the above-referenced modifications (e.g., phosphate group modifications) can decrease the rate of hydrolysis of, for example, polynucleotides including said modifications in vivo or in vitro.

As used herein, the term “complementary” refers to two nucleotides that form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software.

“Percent (%) sequence complementarity” with respect to a reference polynucleotide sequence is defined as the percentage of nucleic acids in a candidate sequence that are complementary to the nucleic acids in the reference polynucleotide sequence, after aligning the sequences and introducing gaps, if necessary, to achieve the maximum percent sequence complementarity. A given nucleotide is considered to be “complementary” to a reference nucleotide as described herein if the two nucleotides form canonical Watson-Crick base pairs. For the avoidance of doubt, Watson-Crick base pairs in the context of the present disclosure include adenine-thymine, adenine-uracil, and cytosine-guanine base pairs. A proper Watson-Crick base pair is referred to in this context as a “match,” while each unpaired nucleotide, and each incorrectly paired nucleotide, is referred to as a “mismatch.” Alignment for purposes of determining percent nucleic acid sequence complementarity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal complementarity over the full length of the sequences being compared. As an illustration, the percent sequence complementarity of a given nucleic acid sequence, A, to a given nucleic acid sequence, B, (which can alternatively be phrased as a given nucleic acid sequence, A that has a certain percent complementarity to a given nucleic acid sequence, B) is calculated as follows:

$$100 multiplied by ( the fraction X / Y )$$

where X is the number of complementary base pairs in an alignment (e.g., as executed by computer software, such as BLAST) in that program's alignment of A and B, and where Y is the total number of nucleic acids in B. It will be appreciated that where the length of nucleic acid sequence A is not equal to the length of nucleic acid sequence B, the percent sequence complementarity of A to B will not equal the percent sequence complementarity of B to A. As used herein, a query nucleic acid sequence is considered to be “completely complementary” to a reference nucleic acid sequence if the query nucleic acid sequence has 100% sequence complementarity to the reference nucleic acid sequence.

The term “gene silencing” refers to the suppression of gene expression, e.g., transgene, heterologous gene and/or endogenous gene expression, which may be mediated through processes that affect transcription and/or through processes that affect post-transcriptional mechanisms. In some embodiments, gene silencing occurs when an RNAi molecule initiates the inhibition or degradation of the mRNA transcribed from a gene of interest in a sequence-specific manner via RNA interference, thereby preventing translation of the gene's product.

The phrase “overactive disease driver gene,” as used herein, refers to a gene having increased activity and/or expression that contributes to or causes a disease state in a subject (e.g., a human). The disease state may be caused or exacerbated by the overactive disease driver gene directly or by way of an intermediate gene(s).

The term “negative regulator,” as used herein, refers to a gene that negatively regulates (e.g., reduces or inhibits) the expression and/or activity of another gene or set of genes.

The term “positive regulator,” as used herein, refers to a gene that positively regulates (e.g., increases or saturates) the expression and/or activity of another gene or set of genes.

The term “phosphate moiety” as used herein, refers to a terminal phosphate group that includes phosphates as well as modified phosphates. The phosphate moiety can be located at either terminus, e.g., at the 5′-terminal nucleoside. In one aspect, the terminal phosphate is unmodified having the formula —O—P(═O)(OH)OH. In another aspect, the terminal phosphate is modified such that one or more of the O and OH groups are replaced with H, O, S, N(R′) or alkyl where R′ is H, an amino protecting group or unsubstituted or substituted alkyl. In some embodiments, the 5′ and or 3′ terminal group can include from 1 to 3 phosphate moieties that are each, independently, unmodified (di or tri-phosphates) or modified.

In the context of this disclosure, the term “oligonucleotide” refers to an oligomer or polymer of ribonucleic acid (RNA) or deoxyribonucleic acid (DNA) or other nucleic acids. The term includes oligonucleotides composed of naturally-occurring nucleobases, sugars and covalent internucleoside (backbone) linkages as well as oligonucleotides having non-naturally-occurring (e.g., chemically modified) portions that function similarly. Such chemically modified oligonucleotides can exhibit desirable properties such as, for example, enhanced cellular uptake, enhanced affinity for nucleic acid target and increased stability in the presence of nucleases.

The term “treatment” refers to clinical intervention designed to alter the natural course of the patient or cell being treated during the course of clinical pathology. Desirable effects of treatment include decreasing the rate of disease progression, ameliorating or palliating the disease state, and remission or improved prognosis. For example, a patient is successfully “treated” if one or more symptoms associated with a synucleinopathy described herein are mitigated or eliminated, including, but are not limited to, decreasing symptoms resulting from the disease, increasing the quality of life of those suffering from the disease, decreasing the dose of other medications required to treat the disease, and/or prolonging survival of patients.

The term “delaying progression” of a disease refers to deferring, hindering, slowing, retarding, stabilizing, and/or postponing development of a cancer described herein. This delay can be of varying lengths of time, depending on the history of the synucleinopathy and/or patient being treated. As is evident to one skilled in the art, a sufficient or significant delay can, in effect, encompass prevention, in that the patient does not develop a synucleinopathy or relapse.

### 5.2. Anti-Alpha-Synuclein siRNAs

The instant application is directed to single- or double-stranded interfering RNA molecules (e.g., siRNA) targeting an SNCA gene. For example, but not by way of limitation, siRNA molecules targeting an SNCA gene can be designed to target an SNCA mRNA sequence.

In certain embodiments, the SNCA gene targeted by an siRNA of the instant disclosure expresses a mRNA comprising the sequence of NM_000345.3 (Homo sapiens synuclein, alpha (non A4 component of amyloid precursor) (SNCA), transcript variant 1, mRNA). The NM_000345.3 is: AGGAGAAGGAGAAGGAGGAGGACTAGGAGGAGGAGGACGG CGACGACCAGAAGGGGCCCAAGAGAGGGGGCGAGCGACCGAGCGCCGCGACGC GGAAGTGAGGTGCGTGCGGGCTGCAGCGCAGACCCCGGCCCGGCCCCTCCGAGA GCGTCCTGGGCGCTCCCTCACGCCTTGCCTTCAAGCCTTCTGCCTTTCCACCCTCG TGAGCGGAGAACTGGGAGTGGCCATTCGACGACAGTGTGGTGTAAAGGAATTCA TTAGCCATGGATGTATTCATGAAAGGACTTTCAAAGGCCAAGGAGGGAGTTGTG GCTGCTGCTGAGAAAACCAAACAGGGTGTGGCAGAAGCAGCAGGAAAGACAAA AGAGGGTGTTCTCTATGTAGGCTCCAAAACCAAGGAGGGAGTGGTGCATGGTGT GGCAACAGTGGCTGAGAAGACCAAAGAGCAAGTGACAAATGTTGGAGGAGCAG TGGTGACGGGTGTGACAGCAGTAGCCCAGAAGACAGTGGAGGGAGCAGGGAGC ATTGCAGCAGCCACTGGCTTTGTCAAAAAGGACCAGTTGGGCAAGAATGAAGAA GGAGCCCCACAGGAAGGAATTCTGGAAGATATGCCTGTGGATCCTGACAATGAG GCTTATGAAATGCCTTCTGAGGAAGGGTATCAAGACTACGAACCTGAAGCCTAA GAAATATCTTTGCTCCCAGTTTCTTGAGATCTGCTGACAGATGTTCCATCCTGTAC AAGTGCTCAGTTCCAATGTGCCCAGTCATGACATTTCTCAAAGTTTTTACAGTGT ATCTCGAAGTCTTCCATCAGCAGTGATTGAAGTATCTGTACCTGCCCCCACTCAG CATTTCGGTGCTTCCCTTTCACTGAAGTGAATACATGGTAGCAGGGTCTTTGTGTG CTGTGGATTTTGTGGCTTCAATCTACGATGTTAAAACAAATTAAAAACACCTAAG TGACTACCACTTATTTCTAAATCCTCACTATTTTTTTGTTGCTGTTGTTCAGAAGTT GTTAGTGATTTGCTATCATATATTATAAGATTTTTAGGTGTCTTTTAATGATACTG TCTAAGAATAATGACGTATTGTGAAATTTGTTAATATATATAATACTTAAAAATA TGTGAGCATGAAACTATGCACCTATAAATACTAAATATGAAATTTTACCATTTTG CGATGTGTTTTATTCACTTGTGTTTGTATATAAATGGTGAGAATTAAAATAAAAC GTTATCTCATTGCAAAAATATTTTATTTTTATCCCATCTCACTTTAATAATAAAAA TCATGCTTATAAGCAACATGAATTAAGAACTGACACAAAGGACAAAAATATAAA GTTATTAATAGCCATTTGAAGAAGGAGGAATTTTAGAAGAGGTAGAGAAAATGG AACATTAACCCTACACTCGGAATTCCCTGAAGCAACACTGCCAGAAGTGTGTTTT GGTATGCACTGGTTCCTTAAGTGGCTGTGATTAATTATTGAAAGTGGGGTGTTGA AGACCCCAACTACTATTGTAGAGTGGTCTATTTCTCCCTTCAATCCTGTCAATGTT TGCTTTACGTATTTTGGGGAACTGTTGTTTGATGTGTATGTGTTTATAATTGTTAT ACATTTTTAATTGAGCCTTTTATTAACATATATTGTTATTTTTGTCTCGAAATAATT TTTTAGTTAAAATCTATTTTGTCTGATATTGGTGTGAATGCTGTACCTTTCTGACA ATAAATAATATTCGACCATGAATAAAAAAAAAAAAAAAGTGGGTTCCCGGGAAC TAAGCAGTGTAGAAGATGATTTTGACTACACCCTCCTTAGAGAGCCATAAGACA CATTAGCACATATTAGCACATTCAAGGCTCTGAGAGAATGTGGTTAACTTTGTTT AACTCAGCATTCCTCACTTTTTTTTTTTAATCATCAGAAATTCTCTCTCTCTCTCTC TCTTTTTCTCTCGCTCTCTTTTTTTTTTTTTTTTTACAGGAAATGCCTTTAAACATC GTTGGAACTACCAGAGTCACCTTAAAGGAGATCAATTCTCTAGACTGATAAAAA TTTCATGGCCTCCTTTAAATGTTGCCAAATATATGAATTCTAGGATTTTTCCTTAG GAAAGGTTTTTCTCTTTCAGGGAAGATCTATTAACTCCCCATGGGTGCTGAAAAT AAACTTGATGGTGAAAAACTCTGTATAAATTAATTTAAAAATTATTTGGTTTCTCT TTTTAATTATTCTGGGGCATAGTCATTTCTAAAAGTCACTAGTAGAAAGTATAAT TTCAAGACAGAATATTCTAGACATGCTAGCAGTTTATATGTATTCATGAGTAATG TGATATATATTGGGCGCTGGTGAGGAAGGAAGGAGGAATGAGTGACTATAAGGA TGGTTACCATAGAAACTTCCTTTTTTACCTAATTGAAGAGAGACTACTACAGAGT GCTAAGCTGCATGTGTCATCTTACACTAGAGAGAAATGGTAAGTTTCTTGTTTTA TTTAAGTTATGTTTAAGCAAGGAAAGGATTTGTTATTGAACAGTATATTTCAGGA AGGTTAGAAAGTGGCGGTTAGGATATATTTTAAATCTACCTAAAGCAGCATATTT TAAAAATTTAAAAGTATTGGTATTAAATTAAGAAATAGAGGACAGAACTAGACT GATAGCAGTGACCTAGAACAATTTGAGATTAGGAAAGTTGTGACCATGAATTTA AGGATTTATGTGGATACAAATTCTCCTTTAAAGTGTTTCTTCCCTTAATATTTATC TGACGGTAATTTTTGAGCAGTGAATTACTTTATATATCTTAATAGTTTATTTGGGA CCAAACACTTAAACAAAAAGTTCTTTAAGTCATATAAGCCTTTTCAGGAAGCTTG TCTCATATTCACTCCCGAGACATTCACCTGCCAAGTGGCCTGAGGATCAATCCAG TCCTAGGTTTATTTTGCAGACTTACATTCTCCCAAGTTATTCAGCCTCATATGACT CCACGGTCGGCTTTACCAAAACAGTTCAGAGTGCACTTTGGCACACAATTGGGA ACAGAACAATCTAATGTGTGGTTTGGTATTCCAAGTGGGGTCTTTTTCAGAATCT CTGCACTAGTGTGAGATGCAAACATGTTTCCTCATCTTTCTGGCTTATCCAGTATG TAGCTATTTGTGACATAATAAATATATACATATATGAAAATA (SEQ ID NO: 858).

In certain embodiments, the SNCA gene targeted by an siRNA of the present disclosure expresses a mRNA comprising the sequence of NM_001146054.1 (Homo sapiens synuclein, alpha (non A4 component of amyloid precursor) (SNCA), transcript variant 2, mRNA). The NM_001146054.1 sequence is: GCCATTCGACGACAGGTTAGCGGGTTTGCCTCCCACT CCCCCAGCCTCGCGTCGCCGGCTCACAGCGGCCTCCTCTGGGGACAGTCCCCCCC GGGTGCCGCCTCCGCCCTTCCTGTGCGCTCCTTTTCCTTCTTCTTTCCTATTAAATA TTATTTGGGAATTGTTTAAATTTTTTTTTTAAAAAAAGAGAGAGGCGGGGAGGAG TCGGAGTTGTGGAGAAGCAGAGGGACTCAGTGTGGTGTAAAGGAATTCATTAGC CATGGATGTATTCATGAAAGGACTTTCAAAGGCCAAGGAGGGAGTTGTGGCTGC TGCTGAGAAAACCAAACAGGGTGTGGCAGAAGCAGCAGGAAAGACAAAAGAGG GTGTTCTCTATGTAGGCTCCAAAACCAAGGAGGGAGTGGTGCATGGTGTGGCAA CAGTGGCTGAGAAGACCAAAGAGCAAGTGACAAATGTTGGAGGAGCAGTGGTG ACGGGTGTGACAGCAGTAGCCCAGAAGACAGTGGAGGGAGCAGGGAGCATTGC AGCAGCCACTGGCTTTGTCAAAAAGGACCAGTTGGGCAAGAATGAAGAAGGAGC CCCACAGGAAGGAATTCTGGAAGATATGCCTGTGGATCCTGACAATGAGGCTTA TGAAATGCCTTCTGAGGAAGGGTATCAAGACTACGAACCTGAAGCCTAAGAAAT ATCTTTGCTCCCAGTTTCTTGAGATCTGCTGACAGATGTTCCATCCTGTACAAGTG CTCAGTTCCAATGTGCCCAGTCATGACATTTCTCAAAGTTTTTACAGTGTATCTCG AAGTCTTCCATCAGCAGTGATTGAAGTATCTGTACCTGCCCCCACTCAGCATTTC GGTGCTTCCCTTTCACTGAAGTGAATACATGGTAGCAGGGTCTTTGTGTGCTGTG GATTTTGTGGCTTCAATCTACGATGTTAAAACAAATTAAAAACACCTAAGTGACT ACCACTTATTTCTAAATCCTCACTATTTTTTTGTTGCTGTTGTTCAGAAGTTGTTAG TGATTTGCTATCATATATTATAAGATTTTTAGGTGTCTTTTAATGATACTGTCTAA GAATAATGACGTATTGTGAAATTTGTTAATATATATAATACTTAAAAATATGTGA GCATGAAACTATGCACCTATAAATACTAAATATGAAATTTTACCATTTTGCGATG TGTTTTATTCACTTGTGTTTGTATATAAATGGTGAGAATTAAAATAAAACGTTATC TCATTGCAAAAATATTTTATTTTTATCCCATCTCACTTTAATAATAAAAATCATGC TTATAAGCAACATGAATTAAGAACTGACACAAAGGACAAAAATATAAAGTTATT AATAGCCATTTGAAGAAGGAGGAATTTTAGAAGAGGTAGAGAAAATGGAACATT AACCCTACACTCGGAATTCCCTGAAGCAACACTGCCAGAAGTGTGTTTTGGTATG CACTGGTTCCTTAAGTGGCTGTGATTAATTATTGAAAGTGGGGTGTTGAAGACCC CAACTACTATTGTAGAGTGGTCTATTTCTCCCTTCAATCCTGTCAATGTTTGCTTT ACGTATTTTGGGGAACTGTTGTTTGATGTGTATGTGTTTATAATTGTTATACATTT TTAATTGAGCCTTTTATTAACATATATTGTTATTTTTGTCTCGAAATAATTTTTTAG TTAAAATCTATTTTGTCTGATATTGGTGTGAATGCTGTACCTTTCTGACAATAAAT AATATTCGACCATGAATAAAAAAAAAAAAAAAGTGGGTTCCCGGGAACTAAGCA GTGTAGAAGATGATTTTGACTACACCCTCCTTAGAGAGCCATAAGACACATTAGC ACATATTAGCACATTCAAGGCTCTGAGAGAATGTGGTTAACTTTGTTTAACTCAG CATTCCTCACTTTTTTTTTTTAATCATCAGAAATTCTCTCTCTCTCTCTCTCTTTTTC TCTCGCTCTCTTTTTTTTTTTTTTTTTACAGGAAATGCCTTTAAACATCGTTGGAAC TACCAGAGTCACCTTAAAGGAGATCAATTCTCTAGACTGATAAAAATTTCATGGC CTCCTTTAAATGTTGCCAAATATATGAATTCTAGGATTTTTCCTTAGGAAAGGTTT TTCTCTTTCAGGGAAGATCTATTAACTCCCCATGGGTGCTGAAAATAAACTTGAT GGTGAAAAACTCTGTATAAATTAATTTAAAAATTATTTGGTTTCTCTTTTTAATTA TTCTGGGGCATAGTCATTTCTAAAAGTCACTAGTAGAAAGTATAATTTCAAGACA GAATATTCTAGACATGCTAGCAGTTTATATGTATTCATGAGTAATGTGATATATA TTGGGCGCTGGTGAGGAAGGAAGGAGGAATGAGTGACTATAAGGATGGTTACCA TAGAAACTTCCTTTTTTACCTAATTGAAGAGAGACTACTACAGAGTGCTAAGCTG CATGTGTCATCTTACACTAGAGAGAAATGGTAAGTTTCTTGTTTTATTTAAGTTAT GTTTAAGCAAGGAAAGGATTTGTTATTGAACAGTATATTTCAGGAAGGTTAGAA AGTGGCGGTTAGGATATATTTTAAATCTACCTAAAGCAGCATATTTTAAAAATTT AAAAGTATTGGTATTAAATTAAGAAATAGAGGACAGAACTAGACTGATAGCAGT GACCTAGAACAATTTGAGATTAGGAAAGTTGTGACCATGAATTTAAGGATTTATG TGGATACAAATTCTCCTTTAAAGTGTTTCTTCCCTTAATATTTATCTGACGGTAAT TTTTGAGCAGTGAATTACTTTATATATCTTAATAGTTTATTTGGGACCAAACACTT AAACAAAAAGTTCTTTAAGTCATATAAGCCTTTTCAGGAAGCTTGTCTCATATTC ACTCCCGAGACATTCACCTGCCAAGTGGCCTGAGGATCAATCCAGTCCTAGGTTT ATTTTGCAGACTTACATTCTCCCAAGTTATTCAGCCTCATATGACTCCACGGTCGG CTTTACCAAAACAGTTCAGAGTGCACTTTGGCACACAATTGGGAACAGAACAAT CTAATGTGTGGTTTGGTATTCCAAGTGGGGTCTTTTTCAGAATCTCTGCACTAGTG TGAGATGCAAACATGTTTCCTCATCTTTCTGGCTTATCCAGTATGTAGCTATTTGT GACATAATAAATATATACATATATGAAAATA (SEQ ID NO: 859).

In certain embodiments, the SNCA gene targeted by an siRNA of the present disclosure expresses a mRNA comprising the sequence of NM_001146055.1 (Homo sapiens synuclein, alpha (non A4 component of amyloid precursor) (SNCA), transcript variant 3, mRNA). The NM_001146055.1sequence is:

(SEQ ID NO: 860)
ATTCTGGTGTGATCCAGGAACAGCTGTCTTCCAGCTCTG
AAAGAGTGTGGTGTAAAGGAATTCATTAGCCATGGATGTATTCATGAAAGGACT
TTCAAAGGCCAAGGAGGGAGTTGTGGCTGCTGCTGAGAAAACCAAACAGGGTGT
GGCAGAAGCAGCAGGAAAGACAAAAGAGGGTGTTCTCTATGTAGGCTCCAAAAC
CAAGGAGGGAGTGGTGCATGGTGTGGCAACAGTGGCTGAGAAGACCAAAGAGC
AAGTGACAAATGTTGGAGGAGCAGTGGTGACGGGTGTGACAGCAGTAGCCCAGA
AGACAGTGGAGGGAGCAGGGAGCATTGCAGCAGCCACTGGCTTTGTCAAAAAGG
ACCAGTTGGGCAAGAATGAAGAAGGAGCCCCACAGGAAGGAATTCTGGAAGAT
ATGCCTGTGGATCCTGACAATGAGGCTTATGAAATGCCTTCTGAGGAAGGGTATC
AAGACTACGAACCTGAAGCCTAAGAAATATCTTTGCTCCCAGTTTCTTGAGATCT
GCTGACAGATGTTCCATCCTGTACAAGTGCTCAGTTCCAATGTGCCCAGTCATGA
CATTTCTCAAAGTTTTTACAGTGTATCTCGAAGTCTTCCATCAGCAGTGATTGAAG
TATCTGTACCTGCCCCCACTCAGCATTTCGGTGCTTCCCTTTCACTGAAGTGAATA
CATGGTAGCAGGGTCTTTGTGTGCTGTGGATTTTGTGGCTTCAATCTACGATGTTA
AAACAAATTAAAAACACCTAAGTGACTACCACTTATTTCTAAATCCTCACTATTT
TTTTGTTGCTGTTGTTCAGAAGTIGTTAGTGATTTGCTATCATATATTATAAGATT
TTTAGGTGTCTTTTAATGATACTGTCTAAGAATAATGACGTATTGTGAAATTTGTT
AATATATATAATACTTAAAAATATGTGAGCATGAAACTATGCACCTATAAATACT
AAATATGAAATTTTACCATTTTGCGATGTGTTTTATTCACTTGTGTTTGTATATAA
ATGGTGAGAATTAAAATAAAACGTTATCTCATTGCAAAAATATTTTATTTTTATC
CCATCTCACTTTAATAATAAAAATCATGCTTATAAGCAACATGAATTAAGAACTG
ACACAAAGGACAAAAATATAAAGTTATTAATAGCCATTTGAAGAAGGAGGAATT
TTAGAAGAGGTAGAGAAAATGGAACATTAACCCTACACTCGGAATTCCCTGAAG
CAACACTGCCAGAAGTGTGTTTTGGTATGCACTGGTTCCTTAAGTGGCTGTGATT
AATTATTGAAAGTGGGGTGTTGAAGACCCCAACTACTATTGTAGAGTGGTCTATT
TCTCCCTTCAATCCTGTCAATGTTTGCTTTACGTATTTTGGGGAACTGTTGTTTGA
TGTGTATGTGTTTATAATTGTTATACATTTTTAATTGAGCCTTTTATTAACATATAT
TGTTATTTTTGTCTCGAAATAATTTTTTAGTTAAAATCTATTTTGTCTGATATTGGT
GTGAATGCTGTACCTTTCTGACAATAAATAATATTCGACCATGAATAAAAAAAAA
AAAAAAGTGGGTTCCCGGGAACTAAGCAGTGTAGAAGATGATTTTGACTACACC
CTCCTTAGAGAGCCATAAGACACATTAGCACATATTAGCACATTCAAGGCTCTGA
GAGAATGTGGTTAACTTTGTTTAACTCAGCATTCCTCACTTTTTTTTTTTAATCATC
AGAAATTCTCTCTCTCTCTCTCTCTTTTTCTCTCGCTCTCTTTTTTTTTTTTTTTTTA
CAGGAAATGCCTTTAAACATCGTTGGAACTACCAGAGTCACCTTAAAGGAGATC
AATTCTCTAGACTGATAAAAATTTCATGGCCTCCTTTAAATGTTGCCAAATATAT
GAATTCTAGGATTTTTCCTTAGGAAAGGTTTTTCTCTTTCAGGGAAGATCTATTAA
CTCCCCATGGGTGCTGAAAATAAACTTGATGGTGAAAAACTCTGTATAAATTAAT
TTAAAAATTATTTGGTTTCTCTTTTTAATTATTCTGGGGCATAGTCATTTCTAAAA
GTCACTAGTAGAAAGTATAATTTCAAGACAGAATATTCTAGACATGCTAGCAGTT
TATATGTATTCATGAGTAATGTGATATATATTGGGCGCTGGTGAGGAAGGAAGG
AGGAATGAGTGACTATAAGGATGGTTACCATAGAAACTTCCTTTTTTACCTAATT
GAAGAGAGACTACTACAGAGTGCTAAGCTGCATGTGTCATCTTACACTAGAGAG
AAATGGTAAGTTTCTTGTTTTATTTAAGTTATGTTTAAGCAAGGAAAGGATTTGTT
ATTGAACAGTATATTTCAGGAAGGTTAGAAAGTGGCGGTTAGGATATATTTTAAA
TCTACCTAAAGCAGCATATTTTAAAAATTTAAAAGTATTGGTATTAAATTAAGAA
ATAGAGGACAGAACTAGACTGATAGCAGTGACCTAGAACAATTTGAGATTAGGA
AAGTTGTGACCATGAATTTAAGGATTTATGTGGATACAAATTCTCCTTTAAAGTG
TTTCTTCCCTTAATATTTATCTGACGGTAATTTTTGAGCAGTGAATTACTTTATAT
ATCTTAATAGTTTATTTGGGACCAAACACTTAAACAAAAAGTTCTTTAAGTCATA
TAAGCCTTTTCAGGAAGCTTGTCTCATATTCACTCCCGAGACATTCACCTGCCAA
GTGGCCTGAGGATCAATCCAGTCCTAGGTTTATTTTGCAGACTTACATTCTCCCA
AGTTATTCAGCCTCATATGACTCCACGGTCGGCTTTACCAAAACAGTTCAGAGTG
CACTTTGGCACACAATTGGGAACAGAACAATCTAATGTGTGGTTTGGTATTCCAA
GTGGGGTCTTTTTCAGAATCTCTGCACTAGTGTGAGATGCAAACATGTTTCCTCAT
CTTTCTGGCTTATCCAGTATGTAGCTATTTGTGACATAATAAATATATACATATAT
GAAAATA

In certain embodiments, the SNCA gene targeted by an siRNA of the present disclosure expresses a mRNA comprising the sequence of NM_007308.2 (Homo sapiens synuclein, alpha (non A4 component of amyloid precursor) (SNCA), transcript variant 4, mRNA). NM_007308.2 sequence is:

(SEQ ID NO: 861)
GCCATTCGACGACAGGTTAGCGGGTTTGCCTCCCACTCCCCCAGCCTC
GCGTCGCCGGCTCACAGCGGCCTCCTCTGGGGACAGTCCCCCCCGGGTGCCGCCT
CCGCCCTTCCTGTGCGCTCCTTTTCCTTCTTCTTTCCTATTAAATATTATTTGGGAA
TTGTTTAAATTTTTTTTTTAAAAAAAGAGAGAGGCGGGGAGGAGTCGGAGTTGTG
GAGAAGCAGAGGGACTCAGTGTGGTGTAAAGGAATTCATTAGCCATGGATGTAT
TCATGAAAGGACTTTCAAAGGCCAAGGAGGGAGTTGTGGCTGCTGCTGAGAAAA
CCAAACAGGGTGTGGCAGAAGCAGCAGGAAAGACAAAAGAGGGTGTTCTCTAT
GTAGGCTCCAAAACCAAGGAGGGAGTGGTGCATGGTGTGGCAACAGTGGCTGAG
AAGACCAAAGAGCAAGTGACAAATGTTGGAGGAGCAGTGGTGACGGGTGTGAC
AGCAGTAGCCCAGAAGACAGTGGAGGGAGCAGGGAGCATTGCAGCAGCCACTG
GCTTTGTCAAAAAGGACCAGTTGGGCAAGGAAGGGTATCAAGACTACGAACCTG
AAGCCTAAGAAATATCTTTGCTCCCAGTTTCTTGAGATCTGCTGACAGATGTTCC
ATCCTGTACAAGTGCTCAGTTCCAATGTGCCCAGTCATGACATTTCTCAAAGTTTT
TACAGTGTATCTCGAAGTCTTCCATCAGCAGTGATTGAAGTATCTGTACCTGCCC
CCACTCAGCATTTCGGTGCTTCCCTTTCACTGAAGTGAATACATGGTAGCAGGGT
CTTTGTGTGCTGTGGATTTTGTGGCTTCAATCTACGATGTTAAAACAAATTAAAA
ACACCTAAGTGACTACCACTTATTTCTAAATCCTCACTATTTTTTTGTTGCTGTTG
TTCAGAAGTTGTTAGTGATTTGCTATCATATATTATAAGATTTTTAGGTGTCTTTT
AATGATACTGTCTAAGAATAATGACGTATTGTGAAATTTGTTAATATATATAATA
CTTAAAAATATGTGAGCATGAAACTATGCACCTATAAATACTAAATATGAAATTT
TACCATTTTGCGATGTGTTTTATTCACTTGTGTTTGTATATAAATGGTGAGAATTA
AAATAAAACGTTATCTCATTGCAAAAATATTTTATTTTTATCCCATCTCACTTTAA
TAATAAAAATCATGCTTATAAGCAACATGAATTAAGAACTGACACAAAGGACAA
AAATATAAAGTTATTAATAGCCATTTGAAGAAGGAGGAATTTTAGAAGAGGTAG
AGAAAATGGAACATTAACCCTACACTCGGAATTCCCTGAAGCAACACTGCCAGA
AGTGTGTTTTGGTATGCACTGGTTCCTTAAGTGGCTGTGATTAATTATTGAAAGTG
GGGTGTTGAAGACCCCAACTACTATTGTAGAGTGGTCTATTTCTCCCTTCAATCCT
GTCAATGTTTGCTTTACGTATTTTGGGGAACTGTTGTTTGATGTGTATGTGTTTAT
AATTGTTATACATTTTTAATTGAGCCTTTTATTAACATATATTGTTATTTTTGTCTC
GAAATAATTTTTTAGTTAAAATCTATTTTGTCTGATATTGGTGTGAATGCTGTACC
TTTCTGACAATAAATAATATTCGACCATGAATAAAAAAAAAAAAAAAGTGGGTT
CCCGGGAACTAAGCAGTGTAGAAGATGATTTTGACTACACCCTCCTTAGAGAGC
CATAAGACACATTAGCACATATTAGCACATTCAAGGCTCTGAGAGAATGTGGTT
AACTTTGTTTAACTCAGCATTCCTCACTTTTTTTTTTTAATCATCAGAAATTCTCTC
TCTCTCTCTCTCTTTTTCTCTCGCTCTCTTTTTTTTTTTTTTTTTACAGGAAATGCCT
TTAAACATCGTTGGAACTACCAGAGTCACCTTAAAGGAGATCAATTCTCTAGACT
GATAAAAATTTCATGGCCTCCTTTAAATGTTGCCAAATATATGAATTCTAGGATT
TTTCCTTAGGAAAGGTTTTTCTCTTTCAGGGAAGATCTATTAACTCCCCATGGGTG
CTGAAAATAAACTTGATGGTGAAAAACTCTGTATAAATTAATTTAAAAATTATTT
GGTTTCTCTTTTTAATTATTCTGGGGCATAGTCATTTCTAAAAGTCACTAGTAGAA
AGTATAATTTCAAGACAGAATATTCTAGACATGCTAGCAGTTTATATGTATTCAT
GAGTAATGTGATATATATTGGGCGCTGGTGAGGAAGGAAGGAGGAATGAGTGAC
TATAAGGATGGTTACCATAGAAACTTCCTTTTTTACCTAATTGAAGAGAGACTAC
TACAGAGTGCTAAGCTGCATGTGTCATCTTACACTAGAGAGAAATGGTAAGTTTC
TTGTTTTATTTAAGTTATGTTTAAGCAAGGAAAGGATTTGTTATTGAACAGTATAT
TTCAGGAAGGTTAGAAAGTGGCGGTTAGGATATATTTTAAATCTACCTAAAGCAG
CATATTTTAAAAATTTAAAAGTATTGGTATTAAATTAAGAAATAGAGGACAGAA
CTAGACTGATAGCAGTGACCTAGAACAATTTGAGATTAGGAAAGTTGTGACCAT
GAATTTAAGGATTTATGTGGATACAAATTCTCCTTTAAAGTGTTTCTTCCCTTAAT
ATTTATCTGACGGTAATTTTTGAGCAGTGAATTACTTTATATATCTTAATAGTTTA
TTTGGGACCAAACACTTAAACAAAAAGTTCTTTAAGTCATATAAGCCTTTTCAGG
AAGCTTGTCTCATATTCACTCCCGAGACATTCACCTGCCAAGTGGCCTGAGGATC
AATCCAGTCCTAGGTTTATTTTGCAGACTTACATTCTCCCAAGTTATTCAGCCTCA
TATGACTCCACGGTCGGCTTTACCAAAACAGTTCAGAGTGCACTTTGGCACACAA
TTGGGAACAGAACAATCTAATGTGTGGTTTGGTATTCCAAGTGGGGTCTTTTTCA
GAATCTCTGCACTAGTGTGAGATGCAAACATGTTTCCTCATCTTTCTGGCTTATCC
AGTATGTAGCTATTTGTGACATAATAAATATATACATATATGAAAATA.

In certain embodiments, the present disclosure is directed to an siRNA molecule comprising an antisense strand and sense strand having complementarity to the antisense strand, wherein the antisense strand is from 10 to 30 nucleotides in length and has complementarity sufficient to hybridize to a region within an SNCA mRNA transcript having the nucleic acid sequence of any one of SEQ ID NOs: 421-840. In certain embodiments, the antisense strand has at least 70% complementarity to a region of 21 contiguous nucleobases within the SNCA mRNA transcript having the nucleic acid sequence of any one of SEQ ID NOs: 421-840. In certain embodiments, the antisense strand has at least 75% complementarity to a region of 21 contiguous nucleobases within the SNCA mRNA transcript having the nucleic acid sequence of any one of SEQ ID NOs: 421-840. For example, but not by way of limitation, the antisense strand can have at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the SNCA mRNA transcript having the nucleic acid sequence of any one of SEQ ID Nos: 421-840.

In certain embodiments, the antisense strand comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the SNCA RNA transcript having the nucleic acid sequence of any one of SEQ ID NOs: 421-840. In certain embodiments, the antisense strand comprises from 10 to 30 contiguous nucleotides that are fully complementary to a contiguous polynucleotide segment of equal length within the region of the SNCA RNA transcript having the nucleic acid sequence of any one of SEQ ID NOs: 421-840. In certain embodiments, the antisense strand comprises from 12 to 30 contiguous nucleotides that are fully complementary to a contiguous polynucleotide segment of equal length within the region of the SNCA RNA transcript having the nucleic acid sequence of any one of SEQ ID NOs: 421-840. In certain embodiments, the antisense strand comprises from 15 to 30 contiguous nucleotides that are fully complementary to a contiguous polynucleotide segment of equal length within the region of the SNCA RNA transcript having the nucleic acid sequence of any one of SEQ ID NOs: 421-840. In certain embodiments, the antisense strand comprises from 18 to 30 contiguous nucleotides that are fully complementary to a contiguous polynucleotide segment of equal length within the region of the SNCA RNA transcript having the nucleic acid sequence of any one of SEQ ID NOs: 421-840. In certain embodiments, the antisense strand comprises from 21 to 30 contiguous nucleotides that are fully complementary to a contiguous polynucleotide segment of equal length within the region of the SNCA RNA transcript having the nucleic acid sequence of any one of SEQ ID NOs: 421-840. In certain embodiments, the antisense strand comprises from 24 to 30 contiguous nucleotides that are fully complementary to a contiguous polynucleotide segment of equal length within the region of the SNCA RNA transcript having the nucleic acid sequence of any one of SEQ ID NOs: 421-840. In certain embodiments, the antisense strand comprises 30 contiguous nucleotides that are fully complementary to a contiguous polynucleotide segment of equal length within the region of the SNCA RNA transcript having the nucleic acid sequence of any one of SEQ ID NOs: 421-840.

In certain embodiments, the antisense strand comprises 9 or fewer nucleotide mismatches relative to a region of 21 contiguous nucleobases of the SNCA RNA transcript having the nucleic acid sequence of any one of SEQ ID NOs: 421-840. For example, but not by way of limitation, the antisense strand can comprise 8 or fewer, 7 or fewer, 6 or fewer, 5 or fewer, 4 or fewer, 3 or fewer, 2 or fewer, or only 1 mismatch relative to the region of the SNCA RNA transcript having the nucleic acid sequence of any one of SEQ ID NOs: 421-840.

In certain embodiments, the siRNA molecule of the present disclosure targets a region of an SNCA RNA transcript that has the nucleic acid sequence of any one of SEQ ID NOs: 459, 535, 551, 599, 631, 663, 664, 666, 667, 671, 748, 750, 751, 752, 760, 762, and 777.

In certain embodiments, the present disclosure is directed to an siRNA molecule comprising a sense strand comprising an unmodified sense strand of Table 1A. In certain embodiments, the present disclosure is directed to an siRNA molecule comprising an antisense strand comprising an unmodified antisense strand of Table 1A. In certain embodiments the sense strand is UACCACUUAUUUCUAA (SEQ ID NO: 760) and the antisense strand is UUAGAAAUAAGUGGUAGUCAC (SEQ ID NO: 340). In certain embodiments the sense strand is CAAGUGCUCAGUUCCA (SEQ ID NO: 664) and the antisense strand is UGGAACUGAGCACUUGUACAG (SEQ ID NO: 244). In certain embodiments the sense strand is AGUGGUGCAUGGUGUA (SEQ ID NO: 535) and the antisense strand is UACACCAUGCACCACUCCCUC (SEQ ID NO: 115). In certain embodiments the sense strand is CAAUGAGGCUUAUGAA (SEQ ID NO: 599) and the antisense strand is UUCAUAAGCCUCAUUGUCAGG (SEQ ID NO: 179). In certain embodiments the sense strand is AGUGCUCAGUUCCAAA (SEQ ID NO: 666) and the antisense strand is UUUGGAACUGAGCACUUGUAC (SEQ ID NO: 246). In certain embodiments the sense strand is UCAGUUCCAAUGUGCA (SEQ ID NO: 671) and the antisense strand is UGCACAUUGGAACUGAGCACU (SEQ ID NO: 251). In certain embodiments the sense strand is CUACGAUGUUAAAACA (SEQ ID NO: 748) and the antisense strand is UGUUUUAACAUCGUAGAUUGA (SEQ ID NO: 328).

In certain embodiments, the present disclosure is directed to an siRNA molecule comprising a sense strand comprising a modified sense strand of Table 1B. In certain embodiments, the present disclosure is directed to an siRNA molecule comprising an antisense strand comprising a modified antisense strand of Table 1B. In certain embodiments the sense strand is (mU) #(mA) #(mC)(fC)(mA)(fC)(mU)(fU)(mA)(fU)(mU)(mU)(mC)(fU) #(mA) #(mA)-DIO (SEQ ID NO: 841) and the antisense strand is V(mU) #(fU) #(mA)(fG)(fA)(fA)(mA)(fU)(mA)(fA) (mG)(fU)(mG)(fG)(mU)(fA) #(mG) #(mU) #(mC) #(mA) #(mC) (SEQ ID NO: 842) (where m=2′Ome; f=2′F; #=phosphorothioate; -DIO=a divalent oligonucleotide (DIO) linker; V=vinyl phosphonate. In certain embodiments the sense strand is (mC) #(mA) #(mA)(fG)(mU)(fG)(mC)(fU) (mC)(fA)(mG)(mU)(mU)(fC) #(mC) #(mA)-DIO (SEQ ID NO: 843) and the antisense strand is V(mU) #(fG) #(mG)(fA)(fA)(fC)(mU)(fG)(mA)(fG)(mC)(fA)(mC)(fU)(mU)(fG) #(mU) #(mA) #(mC) #(mA) #(mG) (SEQ ID NO: 844). In certain embodiments the sense strand is (mA) #(mG) #(mU) (fG)(mG)(fU)(mG)(fC)(mA)(fU)(mG)(mG)(mU)(fG) #(mU) #(mA)-DIO (SEQ ID NO: 845) and the antisense strand is V(mU) #(fA) #(mC)(fA)(fC)(fC)(mA)(fU)(mG)(fC)(mA)(fC)(mC)(fA)(mC)(fU) #(mC) #(mC) #(mC) #(mU) #(mC) (SEQ ID NO: 846). In certain embodiments the sense strand is (mC) #(mA) #(mA)(fU)(mG)(fA)(mG)(fG)(mC)(fU)(mU)(mA)(mU)(fG) #(mA) #(mA)-DIO (SEQ ID NO: 847) and the antisense strand is V(mU) #(fU) #(mU)(fG)(fG)(fA)(mA)(fC)(mU)(fG)(mA)(fG)(mC)(fA) (mC)(fU) #(mU) #(mG) #(mU) #(mA) #(mC) (SEQ ID NO: 848). In certain embodiments the sense strand is (mU) #(mC) #(mA)(fG)(mU)(fU)(mC)(fC)(mA)(fA)(mU)(mG)(mU)(fG) #(mC) #(mA)-DIO (SEQ ID NO: 849) and the antisense strand is V(mU) #(fU) #(mU)(fG)(fG)(fA)(mA)(fC)(mU)(fG)(mA)(fG)(mC)(fA) (mC)(fU) #(mU) #(mG) #(mU) #(mA) #(mC) (SEQ ID NO: 850). In certain embodiments the sense strand is (mC) #(mU) #(mA)(fC)(mG)(fA)(mU)(fG)(mU)(fU)(mA)(mA)(mA)(fA) #(mC) #(mA)-DIO (SEQ ID NO: 851) and the antisense strand is V(mU) #(fG) #(mC)(fA)(fC)(fA)(mU)(fU)(mG)(fG)(mA)(fA)(mC)(fU) (mG)(fA) #(mG) #(mC) #(mA) #(mC) #(mU) (SEQ ID NO: 852). In certain embodiments the sense strand is (mC) #(mU) #(mA)(fC)(mG)(fA)(mU)(fG)(mU)(fU)(mA)(mA)(mA)(fA) #(mC) #(mA)-DIO (SEQ ID NO: 853) and the antisense strand is V(mU) #(fG) #(mU)(fU)(fU)(fU)(mA)(fA)(mC)(fA)(mU)(fC)(mG)(fJ) (mA)(fG) #(mA) #(mU) #(mU) #(mG) #(mA) (SEQ ID NO: 854). In certain embodiments, the sense strand is (mC) #(mA) #(mA)(fU)(mG)(fA)(mG)(fG)(mC) (fU)(mU)(mA)(mU)(fG) #(mA) #(mA)-DIO (SEQ ID NO: 847) and the antisense strand is V(mU) #(fU) #(mC)(fA)(fU)(fA)(mA)(fG)(mC)(fC)(mU)(fC)(mA)(fU)(mU)(fG) #(mU) #(mC) #(mA) #(mG) #(mG) (SEQ ID NO: 855). In certain embodiments, the sense strand is (mA) #(mG) #(mU)(fG)(mC)(fU) (mC)(fA)(mG)(fU)(mU)(mC)(mC)(fA) #(mA) #(mA)-DIO (SEQ ID NO: 856) and the antisense strand is V(mU) #(fU) #(mU)(fG)(fG)(fA)(mA)(fC)(mU)(fG)(mA)(fG)(mC)(fA)(mC)(fU) #(mU) #(mG) #(mU) #(mA) #(mC) (SEQ ID NO: 850). In certain embodiments, the sense strand is (mU) #(mC) #(mA)(fG)(mU) (fU)(mC)(fC)(mA)(fA)(mU)(mG)(mU)(fG) #(mC) #(mA)-DIO (SEQ ID NO: 857) and the antisense strand is V(mU) #(fG) #(mC)(fA)(fC)(fA)(mU)(fU)(mG)(fG)(mA)(fA)(mC)(fU)(mG) (fA) #(mG) #(mC) #(mA) #(mC) #(mU) (SEQ ID NO: 852).

TABLE 1A

| Position | AT | | Unmodified | Unmodified |
|---|---|---|---|---|
| name | numbers | mRNA target sequence | 16 mer sense | 21 mer antisense |
| SNCA_963 | AT4512-4509 | GUGACUACCACUUAUUUCUAA | UACCACUUAUUUCUAA | UUAGAAAUAAGUGGUAGUCAC |
| | | (SEQ ID NO: 862) | (SEQ ID NO: 760) | (SEQ ID NO: 340) |
| SNCA_737 | AT4511-4514 | CUGUACAAGUGCUCAGUUCCA | CAAGUGCUCAGUUCCA | UGGAACUGAGCACUUGUACAG |
| | | (SEQ ID NO: 863) | (SEQ ID NO: 644) | (SEQ ID NO: 244) |
| SNCA_399 | AT4524-4521 | GAGGGAGUGGUGCAUGGUGUG | AGUGGUGCAUGGUGUA | UACACCAUGCACCACUCCCUC |
| | | (SEQ ID NO: 864) | (SEQ ID NO: 535) | (SEQ ID NO: 115) |
| SNCA_621 | AT4547-4546 | CCUGACAAUGAGGCUUAUGAA | CAAUGAGGCUUAUGAA | UUCAUAAGCCUCAUUGUCAGG |
| | | (SEQ ID NO: 865) | (SEQ ID NO: 599) | (SEQ ID NO: 179) |
| SNCA_739 | AT4516-4513 | GUACAAGUGCUCAGUUCCAAU | AGUGCUCAGUUCCAAA | UUUGGAACUGAGCACUUGUAC |
| | | (SEQ ID NO: 866) | (SEQ ID NO: 666) | (SEQ ID NO: 246) |
| SNCA_744 | AT4555-4554 | AGUGCUCAGUUCCAAUGUGCC | UCAGUUCCAAUGUGCA | UGCACAUUGGAACUGAGCACU |
| | | (SEQ ID NO: 867) | (SEQ ID NO 671) | (SEQ ID NO: 251) |
| SNCA_926 | AT4520-4517 | UCAAUCUACGAUGUUAAAACA | CUACGAUGUUAAAACA | UGUUUUAACAUCGUAGAUUGA |
| | | (SEQ ID NO: 868) | (SEQ ID NO: 748) | (SEQ ID NO: 328) |

TABLE 1B

| Position | AT | | |
|---|---|---|---|
| name | numbers | Modified 16 mer sense | Modified 21mer antisense |
| SNCA_963 | AT4512- | (mU)#(mA)#(mC)(fC)(mA)(fC)(mU)(fU) | V(mU)#(fU)#(mA)(fG)(fA)(fA)(mA)(fU) |
| | 4509 | (mA)(fU)(mU)(mU)(mC)(fU)#(mA)#(mA)- | (mA)(fA)(mG)(fU)(mG)(fG)(mU)(fA)#(mG) |
| | | DIO (SEQ ID NO: 841) | #(mU)#(mC)#(mA)#(mC) (SEQ ID NO: 842) |
| SNCA_737 | AT4511- | (mC)#(mA)#(mA)(fG)(mU)(fG)(mC)(fU) | V(mU)#(fG)#(mG)(fA)(fA)(fC)(mU)(fG) |
| | 4514 | (mC)(fA)(mG)(mU)(mU)(fC)#(mC)#(mA)- | (mA)(fG)(mC)(fA)(mC)(fU)(mU)(fG)#(mU) |
| | | DIO (SEQ ID NO: 843) | #(mA)#(mC)#(mA)#(mG) (SEQ ID NO: 844) |
| SNCA_399 | AT4524- | (mA)#(mG)#(mU)(fG)(mG)(fU)(mG)(fC) | V(mU)#(fA)#(mC)(fA)(fC)(fC)(mA)(fU) |
| | 4521 | (mA)(fU)(mG)(mG)(mU)(fG)#(mU)#(mA)- | (mG)(fC)(mA)(fC)(mC)(fA)(mC)(fU)#(mC) |
| | | DIO (SEQ ID NO: 845) | #(mC)#(mC)#(mU)#(mC) (SEQ ID NO: 846) |
| SNCA_621 | AT4547- | (mC)#(mA)#(mA)(fU)(mG)(fA)(mG)(fG) | V(mU)#(fU)#(mC)(fA)(fU)(fA)(mA)(fG) |
| | 4546 | (mC)(fU)(mU)(mA)(mU)(fG)#(mA)#(mA)- | (mC)(fC)(mU)(fC)(mA)(fU)(mU)(fG)#(mU) |
| | | DIO (SEQ ID NO: 847) | #(mC)#(mA)#(mG)#(mG) (SEQ ID NO: 855) |
| SNCA_739 | AT4516- | (mA)#(mG)#(mU)(fG)(mC)(fU)(mC)(fA) | V(mU)#(fU)#(mU)(fG)(fG)(fA)(mA)(fC) |
| | 4513 | (mG)(fU)(mU)(mC)(mC)(fA)#(mA)#(mA)- | (mU)(fG)(mA)(fG)(mC)(fA)(mC)(fU)#(mU) |
| | | DIO (SEQ ID NO: 856) | #(mG)#(mU)#(mA)#(mC) (SEQ ID NO: 850) |
| SNCA_744 | AT4555- | (mU)#(mC)#(mA)(fG)(mU)(fU)(mC)(fC) | V(mU)#(fG)#(mC)(fA)(fC)(fA)(mU)(fU) |
| | 4554 | (mA)(fA)(mU)(mG)(mU)(fG)#(mC)#(mA)- | (mG)(fG)(mA)(fA)(mC)(fU)(mG)(fA)#(mG) |
| | | DIO (SEQ ID NO: 857) | #(mC)#(mA)#(mC)#(mU) (SEQ ID NO: 852) |
| SNCA_926 | AT4520- | (mC)#(mU)#(mA)(fC)(mG)(fA)(mU)(fG) | V(mU)#(fG)#(mU)(fU)(fU)(fU)(mA)(fA) |
| | 4517 | (mU)(fU)(mA)(mA)(mA)(fA)#(mC)#(mA)- | (mC)(fA)(mU)(fC)(mG)(fU)(mA)(fG)#(mA) |
| | | DIO (SEQ ID NO: 853) | #(mU)#(mU)#(mG)#(mA) (SEQ ID NO: 854) |

Note:
m = 2′Ome; f = 2′F; # = phosphorothioate; -DIO = a divalent oligonucleotide (DIO) linker; V = vinyl phosphonate.

### 5.3. siRNA Structure

The siRNA molecules of the disclosure may be in the form of a single-stranded (ss) or double-stranded (ds) oligonucleotide structure. In some embodiments, the siRNA molecules may be di-branched, tri-branched, or tetra-branched molecules. Furthermore, the siRNA molecules of the disclosure may contain one or more phosphodiester internucleoside linkages and/or an analog thereof, such as a phosphorothioate internucleoside linkage. The siRNA molecules of the disclosure may further contain chemically modified nucleosides having 2′ sugar modifications.

The simplest siRNAs consist of a ribonucleic acid, including a ss- or ds-structure, formed by a first strand (i.e., antisense strand), and in the case of a ds-siRNA, a second strand (i.e., sense strand). The first strand includes a stretch of contiguous nucleotides that is at least partially complementary to a target nucleic acid. The second strand also includes a stretch of contiguous nucleotides where the second stretch is at least partially identical to a target nucleic acid. The first strand and said second strand may be hybridized to each other to form a double-stranded structure. The hybridization typically occurs by Watson Crick base pairing.

Depending on the sequence of the first and second strand, the hybridization or base pairing is not necessarily complete or perfect, which means that the first and second strand are not 100% base-paired due to mismatches. One or more mismatches may also be present within the duplex without necessarily impacting the siRNA RNAi activity.

The first strand contains a stretch of contiguous nucleotides which is essentially complementary to a target nucleic acid. Typically, the target nucleic acid sequence is, in accordance with the mode of action of interfering ribonucleic acids, a ss-RNA, e.g., an mRNA. Such hybridization occurs most likely through Watson Crick base pairing but is not necessarily limited thereto. The extent to which the first strand has a complementary stretch of contiguous nucleotides to a target nucleic acid sequence may be between 80% and 100%, e.g., 80%, 85%, 90%, 95%, or 100% complementary.

The siRNA molecules described herein may employ modifications to the nucleobase, phosphate backbone, ribose core, 5′- and 3′-ends, and branching, wherein multiple strands of siRNA may be covalently linked.

The siRNA molecules described herein may contain one or more phosphodiester internucleoside linkages and/or an analog thereof, such as a phosphorothioate internucleoside linkage, in which oxyanion moieties are electrostatically neutralized by ionic bonding to a divalent metal cation, such as Ba²⁺, Be²⁺, Ca²⁺, Cu²⁺, Mg²⁺, Mn²⁺, Ni²⁺, or Zn²⁺. In certain embodiments, the one or more divalent cations includes Ca²⁺ and Mg²⁺, optionally wherein the ratio of Ca²⁺ to Mg²⁺ is from 1:100 to 100:1 (e.g., 1:75, 1:50, 1:25, 1:10, 1:5, 1:1, 5:1, 10:1, 25:1, 50:1, 75:1, or 100:1). In certain embodiments, the Ca²⁺ and Mg²⁺ are present in a 1:1 ratio. In certain embodiments, the one or more divalent cations displace water from a cationic binding site of the siRNA molecule. In certain embodiments, the degree of saturation of the cationic binding sites by the one or more divalent cations is from about 10% to about 100% (e.g., from about 20% to about 100%, from about 30% to about 100%, from about 40% to about 100%, from about 50% to about 100%, from about 60% to about 100%, from about 70% to about 100%, from about 80% to about 100%, or from about 90% to about 100%). In certain embodiments, the cationic binding site is located within an internucleoside linkage, such as a phosphodiester linkage and/or a phosphorothioate linkage. For example, the cationic binding site may be an oxyanion moiety within a phosphodiester linage or phosphorothioate linkage. In certain embodiments, the one or more divalent cations are characterized as having an ionic radius ranging from about 30 picometers to about 150 picometers (e.g., from about 30 picometers to about 140 picometers, from about 40 picometers to about 130 picometers, from about 50 picometers to about 120 picometers, from about 60 picometers to about 110 picometers, from about 60 picometers to about 100 picometers, or from about 60 picometers to about 90 picometers).

5.3.1. Lengths of siRNA Molecules

It is within the scope of the disclosure that any length, known and previously unknown in the art, may be employed for the current invention. As described herein, potential lengths for an antisense strand of the siRNA molecules of the present disclosure is between 10 and 30 nucleotides (e.g., 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), 15 and 25 nucleotides (e.g., 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides), or 18 and 23 nucleotides (e.g., 18 nucleotides, 19 nucleotides, 20 nucleotides, 21 nucleotides, 22 nucleotides, or 23 nucleotides). In some embodiments, the antisense strand is 20 nucleotides. In some embodiments, the antisense strand is 21 nucleotides. In some embodiments, the antisense strand is 22 nucleotides. In some embodiments, the antisense strand is 23 nucleotides. In some embodiments, the antisense strand is 24 nucleotides. In some embodiments, the antisense strand is 25 nucleotides. In some embodiments, the antisense strand is 26 nucleotides. In some embodiments, the antisense strand is 27 nucleotides. In some embodiments, the antisense strand is 28 nucleotides. In some embodiments, the antisense strand is 29 nucleotides. In some embodiments, the antisense strand is 30 nucleotides.

In some embodiments, the sense strand of the siRNA molecules of the present disclosure is between 12 and 30 nucleotides (e.g., 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides), or 14 and 23 nucleotides (e.g., 14 nucleotides, 15 nucleotides, 16 nucleotides, 17 nucleotides, 18 nucleotides, 19 nucleotides, 20 nucleotides, 21 nucleotides, 22 nucleotides, or 23 nucleotides). In some embodiments, the sense strand is 15 nucleotides. In some embodiments, the sense strand is 16 nucleotides. In some embodiments, the sense strand is 17 nucleotides. In some embodiments, the sense strand is 18 nucleotides. In some embodiments, the sense strand is 19 nucleotides. In some embodiments, the sense strand is 20 nucleotides. In some embodiments, the sense strand is 21 nucleotides. In some embodiments, the sense strand is 22 nucleotides. In some embodiments, the sense strand is 23 nucleotides. In some embodiments, the sense strand is 24 nucleotides. In some embodiments, the sense strand is 25 nucleotides. In some embodiments, the sense strand is 26 nucleotides. In some embodiments, the sense strand is 27 nucleotides. In some embodiments, the sense strand is 28 nucleotides. In some embodiments, the sense strand is 29 nucleotides. In some embodiments, the sense strand is 30 nucleotides.

### 5.3.2. 2′ Sugar Modifications

The present disclosure may include ss- and ds-siRNA molecule compositions including at least one (e.g., at least 1, 2, 3, 4, 5, 6, 7, 8, 9, 10, 11, or more) nucleosides having 2′ sugar modifications. Possible 2′-modifications include all possible orientations of OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C1 to C10 alkyl or C2 to C10 alkenyl and alkynyl. In some embodiments, the modification includes a 2′-O-methyl (2′-O-Me) modification. Other potential sugar substituent groups include: C1 to C10 lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′-O—CH₂CH₂OCH₃, also known as 2′-O-(2-methoxyethyl) or 2′-MOE). In some embodiments, the modification includes 2′-dimethylaminooxyethoxy, i.e., a O(CH₂)₂ON(CH₃)₂ group, also known as 2′-DMAOE, and 2′-dimethylaminoethoxyethoxy (also known in the art as 2′-O-dimethylamino-ethoxy-ethyl or 2′-DMAEOE), i.e., 2′-O—CH₂OCH₂N(CH₃)₂. Other potential sugar substituent groups include, e.g., aminopropoxy (—OCH₂CH₂CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. In some embodiments, the 2′-arabino modification is 2′-F. Similar modifications may also be made at other positions on the siRNA molecule, particularly the 3′ position of the sugar on the 3′ terminal nucleoside or in 2′-5′ linked oligonucleotides and the 5′ position of 5′ terminal nucleotide. Oligonucleotides may also have sugar mimetics such as cyclobutyl moieties in place of the pentofuranosyl sugar.

### 5.3.3. Nucleobase Modifications

The siRNA molecules of the disclosure may also include nucleosides or other surrogate or mimetic monomeric subunits that include a nucleobase (often referred to in the art simply as “base” or “heterocyclic base moiety”). The nucleobase is another moiety that has been extensively modified or substituted and such modified and or substituted nucleobases are amenable to the present disclosure. As used herein, “unmodified” or “natural” nucleobases include the purine bases adenine (A) and guanine (G), and the pyrimidine bases thymine (T), cytosine (C) and uracil (U). Chemically modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in Kroschwitz, J. I., ed. The Concise Encyclopedia of Polymer Science and Engineering, New York, John Wiley & Sons, 1990, pp. 858-859; those disclosed by Englisch et al., Angewandte Chemie, International Edition 30:613, 1991; and those disclosed by Sanghvi, Y. S., Chapter 16, Antisense Research and Applications, CRC Press, Gait, M. J. ed., 1993, pp. 289-302. The siRNA molecules of the present disclosure may also include polycyclic heterocyclic compounds in place of one or more heterocyclic base moieties. A number of tricyclic heterocyclic compounds have been previously reported. These compounds are routinely used in antisense applications to increase the binding properties of the modified strand to a target strand.

Representative cytosine analogs that make three hydrogen bonds with a guanosine in a second strand include 1,3-diazaphenoxazine-2-one (Kurchavov et al., Nucleosides and Nucleotides, 16:1837-46, 1997), 1,3-diazaphenothiazine-2-one (Lin et al. Am. Chem. Soc., 117:3873-4, 1995), and 6,7,8,9-tetrafluoro-1,3-diazaphenoxazine-2-one (Wang et al., Tetrahedron Lett., 39:8385-8, 1998). Incorporated into oligonucleotides, these base modifications were shown to hybridize with complementary guanine and the latter was also shown to hybridize with adenine and to enhance helical thermal stability by extended stacking interactions (also see U.S. Ser. No. 10/155,920 and U.S. Ser. No. 10/013,295, both of which are herein incorporated by reference in their entirety). Further helix-stabilizing properties have been observed when a cytosine analog/substitute has an aminoethoxy moiety attached to the rigid 1,3-diazaphenoxazine-2-one scaffold (Lin et al., Am. Chem. Soc., 120:8531-2, 1998).

### 5.3.4. Internucleoside Linkage Modifications

Another variable in the design of the present disclosure is the internucleoside linkage making up the phosphate backbone of the siRNA molecule. Although the natural RNA phosphate backbone may be employed here, derivatives thereof may be used which enhance desirable characteristics of the siRNA molecule. In certain embodiments, the siRNA molecule will be modified to exhibit reduced hydrolysis relative to the unmodified siRNA. One example of a modification that decreases the rate of hydrolysis is phosphorothioates. Any portion or the whole of the backbone may contain phosphate substitutions (e.g., phosphorothioates). For instance, the internucleoside linkages may be between 0 and 100% phosphorothioate, e.g., between 0 and 100%, 10 and 100%, 20 and 100%, 30 and 100%, 40 and 100%, 50 and 100%, 60 and 100%, 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphorothioate linkages. Similarly, the internucleoside linkages may be between 0 and 100% phosphodiester linkages, e.g., between 0 and 100%, 10 and 100%, 20 and 100%, 30 and 100%, 40 and 100%, 50 and 100%, 60 and 100% 70 and 100%, 80 and 100%, 90 and 100%, 0 and 90%, 0 and 80%, 0 and 70%, 0 and 60%, 0 and 50%, 0 and 40%, 0 and 30%, 0 and 20%, 0 and 10%, 10 and 90%, 20 and 80%, 30 and 70%, 40 and 60%, 10 and 40%, 20 and 50%, 30 and 60%, 40 and 70%, 50 and 80%, or 60 and 90% phosphodiester linkages.

Specific examples of some potential siRNA molecules useful in this invention include oligonucleotides containing modified e.g., non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides. An exemplary phosphorus containing modified internucleoside linkage is the phosphorothioate internucleoside linkage. In some embodiments, the modified oligonucleotide backbones containing a phosphorus atom therein include, for example, phosphorothioates, phosphorodithioates, phosphotriesters, aminoalkylphosphotriesters, methyl and other alkyl phosphonates including 3′-alkylene phosphonates, 5′-alkylene phosphonates, phosphinates, phosphoramidates including 3′-amino phosphoramidate and aminoalkylphosphoramidates, thionophosphoramidates, thionoalkyl-phosphonates, thionoalkylphosphotriesters, selenophosphates, and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Exemplary U.S. patents describing the preparation of phosphorus-containing linkages include but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. RE39,464, the entire contents of each of which are hereby incorporated herein by reference.

In some embodiments, the modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. Non-limiting examples of U.S. patents that teach the preparation of non-phosphorus backbones include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and 5,677,439, the entire contents of each of which are hereby incorporated herein by reference.

### 5.4. Patterns of Modifications of siRNA Molecules

The following section provides a set of exemplary scaffolds into which the siRNA molecules of the disclosure may be incorporated.

In some embodiments of the disclosure, the siRNA may contain an antisense strand including a region represented by Formula I, wherein Formula I is, in the 5′-to-3′ direction

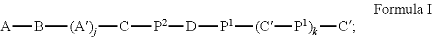

wherein A is represented by the formula C—P¹-D-P¹; each A′ is represented by the formula C—P²-D-P²; B is represented by the formula C—P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments, the antisense strand includes a structure represented by Formula A1, wherein Formula A1 is, in the 5′-to-3′ direction:

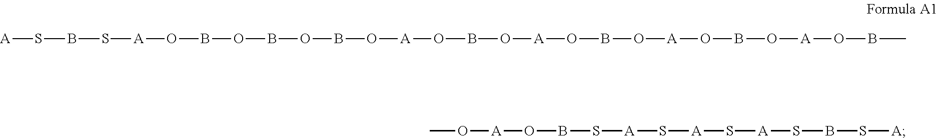

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula II, wherein Formula II is, in the 5′-to-3′ direction:

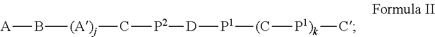

wherein A is represented by the formula C—P¹-D-P¹; each A′ is represented by the formula C—P²-D-P²; B is represented by the formula C—P²-D-P²-D-P²-D-P²; each C is a 2′-O-methyl (2′-O-Me) ribonucleoside; each C′, independently, is a 2′-O-Me ribonucleoside or a 2′-fluoro (2′-F) ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 4. In some embodiments, k is 4. In some embodiments, j is 4 and k is 4. The antisense is complementary (e.g., fully or partially complementary) to a target nucleic acid sequence.

In some embodiments of the disclosure, the antisense strand includes a structure represented by Formula A2, wherein Formula A2 is, in the 5′-to-3′ direction:

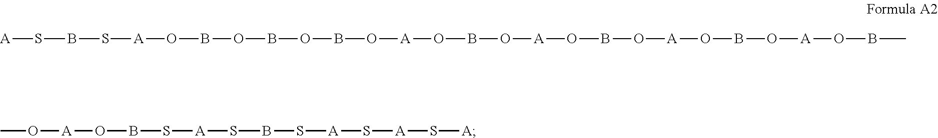

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula III, wherein Formula III is, in the 5′-to-3′ direction:

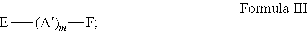

wherein E is represented by the formula (C—P¹)₂; F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D; A′, C, D, P¹, and P² are as defined in Formula I; and m is an integer from 1 to 7 (e.g., 1, 2, 3, 4, 5, 6, or 7). In some embodiments, m is 4. The sense strand is complementary (e.g., fully or partially complementary) to the antisense strand.

In some embodiments of the disclosure, the sense strand includes a structure represented by Formula S1, wherein Formula S1 is, in the 5′-to-3′ direction:

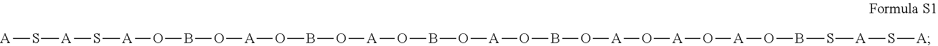

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S2, wherein Formula S2 is, in the 5′-to-3′ direction:

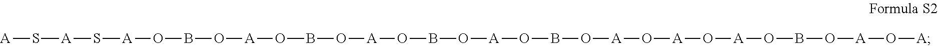

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S3, wherein Formula S3 is, in the 5′-to-3′ direction:

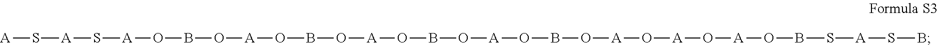

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S4, wherein Formula S4 is, in the 5′-to-3′ direction:

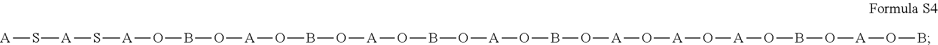

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula IV, wherein Formula IV is, in the 5′-to-3′ direction:

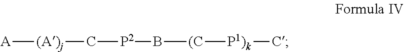

wherein A is represented by the formula C—P¹-D-P¹; each A′ is represented by the formula C—P²-D-P²; B is represented by the formula D-P¹—C—P¹-D-P¹; each C is a 2′-O-Me ribonucleoside; each C′, independently, is a 2′-O-Me ribonucleoside or a 2′-F ribonucleoside; each D is a 2′-F ribonucleoside; each P¹ is a phosphorothioate internucleoside linkage; each P² is a phosphodiester internucleoside linkage; j is an integer from 1 to 7 (e.g., 1, 2, 3, 4, 5, 6, or 7); and k is an integer from 1 to 7 (e.g., 1, 2, 3, 4, 5, 6, or 7). In some embodiments, j is 6. In some embodiments, k is 4. In some embodiments, j is 6 and k is 4. The antisense strand is complementary (e.g., fully or partially complementary) to a target nucleic acid.

In some embodiments of the disclosure, the antisense strand includes a structure represented by Formula A3, wherein Formula A3 is, in the 5′-to-3′ direction:

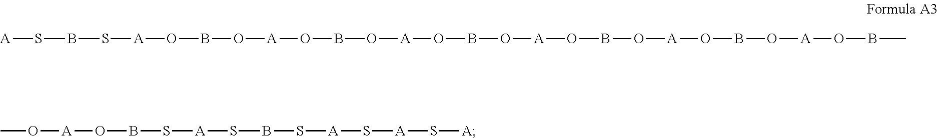

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA of the disclosure may have a sense strand represented by Formula V, wherein Formula V is, in the 5′-to-3′ direction:

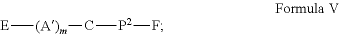

wherein E is represented by the formula (C—P¹)₂; F is represented by the formula D-P¹—C—P¹—C, D-P²—C—P²—C, D-P¹—C—P¹-D, or D-P²—C—P²-D; A′, C, D, P¹, and P² are as defined in Formula IV; and m is an integer from 1 to 7 (e.g., 1, 2, 3, 4, 5, 6, or 7). In some embodiments, m is 5. The sense strand is complementary (e.g., fully or partially complementary) to the antisense strand.

In some embodiments of the disclosure, the sense strand includes a structure represented by Formula S5, wherein Formula S5 is, in the 5′-to-3′ direction:

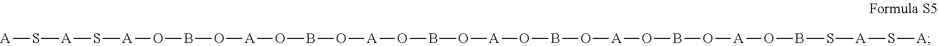

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S6, wherein Formula S6 is, in the 5′-to-3′ direction:

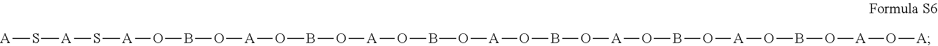

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S7, wherein Formula S7 is, in the 5′-to-3′ direction:

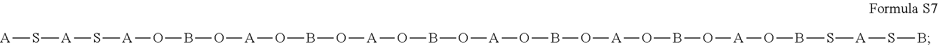

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the sense strand includes a structure represented by Formula S8, wherein Formula S8 is, in the 5′-to-3′ direction:

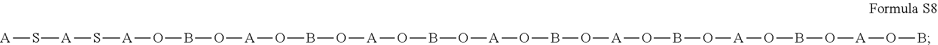

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region represented by Formula VI, wherein Formula VI is, in the 5′-to-3′ direction:

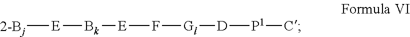

wherein A is represented by the formula C—P¹-D-P¹; each B is represented by the formula C—P²; each C is a 2′-O-Me ribonucleoside; each C′, independently, is a 2′-O-Me ribonucleoside or a 2′-F ribonucleoside; each D is a 2′-F ribonucleoside; each E is represented by the formula D-P²—C—P²; F is represented by the formula D-P¹—C—P¹; each G is represented by the formula C—P¹; each P¹ is a phosphorothioate internucleoside linkage; each P² is a phosphodiester internucleoside linkage; j is an integer from 1 to 7 (e.g., 1, 2, 3, 4, 5, 6, or 7); k is an integer from 1 to 7 (e.g., 1, 2, 3, 4, 5, 6, or 7); and 1 is an integer from 1 to 7 (e.g., 1, 2, 3, 4, 5, 6, or 7). In some embodiments, j is 3. In some embodiments, k is 6. In some embodiments, 1 is 2. In some embodiments, j is 3, k is 6, and 1 is 2. The antisense strand is complementary (e.g., fully or partially complementary) to a target nucleic acid.

In some embodiments of the disclosure, the antisense strand includes a structure represented by Formula A4, wherein Formula A4 is, in the 5′-to-3′ direction:

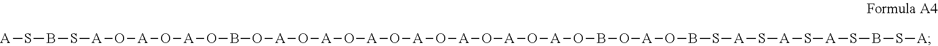

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain a sense strand including a region represented by Formula VII, wherein Formula VII is, in the 5′-to-3′ direction:

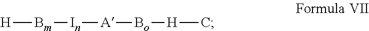

wherein A′ is represented by the formula C—P²-D-P²; each H is represented by the formula (C—P¹)₂; each I is represented by the formula (D-P²); B, C, D, P¹, and P² are as defined in Formula VI; m is an integer from 1 to 7 (e.g., 1, 2, 3, 4, 5, 6, or 7); n is an integer from 1 to 7 (e.g., 1, 2, 3, 4, 5, 6, or 7); and o is an integer from 1 to 7 (e.g., 1, 2, 3, 4, 5, 6, or 7). In some embodiments, m is 3. In some embodiments, n is 3. In some embodiments, o is 3. In some embodiments, m is 3, n is 3, and o is 3. The sense strand is complementary (e.g., fully or partially complementary) to the antisense strand.

In some embodiments of the disclosure, the sense strand includes a structure represented by Formula S9, wherein Formula S9 is, in the 5′-to-3′ direction:

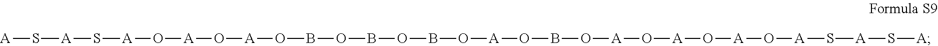

wherein A represents a 2′-O-Me ribonucleoside, B represents a 2′-F ribonucleoside, O represents a phosphodiester internucleoside linkage, and S represents a phosphorothioate internucleoside linkage.

In some embodiments of the disclosure, the siRNA may contain an antisense strand including a region that is represented by Formula VIII:

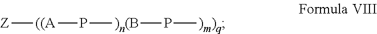

wherein Z is a 5′ phosphorus stabilizing moiety; each A is a 2′-O-methyl (2′-O-Me) ribonucleoside; each B is a 2′-fluoro-ribonucleoside; each P is, independently, an internucleoside linkage selected from a phosphodiester linkage and a phosphorothioate linkage; n is an integer from 1 to 5 (e.g., 1, 2, 3, 4, or 5); m is an integer from 1 to 5 (e.g., 1, 2, 3, 4, or 5); and q is an integer between 1 and 30 (1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).

### 5.5. Methods of siRNA Synthesis

The siRNA molecules of the disclosure can be synthesized by standard methods known in the art as further discussed below, e.g., by use of an automated DNA synthesizer, such as are commercially available from, for example, Biosearch, Applied Biosystems, Inc.

The siRNA agent can be prepared using solution-phase or solid-phase organic synthesis or both. Organic synthesis offers the advantage that the oligonucleotide including unnatural or chemically modified nucleotides can be easily prepared. siRNA molecules of the disclosure can be prepared using solution-phase or solid-phase organic synthesis or both.

Further, it is contemplated that for any siRNA agent disclosed herein, further optimization could be achieved by systematically either adding or removing linked nucleosides to generate longer or shorter sequences. Further still, such optimized sequences can be adjusted by, e.g., the introduction of chemically modified nucleosides, and/or modified internucleoside linkages as described herein or as known in the art, including alternative nucleosides, alternative sugar moieties, and/or alternative internucleoside linkages as known in the art and/or discussed herein to further optimize the molecule (e.g., increasing serum stability or circulating half-life, increasing thermal stability, enhancing transmembrane delivery, and/or targeting to a particular location or cell type).

### 5.6. 5′ Phosphorus Stabilizing Moieties

To further protect the siRNA molecules of this disclosure from degradation, a 5′-phosphorus stabilizing moiety may be employed. A 5′-phosphorus stabilizing moiety replaces the 5′-phosphate to prevent hydrolysis of the phosphate. Hydrolysis of the 5′-phosphate prevents binding to RISC, a necessary step in gene silencing. Any replacement for phosphate that does not impede binding to RISC is contemplated in this disclosure. In some embodiments, the replacement for the 5′-phosphate is also stable to in vivo hydrolysis. Each strand of a siRNA molecule may independently and optionally employ any suitable 5′-phosphorus stabilizing moiety.

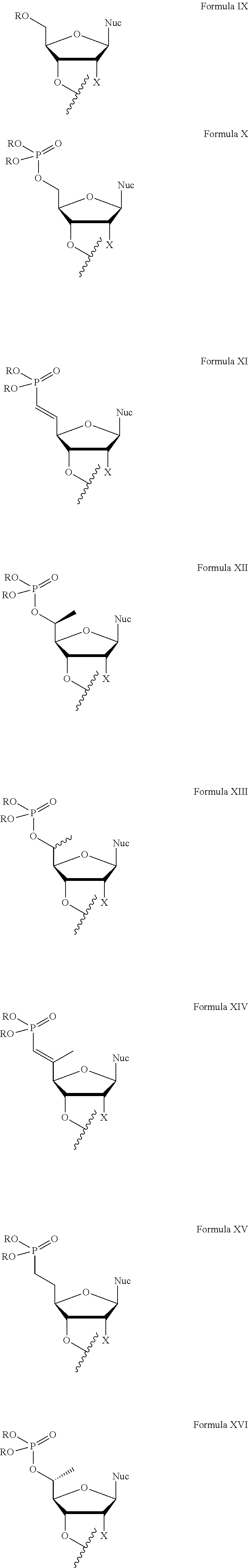

Some exemplary endcaps are demonstrated in Formulas IX-XVI. Nuc in Formulas IX-XVI represents a nucleobase or nucleobase derivative or replacement as described herein. X in formula IX-XVI represents a 2′-modification as described herein. Some embodiments employ hydroxy as in Formula IX, phosphate as in Formula X, vinylphosphonates as in Formula XI and XIV, 5′-methyl-substituted phosphates as in Formula XII, XIII, and XVI, methylenephosphonates as in Formula XV, or vinyl 5′-vinylphsophonate as a 5′-phosphorus stabilizing moiety as demonstrated in Formula XI.

The present disclosure further provides siRNA molecules having one or more hydrophobic moieties attached thereto. The hydrophobic moiety may be covalently attached to the 5′ end or the 3′ end of the siRNA molecules of the disclosure. Non-limiting examples of hydrophobic moieties suitable for use with the siRNA molecules of the disclosure may include cholesterol, vitamin D, tocopherol, phosphatidylcholine (PC), docosahexaenoic acid, docosanoic acid, PC-docosanoic acid, eicosapentaenoic acid, lithocholic acid or any combination of the aforementioned hydrophobic moieties with PC.

### 5.7. siRNA Branching

The siRNA molecules of the disclosure may be branched. For example, the siRNA molecules of the disclosure may have one of several branching patterns, as described herein.

According to the present disclosure, the siRNA molecules disclosed herein may be branched siRNA molecules. The siRNA molecule may not be branched, or may be di-branched, tri-branched, or tetra-branched, connected through a linker. Each main branch may be further branched to allow for 2, 3, 4, 5, 6, 7, or 8 separate RNA single- or double-strands. The branch points on the linker may stem from the same atom, or separate atoms along the linker. Some exemplary embodiments are listed in Table 2.

TABLE 2
Branched siRNA structures

| Di-branched | Tri-branched | Tetra-branched |
|---|---|---|
| RNA-L-RNA Formula XVII | 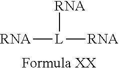 | 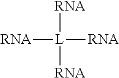 |
| | | Formula XXIV |
| 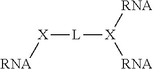 | 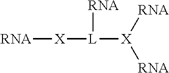 | 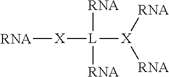 |
| Formula XVIII | Formula XXI | Formula XXV |
| 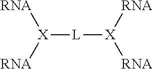 | 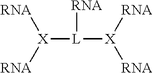 | 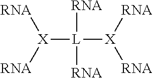 |
| Formula XIX | Formula XXII | Formula XXVI |
| | 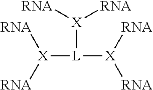 | 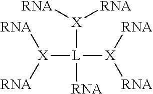 |
| | Formula XXIII | Formula XXVII |
| | | 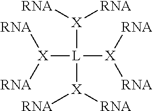 |
| | | Formula XXVIII |

In some embodiments, the siRNA molecule is a branched siRNA molecule. In some embodiments, the branched siRNA molecule is di-branched, tri-branched, or tetra-branched. In some embodiments, the di-branched siRNA molecule is represented by any one of Formulas XVII-XIX, wherein each RNA, independently, is an siRNA molecule, L is a linker, and each X, independently, represents a branch point moiety (e.g., phosphoroamidite, tosylated solketal, 1,3-diaminopropanol, pentaerythritol, or any one of the branch point moieties described in U.S. Pat. No. 10,478,503).

In some embodiments, the tri-branched siRNA molecule represented by any one of Formulas XX-XXIII, wherein each RNA, independently, is an siRNA molecule, L is a linker, and each X, independently, represents a branch point moiety.

In some embodiments, the tetra-branched siRNA molecule represented by any one of Formulas XXIV-XXVIII, wherein each RNA, independently, is an siRNA molecule, L is a linker, and each X, independently, represents a branch point moiety.

Multiple strands of siRNA described herein may be covalently attached by way of a linker. The effect of this branching improves, inter alia, cell permeability allowing better access into cells (e.g., neurons or glial cells) in the CNS. Any linking moiety may be employed which is not incompatible with the siRNAs of the present invention. Linkers include ethylene glycol chains of 2 to 10 subunits (e.g., 2, 3, 4, 5, 6, 7, 8, 9, or 10 subunits), alkyl chains, carbohydrate chains, block copolymers, peptides, RNA, DNA, and others. In some embodiments, any carbon or oxygen atom of the linker is optionally replaced with a nitrogen atom, bears a hydroxyl substituent, or bears an oxo substituent. In some embodiments, the linker is a poly-ethylene glycol (PEG) linker. The PEG linkers suitable for use with the disclosed compositions and methods include linear or non-linear PEG linkers. Examples of non-linear PEG linkers include branched PEGs, linear forked PEGs, or branched forked PEGs.

PEG linkers of various weights may be used with the disclosed compositions and methods. For example, the PEG linker may have a weight that is between 5 and 500 Daltons. In some embodiments, a PEG linker having a weight that is between 500 and 1,000 Dalton may be used. In some embodiments, a PEG linker having a weight that is between 1,000 and 10,000 Dalton may be used. In some embodiments, a PEG linker having a weight that is between 200 and 20,000 Dalton may be used. In some embodiments, the linker is covalently attached to a sense strand of the siRNA. In some embodiments, the linker is covalently attached to an antisense strand of the siRNA. In some embodiments, the PEG linker is a triethylene glycol (TrEG) linker. In some embodiments, the PEG linker is a tetraethylene glycol (TEG) linker.

In some embodiments, the linker is an alkyl chain linker. In some embodiments, the linker is a peptide linker. In some embodiments, the linker is an RNA linker. In some embodiments, the linker is a DNA linker.

Linkers may covalently link 2, 3, 4, or 5 unique siRNA strands. The linker may covalently bind to any part of the siRNA oligomer. In some embodiments, the linker attaches to the 3′ end of nucleosides of each siRNA strand. In some embodiments, the linker attaches to the 5′ end of nucleosides of each siRNA strand. In some embodiments, the linker attaches to a nucleoside of an siRNA strand (e.g., sense or antisense strand) by way of a covalent bond-forming moiety. In some embodiments, the covalent-bond-forming moiety is selected from the group consisting of an alkyl, ester, amide, carbonate, carbamate, triazole, urea, formacetal, phosphonate, phosphate, and phosphate derivative (e.g., phosphorothioate, phosphoramidate, etc.). In certain embodiments the linker is a divalent oligonucleotide (DIO) linker.

In some embodiments, the linker has a structure of Formula L1:

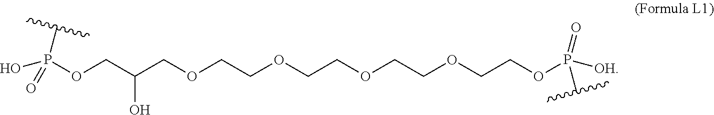 [SMILES: *P(=O)(O)OCCOCCOCCOCCOCC(O)COP(*)(=O)O]

In some embodiments, the linker has a structure of Formula L2:

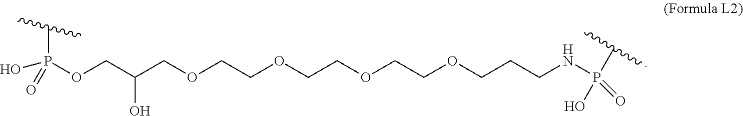

In some embodiments, the linker has a structure of Formula L3:

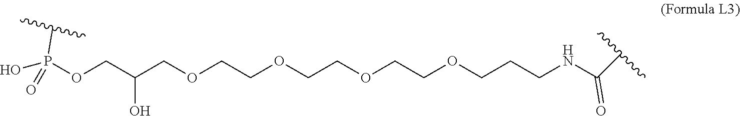 [SMILES: *C(=O)NCCCOCCOCCOCCOCC(O)COP(*)(=O)O]

In some embodiments, the linker has a structure of Formula L4:

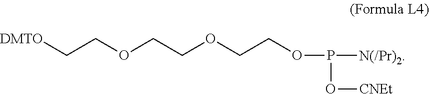

In some embodiments, the linker has a structure of Formula L5:

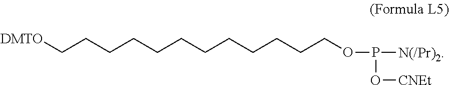

In some embodiments, the linker has a structure of Formula L6:

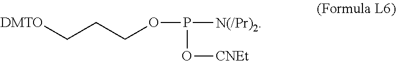

In some embodiments, the linker has a structure of Formula L7, as is shown below:

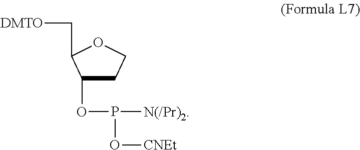

In some embodiments, the linker has a structure of Formula L8:

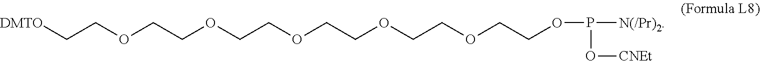

In some embodiments, the linker has a structure of Formula L9:

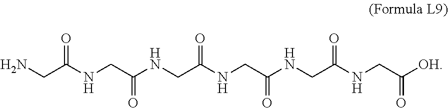 [SMILES: NCC(=O)NCC(=O)NCC(=O)NCC(=O)NCC(=O)NCC(=O)O]

In some embodiments, the selection of a linker for use with one or more of the branched siRNA molecules disclosed herein may be based on the hydrophobicity of the linker, such that, e.g., desirable hydrophobicity is achieved for the one or more branched siRNA molecules of the disclosure. For example, a linker containing an alkyl chain may be used to increase the hydrophobicity of the branched siRNA molecule as compared to a branched siRNA molecule having a less hydrophobic linker or a hydrophilic linker.

The siRNA agents disclosed herein may be synthesized and/or modified by methods well established in the art, such as those described in Beaucage, S. L. et al. (edrs.), Current Protocols in Nucleic Acid Chemistry, John Wiley & Sons, Inc., New York, N.Y., 2000, which is hereby incorporated herein by reference.

### 5.8. Methods of Treatment

The SNCA-targeting siRNA molecules of the disclosure may be delivered to a subject, thereby treating a synucleinopathy. In certain embodiments, the synucleinopathy is Parkinson's disease, Alzheimer's disease, Lewy body dementia, multiple symptom atrophy, or any other medical risk(s) associated with the SNCA gene. For example, the siRNA molecules may be delivered to a subject, thereby treating a synucleinopathy and/or mitigating synucleinopathy associated phenotypes. Exemplary synucleinopathies include Parkinson's disease, Alzheimer's disease, Lewy body dementia, multiple symptom atrophy, or any other medical risk(s) associated with the SNCA gene. Furthermore, the siRNA molecules of the disclosure may also be delivered to a subject having a variant of the SNCA gene for which siRNA-mediated gene silencing of the SNCA variant gene reduces the expression level of SNCA transcript, thereby treating a synucleinopathy, e.g., Parkinson's disease, Alzheimer's disease, Lewy body dementia, multiple symptom atrophy, or any other medical risk(s) associated with the SNCA gene.

The disclosure provides methods of treating a subject by way of SNCA gene silencing with one or more of the siRNA molecules described herein. The gene silencing may be performed in a subject to silence wild type SNCA transcripts, mutant SNCA transcripts, splice isoforms of SNCA transcripts, and/or overexpressed SNCA transcripts thereof, relative to a healthy subject. The method may include delivering to the CNS or affected tissues of the subject (e.g., a human) the siRNA molecules of the disclosure or a pharmaceutical composition containing the same by any appropriate route of administration (e.g., intracerebroventricular, intrathecal injection, intrastriatal injection, intra-cisterna magna injection by catheterization, intraparenchymal injection, intravenous injection, subcutaneous injection, or intramuscular injection). The active compound can be administered in any suitable dose. The actual dosage amount of a composition of the present disclosure administered to a patient can be determined by physical and physiological factors such as body weight, severity of condition, previous or concurrent therapeutic interventions, idiopathy of the patient and on the route of administration. Depending upon the dosage and the route of administration, the number of administrations of an exemplary dosage and/or an effective amount may vary according to the response of the subject. The practitioner responsible for administration will, in any event, determine the concentration of active ingredient(s) in a composition and appropriate dose(s) for the individual subject. Administration may occur any suitable number of times per day, and for as long as necessary. Subjects may be adult or pediatric humans, with or without comorbid diseases.

### 5.8.1. Selection of Subjects

Subjects that may be treated with the siRNA molecules disclosed herein are subjects in need of treatment of, for example, a synucleinopathy. In certain embodiments, the synucleinopathy is Parkinson's disease, Alzheimer's disease, Lewy body dementia, multiple symptom atrophy, or any other medical risk(s) associated with the SNCA gene. Subjects that may be treated with the siRNA molecules disclosed herein may include, for example, humans, monkeys, rats, mice, pigs, and other mammals containing at least one orthologous copy of the SNCA gene. Subjects may be adult or pediatric humans, with or without comorbid diseases.

### 5.8.2. Pharmaceutical Compositions

The siRNA molecules in the present disclosure may be formulated into a pharmaceutical composition for administration to a subject in a biologically compatible form suitable for administration in vivo. Accordingly, the present disclosure provides a pharmaceutical composition containing a siRNA molecule of the disclosure in admixture with a suitable diluent, carrier, or excipient. The siRNA molecules may be administered, for example, directly into the CNS or affected tissues of the subject (e.g., by way of intracerebroventricular, intrastriatally, intrathecal injection, intra-cisterna magna injection by catheterization, intraparenchymal injection, intravenous injection, subcutaneous injection, or intramuscular injection).

Conventional procedures and ingredients for the selection and preparation of suitable formulations are described, for example, in Remington, J. P. The Science and Practice of Pharmacy, Easton, PA. Mack Publishers, 2012, 22^{nd} ed. and in The United States Pharmacopeial Convention, The National Formulary, United States Pharmacopeial, 2015, USP 38 NF 33).

Under ordinary conditions of storage and use, a pharmaceutical composition may contain a preservative, e.g., to prevent the growth of microorganisms. Pharmaceutical compositions may include sterile aqueous solutions, dispersions, or powders, e.g., for the extemporaneous preparation of sterile solutions or dispersions. In all cases the form may be sterilized using techniques known in the art and may be fluidized to the extent that may be easily administered to a subject in need of treatment.

A pharmaceutical composition may be administered to a subject, e.g., a human subject, alone or in combination with pharmaceutically acceptable carriers, as noted herein, the proportion of which may be determined by the solubility and/or chemical nature of the compound, chosen route of administration, and standard pharmaceutical practice.

### 5.8.3. Dosing Regimens

A physician having ordinary skill in the art can readily determine an effective amount of the siRNA molecule for administration to a mammalian subject (e.g., a human) in need thereof. For example, a physician could start prescribing doses of one the siRNA molecules of the disclosure at levels lower than that required to achieve the desired therapeutic effect and gradually increase the dosage until the desired effect is achieved. Alternatively, a physician may begin a treatment regimen by administering one of the siRNA molecules of the disclosure at a high dose and subsequently administer progressively lower doses until reaching a minimal dosage at which a therapeutic effect is achieved (e.g., a reduction in expression of a target gene sequence). In general, a suitable daily dose of one of the siRNA molecules of the disclosure will be an amount of the siRNA molecule which is the lowest dose effective to produce a therapeutic effect. The ss- or ds-siRNA molecules of the disclosure may be administered by injection, e.g., intrathecally, intracerebroventricularly, by intra-cisterna magna injection by catheterization, intraparenchymally, intravenously, subcutaneously, or intramuscularly. A daily dose of a therapeutic composition of the siRNA molecules of the disclosure may be administered as a single dose or as two, three, four, five, six or more doses administered separately at appropriate intervals throughout the day, week, month, or year, optionally, in unit dosage forms. While it is possible for the siRNA molecules of the disclosure to be administered alone, it may also be administered as a pharmaceutical formulation in combination with excipients, carriers, and optionally, additional therapeutic agents.

### 5.8.4. Routes of Administration

The method of the disclosure contemplates any route of administration tolerated by the therapeutic composition. Some embodiments of the method include injection intrathecally, intracerebroventricularly, intrastriatally, intraparenchymally, or by intra-cisterna magna injection by catheterization.

Intrathecal injection is the direct injection into the spinal column or subarachnoid space. By injecting directly into the CSF of the spinal column the siRNA molecules of the disclosure have direct access to cells (e.g., neurons and glial cells) in the spinal column and a route to access the cells in the brain by bypassing the blood brain barrier.

Intracerebroventricular (ICV) injection is a method to directly inject into the CSF of the cerebral ventricles. Similar to intrathecal injection, ICV is a method of injection which bypasses the blood brain barrier. Using ICV allows the advantage of access to the cells of the brain and spinal column without the danger of the therapeutic being degraded in the blood.

Intrastriatal injection is the direct injection into the striatum, or corpus striatum. The striatum is an area in the subcortical basal ganglia in the brain. Injecting into the striatum bypasses the blood brain barrier and the pharmacokinetic challenges of injection into the blood stream and allows for direct access to the cells of the brain.

Intraparenchymal administration is the direct injection into the parenchyma (e.g., the brain parenchyma). Injection into the brain parenchyma allows for injection directly into brain regions affected by a disease or disorder while bypassing the blood brain barrier.

Intra-cisterna magna injection by catheterization is the direct injection into the cisterna magna. The cisterna magna is the area of the brain located between the cerebellum and the dorsal surface of the medulla oblongata. Injecting into the cisterna magna results in more direct delivery to the cells of the cerebellum, brainstem, and spinal cord.

In some embodiments of the methods described herein, the therapeutic composition may be delivered to the subject by way of systemic administration, e.g., intravenously, intramuscularly, or subcutaneously.

Intravenous (IV) injection is a method to directly inject into the bloodstream of a subject. The IV administration may be in the form of a bolus dose or by way of continuous infusion, or any other method tolerated by the therapeutic composition.

Intramuscular (IM) injection is injection into a muscle of a subject, such as the deltoid muscle or gluteal muscle. IM may allow for rapid absorption of the therapeutic composition.

Subcutaneous injection is injection into subcutaneous tissue. Absorption of compositions delivered subcutaneously may be slower than IV or IM injection, which may be beneficial for compositions requiring continuous absorption.

### 6. ENUMERATED EMBODIMENTS OF THE DISCLOSURE

The compositions, methods, and kits described herein include the following non-limiting, exemplary, enumerated embodiments of the disclosure. In some embodiments, the disclosure provides:
Embodiment 1. A small interfering RNA (siRNA) molecule comprising an antisense strand and sense strand having complementarity to the antisense strand, wherein the antisense strand is from 10 to 30 nucleotides in length and has complementarity sufficient to hybridize to a region within an alpha-synuclein (SNCA) mRNA transcript having the nucleic acid sequence of any one of SEQ ID NOs: 421-840.
Embodiment 2. The siRNA molecule of embodiment 1, wherein the antisense strand has at least 70% complementarity to a region of 21 contiguous nucleobases within the SNCA mRNA transcript having the nucleic acid sequence of any one of SEQ ID NOs: 421-840.
Embodiment 3. The siRNA molecule of embodiment 2, wherein the antisense strand has at least 75% complementarity to a region of 21 contiguous nucleobases within the SNCA mRNA transcript having the nucleic acid sequence of any one of SEQ ID NOs: 421-840, optionally wherein the antisense strand has at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementarity to the region within the 421-840 mRNA transcript having the nucleic acid sequence of any one of SEQ ID Nos: 421-840.
Embodiment 4. The siRNA molecule of any one of embodiments 1-3, wherein the antisense strand comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the SNCA mRNA transcript having the nucleic acid sequence of any one of SEQ ID NOs: 421-840.
Embodiment 5. The siRNA molecule of embodiment 4, wherein the antisense strand comprises from 10 to 30 contiguous nucleotides that are fully complementary to a contiguous polynucleotide segment of equal length within the region of the SNCA mRNA transcript having the nucleic acid sequence of any one of SEQ ID NOs: 421-840.
Embodiment 6. The siRNA molecule of embodiment 5, wherein the antisense strand comprises from 12 to 30 contiguous nucleotides that are fully complementary to a contiguous polynucleotide segment of equal length within the region of the SNCA mRNA transcript having the nucleic acid sequence of any one of SEQ ID NOs: 421-840.
Embodiment 7. The siRNA molecule of embodiment 6, wherein the antisense strand comprises from 15 to 30 contiguous nucleotides that are fully complementary to a contiguous polynucleotide segment of equal length within the region of the SNCA mRNA transcript having the nucleic acid sequence of any one of SEQ ID NOs: 421-840.
Embodiment 8. The siRNA molecule of embodiment 7, wherein the antisense strand comprises from 18 to 30 contiguous nucleotides that are fully complementary to a contiguous polynucleotide segment of equal length within the region of the SNCA mRNA transcript having the nucleic acid sequence of any one of SEQ ID NOs: 421-840.
Embodiment 9. The siRNA molecule of embodiment 8, wherein the antisense strand comprises from 21 to 30 contiguous nucleotides that are fully complementary to a contiguous polynucleotide segment of equal length within the region of the SNCA mRNA transcript having the nucleic acid sequence of any one of SEQ ID NOs: 421-840.
Embodiment 10. The siRNA molecule of any one of embodiments 1-9, wherein the antisense strand comprises from 24 to 30 contiguous nucleotides that are fully complementary to a contiguous polynucleotide segment of equal length within the region of the SNCA mRNA transcript having the nucleic acid sequence of any one of SEQ ID NOs: 421-840.
Embodiment 11. The siRNA molecule of embodiment 10, wherein the antisense strand comprises 30 contiguous nucleotides that are fully complementary to a contiguous polynucleotide segment of equal length within the region of the SNCA mRNA transcript having the nucleic acid sequence of any one of SEQ ID NOs: 421-840.
Embodiment 12. The siRNA molecule of any one of embodiments 1-11, wherein the antisense strand comprises 9 or fewer nucleotide mismatches relative to a region of 21 contiguous nucleobases of the SNCA mRNA transcript having the nucleic acid sequence of any one of SEQ ID NOs: 421-840, optionally wherein the antisense strand comprises 8 or fewer, 7 or fewer, 6 or fewer, 5 or fewer, 4 or fewer, 3 or fewer, 2 or fewer, or only 1 mismatch relative to the region of the SNCA mRNA transcript having the nucleic acid sequence of any one of SEQ ID NOs: 421-840.
Embodiment 13. The siRNA molecule of any one of embodiments 1-12, wherein the region of the SNCA mRNA transcript has the nucleic acid sequence of any one of SEQ ID NOs: 459, 535, 551, 599, 631, 663, 664, 666, 667, 671, 748, 750, 751, 752, 760, 762, and 777.
Embodiment 14. The siRNA molecule of embodiment 13, wherein the region of the SNCA mRNA transcript has the nucleic acid sequence of any one of SEQ ID NOs: 535, 599, 664, 666, 671, 750, 752, 760, and 762.
Embodiment 15. The siRNA molecule of any one of embodiments 1-14, wherein the antisense strand has a nucleic acid sequence that is at least 85% identical to the nucleic acid sequence of any one of SEQ ID NOs: 1-420.
Embodiment 16. The siRNA molecule of embodiment 15, wherein the antisense strand has a nucleic acid sequence that is at least 90% identical to the nucleic acid sequence of any one of SEQ ID NOs: 1-420.
Embodiment 17. The siRNA molecule of embodiment 16, wherein the antisense strand has a nucleic acid sequence that is at least 95% identical to the nucleic acid sequence of SEQ ID NOs: 1-420, optionally wherein the antisense strand has a nucleic acid sequence that is at least 96%, 97%, 98%, or 99% identical to the nucleic acid sequence of any one of SEQ ID NOs: 1-420.
Embodiment 18. The siRNA molecule of embodiment 17, wherein the antisense strand has the nucleic acid sequence of any one of SEQ ID NOs: 1-420.
Embodiment 19. The siRNA molecule of any one of embodiments 15-18, wherein the nucleic acid sequence is any one of SEQ ID NOs: 39, 115, 131, 179, 211, 243, 244, 246, 247, 251, 328, 330, 331, 332, 340, 342, and 357.
Embodiment 20. The siRNA molecule of embodiment 19, wherein the nucleic acid sequence is any one of SEQ ID NOs: 115, 179, 244, 246, 251, 330, 332, 340, and 342.
Embodiment 21. The siRNA molecule of any one of embodiments 1-20, wherein the sense strand has a nucleic acid sequence that is at least 85% identical to the nucleic acid sequence of any one of SEQ ID NOs: 421-840.
Embodiment 22. The siRNA molecule of embodiment 21, wherein the sense strand has a nucleic acid sequence that is at least 90% identical to the nucleic acid sequence of any one of SEQ ID NOs: 421-840.
Embodiment 23. The siRNA molecule of embodiment 22, wherein the sense strand has a nucleic acid sequence that is at least 95% identical to the nucleic acid sequence of SEQ ID NOs: 421-840, optionally wherein the sense strand has a nucleic acid sequence that is at least 96%, 97%, 98%, or 99% identical to the nucleic acid sequence of any one of SEQ ID NOs: 421-840.
Embodiment 24. The siRNA molecule of embodiment 23, wherein the sense strand has the nucleic acid sequence of any one of SEQ ID NOs: 421-840.
Embodiment 25. The siRNA molecule of any one of embodiments 21-24, wherein the nucleic acid sequence is any one of SEQ ID NOs: 459, 535, 551, 599, 631, 663, 664, 666, 667, 671, 748, 750, 751, 752, 760, 762, and 777.
Embodiment 26. The siRNA molecule of embodiment 25, wherein the nucleic acid sequence is any one of SEQ ID NOs: 535, 599, 664, 666, 671, 750, 752, 760, and 762.
Embodiment 27. The siRNA molecule of any one of embodiments 1-26, wherein the antisense strand comprises a structure represented by Formula I, wherein Formula I is, in the 5′-to-3′ direction:

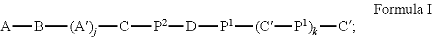

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.
Embodiment 28. The siRNA molecule of embodiment 27, wherein the antisense strand comprises a structure represented by Formula A1, wherein Formula A1 is, in the 5′-to-3′ direction:

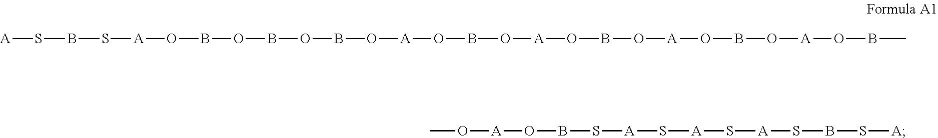

wherein A represents a 2′-O-Me ribonucleoside, B represents a 2′-F ribonucleoside, O represents a phosphodiester internucleoside linkage, and S represents a phosphorothioate internucleoside linkage.
Embodiment 29. The siRNA molecule of any one of embodiments 1-26, wherein the antisense strand comprises a structure represented by Formula II, wherein Formula II is, in the 5′-to-3′ direction:

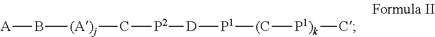

wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.
Embodiment 30. The siRNA molecule of embodiment 29, wherein the antisense strand comprises a structure represented by Formula A2, wherein Formula A2 is, in the 5′-to-3′ direction:

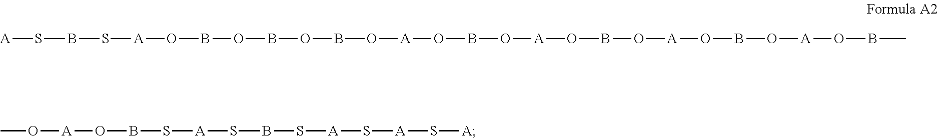

wherein A represents a 2′-O-Me ribonucleoside, B represents a 2′-F ribonucleoside, O represents a phosphodiester internucleoside linkage, and S represents a phosphorothioate internucleoside linkage.
Embodiment 31. The siRNA molecule of any one of embodiments 1-30, wherein the sense strand comprises a structure represented by Formula III, wherein Formula III is, in the 5′-to-3′ direction:

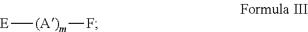

wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7.
Embodiment 32. The siRNA molecule of embodiment 31, wherein the sense strand comprises a structure represented by Formula S1, wherein Formula S1 is, in the 5′-to-3′ direction:

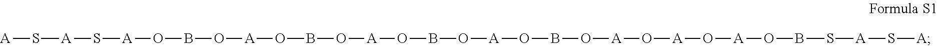

wherein A represents a 2′-O-Me ribonucleoside, B represents a 2′-F ribonucleoside, O represents a phosphodiester internucleoside linkage, and S represents a phosphorothioate internucleoside linkage.
Embodiment 33. The siRNA molecule of embodiment 31, wherein the sense strand comprises a structure represented by Formula S2, wherein Formula S2 is, in the 5′-to-3′ direction:

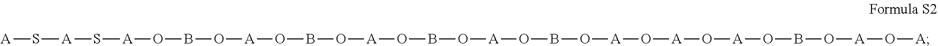

wherein A represents a 2′-O-Me ribonucleoside, B represents a 2′-F ribonucleoside, O represents a phosphodiester internucleoside linkage, and S represents a phosphorothioate internucleoside linkage.
Embodiment 34. The siRNA molecule of embodiment 31, wherein the sense strand comprises a structure represented by Formula S3, wherein Formula S3 is, in the 5′-to-3′ direction:

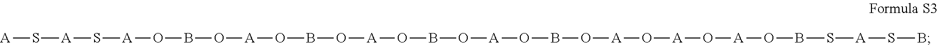

wherein A represents a 2′-O-Me ribonucleoside, B represents a 2′-F ribonucleoside, O represents a phosphodiester internucleoside linkage, and S represents a phosphorothioate internucleoside linkage.
Embodiment 35. The siRNA molecule of embodiment 31, wherein the sense strand comprises a structure represented by Formula S4, wherein Formula S4 is, in the 5′-to-3′ direction:

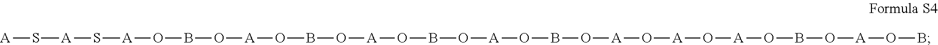

wherein A represents a 2′-O-Me ribonucleoside, B represents a 2′-F ribonucleoside, O represents a phosphodiester internucleoside linkage, and S represents a phosphorothioate internucleoside linkage.
Embodiment 36. The siRNA molecule of any one of embodiments 1-26 and 31-35, wherein the antisense strand comprises a structure represented by Formula IV, wherein Formula IV is, in the 5′-to-3′ direction:

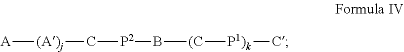

wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7.
Embodiment 37. The siRNA molecule of embodiment 36, wherein the antisense strand comprises a structure represented by Formula A3, wherein Formula A3 is, in the 5′-to-3′ direction:

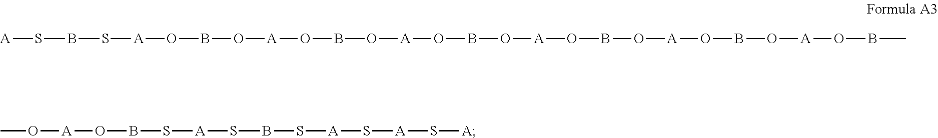

wherein A represents a 2′-O-Me ribonucleoside, B represents a 2′-F ribonucleoside, O represents a phosphodiester internucleoside linkage, and S represents a phosphorothioate internucleoside linkage.
Embodiment 38. The siRNA molecule of any one of embodiments 1-30, 36, and 37, wherein the sense strand comprises a structure represented by Formula V, wherein Formula V is, in the 5′-to-3′ direction:

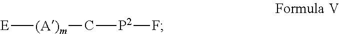

wherein E is represented by the formula (C—P¹)₂;
F is represented by the formula D-P¹—C—P¹—C, D-P²—C—P²—C, D-P¹—C—P¹-D, or D-P²—C—P²-D;
A′, C, D, P¹ and P² are as defined in Formula IV; and
m is an integer from 1 to 7.
Embodiment 39. The siRNA molecule of embodiment 38, wherein the sense strand comprises a structure represented by Formula S5, wherein Formula S5 is, in the 5′-to-3′ direction:

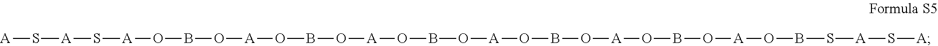

wherein A represents a 2′-O-Me ribonucleoside, B represents a 2′-F ribonucleoside, O represents a phosphodiester internucleoside linkage, and S represents a phosphorothioate internucleoside linkage.
Embodiment 40. The siRNA molecule of embodiment 38, wherein the sense strand comprises a structure represented by Formula S6, wherein Formula S6 is, in the 5′-to-3′ direction:

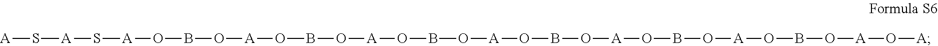

wherein A represents a 2′-O-Me ribonucleoside, B represents a 2′-F ribonucleoside, O represents a phosphodiester internucleoside linkage, and S represents a phosphorothioate internucleoside linkage.
Embodiment 41. The siRNA molecule of embodiment 38, wherein the sense strand comprises a structure represented by Formula S7, wherein Formula S7 is, in the 5′-to-3′ direction:

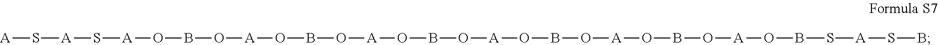

wherein A represents a 2′-O-Me ribonucleoside, B represents a 2′-F ribonucleoside, O represents a phosphodiester internucleoside linkage, and S represents a phosphorothioate internucleoside linkage.
Embodiment 42. The siRNA molecule of embodiment 38, wherein the sense strand comprises a structure represented by Formula S8, wherein Formula S8 is, in the 5′-to-3′ direction:

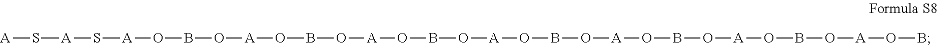

wherein A represents a 2′-O-Me ribonucleoside, B represents a 2′-F ribonucleoside, O represents a phosphodiester internucleoside linkage, and S represents a phosphorothioate internucleoside linkage.
Embodiment 43. The siRNA molecule of any one of embodiments 1-26, 31-35 and 38-42, wherein the antisense strand comprises a structure represented by Formula VI, wherein Formula VI is, in the 5′-to-3′ direction:

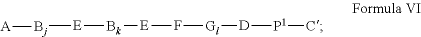

wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7;
k is an integer from 1 to 7; and 1 is an integer from 1 to 7.
Embodiment 44. The siRNA molecule of embodiment 43, wherein the antisense strand comprises a structure represented by Formula A4, wherein Formula A4 is, in the 5′-to-3′ direction:

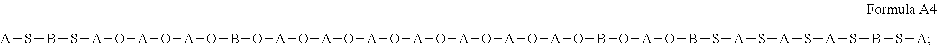

wherein A represents a 2′-O-Me ribonucleoside, B represents a 2′-F ribonucleoside, O represents a phosphodiester internucleoside linkage, and S represents a phosphorothioate internucleoside linkage.
Embodiment 45. The siRNA molecule of any one of embodiments 1-30, 36, 37, 43, and 44, wherein the sense strand comprises a structure represented by Formula VII, wherein Formula VII is, in the 5′-to-3′ direction:

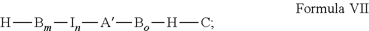

wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7;
n is an integer from 1 to 7; and
is an integer from 1 to 7.
Embodiment 46. The siRNA molecule of embodiment 45, wherein the sense strand comprises a structure represented by Formula S9, wherein Formula S9 is, in the 5′-to-3′ direction:

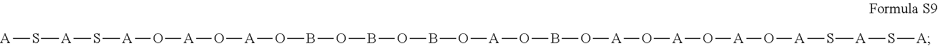

wherein A represents a 2′-O-Me ribonucleoside, B represents a 2′-F ribonucleoside, O represents a phosphodiester internucleoside linkage, and S represents a phosphorothioate internucleoside linkage.
Embodiment 47. The siRNA molecule of any one of embodiments 1-46, wherein the antisense strand further comprises a 5′ phosphorus stabilizing moiety at the 5′ end of the antisense strand.
Embodiment 48. The siRNA molecule of any one of embodiments 1-47, wherein the sense strand further comprises a 5′ phosphorus stabilizing moiety at the 5′ end of the sense strand.
Embodiment 49. The siRNA molecule of embodiment 47 or 48, wherein each 5′ phosphorus stabilizing moiety is, independently, represented by any one of Formulas IX-XVI:

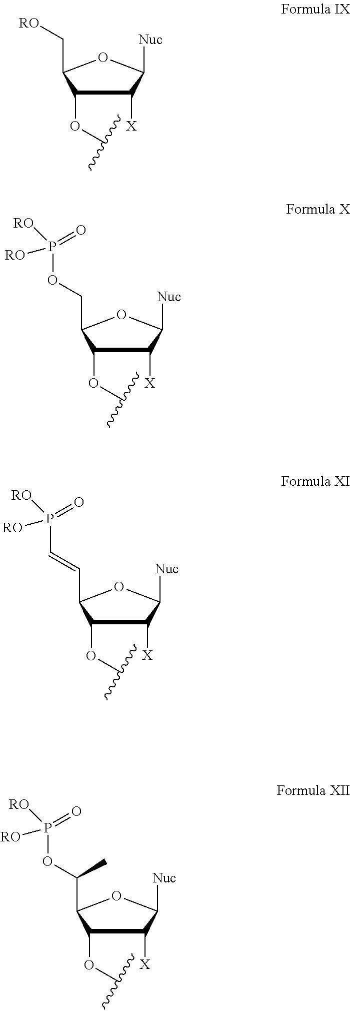

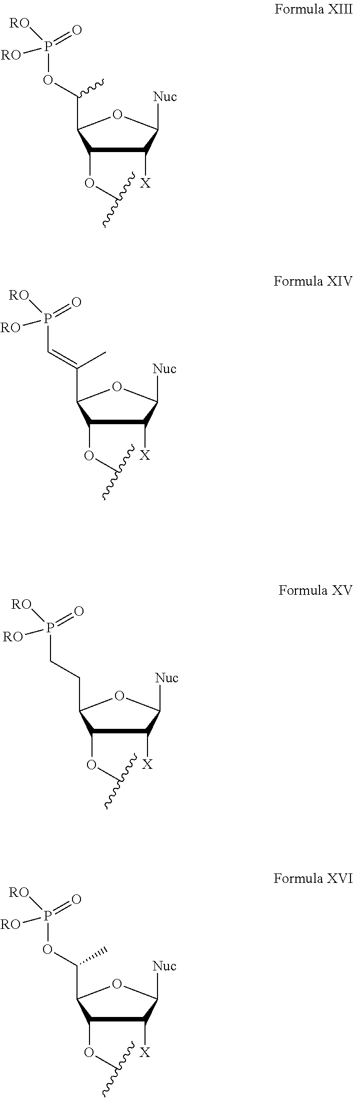

wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, hydroxy, or hydrogen.
Embodiment 50. The siRNA molecule of embodiment 49, wherein the nucleobase is an adenine, uracil, guanine, thymine, or cytosine.
Embodiment 51. The siRNA molecule of any one of embodiments 47-50, wherein the 5′ phosphorus stabilizing moiety is (E)-vinylphosphonate represented by Formula XI.
Embodiment 52. The siRNA molecule of any one of embodiments 1-51, wherein the siRNA molecule further comprises a hydrophobic moiety at the 5′ or the 3′ end of the siRNA molecule.
Embodiment 53. The siRNA molecule of embodiment 52, wherein the hydrophobic moiety is selected from a group consisting of cholesterol, vitamin D, or tocopherol.
Embodiment 54. The siRNA molecule of any one of embodiments 1-53, wherein the length of the sense strand is between 12 and 30 nucleotides.
Embodiment 55. The siRNA molecule of any one of embodiments 1-54, wherein the siRNA molecule is a branched siRNA molecule.
Embodiment 56. The siRNA molecule of embodiment 55, wherein the branched siRNA molecule is di-branched, tri-branched, or tetra-branched.
Embodiment 57. The siRNA molecule of embodiment 56, wherein the siRNA molecule is a di-branched siRNA molecule, optionally wherein the di-branched siRNA molecule is represented by any one of Formulas XVII-XIX:

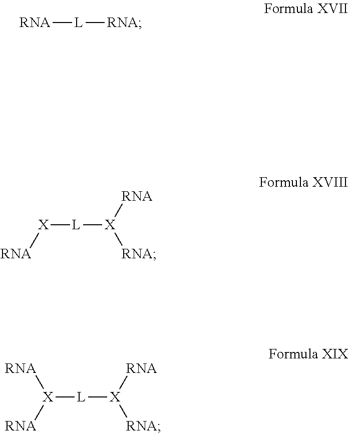

wherein each RNA is, independently, an siRNA molecule, L is a linker, and each X, independently, represents a branch point moiety.
Embodiment 58. The siRNA molecule of embodiment 56, wherein the siRNA molecule is a tri-branched siRNA molecule, optionally wherein the tri-branched siRNA molecule is represented by any one of Formulas XX-XXIII:

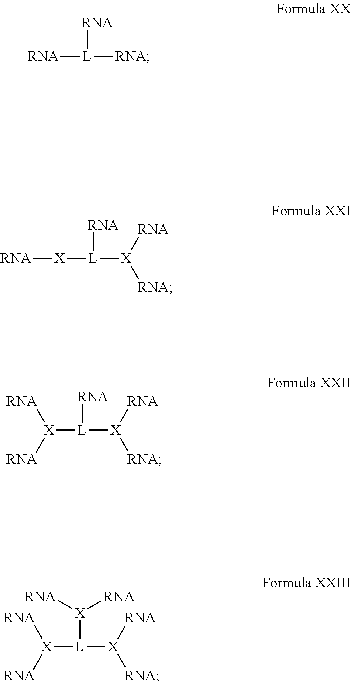

wherein each RNA is, independently, an siRNA molecule, L is a linker, and each X, independently, represents a branch point moiety.
Embodiment 59. The siRNA molecule of embodiment 56, wherein the siRNA molecule is a tetra-branched siRNA molecule, optionally wherein the tetra-branched siRNA molecule is represented by any one of Formulas XXIV-XXVIII:

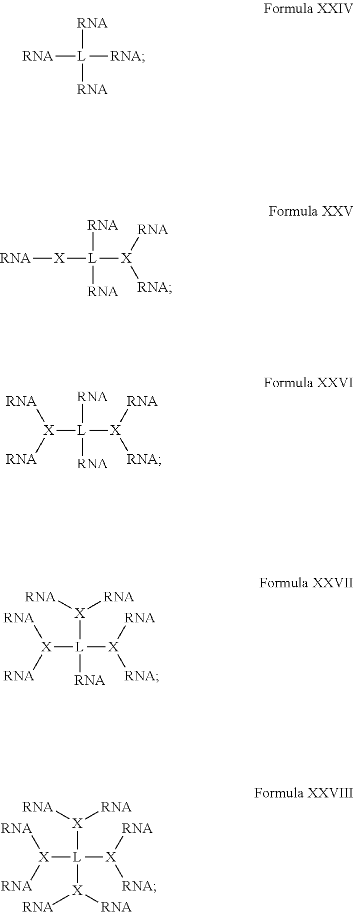

wherein each RNA is, independently, an siRNA molecule, L is a linker, and each X, independently, represents a branch point moiety.
Embodiment 60. The siRNA molecule of any one of embodiments 57-59, wherein the linker is selected from a group consisting of one or more contiguous subunits of an ethylene glycol, alkyl, carbohydrate, block copolymer, peptide, RNA, and DNA.
Embodiment 61. The siRNA molecule of embodiment 60, wherein the one or more contiguous subunits is 2 to 20 contiguous subunits.
Embodiment 62. An siRNA molecule comprising:

(I)
a) a sense strand comprising the sequence
(SEQ ID NO: 841)
(mU)#(mA)#(mC)(fC)(mA)(fC)(mU)(fU)(mA)
(fU)(mU)(mU)(mC)(fU)#(mA)#(mA)-DIO
and an antisense strand comprising the sequence
(SEQ ID NO: 842)
V(mU)#(fU)#(mA)(fG)(fA)(fA)(mA)(fU)(mA)
(fA)(mG)(fU)(mG)(fG)(mU)(fA)#(mG)#(mU)
#(mC)#(mA)#(mC);
b) a sense strand comprising the sequence
(SEQ ID NO: 843)
(mC)#(mA)#(mA)(fG)(mU)(fG)(mC)(fU)(mC
)(fA)(mG)(mU)(mU)(fC)#(mC)#(mA)-DIO
and an antisense strand comprising the sequence
(SEQ ID NO: 844)
V(mU)#(fG)#(mG)(fA)(fA)(fC)(mU)(fG)(mA)
(fG)(mC)(fA)(mC)(fU)(mU)(fG)#(mU)#(mA)
#(mC)#(mA)#(mG);
c) a sense strand comprising the sequence
(SEQ ID NO: 845)
(mA)#(mG)#(mU)(fG)(mG)(fU)(mG)(fC)(mA)
(fU)(mG)(mG)(mU)(fG)#(mU)#(mA)-DIO
and an antisense strand comprising the sequence
(SEQ ID NO: 846)
V(mU)#(fA)#(mC)(fA)(fC)(fC)(mA)(fU)(mG)
(fC)(mA)(fC)(mC)(fA)(mC)(fU)#(mC)#
(mC)#(mC)#(mU)#(mC).
d) a sense strand comprising the sequence
(SEQ ID NO: 847)
(mC)#(mA)#(mA)(fU)(mG)(fA)(mG)(fG)(mC)
(fU)(mU)(mA)(mU)(fG)#(mA)#(mA)-DIO
and an antisense strand comprising the sequence
(SEQ ID NO: 848)
V(mU)#(fU)#(mU)(fG)(fG)(fA)(mA)(fC)(mU)
(fG)(mA)(fG)(mC)(fA)(mC)(fU)#(mU)#
(mG)#(mU)#(mA)#(mC);
e) a sense strand comprising the sequence
(SEQ ID NO: 849)
(mU)#(mC)#(mA)(fG)(mU)(fU)(mC)(fC)(mA)
(fA)(mU)(mG)(mU)(fG)#(mC)#(mA)-DIO
and an antisense strand comprising the sequence
(SEQ ID NO: 850)
V(mU)#(fU)#(mU)(fG)(fG)(fA)(mA)(fC)(mU)
(fG)(mA)(fG)(mC)(fA)(mC)(fU)#(mU)#
(mG)#(mU)#(mA)#(mC).
f) a sense strand comprising the sequence
(SEQ ID NO: 851)
(mC)#(mU)#(mA)(fC)(mG)(fA)(mU)(fG)(mU)
(fU)(mA)(mA)(mA)(fA)#(mC)#(mA)-DIO
and an antisense strand comprising the sequence
(SEQ ID NO: 852)
V(mU)#(fG)#(mC)(fA)(fC)(fA)(mU)(fU)(mG)
(fG)(mA)(fA)(mC)(fU)(mG)(fA)#(mG)#
(mC)#(mA)#(mC)#(mU);
or
g) a sense strand comprising the sequence
(SEQ ID NO: 853)
(mC)#(mU)#(mA)(fC)(mG)(fA)(mU)(fG)(mU)
(fU)(mA)(mA)(mA)(fA)#(mC)#(mA)-DIO
and an antisense strand comprising the sequence
(SEQ ID NO: 854)
V(mU)#(fG)#(mU)(fU)(fU)(fU)(mA)(fA)(mC)
(fA)(mU)(fC)(mG)(fU)(mA)(fG)#(mA)#
(mU)#(mU)#(mG)#(mA);
wherein m represents a 2′-O-Me ribonucleoside,
f represents a 2′-F ribonucleoside,
# represents a phosphorothioate
internucleoside linkage,
-DIO represents a divalent
oligonucleotide (DIO) linker;
and V represents a vinyl phosphonate;
or
(II)
a) a sense strand comprising the sequence
(SEQ ID NO: 841)
(mU)#(mA)#(mC)(fC)(mA)(fC)(mU)(fU)(mA)
(fU)(mU)(mU)(mC)(fU)#(mA)#(mA)-DIO
and an antisense strand comprising the sequence
(SEQ ID NO: 842)
V(mU)#(fU)#(mA)(fG)(fA)(fA)(mA)(fU)(mA)
(fA)(mG)(fU)(mG)(fG)(mU)(fA)#(mG)#
(mU)#(mC)#(mA)#(mC);
b) a sense strand comprising the sequence
(SEQ ID NO: 843)
(mC)#(mA)#(mA)(fG)(mU)(fG)(mC)(fU)(mC)
(fA)(mG)(mU)(mU)(fC)#(mC)#(mA)-DIO
and an antisense strand comprising the sequence
(SEQ ID NO: 844)
V(mU)#(fG)#(mG)(fA)(fA)(fC)(mU)(fG)(mA)
(fG)(mC)(fA)(mC)(fU)(mU)(fG)#(mU)#
(mA)#(mC)#(mA)#(mG);
c) a sense strand comprising the sequence
(SEQ ID NO: 845)
(mA)#(mG)#(mU)(fG)(mG)(fU)(mG)(fC)(mA)
(fU)(mG)(mG)(mU)(fG)#(mU)#(mA)-DIO
and an antisense strand comprising the sequence
(SEQ ID NO: 846)
V(mU)#(fA)#(mC)(fA)(fC)(fC)(mA)(fU)(mG)
(fC)(mA)(fC)(mC)(fA)(mC)(fU)#(mC)#
(mC)#(mC)#(mU)#(mC).
d) a sense strand comprising the sequence
(SEQ ID NO: 847)
(mC)#(mA)#(mA)(fU)(mG)(fA)(mG)(fG)(mC)
(fU)(mU)(mA)(mU)(fG)#(mA)#(mA)-DIO
and an antisense strand comprising the sequence
(SEQ ID NO: 855)
V(mU)#(fU)#(mC)(fA)(fU)(fA)(mA)(fG)(mC)
(fC)(mU)(fC)(mA)(fU)(mU)(fG)#(mU)#
(mC)#(mA)#(mG)#(mG)
e) a sense strand comprising the sequence
(SEQ ID NO: 856)
(mA)#(mG)#(mU)(fG)(mC)(fU)(mC)(fA)(mG)
(fU)(mU)(mC)(mC)(fA)#(mA)#(mA)-DIO
and an antisense strand comprising the sequence
(SEQ ID NO: 850)
V(mU)#(fU)#(mU)(fG)(fG)(fA)(mA)(fC)(mU)
(fG)(mA)(fG)(mC)(fA)(mC)(fU)#(mU)#
(mG)#(mU)#(mA)#(mC).
f) a sense strand comprising the sequence
(SEQ ID NO: 857)
(mU)#(mC)#(mA)(fG)(mU)(fU)(mC)(fC)(mA)
(fA)(mU)(mG)(mU)(fG)#(mC)#(mA)-DIO
and an antisense strand comprising
the sequence
(SEQ ID NO: 852)
V(mU)#(fG)#(mC)(fA)(fC)(fA)(mU)(fU)(mG)
(fG)(mA)(fA)(mC)(fU)(mG)(fA)#(mG)#
(mC)#(mA)#(mC)#(mU);
or
g) a sense strand comprising the sequence
(SEQ ID NO: 853)
(mC)#(mU)#(mA)(fC)(mG)(fA)(mU)(fG)(mU)
(fU)(mA)(mA)(mA)(fA)#(mC)#(mA)-DIO
and an antisense strand comprising the sequence
(SEQ ID NO: 854)
V(mU)#(fG)#(mU)(fU)(fU)(fU)(mA)(fA)(mC)
(fA)(mU)(fC)(mG)(fU)(mA)(fG)#(mA)#
(mU)#(mU)#(mG)#(mA);
wherein m represents a 2′-O-Me ribonucleoside,
f represents a 2′-F ribonucleoside,
# represents a phosphorothioate
internucleoside linkage,
-DIO represents a divalent
oligonucleotide (DIO) linker;
and V represents a vinyl phosphonate

Embodiment 63. A pharmaceutical composition comprising the siRNA molecule of any one of embodiments 1-62 and a pharmaceutically acceptable excipient, carrier, or diluent.
Embodiment 64. A method of delivering an siRNA molecule to a subject diagnosed as having an, the method comprising administering a therapeutically effective amount of the siRNA molecule of any one of embodiments 1-62 or the pharmaceutical composition of embodiment 63 to the subject.
Embodiment 65. A method of treating a synucleinopathy in a subject in need thereof, the method comprising administering a therapeutically effective amount of the siRNA molecule of any one of embodiments 1-62 or the pharmaceutical composition of embodiment 63 to the subject.
Embodiment 66. The method of embodiment 65, wherein the synucleinopathy is Parkinson's disease.
Embodiment 67. The method of embodiment 65, wherein the synucleinopathy is Alzheimer's disease.
Embodiment 68. The method of embodiment 65, wherein the synucleinopathy is Lewy body dementia.
Embodiment 69. The method of embodiment 65, wherein the synucleinopathy is a multiple symptom atrophy.
Embodiment 70. A method of reducing SNCA expression in a subject in need thereof, the method comprising administering a therapeutically effective amount of the siRNA molecule of any one of embodiments 1-62 or the pharmaceutical composition of embodiment 63 to the subject.
Embodiment 71. The method of any one of embodiments 64-70, wherein the siRNA molecule or the pharmaceutical composition is administered to the subject by way of intracerebroventricular, intrastriatal, intraparenchymal, or intrathecal injection.
Embodiment 72. The method of any one of embodiments 64-71, wherein the siRNA molecule or the pharmaceutical composition is administered to the subject by way of intravenous, intramuscular, or subcutaneous injection.
Embodiment 73. The method of any one of embodiments 64-72, wherein the subject is a human.
Embodiment 74. A kit comprising the siRNA molecule of any one of embodiments 1-62, or the pharmaceutical composition of embodiment 63, and a package insert, wherein the package insert instructs a user of the kit to perform the method of any one of embodiments 64-73.

### 7. EXAMPLES

The following examples are put forth so as to provide those of ordinary skill in the art with a description of how the compositions and methods described herein may be used, made, and evaluated, and are intended to be purely exemplary of the disclosure and are not intended to limit the scope of what the inventors regard as their disclosure

### Example 1. In Vitro Screening

In vitro screening was performed using human MeWo cells in a 96-well format. Briefly, cholesterol-conjugated, mono-siRNA was passively administered to cells in culture for 72 hours at 2 uM or 0.5 uM concentrations (single point format) or at serial 1:2.5 dilutions from 3 uM to 0.00196608 uM (IC50 format). Cells were lysed using the Cells-to-Ct kit (Thermo) and quantitative reverse transcriptase polymerase change reaction (QRT-PCR) was performed to assess target (SNCA) gene expression relative to housekeeping (ATP5B) gene expression using TaqMan primer/probes and Fast Advanced master mix (Thermo). Data are presented as mean % SNCA target expression (N=2-3 biological replicates, 2 technical replicates each) relative to untreated control cells, where 100% represents no knockdown and 0% represents complete target knockdown.

TABLE 2
In Vitro Screening

| | | | | | | | % UNT | | % UNT | | |
|---|---|---|---|---|---|---|---|---|---|---|---|
| | | | | | | | SNCA | | SNCA | | |
| | | | | | | | mRNA | | mRNA | | |
| | Posi- | 21 mer | | | | | ex- | | ex- | | |
| | tion | anti- | | 16 mer | | In | pres- | | pres- | | |
| | in | sense | SEQ | sense | SEQ | vitro | sion | | sion | | |
| | NM_ | se- | ID | se- | ID | duplex | @ | ST | @ | ST | IC50 |
| Name | 00345.3 | quence | NO. | quence | NO. | ID# | 2 uM | DEV | 0.5 uM | DEV | (nM) |
| SNCA_ | 166 | UAGGCU | 1 | CUUGCC | 421 | AT1146- | 60.79 | 0.65 | 86.06 | 1.65 | |
| 166 | | UGAAGG | | UUCAAG | | 1147 | | | | | |
| | | CAAGGC | | CCUA | | | | | | | |
| | | GUG | | | | | | | | | |
| SNCA_ | 171 | UGCAGA | 2 | CUUCAA | 422 | AT1148- | 90.93 | 5.08 | 108.60 | 6.30 | |
| 171 | | AGGCUU | | GCCUUC | | 1149 | | | | | |
| | | GAAGGC | | UGCA | | | | | | | |
| | | AAG | | | | | | | | | |
| SNCA_ | 173 | UAGGCA | 3 | UCAAGC | 423 | AT1150- | 85.96 | 1.71 | 108.24 | 2.21 | |
| 173 | | GAAGGC | | CUUCUG | | 1151 | | | | | |
| | | UUGAAG | | CCUA | | | | | | | |
| | | GCA | | | | | | | | | |
| SNCA_ | 174 | UAAGGC | 4 | CAAGCC | 424 | AT1098- | 34.26 | 1.08 | | | |
| 174 | | AGAAGG | | UUCUGC | | 1099 | | | | | |
| | | CUUGAA | | CUUA | | | | | | | |
| | | GGC | | | | | | | | | |
| SNCA_ | 175 | UAAAGG | 5 | AAGCCU | 425 | AT1152- | 93.45 | 0.11 | 120.23 | 1.66 | |
| 175 | | CAGAAG | | UCUGCC | | 1153 | | | | | |
| | | GCUUGA | | UUUA | | | | | | | |
| | | AGG | | | | | | | | | |
| SNCA_ | 176 | UGAAAG | 6 | AGCCUU | 426 | AT1154- | 205.29 | 2.58 | 111.23 | 2.90 | |
| 176 | | GCAGAA | | CUGCCU | | 1155 | | | | | |
| | | GGCUUG | | UUCA | | | | | | | |
| | | AAG | | | | | | | | | |
| SNCA_ | 178 | UUGGAA | 7 | CCUUCU | 427 | AT1156- | 94.92 | 2.50 | 90.56 | 0.32 | |
| 178 | | AGGCAG | | GCCUUU | | 1157 | | | | | |
| | | AAGGCU | | CCAA | | | | | | | |
| | | UGA | | | | | | | | | |
| SNCA_ | 179 | UGUGGA | 8 | CUUCUG | 428 | AT1100- | 56.88 | 0.11 | | | |
| 179 | | AAGGCA | | CCUUUC | | 1101 | | | | | |
| | | GAAGGC | | CACA | | | | | | | |
| | | UUG | | | | | | | | | |
| SNCA_ | 187 | UUCACG | 9 | UUUCCA | 429 | AT1102- | 69.08 | 1.96 | | | |
| 187 | | AGGGUG | | CCCUCG | | 1103 | | | | | |
| | | GAAAGG | | UGAA | | | | | | | |
| | | CAG | | | | | | | | | |
| SNCA_ | 189 | UGCUCA | 10 | UCCACC | 430 | AT1158- | 94.08 | 1.55 | 104.85 | 0.21 | |
| 189 | | CGAGGG | | CUCGUG | | 1159 | | | | | |
| | | UGGAAA | | AGCA | | | | | | | |
| | | GGC | | | | | | | | | |
| SNCA_ | 217 | UGUCGU | 11 | UGGCCA | 431 | AT1160- | 65.34 | 0.43 | 92.52 | 2.97 | |
| 217 | | CGAAUG | | UUCGAC | | 1161 | | | | | |
| | | GCCACU | | GACA | | | | | | | |
| | | CCC | | | | | | | | | |
| SNCA_ | 234 | UUUCCU | 12 | UGUGGU | 432 | AT1162- | 23.57 | 0.09 | 57.33 | 0.85 | 716 |
| 234 | | UUACAC | | GUAAAG | | 1163 | | | | | |
| | | CACACU | | GAAA | | | | | | | |
| | | GUC | | | | | | | | | |
| SNCA_ | 235 | UAUUCC | 13 | GUGGUG | 433 | AT2116- | 106.19 | 4.42 | 105.07 | 5.23 | |
| 235 | | UUUACA | | UAAAGG | | 2117 | | | | | |
| | | CCACAC | | AAUA | | | | | | | |
| | | UGU | | | | | | | | | |
| SNCA_ | 236 | UAAUUC | 14 | UGGUGU | 434 | AT2118- | 47.75 | 2.11 | 72.51 | 6.30 | |
| 236 | | CUUUAC | | AAAGGA | | 2119 | | | | | |
| | | ACCACA | | AUUA | | | | | | | |
| | | CUG | | | | | | | | | |
| SNCA_ | 239 | UAUGAA | 15 | UGUAAA | 435 | AT2120- | 64.98 | 6.66 | 77.69 | 9.36 | |
| 239 | | UUCCUU | | GGAAUU | | 2121 | | | | | |
| | | UACACC | | CAUA | | | | | | | |
| | | ACA | | | | | | | | | |
| SNCA_ | 240 | UAAUGA | 16 | GUAAAG | 436 | AT2122- | 48.02 | 3.20 | 71.47 | 9.83 | |
| 240 | | AUUCCU | | GAAUUC | | 2123 | | | | | |
| | | UUACAC | | AUUA | | | | | | | |
| | | CAC | | | | | | | | | |
| SNCA_ | 241 | UUAAUG | 17 | UAAAGG | 437 | AT1164- | 35.89 | 1.58 | 58.00 | 1.56 | |
| 241 | | AAUUCC | | AAUUCA | | 1165 | | | | | |
| | | UUUACA | | UUAA | | | | | | | |
| | | CCA | | | | | | | | | |
| SNCA_ | 242 | UCUAAU | 18 | AAAGGA | 438 | AT1166- | 21.20 | 0.14 | 47.77 | 0.27 | 457 |
| 242 | | GAAUUC | | AUUCAU | | 1167 | | | | | |
| | | CUUUAC | | UAGA | | | | | | | |
| | | ACC | | | | | | | | | |
| SNCA_ | 243 | UGCUAA | 19 | AAGGAA | 439 | AT2124- | 63.15 | 6.66 | 96.76 | 0.17 | |
| 243 | | UGAAUU | | UUCAUU | | 2125 | | | | | |
| | | CCUUUA | | AGCA | | | | | | | |
| | | CAC | | | | | | | | | |
| SNCA_ | 244 | UGGCUA | 20 | AGGAAU | 440 | AT2126- | 75.07 | 6.61 | 92.32 | 15.47 | |
| 244 | | AUGAAU | | UCAUUA | | 2127 | | | | | |
| | | UCCUUU | | GCCA | | | | | | | |
| | | ACA | | | | | | | | | |
| SNCA_ | 245 | UUGGCU | 21 | GGAAUU | 441 | AT2128- | 77.66 | 4.49 | 95.06 | 8.84 | |
| 245 | | AAUGAA | | CAUUAG | | 2129 | | | | | |
| | | UUCCUU | | CCAA | | | | | | | |
| | | UAC | | | | | | | | | |
| SNCA_ | 246 | UAUGGC | 22 | GAAUUC | 442 | AT1650- | 58.23 | 0.66 | 90.15 | 1.29 | |
| 246 | | UAAUGA | | AUUAGC | | 1651 | | | | | |
| | | AUUCCU | | CAUA | | | | | | | |
| | | UUA | | | | | | | | | |
| SNCA_ | 249 | UUCCAU | 23 | UUCAUU | 443 | AT1168- | 63.13 | 0.70 | 79.57 | 1.16 | |
| 249 | | GGCUAA | | AGCCAU | | 1169 | | | | | |
| | | UGAAUU | | GGAA | | | | | | | |
| | | CCU | | | | | | | | | |
| SNCA_ | 250 | UAUCCA | 24 | UCAUUA | 444 | AT1170- | 54.85 | 1.88 | 85.68 | 0.63 | |
| 250 | | UGGCUA | | GCCAUG | | 1171 | | | | | |
| | | AUGAAU | | GAUA | | | | | | | |
| | | UCC | | | | | | | | | |
| SNCA_ | 253 | UUACAU | 25 | UUAGCC | 445 | AT2130- | 64.82 | 6.79 | 93.12 | 15.20 | |
| 253 | | CCAUGG | | AUGGAU | | 2131 | | | | | |
| | | CUAAUG | | GUAA | | | | | | | |
| | | AAU | | | | | | | | | |
| SNCA_ | 254 | UAUACA | 26 | UAGCCA | 446 | AT2132- | 66.27 | 5.24 | 87.78 | 0.06 | |
| 254 | | UCCAUG | | UGGAUG | | 2133 | | | | | |
| | | GCUAAU | | UAUA | | | | | | | |
| | | GAA | | | | | | | | | |
| SNCA_ | 255 | UAAUAC | 27 | AGCCAU | 447 | AT2134- | 116.47 | 0.80 | 105.35 | 11.75 | |
| 255 | | AUCCAU | | GGAUGU | | 2135 | | | | | |
| | | GGCUAA | | AUUA | | | | | | | |
| | | UGA | | | | | | | | | |
| SNCA_ | 256 | UGAAUA | 28 | GCCAUG | 448 | AT2136- | 119.83 | 5.61 | 106.24 | 12.00 | |
| 256 | | CAUCCA | | GAUGUA | | 2137 | | | | | |
| | | UGGCUA | | UUCA | | | | | | | |
| | | AUG | | | | | | | | | |
| SNCA_ | 257 | UUGAAU | 29 | CCAUGG | 449 | AT1104- | 40.90 | 1.33 | | | |
| 257 | | ACAUCC | | AUGUAU | | 1105 | | | | | |
| | | AUGGCU | | UCAA | | | | | | | |
| | | AAU | | | | | | | | | |
| SNCA_ | 258 | UAUGAA | 30 | CAUGGA | 450 | AT2138- | 46.61 | 0.29 | 74.07 | 2.07 | |
| 258 | | UACAUC | | UGUAUU | | 2139 | | | | | |
| | | CAUGGC | | CAUA | | | | | | | |
| | | UAA | | | | | | | | | |
| SNCA_ | 259 | UCAUGA | 31 | AUGGAU | 451 | AT2140- | 136.51 | 1.61 | 112.33 | 6.00 | |
| 259 | | AUACAU | | GUAUUC | | 2141 | | | | | |
| | | CCAUGG | | AUGA | | | | | | | |
| | | CUA | | | | | | | | | |
| SNCA_ | 260 | UUCAUG | 32 | UGGAUG | 452 | AT1172- | 31.88 | 0.22 | 50.79 | 0.08 | |
| 260 | | AAUACA | | UAUUCA | | 1173 | | | | | |
| | | UCCAUG | | UGAA | | | | | | | |
| | | GCU | | | | | | | | | |
| SNCA_ | 261 | UUUCAU | 33 | GGAUGU | 453 | AT1106- | 50.77 | 2.25 | | | |
| 261 | | GAAUAC | | AUUCAU | | 1107 | | | | | |
| | | AUCCAU | | GAAA | | | | | | | |
| | | GGC | | | | | | | | | |
| SNCA_ | 262 | UUUUCA | 34 | GAUGUA | 454 | AT2142- | 100.59 | 2.74 | 67.02 | 1.17 | |
| 262 | | UGAAUA | | UUCAUG | | 2143 | | | | | |
| | | CAUCCA | | AAAA | | | | | | | |
| | | UGG | | | | | | | | | |
| SNCA_ | 266 | UGUCCU | 35 | UAUUCA | 455 | AT2144- | 37.55 | 1.05 | 98.53 | 1.33 | |
| 266 | | UUCAUG | | UGAAAG | | 2145 | | | | | |
| | | AAUACA | | GACA | | | | | | | |
| | | UCC | | | | | | | | | |
| SNCA_ | 267 | UAGUCC | 36 | AUUCAU | 456 | AT2146- | 59.67 | 0.53 | 104.29 | 2.02 | |
| 267 | | UUUCAU | | GAAAGG | | 2147 | | | | | |
| | | GAAUAC | | ACUA | | | | | | | |
| | | AUC | | | | | | | | | |
| SNCA_ | 268 | UAAGUC | 37 | UUCAUG | 457 | AT2148- | 81.69 | 0.62 | 105.51 | 4.50 | |
| 268 | | CUUUCA | | AAAGGA | | 2149 | | | | | |
| | | UGAAUA | | CUUA | | | | | | | |
| | | CAU | | | | | | | | | |
| SNCA_ | 269 | UAAAGU | 38 | UCAUGA | 458 | AT1174- | 25.70 | 0.08 | 45.89 | 0.62 | 708 |
| 269 | | CCUUUC | | AAGGAC | | 1175 | | | | | |
| | | AUGAAU | | UUUA | | | | | | | |
| | | ACA | | | | | | | | | |
| SNCA_ | 270 | UGAAAG | 39 | CAUGAA | 459 | AT1602- | 14.69 | 0.57 | 60.98 | 0.58 | 628 |
| 270 | | UCCUUU | | AGGACU | | 1603 | | | | | |
| | | CAUGAA | | UUCA | | | | | | | |
| | | UAC | | | | | | | | | |
| SNCA_ | 273 | UUUUGA | 40 | GAAAGG | 460 | AT1604- | 57.37 | 0.00 | 78.50 | 0.22 | |
| 273 | | AAGUCC | | ACUUUC | | 1605 | | | | | |
| | | UUUCAU | | AAAA | | | | | | | |
| | | GAA | | | | | | | | | |
| SNCA_ | 274 | UCUUUG | 41 | AAAGGA | 461 | AT2150- | 86.36 | 4.36 | 114.74 | 6.41 | |
| 274 | | AAAGUC | | CUUUCA | | 2151 | | | | | |
| | | CUUUCA | | AAGA | | | | | | | |
| | | UGA | | | | | | | | | |
| SNCA_ | 275 | UCCUUU | 42 | AAGGAC | 462 | AT2152- | 102.04 | 3.18 | 114.68 | 10.27 | |
| 275 | | GAAAGU | | UUUCAA | | 2153 | | | | | |
| | | CCUUUC | | AGGA | | | | | | | |
| | | AUG | | | | | | | | | |
| SNCA_ | 276 | UGCCUU | 43 | AGGACU | 463 | AT2154- | 133.52 | 1.90 | 106.61 | 11.42 | |
| 276 | | UGAAAG | | UUCAAA | | 2155 | | | | | |
| | | UCCUUU | | GGCA | | | | | | | |
| | | CAU | | | | | | | | | |
| SNCA_ | 277 | UGGCCU | 44 | GGACUU | 464 | AT1630- | 88.34 | 0.35 | 100.13 | 2.14 | |
| 277 | | UUGAAA | | UCAAAG | | 1631 | | | | | |
| | | GUCCUU | | GCCA | | | | | | | |
| | | UCA | | | | | | | | | |
| SNCA_ | 278 | UUGGCC | 45 | GACUUU | 465 | AT2156- | 119.41 | 6.64 | 100.60 | 8.08 | |
| 278 | | UUUGAA | | CAAAGG | | 2157 | | | | | |
| | | AGUCCU | | CCAA | | | | | | | |
| | | UUC | | | | | | | | | |
| SNCA_ | 279 | UUUGGC | 46 | ACUUUC | 466 | AT1632- | 66.81 | 1.50 | 85.78 | 0.42 | |
| 279 | | CUUUGA | | AAAGGC | | 1633 | | | | | |
| | | AAGUCC | | CAAA | | | | | | | |
| | | UUU | | | | | | | | | |
| SNCA_ | 280 | UCUUGG | 47 | CUUUCA | 467 | AT2158- | 95.46 | 0.28 | 104.10 | 2.86 | |
| 280 | | CCUUUG | | AAGGCC | | 2159 | | | | | |
| | | AAAGUC | | AAGA | | | | | | | |
| | | CUU | | | | | | | | | |
| SNCA_ | 281 | UCCUUG | 48 | UUUCAA | 468 | AT2160- | 116.94 | 0.49 | 107.85 | 16.32 | |
| 281 | | GCCUUU | | AGGCCA | | 2161 | | | | | |
| | | GAAAGU | | AGGA | | | | | | | |
| | | CCU | | | | | | | | | |
| SNCA_ | 282 | UUCCUU | 49 | UUCAAA | 469 | AT1176- | 96.59 | 0.28 | 82.66 | 2.56 | |
| 282 | | GGCCUU | | GGCCAA | | 1177 | | | | | |
| | | UGAAAG | | GGAA | | | | | | | |
| | | UCC | | | | | | | | | |
| SNCA_ | 283 | UCUCCU | 50 | UCAAAG | 470 | AT1108- | 108.76 | 3.86 | | | |
| 283 | | UGGCCU | | GCCAAG | | 1109 | | | | | |
| | | UUGAAA | | GAGA | | | | | | | |
| | | GUC | | | | | | | | | |
| SNCA_ | 284 | UCCUCC | 51 | CAAAGG | 471 | AT2162- | 140.69 | 0.97 | 115.22 | 8.74 | |
| 284 | | UUGGCC | | CCAAGG | | 2163 | | | | | |
| | | UUUGAA | | AGGA | | | | | | | |
| | | AGU | | | | | | | | | |
| SNCA_ | 285 | UCCCUC | 52 | AAAGGC | 472 | AT2164- | 185.56 | 7.86 | 143.88 | 11.73 | |
| 285 | | CUUGGC | | CAAGGA | | 2165 | | | | | |
| | | CUUUGA | | GGGA | | | | | | | |
| | | AAG | | | | | | | | | |
| SNCA_ | 286 | UUCCCU | 53 | AAGGCC | 473 | AT2166- | 110.71 | 0.62 | 105.21 | 11.12 | |
| 286 | | CCUUGG | | AAGGAG | | 2167 | | | | | |
| | | CCUUUG | | GGAA | | | | | | | |
| | | AAA | | | | | | | | | |
| SNCA_ | 287 | UCUCCC | 54 | AGGCCA | 474 | AT1634- | 107.71 | 0.58 | 85.74 | 1.09 | |
| 287 | | UCCUUG | | AGGAGG | | 1635 | | | | | |
| | | GCCUUU | | GAGA | | | | | | | |
| | | GAA | | | | | | | | | |
| SNCA_ | 288 | UACUCC | 55 | GGCCAA | 475 | AT2168- | 106.40 | 5.58 | 135.71 | 0.13 | |
| 288 | | CUCCUU | | GGAGGG | | 2169 | | | | | |
| | | GGCCUU | | AGUA | | | | | | | |
| | | UGA | | | | | | | | | |
| SNCA_ | 289 | UAACUC | 56 | GCCAAG | 476 | AT2170- | 96.56 | 1.04 | 133.40 | 6.01 | |
| 289 | | CCUCCU | | GAGGGA | | 2171 | | | | | |
| | | UGGCCU | | GUUA | | | | | | | |
| | | UUG | | | | | | | | | |
| SNCA_ | 290 | UCAACU | 57 | CCAAGG | 477 | AT1606- | 48.85 | 0.83 | 92.41 | 0.81 | |
| 290 | | CCCUCC | | AGGGAG | | 1607 | | | | | |
| | | UUGGCC | | UUGA | | | | | | | |
| | | UUU | | | | | | | | | |
| SNCA_ | 291 | UACAAC | 58 | CAAGGA | 478 | AT2172- | 80.42 | 2.58 | 124.00 | 2.22 | |
| 291 | | UCCCUC | | GGGAGU | | 2173 | | | | | |
| | | CUUGGC | | UGUA | | | | | | | |
| | | CUU | | | | | | | | | |
| SNCA_ | 292 | UCACAA | 59 | AAGGAG | 479 | AT2174- | 108.17 | 1.72 | 137.72 | 0.84 | |
| 292 | | CUCCCU | | GGAGUU | | 2175 | | | | | |
| | | CCUUGG | | GUGA | | | | | | | |
| | | CCU | | | | | | | | | |
| SNCA_ | 293 | UCCACA | 60 | AGGAGG | 480 | AT2176- | 96.88 | 2.94 | 134.51 | 7.84 | |
| 293 | | ACUCCC | | GAGUUG | | 2177 | | | | | |
| | | UCCUUG | | UGGA | | | | | | | |
| | | GCC | | | | | | | | | |
| SNCA_ | 294 | UGCCAC | 61 | GGAGGG | 481 | AT2178- | 59.87 | 4.59 | 108.13 | 12.66 | |
| 294 | | AACUCC | | AGUUGU | | 2179 | | | | | |
| | | CUCCUU | | GGCA | | | | | | | |
| | | GGC | | | | | | | | | |
| SNCA_ | 295 | UAGCCA | 62 | GAGGGA | 482 | AT2180- | 97.08 | 0.02 | 118.65 | 11.58 | |
| 295 | | CAACUC | | GUUGUG | | 2181 | | | | | |
| | | CCUCCU | | GCUA | | | | | | | |
| | | UGG | | | | | | | | | |
| SNCA_ | 296 | UCAGCC | 63 | AGGGAG | 483 | AT2182- | 104.02 | 5.15 | 114.71 | 5.20 | |
| 296 | | ACAACU | | UUGUGG | | 2183 | | | | | |
| | | CCCUCC | | CUGA | | | | | | | |
| | | UUG | | | | | | | | | |
| SNCA_ | 297 | UGCAGC | 64 | GGGAGU | 484 | AT2184- | 74.38 | 5.15 | 117.53 | 4.32 | |
| 297 | | CACAAC | | UGUGGC | | 2185 | | | | | |
| | | UCCCUC | | UGCA | | | | | | | |
| | | CUU | | | | | | | | | |
| SNCA_ | 298 | UAGCAG | 65 | GGAGUU | 485 | AT1178- | 40.32 | 0.22 | 64.27 | 2.23 | |
| 298 | | CCACAA | | GUGGCU | | 1179 | | | | | |
| | | CUCCCU | | GCUA | | | | | | | |
| | | CCU | | | | | | | | | |
| SNCA_ | 299 | UCAGCA | 66 | GAGUUG | 486 | AT2186- | 83.82 | 1.70 | 102.30 | 19.14 | |
| 299 | | GCCACA | | UGGCUG | | 2187 | | | | | |
| | | ACUCCC | | CUGA | | | | | | | |
| | | UCC | | | | | | | | | |
| SNCA_ | 300 | UGCAGC | 67 | AGUUGU | 487 | AT1180- | 70.62 | 1.19 | 90.22 | 1.33 | |
| 300 | | AGCCAC | | GGCUGC | | 1181 | | | | | |
| | | AACUCC | | UGCA | | | | | | | |
| | | CUC | | | | | | | | | |
| SNCA_ | 301 | UAGCAG | 68 | GUUGUG | 488 | AT2188- | 77.56 | 8.86 | 119.96 | 11.48 | |
| 301 | | CAGCCA | | GCUGCU | | 2189 | | | | | |
| | | CAACUC | | GCUA | | | | | | | |
| | | CCU | | | | | | | | | |
| SNCA_ | 302 | UCAGCA | 69 | UUGUGG | 489 | AT2190- | 87.50 | 7.88 | 121.88 | 14.18 | |
| 302 | | GCAGCC | | CUGCUG | | 2191 | | | | | |
| | | ACAACU | | CUGA | | | | | | | |
| | | CCC | | | | | | | | | |
| SNCA_ | 303 | UUCAGC | 70 | UGUGGC | 490 | AT2192- | 49.26 | 3.64 | 96.78 | 5.83 | 695 |
| 303 | | AGCAGC | | UGCUGC | | 2193 | | | | | |
| | | CACAAC | | UGAA | | | | | | | |
| | | UCC | | | | | | | | | |
| SNCA_ | 304 | UCUCAG | 71 | GUGGCU | 491 | AT1608- | 93.46 | 0.57 | 99.24 | 3.63 | |
| 304 | | CAGCAG | | GCUGCU | | 1609 | | | | | |
| | | CCACAA | | GAGA | | | | | | | |
| | | CUC | | | | | | | | | |
| SNCA_ | 305 | UUCUCA | 72 | UGGCUG | 492 | AT2194- | 118.36 | 9.27 | 143.32 | 0.11 | |
| 305 | | GCAGCA | | CUGCUG | | 2195 | | | | | |
| | | GCCACA | | AGAA | | | | | | | |
| | | ACU | | | | | | | | | |
| SNCA_ | 306 | UUUCUC | 73 | GGCUGC | 493 | AT2196- | 88.32 | 1.45 | 123.83 | 6.95 | |
| 306 | | AGCAGC | | UGCUGA | | 2197 | | | | | |
| | | AGCCAC | | GAAA | | | | | | | |
| | | AAC | | | | | | | | | |
| SNCA_ | 307 | UUUUCU | 74 | GCUGCU | 494 | AT1610- | 37.30 | 0.71 | 75.14 | 1.14 | |
| 307 | | CAGCAG | | GCUGAG | | 1611 | | | | | |
| | | CAGCCA | | AAAA | | | | | | | |
| | | CAA | | | | | | | | | |
| SNCA_ | 309 | UGUUUU | 75 | UGCUGC | 495 | AT2198- | 84.47 | 6.37 | 116.01 | 16.74 | |
| 309 | | CUCAGC | | UGAGAA | | 2199 | | | | | |
| | | AGCAGC | | AACA | | | | | | | |
| | | CAC | | | | | | | | | |
| SNCA_ | 310 | UGGUUU | 76 | GCUGCU | 496 | AT1182- | 57.16 | 0.04 | 85.79 | 0.89 | |
| 310 | | UCUCAG | | GAGAAA | | 1183 | | | | | |
| | | CAGCAG | | ACCA | | | | | | | |
| | | CCA | | | | | | | | | |
| SNCA_ | 311 | UUGGUU | 77 | CUGCUG | 497 | AT1184- | 72.29 | 0.68 | 77.04 | 0.49 | |
| 311 | | UUCUCA | | AGAAAA | | 1185 | | | | | |
| | | GCAGCA | | CCAA | | | | | | | |
| | | GCC | | | | | | | | | |
| SNCA_ | 312 | UUUGGU | 78 | UGCUGA | 498 | AT1636- | 123.35 | 6.80 | 91.92 | 0.09 | |
| 312 | | UUUCUC | | GAAAAC | | 1637 | | | | | |
| | | AGCAGC | | CAAA | | | | | | | |
| | | AGC | | | | | | | | | |
| SNCA_ | 313 | UUUUGG | 79 | GCUGAG | 499 | AT1186- | 34.99 | 0.95 | 66.13 | 1.19 | |
| 313 | | UUUUCU | | AAAACC | | 1187 | | | | | |
| | | CAGCAG | | AAAA | | | | | | | |
| | | CAG | | | | | | | | | |
| SNCA_ | 314 | UGUUUG | 80 | CUGAGA | 500 | AT2200- | 85.71 | 0.50 | 112.04 | 10.25 | |
| 314 | | GUUUUC | | AAACCA | | 2201 | | | | | |
| | | UCAGCA | | AACA | | | | | | | |
| | | GCA | | | | | | | | | |
| SNCA_ | 315 | UUGUUU | 81 | UGAGAA | 501 | AT1188- | 24.68 | 0.55 | 83.60 | 0.88 | 59500 |
| 315 | | GGUUUU | | AACCAA | | 1189 | | | | | |
| | | CUCAGC | | ACAA | | | | | | | |
| | | AGC | | | | | | | | | |
| SNCA_ | 316 | UCUGUU | 82 | GAGAAA | 502 | AT1190- | 49.23 | 2.88 | 79.77 | 0.13 | |
| 316 | | UGGUUU | | ACCAAA | | 1191 | | | | | |
| | | UCUCAG | | CAGA | | | | | | | |
| | | CAG | | | | | | | | | |
| SNCA_ | 317 | UCCUGU | 83 | AGAAAA | 503 | AT2202- | 71.67 | 7.80 | 95.59 | 11.99 | |
| 317 | | UUGGUU | | CCAAAC | | 2203 | | | | | |
| | | UUCUCA | | AGGA | | | | | | | |
| | | GCA | | | | | | | | | |
| SNCA_ | 318 | UCCCUG | 84 | GAAAAC | 504 | AT2204- | 69.74 | 5.84 | 95.32 | 1.47 | |
| 318 | | UUUGGU | | CAAACA | | 2205 | | | | | |
| | | UUUCUC | | GGGA | | | | | | | |
| | | AGC | | | | | | | | | |
| SNCA_ | 324 | UGCCAC | 85 | CAAACA | 505 | AT1192- | 60.09 | 0.35 | 74.69 | 1.87 | |
| 324 | | ACCCUG | | GGGUGU | | 1193 | | | | | |
| | | UUUGGU | | GGCA | | | | | | | |
| | | UUU | | | | | | | | | |
| SNCA_ | 325 | UUGCCA | 86 | AAACAG | 506 | AT2206- | 121.83 | 6.95 | 131.25 | 1.25 | |
| 325 | | CACCCU | | GGUGUG | | 2207 | | | | | |
| | | GUUUGG | | GCAA | | | | | | | |
| | | UUU | | | | | | | | | |
| SNCA_ | 326 | UCUGCC | 87 | AACAGG | 507 | AT2208- | 111.67 | 4.13 | 120.59 | 13.95 | |
| 326 | | ACACCC | | GUGUGG | | 2209 | | | | | |
| | | UGUUUG | | CAGA | | | | | | | |
| | | GUU | | | | | | | | | |
| SNCA_ | 327 | UUCUGC | 88 | ACAGGG | 508 | AT1194- | 48.13 | 5.53 | 72.80 | 1.86 | |
| 327 | | CACACC | | UGUGGC | | 1195 | | | | | |
| | | CUGUUU | | AGAA | | | | | | | |
| | | GGU | | | | | | | | | |
| SNCA_ | 328 | UUUCUG | 89 | CAGGGU | 509 | AT2210- | 103.50 | 4.11 | 107.86 | 2.43 | |
| 328 | | CCACAC | | GUGGCA | | 2211 | | | | | |
| | | CCUGUU | | GAAA | | | | | | | |
| | | UGG | | | | | | | | | |
| SNCA_ | 341 | UUCUUU | 90 | AAGCAG | 510 | AT1196- | 94.67 | 2.25 | 103.83 | 0.76 | |
| 341 | | CCUGCU | | CAGGAA | | 1197 | | | | | |
| | | GCUUCU | | AGAA | | | | | | | |
| | | GCC | | | | | | | | | |
| SNCA_ | 347 | UCUUUU | 91 | CAGGAA | 511 | AT2212- | 99.20 | 2.50 | 104.16 | 0.18 | |
| 347 | | GUCUUU | | AGACAA | | 2213 | | | | | |
| | | CCUGCU | | AAGA | | | | | | | |
| | | GCU | | | | | | | | | |
| SNCA_ | 348 | UUCUUU | 92 | AGGAAA | 512 | AT2214- | 96.36 | 0.57 | 103.60 | 6.70 | |
| 348 | | UGUCUU | | GACAAA | | 2215 | | | | | |
| | | UCCUGC | | AGAA | | | | | | | |
| | | UGC | | | | | | | | | |
| SNCA_ | 349 | UCUCUU | 93 | GGAAAG | 513 | AT1198- | 23.99 | 0.14 | 59.54 | 1.15 | 721 |
| 349 | | UUGUCU | | ACAAAA | | 1199 | | | | | |
| | | UUCCUG | | GAGA | | | | | | | |
| | | CUG | | | | | | | | | |
| SNCA_ | 350 | UCCUCU | 94 | GAAAGA | 514 | AT2216- | 63.26 | 0.14 | 88.33 | 1.84 | |
| 350 | | UUUGUC | | CAAAAG | | 2217 | | | | | |
| | | UUUCCU | | AGGA | | | | | | | |
| | | GCU | | | | | | | | | |
| SNCA_ | 351 | UCCCUC | 95 | AAAGAC | 515 | AT2218- | 75.11 | 5.81 | 114.33 | 10.69 | |
| 351 | | UUUUGU | | AAAAGA | | 2219 | | | | | |
| | | CUUUCC | | GGGA | | | | | | | |
| | | UGC | | | | | | | | | |
| SNCA_ | 352 | UACCCU | 96 | AAGACA | 516 | AT2220- | 105.91 | 3.04 | 106.42 | 7.35 | |
| 352 | | CUUUUG | | AAAGAG | | 2221 | | | | | |
| | | UCUUUC | | GGUA | | | | | | | |
| | | CUG | | | | | | | | | |
| SNCA_ | 354 | UACACC | 97 | GACAAA | 517 | AT2222- | 71.70 | 0.16 | 97.99 | 5.66 | |
| 354 | | CUCUUU | | AGAGGG | | 2223 | | | | | |
| | | UGUCUU | | UGUA | | | | | | | |
| | | UCC | | | | | | | | | |
| SNCA_ | 355 | UAACAC | 98 | ACAAAA | 518 | AT1200- | 22.37 | 0.20 | 49.96 | 2.04 | 394 |
| 355 | | CCUCUU | | GAGGGU | | 1201 | | | | | |
| | | UUGUCU | | GUUA | | | | | | | |
| | | UUC | | | | | | | | | |
| SNCA_ | 356 | UGAACA | 99 | CAAAAG | 519 | AT2224- | 34.44 | 1.66 | 71.19 | 7.02 | |
| 356 | | CCCUCU | | AGGGUG | | 2225 | | | | | |
| | | UUUGUC | | UUCA | | | | | | | |
| | | UUU | | | | | | | | | |
| SNCA_ | 357 | UAGAAC | 100 | AAAAGA | 520 | AT2226- | 34.50 | 0.86 | 70.23 | 3.70 | |
| 357 | | ACCCUC | | GGGUGU | | 2227 | | | | | |
| | | UUUUGU | | UCUA | | | | | | | |
| | | CUU | | | | | | | | | |
| SNCA_ | 369 | UGAGCC | 101 | UCUCUA | 521 | AT2228- | 94.35 | 0.67 | 102.82 | 15.41 | |
| 369 | | UACAUA | | UGUAGG | | 2229 | | | | | |
| | | GAGAAC | | CUCA | | | | | | | |
| | | ACC | | | | | | | | | |
| SNCA_ | 370 | UGGAGC | 102 | CUCUAU | 522 | AT2230- | 118.81 | 4.08 | 112.56 | 0.08 | |
| 370 | | CUACAU | | GUAGGC | | 2231 | | | | | |
| | | AGAGAA | | UCCA | | | | | | | |
| | | CAC | | | | | | | | | |
| SNCA_ | 371 | UUGGAG | 103 | UCUAUG | 523 | AT1202- | 78.33 | 0.46 | 95.33 | 1.48 | |
| 371 | | CCUACA | | UAGGCU | | 1203 | | | | | |
| | | UAGAGA | | CCAA | | | | | | | |
| | | ACA | | | | | | | | | |
| SNCA_ | 372 | UUUGGA | 104 | CUAUGU | 524 | AT1204- | 35.36 | 1.56 | 71.23 | 1.03 | |
| 372 | | GCCUAC | | AGGCUC | | 1205 | | | | | |
| | | AUAGAG | | CAAA | | | | | | | |
| | | AAC | | | | | | | | | |
| SNCA_ | 373 | UUUUGG | 105 | UAUGUA | 525 | AT2232- | 125.42 | 1.29 | 109.09 | 18.57 | |
| 373 | | AGCCUA | | GGCUCC | | 2233 | | | | | |
| | | CAUAGA | | AAAA | | | | | | | |
| | | GAA | | | | | | | | | |
| SNCA_ | 375 | UGUUUU | 106 | UGUAGG | 526 | AT2234- | 137.15 | 1.41 | 108.07 | 16.93 | |
| 375 | | GGAGCC | | CUCCAA | | 2235 | | | | | |
| | | UACAUA | | AACA | | | | | | | |
| | | GAG | | | | | | | | | |
| SNCA_ | 380 | UCCUUG | 107 | GCUCCA | 527 | AT2236- | 109.37 | 2.55 | 102.44 | 5.29 | |
| 380 | | GUUUUG | | AAACCA | | 2237 | | | | | |
| | | GAGCCU | | AGGA | | | | | | | |
| | | ACA | | | | | | | | | |
| SNCA_ | 381 | UUCCUU | 108 | CUCCAA | 528 | AT2238- | 132.79 | 2.25 | 104.19 | 12.20 | |
| 381 | | GGUUUU | | AACCAA | | 2239 | | | | | |
| | | GGAGCC | | GGAA | | | | | | | |
| | | UAC | | | | | | | | | |
| SNCA_ | 382 | UCUCCU | 109 | UCCAAA | 529 | AT1206- | 47.41 | 1.14 | 72.46 | 1.78 | |
| 382 | | UGGUUU | | ACCAAG | | 1207 | | | | | |
| | | UGGAGC | | GAGA | | | | | | | |
| | | CUA | | | | | | | | | |
| SNCA_ | 383 | UCCUCC | 110 | CCAAAA | 530 | AT2240- | 77.22 | 0.32 | 98.42 | 0.92 | |
| 383 | | UUGGUU | | CCAAGG | | 2241 | | | | | |
| | | UUGGAG | | AGGA | | | | | | | |
| | | CCU | | | | | | | | | |
| SNCA_ | 384 | UCCCUC | 111 | CAAAAC | 531 | AT2242- | 111.43 | 2.38 | 112.37 | 1.57 | |
| 384 | | CUUGGU | | CAAGGA | | 2243 | | | | | |
| | | UUUGGA | | GGGA | | | | | | | |
| | | GCC | | | | | | | | | |
| SNCA_ | 389 | UCCACU | 112 | CCAAGG | 532 | AT2244- | 87.32 | 5.00 | 102.25 | 8.66 | |
| 389 | | CCCUCC | | AGGGAG | | 2245 | | | | | |
| | | UUGGUU | | UGGA | | | | | | | |
| | | UUG | | | | | | | | | |
| SNCA_ | 397 | UACCAU | 113 | GGAGUG | 533 | AT2246- | 95.66 | 3.35 | 112.02 | 3.24 | |
| 397 | | GCACCA | | GUGCAU | | 2247 | | | | | |
| | | CUCCCU | | GGUA | | | | | | | |
| | | CCU | | | | | | | | | |
| SNCA_ | 398 | UCACCA | 114 | GAGUGG | 534 | AT2248- | 65.99 | 0.06 | 95.82 | 9.68 | |
| 398 | | UGCACC | | UGCAUG | | 2249 | | | | | |
| | | ACUCCC | | GUGA | | | | | | | |
| | | UCC | | | | | | | | | |
| SNCA_ | 399 | UACACC | 115 | AGUGGU | 535 | AT1208- | 19.38 | 0.10 | 57.90 | 0.20 | 1380 |
| 399 | | AUGCAC | | GCAUGG | | 1209 | | | | | |
| | | CACUCC | | UGUA | | | | | | | |
| | | CUC | | | | | | | | | |
| SNCA_ | 400 | UCACAC | 116 | GUGGUG | 536 | AT2250- | 117.38 | 2.85 | 114.61 | 11.72 | |
| 400 | | CAUGCA | | CAUGGU | | 2251 | | | | | |
| | | CCACUC | | GUGA | | | | | | | |
| | | CCU | | | | | | | | | |
| SNCA_ | 401 | UCCACA | 117 | UGGUGC | 537 | AT2252- | 85.46 | 3.87 | 101.55 | 14.24 | |
| 401 | | CCAUGC | | AUGGUG | | 2253 | | | | | |
| | | ACCACU | | UGGA | | | | | | | |
| | | CCC | | | | | | | | | |
| SNCA_ | 405 | UGUUGC | 118 | GCAUGG | 538 | AT2254- | 57.98 | 0.98 | 96.07 | 9.43 | |
| 405 | | CACACC | | UGUGGC | | 2255 | | | | | |
| | | AUGCAC | | AACA | | | | | | | |
| | | CAC | | | | | | | | | |
| SNCA_ | 406 | UUGUUG | 119 | CAUGGU | 539 | AT2256- | 48.00 | 2.52 | 82.00 | 4.16 | |
| 406 | | CCACAC | | GUGGCA | | 2257 | | | | | |
| | | CAUGCA | | ACAA | | | | | | | |
| | | CCA | | | | | | | | | |
| SNCA_ | 407 | UCUGUU | 120 | AUGGUG | 540 | AT1210- | 46.24 | 0.61 | 74.39 | 2.11 | |
| 407 | | GCCACA | | UGGCAA | | 1211 | | | | | |
| | | CCAUGC | | CAGA | | | | | | | |
| | | ACC | | | | | | | | | |
| SNCA_ | 408 | UACUGU | 121 | UGGUGU | 541 | AT2258- | 102.83 | 1.26 | 99.08 | 5.63 | |
| 408 | | UGCCAC | | GGCAAC | | 2259 | | | | | |
| | | ACCAUG | | AGUA | | | | | | | |
| | | CAC | | | | | | | | | |
| SNCA_ | 409 | UCACUG | 122 | GGUGUG | 542 | AT2260- | 97.41 | 1.65 | 100.50 | 15.82 | |
| 409 | | UUGCCA | | GCAACA | | 2261 | | | | | |
| | | CACCAU | | GUGA | | | | | | | |
| | | GCA | | | | | | | | | |
| SNCA_ | 413 | UCAGCC | 123 | UGGCAA | 543 | AT2262- | 93.29 | 4.84 | 103.94 | 6.85 | |
| 413 | | ACUGUU | | CAGUGG | | 2263 | | | | | |
| | | GCCACA | | CUGA | | | | | | | |
| | | CCA | | | | | | | | | |
| SNCA_ | 414 | UUCAGC | 124 | GGCAAC | 544 | AT2264- | 111.86 | 6.60 | 111.96 | 12.79 | |
| 414 | | CACUGU | | AGUGGC | | 2265 | | | | | |
| | | UGCCAC | | UGAA | | | | | | | |
| | | ACC | | | | | | | | | |
| SNCA_ | 415 | UCUCAG | 125 | GCAACA | 545 | AT1212- | 40.83 | 0.23 | 66.73 | 0.08 | |
| 415 | | CCACUG | | GUGGCU | | 1213 | | | | | |
| | | UUGCCA | | GAGA | | | | | | | |
| | | CAC | | | | | | | | | |
| SNCA_ | 416 | UUCUCA | 126 | CAACAG | 546 | AT2266- | 62.18 | 0.59 | 94.75 | 8.26 | |
| 416 | | GCCACU | | UGGCUG | | 2267 | | | | | |
| | | GUUGCC | | AGAA | | | | | | | |
| | | ACA | | | | | | | | | |
| SNCA_ | 417 | UUUCUC | 127 | AACAGU | 547 | AT2268- | 94.25 | 0.25 | 117.57 | 4.61 | |
| 417 | | AGCCAC | | GGCUGA | | 2269 | | | | | |
| | | UGUUGC | | GAAA | | | | | | | |
| | | CAC | | | | | | | | | |
| SNCA_ | 418 | UCUUCU | 128 | ACAGUG | 548 | AT1612- | 19.17 | 0.03 | 61.99 | 0.23 | 2130 |
| 418 | | CAGCCA | | GCUGAG | | 1613 | | | | | |
| | | CUGUUG | | AAGA | | | | | | | |
| | | CCA | | | | | | | | | |
| SNCA_ | 419 | UUCUUC | 129 | CAGUGG | 549 | AT1214- | 45.86 | 0.95 | 79.74 | 1.09 | |
| 419 | | UCAGCC | | CUGAGA | | 1215 | | | | | |
| | | ACUGUU | | AGAA | | | | | | | |
| | | GCC | | | | | | | | | |
| SNCA_ | 420 | UGUCUU | 130 | AGUGGC | 550 | AT2270- | 37.73 | 1.65 | 70.07 | 7.05 | |
| 420 | | CUCAGC | | UGAGAA | | 2271 | | | | | |
| | | CACUGU | | GACA | | | | | | | |
| | | UGC | | | | | | | | | |
| SNCA_ | 421 | UGGUCU | 131 | GUGGCU | 551 | AT1638- | 28.96 | 0.86 | 81.19 | 1.35 | 1810 |
| 421 | | UCUCAG | | GAGAAG | | 1639 | | | | | |
| | | CCACUG | | ACCA | | | | | | | |
| | | UUG | | | | | | | | | |
| SNCA_ | 422 | UUGGUC | 132 | UGGCUG | 552 | AT1640- | 57.17 | 1.91 | 74.20 | 1.02 | |
| 422 | | UUCUCA | | AGAAGA | | 1641 | | | | | |
| | | GCCACU | | CCAA | | | | | | | |
| | | GUU | | | | | | | | | |
| SNCA_ | 423 | UUUGGU | 133 | GGCUGA | 553 | AT2272- | 103.56 | 4.49 | 94.20 | 10.60 | |
| 423 | | CUUCUC | | GAAGAC | | 2273 | | | | | |
| | | AGCCAC | | CAAA | | | | | | | |
| | | UGU | | | | | | | | | |
| SNCA_ | 424 | UUUUGG | 134 | GCUGAG | 554 | AT2274- | 75.69 | 0.54 | 93.94 | 20.60 | |
| 424 | | UCUUCU | | AAGACC | | 2275 | | | | | |
| | | CAGCCA | | AAAA | | | | | | | |
| | | CUG | | | | | | | | | |
| SNCA_ | 425 | UCUUUG | 135 | CUGAGA | 555 | AT2276- | 81.93 | 4.29 | 109.95 | 7.78 | |
| 425 | | GUCUUC | | AGACCA | | 2277 | | | | | |
| | | UCAGCC | | AAGA | | | | | | | |
| | | ACU | | | | | | | | | |
| SNCA_ | 426 | UUCUUU | 136 | UGAGAA | 556 | AT1642- | 31.25 | 0.20 | 68.81 | 0.79 | 1360 |
| 426 | | GGUCUU | | GACCAA | | 1643 | | | | | |
| | | CUCAGC | | AGAA | | | | | | | |
| | | CAC | | | | | | | | | |
| SNCA_ | 427 | UCUCUU | 137 | GAGAAG | 557 | AT1216- | 37.55 | 0.36 | 77.93 | 1.33 | |
| 427 | | UGGUCU | | ACCAAA | | 1217 | | | | | |
| | | UCUCAG | | GAGA | | | | | | | |
| | | CCA | | | | | | | | | |
| SNCA_ | 428 | UGCUCU | 138 | AGAAGA | 558 | AT2278- | 57.44 | 2.59 | 88.01 | 5.11 | |
| 428 | | UUGGUC | | CCAAAG | | 2279 | | | | | |
| | | UUCUCA | | AGCA | | | | | | | |
| | | GCC | | | | | | | | | |
| SNCA_ | 430 | UUUGCU | 139 | AAGACC | 559 | AT1218- | 25.55 | 0.29 | 58.58 | 0.74 | |
| 430 | | CUUUGG | | AAAGAG | | 1219 | | | | | |
| | | UCUUCU | | CAAA | | | | | | | |
| | | CAG | | | | | | | | | |
| SNCA_ | 431 | UCUUGC | 140 | AGACCA | 560 | AT2280- | 117.42 | 2.24 | 119.05 | 9.12 | |
| 431 | | UCUUUG | | AAGAGC | | 2281 | | | | | |
| | | GUCUUC | | AAGA | | | | | | | |
| | | UCA | | | | | | | | | |
| SNCA_ | 432 | UACUUG | 141 | GACCAA | 561 | AT1220- | 37.41 | 0.91 | 62.89 | 0.85 | |
| 432 | | CUCUUU | | AGAGCA | | 1221 | | | | | |
| | | GGUCUU | | AGUA | | | | | | | |
| | | CUC | | | | | | | | | |
| SNCA_ | 433 | UCACUU | 142 | ACCAAA | 562 | AT2282- | 108.85 | 4.56 | 103.56 | 19.33 | |
| 433 | | GCUCUU | | GAGCAA | | 2283 | | | | | |
| | | UGGUCU | | GUGA | | | | | | | |
| | | UCU | | | | | | | | | |
| SNCA_ | 434 | UUCACU | 143 | CCAAAG | 563 | AT2284- | 30.01 | 3.32 | 59.42 | 3.43 | |
| 434 | | UGCUCU | | AGCAAG | | 2285 | | | | | |
| | | UUGGUC | | UGAA | | | | | | | |
| | | UUC | | | | | | | | | |
| SNCA_ | 438 | UUUUGU | 144 | AGAGCA | 564 | AT2286- | 48.55 | 3.36 | 58.16 | 10.29 | 628 |
| 438 | | CACUUG | | AGUGAC | | 2287 | | | | | |
| | | CUCUUU | | AAAA | | | | | | | |
| | | GGU | | | | | | | | | |
| SNCA_ | 439 | UAUUUG | 145 | GAGCAA | 565 | AT2288- | 69.36 | 2.82 | 67.47 | 3.92 | |
| 439 | | UCACUU | | GUGACA | | 2289 | | | | | |
| | | GCUCUU | | AAUA | | | | | | | |
| | | UGG | | | | | | | | | |
| SNCA_ | 440 | UCAUUU | 146 | AGCAAG | 566 | AT2290- | 103.25 | 3.57 | 77.33 | 5.42 | |
| 440 | | GUCACU | | UGACAA | | 2291 | | | | | |
| | | UGCUCU | | AUGA | | | | | | | |
| | | UUG | | | | | | | | | |
| SNCA_ | 441 | UACAUU | 147 | GCAAGU | 567 | AT1222- | 27.41 | 0.56 | 44.61 | 0.27 | |
| 441 | | UGUCAC | | GACAAA | | 1223 | | | | | |
| | | UUGCUC | | UGUA | | | | | | | |
| | | UUU | | | | | | | | | |
| SNCA_ | 442 | UAACAU | 148 | CAAGUG | 568 | AT1224- | 14.67 | 0.46 | 36.24 | 0.38 | 483 |
| 442 | | UUGUCA | | ACAAAU | | 1225 | | | | | |
| | | CUUGCU | | GUUA | | | | | | | |
| | | CUU | | | | | | | | | |
| SNCA_ | 443 | UCAACA | 149 | AAGUGA | 569 | AT2292- | 53.20 | 1.45 | 84.49 | 10.68 | |
| 443 | | UUUGUC | | CAAAUG | | 2293 | | | | | |
| | | ACUUGC | | UUGA | | | | | | | |
| | | UCU | | | | | | | | | |
| SNCA_ | 444 | UCCAAC | 150 | AGUGAC | 570 | AT1644- | 30.43 | 2.55 | 72.82 | 0.19 | 1110 |
| 444 | | AUUUGU | | AAAUGU | | 1645 | | | | | |
| | | CACUUG | | UGGA | | | | | | | |
| | | CUC | | | | | | | | | |
| SNCA_ | 445 | UUCCAA | 151 | GUGACA | 571 | AT2294- | 97.37 | 2.79 | 115.22 | 1.92 | |
| 445 | | CAUUUG | | AAUGUU | | 2295 | | | | | |
| | | UCACUU | | GGAA | | | | | | | |
| | | GCU | | | | | | | | | |
| SNCA_ | 446 | UCUCCA | 152 | UGACAA | 572 | AT2296- | 74.90 | 0.88 | 87.49 | 3.67 | |
| 446 | | ACAUUU | | AUGUUG | | 2297 | | | | | |
| | | GUCACU | | GAGA | | | | | | | |
| | | UGC | | | | | | | | | |
| SNCA_ | 447 | UCCUCC | 153 | GACAAA | 573 | AT2298- | 101.08 | 1.14 | 119.59 | 3.40 | |
| 447 | | AACAUU | | UGUUGG | | 2299 | | | | | |
| | | UGUCAC | | AGGA | | | | | | | |
| | | UUG | | | | | | | | | |
| SNCA_ | 462 | UCCCGU | 154 | AGCAGU | 574 | AT1652- | 81.52 | 0.18 | 103.29 | 0.79 | |
| 462 | | CACCAC | | GGUGAC | | 1653 | | | | | |
| | | UGCUCC | | GGGA | | | | | | | |
| | | UCC | | | | | | | | | |
| SNCA_ | 473 | UCUGCU | 155 | CGGGUG | 575 | AT1226- | 64.77 | 0.24 | 77.39 | 0.67 | |
| 473 | | GUCACA | | UGACAG | | 1227 | | | | | |
| | | CCCGUC | | CAGA | | | | | | | |
| | | ACC | | | | | | | | | |
| SNCA_ | 476 | UCUACU | 156 | GUGUGA | 576 | AT1228- | 67.35 | 0.61 | 86.62 | 0.02 | |
| 476 | | GCUGUC | | CAGCAG | | 1229 | | | | | |
| | | ACACCC | | UAGA | | | | | | | |
| | | GUC | | | | | | | | | |
| SNCA_ | 483 | UUUCUG | 157 | AGCAGU | 577 | AT1230- | 98.33 | 1.26 | 97.34 | 1.80 | |
| 483 | | GGCUAC | | AGCCCA | | 1231 | | | | | |
| | | UGCUGU | | GAAA | | | | | | | |
| | | CAC | | | | | | | | | |
| SNCA_ | 484 | UCUUCU | 158 | GCAGUA | 578 | AT1232- | 70.36 | 2.21 | 98.95 | 0.21 | |
| 484 | | GGGCUA | | GCCCAG | | 1233 | | | | | |
| | | CUGCUG | | AAGA | | | | | | | |
| | | UCA | | | | | | | | | |
| SNCA_ | 494 | UCCUCC | 159 | AGAAGA | 579 | AT2300- | 84.02 | 0.76 | 109.70 | 10.82 | |
| 494 | | ACUGUC | | CAGUGG | | 2301 | | | | | |
| | | UUCUGG | | AGGA | | | | | | | |
| | | GCU | | | | | | | | | |
| SNCA_ | 495 | UCCCUC | 160 | GAAGAC | 580 | AT2302- | 163.41 | 4.20 | 108.67 | 9.02 | |
| 495 | | CACUGU | | AGUGGA | | 2303 | | | | | |
| | | CUUCUG | | GGGA | | | | | | | |
| | | GGC | | | | | | | | | |
| SNCA_ | 496 | UUCCCU | 161 | AAGACA | 581 | AT2304- | 183.89 | 12.29 | 119.75 | 9.94 | |
| 496 | | CCACUG | | GUGGAG | | 2305 | | | | | |
| | | UCUUCU | | GGAA | | | | | | | |
| | | GGG | | | | | | | | | |
| SNCA_ | 497 | UCUCCC | 162 | AGACAG | 582 | AT2306- | 113.54 | 5.06 | 104.13 | 7.04 | |
| 497 | | UCCACU | | UGGAGG | | 2307 | | | | | |
| | | GUCUUC | | GAGA | | | | | | | |
| | | UGG | | | | | | | | | |
| SNCA_ | 498 | UGCUCC | 163 | GACAGU | 583 | AT2308- | 116.30 | 1.34 | 96.30 | 4.18 | |
| 498 | | CUCCAC | | GGAGGG | | 2309 | | | | | |
| | | UGUCUU | | AGCA | | | | | | | |
| | | CUG | | | | | | | | | |
| SNCA_ | 507 | UAUGCU | 164 | GGGAGC | 584 | AT2310- | 99.38 | 4.60 | 85.16 | 1.90 | |
| 507 | | CCCUGC | | AGGGAG | | 2311 | | | | | |
| | | UCCCUC | | CAUA | | | | | | | |
| | | CAC | | | | | | | | | |
| SNCA_ | 510 | UGCAAU | 165 | AGCAGG | 585 | AT2312- | 117.69 | 5.33 | 107.80 | 1.64 | |
| 510 | | GCUCCC | | GAGCAU | | 2313 | | | | | |
| | | UGCUCC | | UGCA | | | | | | | |
| | | CUC | | | | | | | | | |
| SNCA_ | 511 | UUGCAA | 166 | GCAGGG | 586 | AT2314- | 24.99 | 1.34 | 46.38 | 3.46 | 503 |
| 511 | | UGCUCC | | AGCAUU | | 2315 | | | | | |
| | | CUGCUC | | GCAA | | | | | | | |
| | | CCU | | | | | | | | | |
| SNCA_ | 556 | UUUCAU | 167 | UUGGGC | 587 | AT1110- | 97.32 | 1.12 | | | |
| 556 | | UCUUGC | | AAGAAU | | 1111 | | | | | |
| | | CCAACU | | GAAA | | | | | | | |
| | | GGU | | | | | | | | | |
| SNCA_ | 557 | UCUUCA | 168 | UGGGCA | 588 | AT1234- | 82.37 | 0.32 | 95.72 | 0.02 | |
| 557 | | UUCUUG | | AGAAUG | | 1235 | | | | | |
| | | CCCAAC | | AAGA | | | | | | | |
| | | UGG | | | | | | | | | |
| SNCA_ | 559 | UUUCUU | 169 | GGCAAG | 589 | AT1236- | 120.54 | 0.77 | 90.36 | 0.37 | |
| 559 | | CAUUCU | | AAUGAA | | 1237 | | | | | |
| | | UGCCCA | | GAAA | | | | | | | |
| | | ACU | | | | | | | | | |
| SNCA_ | 585 | UUCCAG | 170 | GGAAGG | 590 | AT2316- | 69.74 | 0.96 | 82.28 | 2.70 | |
| 585 | | AAUUCC | | AAUUCU | | 2317 | | | | | |
| | | UUCCUG | | GGAA | | | | | | | |
| | | UGG | | | | | | | | | |
| SNCA_ | 586 | UUUCCA | 171 | GAAGGA | 591 | AT2318- | 91.73 | 3.19 | 98.89 | 12.59 | |
| 586 | | GAAUUC | | AUUCUG | | 2319 | | | | | |
| | | CUUCCU | | GAAA | | | | | | | |
| | | GUG | | | | | | | | | |
| SNCA_ | 594 | UGGCAU | 172 | UCUGGA | 592 | AT2320- | 94.61 | 1.30 | 95.67 | 7.47 | |
| 594 | | AUCUUC | | AGAUAU | | 2321 | | | | | |
| | | CAGAAU | | GCCA | | | | | | | |
| | | UCC | | | | | | | | | |
| SNCA_ | 598 | UCACAG | 173 | GAAGAU | 593 | AT1238- | 92.83 | 0.62 | 89.11 | 0.03 | |
| 598 | | GCAUAU | | AUGCCU | | 1239 | | | | | |
| | | CUUCCA | | GUGA | | | | | | | |
| | | GAA | | | | | | | | | |
| SNCA_ | 611 | UCAUUG | 174 | UGGAUC | 594 | AT1240- | 90.13 | 1.97 | 79.05 | 0.80 | |
| 611 | | UCAGGA | | CUGACA | | 1241 | | | | | |
| | | UCCACA | | AUGA | | | | | | | |
| | | GGC | | | | | | | | | |
| SNCA_ | 613 | UCUCAU | 175 | GAUCCU | 595 | AT1242- | 73.28 | 1.55 | 109.41 | 3.09 | |
| 613 | | UGUCAG | | GACAAU | | 1243 | | | | | |
| | | GAUCCA | | GAGA | | | | | | | |
| | | CAG | | | | | | | | | |
| SNCA_ | 614 | UCCUCA | 176 | AUCCUG | 596 | AT1244- | 81.61 | 0.72 | 115.01 | 0.10 | |
| 614 | | UUGUCA | | ACAAUG | | 1245 | | | | | |
| | | GGAUCC | | AGGA | | | | | | | |
| | | ACA | | | | | | | | | |
| SNCA_ | 619 | UAUAAG | 177 | GACAAU | 597 | AT2322- | 112.50 | 7.58 | 96.31 | 6.27 | |
| 619 | | CCUCAU | | GAGGCU | | 2323 | | | | | |
| | | UGUCAG | | UAUA | | | | | | | |
| | | GAU | | | | | | | | | |
| SNCA_ | 620 | UCAUAA | 178 | ACAAUG | 598 | AT1246- | 77.40 | 1.74 | 101.73 | 3.14 | |
| 620 | | GCCUCA | | AGGCUU | | 1247 | | | | | |
| | | UUGUCA | | AUGA | | | | | | | |
| | | GGA | | | | | | | | | |
| SNCA_ | 621 | UUCAUA | 179 | CAAUGA | 599 | AT1654- | 18.55 | 0.05 | 63.43 | 0.73 | 109 |
| 621 | | AGCCUC | | GGCUUA | | 1655 | | | | | |
| | | AUUGUC | | UGAA | | | | | | | |
| | | AGG | | | | | | | | | |
| SNCA_ | 622 | UUUCAU | 180 | AAUGAG | 600 | AT1656- | 28.80 | 0.18 | 56.07 | 5.18 | |
| 622 | | AAGCCU | | GCUUAU | | 1657 | | | | | |
| | | CAUUGU | | GAAA | | | | | | | |
| | | CAG | | | | | | | | | |
| SNCA_ | 623 | UUUUCA | 181 | AUGAGG | 601 | AT2324- | 57.62 | 1.34 | 76.96 | 2.72 | |
| 623 | | UAAGCC | | CUUAUG | | 2325 | | | | | |
| | | UCAUUG | | AAAA | | | | | | | |
| | | UCA | | | | | | | | | |
| SNCA_ | 624 | UAUUUC | 182 | UGAGGC | 602 | AT2326- | 31.06 | 1.74 | 60.18 | 3.42 | |
| 624 | | AUAAGC | | UUAUGA | | 2327 | | | | | |
| | | CUCAUU | | AAUA | | | | | | | |
| | | GUC | | | | | | | | | |
| SNCA_ | 625 | UCAUUU | 183 | GAGGCU | 603 | AT2328- | 110.24 | 10.33 | 106.19 | 3.75 | |
| 625 | | CAUAAG | | UAUGAA | | 2329 | | | | | |
| | | CCUCAU | | AUGA | | | | | | | |
| | | UGU | | | | | | | | | |
| SNCA_ | 626 | UGCAUU | 184 | AGGCUU | 604 | AT1248- | 107.25 | 4.15 | 112.46 | 2.44 | |
| 626 | | UCAUAA | | AUGAAA | | 1249 | | | | | |
| | | GCCUCA | | UGCA | | | | | | | |
| | | UUG | | | | | | | | | |
| SNCA_ | 627 | UGGCAU | 185 | GGCUUA | 605 | AT2330- | 103.65 | 5.43 | 103.01 | 14.07 | |
| 627 | | UUCAUA | | UGAAAU | | 2331 | | | | | |
| | | AGCCUC | | GCCA | | | | | | | |
| | | AUU | | | | | | | | | |
| SNCA_ | 628 | UAGGCA | 186 | GCUUAU | 606 | AT2332- | 104.43 | 2.87 | 104.03 | 8.30 | |
| 628 | | UUUCAU | | GAAAUG | | 2333 | | | | | |
| | | AAGCCU | | CCUA | | | | | | | |
| | | CAU | | | | | | | | | |
| SNCA_ | 629 | UAAGGC | 187 | CUUAUG | 607 | AT2334- | 106.81 | 0.86 | 108.29 | 8.27 | |
| 629 | | AUUUCA | | AAAUGC | | 2335 | | | | | |
| | | UAAGCC | | CUUA | | | | | | | |
| | | UCA | | | | | | | | | |
| SNCA_ | 630 | UGAAGG | 188 | UUAUGA | 608 | AT1614- | 105.43 | 2.44 | 92.48 | 0.71 | |
| 630 | | CAUUUC | | AAUGCC | | 1615 | | | | | |
| | | AUAAGC | | UUCA | | | | | | | |
| | | CUC | | | | | | | | | |
| SNCA_ | 633 | UUCAGA | 189 | UGAAAU | 609 | AT2336- | 78.98 | 0.52 | 100.46 | 7.28 | |
| 633 | | AGGCAU | | GCCUUC | | 2337 | | | | | |
| | | UUCAUA | | UGAA | | | | | | | |
| | | AGC | | | | | | | | | |
| SNCA_ | 634 | UCUCAG | 190 | GAAAUG | 610 | AT1658- | 76.73 | 0.95 | 104.01 | 2.91 | |
| 634 | | AAGGCA | | CCUUCU | | 1659 | | | | | |
| | | UUUCAU | | GAGA | | | | | | | |
| | | AAG | | | | | | | | | |
| SNCA_ | 637 | UUUCCU | 191 | AUGCCU | 611 | AT1250- | 101.17 | 0.52 | 108.02 | 0.11 | |
| 637 | | CAGAAG | | UCUGAG | | 1251 | | | | | |
| | | GCAUUU | | GAAA | | | | | | | |
| | | CAU | | | | | | | | | |
| SNCA_ | 648 | UUCUUG | 192 | GGAAGG | 612 | AT1252- | 72.11 | 0.03 | 95.93 | 0.33 | |
| 648 | | AUACCC | | GUAUCA | | 1253 | | | | | |
| | | UUCCUC | | AGAA | | | | | | | |
| | | AGA | | | | | | | | | |
| SNCA_ | 649 | UGUCUU | 193 | GAAGGG | 613 | AT1112- | 84.62 | 1.29 | | | |
| 649 | | GAUACC | | UAUCAA | | 1113 | | | | | |
| | | CUUCCU | | GACA | | | | | | | |
| | | CAG | | | | | | | | | |
| SNCA_ | 662 | UCUUCA | 194 | ACUACG | 614 | AT1254- | 111.64 | 1.43 | 105.70 | 0.26 | |
| 662 | | GGUUCG | | AACCUG | | 1255 | | | | | |
| | | UAGUCU | | AAGA | | | | | | | |
| | | UGA | | | | | | | | | |
| SNCA_ | 663 | UGCUUC | 195 | CUACGA | 615 | AT1256- | 53.28 | 0.59 | 86.66 | 0.85 | |
| 663 | | AGGUUC | | ACCUGA | | 1257 | | | | | |
| | | GUAGUC | | AGCA | | | | | | | |
| | | UUG | | | | | | | | | |
| SNCA_ | 664 | UGGCUU | 196 | UACGAA | 616 | AT1258- | 106.42 | 0.67 | 112.79 | 2.44 | |
| 664 | | CAGGUU | | CCUGAA | | 1259 | | | | | |
| | | CGUAGU | | GCCA | | | | | | | |
| | | CUU | | | | | | | | | |
| SNCA_ | 668 | UCUUAG | 197 | AACCUG | 617 | AT2338- | 116.64 | 3.09 | 103.71 | 3.84 | |
| 668 | | GCUUCA | | AAGCCU | | 2339 | | | | | |
| | | GGUUCG | | AAGA | | | | | | | |
| | | UAG | | | | | | | | | |
| SNCA_ | 669 | UUCUUA | 198 | ACCUGA | 618 | AT1260- | 69.33 | 1.21 | 96.46 | 1.55 | |
| 669 | | GGCUUC | | AGCCUA | | 1261 | | | | | |
| | | AGGUUC | | AGAA | | | | | | | |
| | | GUA | | | | | | | | | |
| SNCA_ | 669 | UUCUUA | 199 | ACCUGA | 619 | AT2340- | 94.55 | 3.06 | 98.46 | 1.71 | |
| 669 | | GGCUUC | | AGCCUA | | 2341 | | | | | |
| | | AGGUUC | | AGAA | | | | | | | |
| | | GUA | | | | | | | | | |
| SNCA_ | 670 | UUUCUU | 200 | CCUGAA | 620 | AT1262- | 29.27 | 0.24 | 58.37 | 1.06 | |
| 670 | | AGGCUU | | GCCUAA | | 1263 | | | | | |
| | | CAGGUU | | GAAA | | | | | | | |
| | | CGU | | | | | | | | | |
| SNCA_ | 671 | UUUUCU | 201 | CUGAAG | 621 | AT2342- | 72.19 | 2.90 | 84.19 | 1.75 | |
| 671 | | UAGGCU | | CCUAAG | | 2343 | | | | | |
| | | UCAGGU | | AAAA | | | | | | | |
| | | UCG | | | | | | | | | |
| SNCA_ | 672 | UAUUUC | 202 | UGAAGC | 622 | AT2344- | 31.24 | 1.69 | 60.26 | 4.85 | |
| 672 | | UUAGGC | | CUAAGA | | 2345 | | | | | |
| | | UUCAGG | | AAUA | | | | | | | |
| | | UUC | | | | | | | | | |
| SNCA_ | 673 | UUAUUU | 203 | GAAGCC | 623 | AT2346- | 40.00 | 0.79 | 74.05 | 4.77 | |
| 673 | | CUUAGG | | UAAGAA | | 2347 | | | | | |
| | | CUUCAG | | AUAA | | | | | | | |
| | | GUU | | | | | | | | | |
| SNCA_ | 675 | UGAUAU | 204 | AGCCUA | 624 | AT2348- | 92.34 | 2.40 | 111.65 | 5.14 | |
| 675 | | UUCUUA | | AGAAAU | | 2349 | | | | | |
| | | GGCUUC | | AUCA | | | | | | | |
| | | AGG | | | | | | | | | |
| SNCA_ | 676 | UAGAUA | 205 | GCCUAA | 625 | AT2350- | 49.09 | 0.73 | 90.99 | 4.17 | |
| 676 | | UUUCUU | | GAAAUA | | 2351 | | | | | |
| | | AGGCUU | | UCUA | | | | | | | |
| | | CAG | | | | | | | | | |
| SNCA_ | 677 | UAAGAU | 206 | CCUAAG | 626 | AT1264- | 27.50 | 0.60 | 52.84 | 0.88 | |
| 677 | | AUUUCU | | AAAUAU | | 1265 | | | | | |
| | | UAGGCU | | CUUA | | | | | | | |
| | | UCA | | | | | | | | | |
| SNCA_ | 678 | UAAAGA | 207 | CUAAGA | 627 | AT1266- | 39.96 | 0.68 | 70.02 | 1.05 | |
| 678 | | UAUUUC | | AAUAUC | | 1267 | | | | | |
| | | UUAGGC | | UUUA | | | | | | | |
| | | UUC | | | | | | | | | |
| SNCA_ | 679 | UCAAAG | 208 | UAAGAA | 628 | AT2352- | 52.17 | 2.73 | 97.22 | 7.78 | |
| 679 | | AUAUUU | | AUAUCU | | 2353 | | | | | |
| | | CUUAGG | | UUGA | | | | | | | |
| | | CUU | | | | | | | | | |
| SNCA_ | 680 | UGCAAA | 209 | AAGAAA | 629 | AT2354- | 77.61 | 2.38 | 104.48 | 2.48 | |
| 680 | | GAUAUU | | UAUCUU | | 2355 | | | | | |
| | | UCUUAG | | UGCA | | | | | | | |
| | | GCU | | | | | | | | | |
| SNCA_ | 681 | UAGCAA | 210 | AGAAAU | 630 | AT1268- | 27.87 | 0.67 | 37.13 | 1.00 | |
| 681 | | AGAUAU | | AUCUUU | | 1269 | | | | | |
| | | UUCUUA | | GCUA | | | | | | | |
| | | GGC | | | | | | | | | |
| SNCA_ | 682 | UGAGCA | 211 | GAAAUA | 631 | AT1270- | 18.24 | 0.13 | 49.56 | 0.43 | 554 |
| 682 | | AAGAUA | | UCUUUG | | 1271 | | | | | |
| | | UUUCUU | | CUCA | | | | | | | |
| | | AGG | | | | | | | | | |
| SNCA_ | 683 | UGGAGC | 212 | AAAUAU | 632 | AT2356- | 75.29 | 2.75 | 101.82 | 5.89 | |
| 683 | | AAAGAU | | CUUUGC | | 2357 | | | | | |
| | | AUUUCU | | UCCA | | | | | | | |
| | | UAG | | | | | | | | | |
| SNCA_ | 684 | UGGGAG | 213 | AAUAUC | 633 | AT2358- | 100.11 | 0.76 | 116.00 | 6.34 | |
| 684 | | CAAAGA | | UUUGCU | | 2359 | | | | | |
| | | UAUUUC | | CCCA | | | | | | | |
| | | UUA | | | | | | | | | |
| SNCA_ | 693 | UCAAGA | 214 | GCUCCC | 634 | AT1272- | 78.60 | 1.02 | 87.69 | 2.63 | |
| 693 | | AACUGG | | AGUUUC | | 1273 | | | | | |
| | | GAGCAA | | UUGA | | | | | | | |
| | | AGA | | | | | | | | | |
| SNCA_ | 694 | UUCAAG | 215 | CUCCCA | 635 | AT2360- | 114.92 | 2.06 | 105.73 | 9.44 | |
| 694 | | AAACUG | | GUUUCU | | 2361 | | | | | |
| | | GGAGCA | | UGAA | | | | | | | |
| | | AAG | | | | | | | | | |
| SNCA_ | 695 | UCUCAA | 216 | UCCCAG | 636 | AT2362- | 70.02 | 2.88 | 84.15 | 5.15 | |
| 695 | | GAAACU | | UUUCUU | | 2363 | | | | | |
| | | GGGAGC | | GAGA | | | | | | | |
| | | AAA | | | | | | | | | |
| SNCA_ | 696 | UUCUCA | 217 | CCCAGU | 637 | AT1274- | 32.00 | 0.42 | 48.23 | 0.74 | |
| 696 | | AGAAAC | | UUCUUG | | 1275 | | | | | |
| | | UGGGAG | | AGAA | | | | | | | |
| | | CAA | | | | | | | | | |
| SNCA_ | 697 | UAUCUC | 218 | CCAGUU | 638 | AT1114- | 31.18 | 1.36 | | | |
| 697 | | AAGAAA | | UCUUGA | | 1115 | | | | | |
| | | CUGGGA | | GAUA | | | | | | | |
| | | GCA | | | | | | | | | |
| SNCA_ | 698 | UGAUCU | 219 | CAGUUU | 639 | AT2364- | 43.76 | 0.21 | 76.39 | 1.50 | |
| 698 | | CAAGAA | | CUUGAG | | 2365 | | | | | |
| | | ACUGGG | | AUCA | | | | | | | |
| | | AGC | | | | | | | | | |
| SNCA_ | 699 | UAGAUC | 220 | AGUUUC | 640 | AT2366- | 29.88 | 0.15 | 43.85 | 1.67 | |
| 699 | | UCAAGA | | UUGAGA | | 2367 | | | | | |
| | | AACUGG | | UCUA | | | | | | | |
| | | GAG | | | | | | | | | |
| SNCA_ | 701 | UGCAGA | 221 | UUUCUU | 641 | AT1276- | 54.94 | 0.85 | 66.08 | 1.02 | |
| 701 | | UCUCAA | | GAGAUC | | 1277 | | | | | |
| | | GAAACU | | UGCA | | | | | | | |
| | | GGG | | | | | | | | | |
| SNCA_ | 70! | UGUCAG | 222 | UUGAGA | 642 | AT2368- | 33.46 | 0.57 | 74.92 | 1.10 | |
| 705 | | CAGAUC | | UCUGCU | | 2369 | | | | | |
| | | UCAAGA | | GACA | | | | | | | |
| | | AAC | | | | | | | | | |
| SNCA_ | 706 | UUGUCA | 223 | UGAGAU | 643 | AT2370- | 33.52 | 1.08 | 74.88 | 4.38 | |
| 706 | | GCAGAU | | CUGCUG | | 2371 | | | | | |
| | | CUCAAG | | ACAA | | | | | | | |
| | | AAA | | | | | | | | | |
| SNCA_ | 707 | UCUGUC | 224 | GAGAUC | 644 | AT1660- | 24.05 | 0.16 | 65.00 | 0.32 | 362 |
| 707 | | AGCAGA | | UGCUGA | | 1661 | | | | | |
| | | UCUCAA | | CAGA | | | | | | | |
| | | GAA | | | | | | | | | |
| SNCA_ | 708 | UUCUGU | 225 | AGAUCU | 645 | AT1278- | 43.36 | 0.76 | 70.25 | 1.01 | |
| 708 | | CAGCAG | | GCUGAC | | 1279 | | | | | |
| | | AUCUCA | | AGAA | | | | | | | |
| | | AGA | | | | | | | | | |
| SNCA_ | 709 | UAUCUG | 226 | GAUCUG | 646 | AT2372- | 68.28 | 2.59 | 101.23 | 4.91 | |
| 709 | | UCAGCA | | CUGACA | | 2373 | | | | | |
| | | GAUCUC | | GAUA | | | | | | | |
| | | AAG | | | | | | | | | |
| SNCA_ | 710 | UCAUCU | 227 | AUCUGC | 647 | AT2374- | 55.83 | 0.83 | 91.80 | 3.53 | |
| 710 | | GUCAGC | | UGACAG | | 2375 | | | | | |
| | | AGAUCU | | AUGA | | | | | | | |
| | | CAA | | | | | | | | | |
| SNCA_ | 711 | UACAUC | 228 | UCUGCU | 648 | AT2376- | 36.90 | 2.66 | 62.99 | 2.67 | |
| 711 | | UGUCAG | | GACAGA | | 2377 | | | | | |
| | | CAGAUC | | UGUA | | | | | | | |
| | | UCA | | | | | | | | | |
| SNCA_ | 715 | UUGGAA | 229 | CUGACA | 649 | AT2378- | 39.67 | 1.77 | 79.44 | 1.75 | |
| 715 | | CAUCUG | | GAUGUU | | 2379 | | | | | |
| | | UCAGCA | | CCAA | | | | | | | |
| | | GAU | | | | | | | | | |
| SNCA_ | 716 | UAUGGA | 230 | UGACAG | 650 | AT2380- | 30.77 | 2.62 | 53.24 | 3.75 | |
| 716 | | ACAUCU | | AUGUUC | | 2381 | | | | | |
| | | GUCAGC | | CAUA | | | | | | | |
| | | AGA | | | | | | | | | |
| SNCA_ | 717 | UGAUGG | 231 | GACAGA | 651 | AT1662- | 24.71 | 1.01 | 52.32 | 0.41 | 553 |
| 717 | | AACAUC | | UGUUCC | | 1663 | | | | | |
| | | UGUCAG | | AUCA | | | | | | | |
| | | CAG | | | | | | | | | |
| SNCA_ | 718 | UGGAUG | 232 | ACAGAU | 652 | AT2382- | 54.63 | 2.70 | 77.52 | 4.46 | |
| 718 | | GAACAU | | GUUCCA | | 2383 | | | | | |
| | | CUGUCA | | UCCA | | | | | | | |
| | | GCA | | | | | | | | | |
| SNCA_ | 719 | UAGGAU | 233 | CAGAUG | 653 | AT2384- | 47.82 | 2.86 | 74.84 | 9.26 | |
| 719 | | GGAACA | | UUCCAU | | 2385 | | | | | |
| | | UCUGUC | | CCUA | | | | | | | |
| | | AGC | | | | | | | | | |
| SNCA_ | 720 | UCAGGA | 234 | AGAUGU | 654 | AT1116- | 52.35 | 1.04 | | | |
| 720 | | UGGAAC | | UCCAUC | | 1117 | | | | | |
| | | AUCUGU | | CUGA | | | | | | | |
| | | CAG | | | | | | | | | |
| SNCA_ | 721 | UACAGG | 235 | GAUGUU | 655 | AT2386- | 36.14 | 2.35 | 86.93 | 4.85 | |
| 721 | | AUGGAA | | CCAUCC | | 2387 | | | | | |
| | | CAUCUG | | UGUA | | | | | | | |
| | | UCA | | | | | | | | | |
| SNCA_ | 722 | UUACAG | 236 | AUGUUC | 656 | AT2388- | 68.12 | 2.05 | 103.87 | 10.60 | |
| 722 | | GAUGGA | | CAUCCU | | 2389 | | | | | |
| | | ACAUCU | | GUAA | | | | | | | |
| | | GUC | | | | | | | | | |
| SNCA_ | 725 | UUUGUA | 237 | UUCCAU | 657 | AT1664- | 56.71 | 0.40 | 101.80 | 0.32 | |
| 725 | | CAGGAU | | CCUGUA | | 1665 | | | | | |
| | | GGAACA | | CAAA | | | | | | | |
| | | UCU | | | | | | | | | |
| SNCA_ | 730 | UAGCAC | 238 | UCCUGU | 658 | AT1280- | 30.16 | 0.29 | 40.60 | 0.93 | |
| 730 | | UUGUAC | | ACAAGU | | 1281 | | | | | |
| | | AGGAUG | | GCUA | | | | | | | |
| | | GAA | | | | | | | | | |
| SNCA_ | 732 | UUGAGC | 239 | CUGUAC | 659 | AT1666- | 18.73 | 0.16 | 40.49 | 0.10 | 208 |
| 732 | | ACUUGU | | AAGUGC | | 1667 | | | | | |
| | | ACAGGA | | UCAA | | | | | | | |
| | | UGG | | | | | | | | | |
| SNCA_ | 733 | UCUGAG | 240 | UGUACA | 660 | AT1282- | 33.73 | 5.36 | 56.91 | 3.29 | |
| 733 | | CACUUG | | AGUGCU | | 1283 | | | | | |
| | | UACAGG | | CAGA | | | | | | | |
| | | AUG | | | | | | | | | |
| SNCA_ | 734 | UACUGA | 241 | GUACAA | 661 | AT2390- | 46.73 | 1.60 | 79.08 | 8.22 | |
| 734 | | GCACUU | | GUGCUC | | 2391 | | | | | |
| | | GUACAG | | AGUA | | | | | | | |
| | | GAU | | | | | | | | | |
| SNCA_ | 735 | UAACUG | 242 | UACAAG | 662 | AT2392- | 45.36 | 0.79 | 81.46 | 6.20 | |
| 735 | | AGCACU | | UGCUCA | | 2393 | | | | | |
| | | UGUACA | | GUUA | | | | | | | |
| | | GGA | | | | | | | | | |
| SNCA_ | 736 | UGAACU | 243 | ACAAGU | 663 | AT1646- | 30.52 | 0.75 | 43.27 | 1.70 | 164 |
| 736 | | GAGCAC | | GCUCAG | | 1647 | | | | | |
| | | UUGUAC | | UUCA | | | | | | | |
| | | AGG | | | | | | | | | |
| SNCA_ | 737 | UGGAAC | 244 | CAAGUG | 664 | AT1648- | 25.01 | 0.04 | 26.76 | 0.23 | 90.5 |
| 737 | | UGAGCA | | CUCAGU | | 1649 | | | | | |
| | | CUUGUA | | UCCA | | | | | | | |
| | | CAG | | | | | | | | | |
| SNCA_ | 738 | UUGGAA | 245 | AAGUGC | 665 | AT2394- | 35.16 | 4.26 | 74.06 | 8.85 | 507 |
| 738 | | CUGAGC | | UCAGUU | | 2395 | | | | | |
| | | ACUUGU | | CCAA | | | | | | | |
| | | ACA | | | | | | | | | |
| SNCA_ | 739 | UUUGGA | 246 | AGUGCU | 666 | AT2396- | 22.34 | 0.28 | 33.18 | 2.24 | 94 |
| 739 | | ACUGAG | | CAGUUC | | 2397 | | | | | |
| | | CACUUG | | CAAA | | | | | | | |
| | | UAC | | | | | | | | | |
| SNCA_ | 740 | UAUUGG | 247 | GUGCUC | 667 | AT1284- | 23.68 | 3.33 | 41.78 | 0.37 | 213 |
| 740 | | AACUGA | | AGUUCC | | 1285 | | | | | |
| | | GCACUU | | AAUA | | | | | | | |
| | | GUA | | | | | | | | | |
| SNCA_ | 741 | UCAUUG | 248 | UGCUCA | 668 | AT2398- | 64.53 | 2.99 | 84.95 | 5.41 | |
| 741 | | GAACUG | | GUUCCA | | 2399 | | | | | |
| | | AGCACU | | AUGA | | | | | | | |
| | | UGU | | | | | | | | | |
| SNCA_ | 742 | UACAUU | 249 | GCUCAG | 669 | AT2400- | 50.50 | 5.16 | 71.67 | 3.30 | |
| 742 | | GGAACU | | UUCCAA | | 2401 | | | | | |
| | | GAGCAC | | UGUA | | | | | | | |
| | | UUG | | | | | | | | | |
| SNCA_ | 743 | UCACAU | 250 | CUCAGU | 670 | AT2402- | 43.06 | 2.83 | 71.45 | 2.17 | |
| 743 | | UGGAAC | | UCCAAU | | 2403 | | | | | |
| | | UGAGCA | | GUGA | | | | | | | |
| | | CUU | | | | | | | | | |
| SNCA_ | 744 | UGCACA | 251 | UCAGUU | 671 | AT1286- | 22.19 | 2.66 | 33.39 | 0.25 | 136 |
| 744 | | UUGGAA | | CCAAUG | | 1287 | | | | | |
| | | CUGAGC | | UGCA | | | | | | | |
| | | ACU | | | | | | | | | |
| SNCA_ | 745 | UGGCAC | 252 | CAGUUC | 672 | AT2404- | 36.09 | 0.76 | 71.89 | 7.40 | |
| 745 | | AUUGGA | | CAAUGU | | 2405 | | | | | |
| | | ACUGAG | | GCCA | | | | | | | |
| | | CAC | | | | | | | | | |
| SNCA_ | 746 | UGGGCA | 253 | AGUUCC | 673 | AT2406- | 46.81 | 3.22 | 83.75 | 1.33 | |
| 746 | | CAUUGG | | AAUGUG | | 2407 | | | | | |
| | | AACUGA | | CCCA | | | | | | | |
| | | GCA | | | | | | | | | |
| SNCA_ | 747 | UUGGGC | 254 | GUUCCA | 674 | AT2408- | 48.24 | 3.85 | 84.90 | 7.63 | |
| 747 | | ACAUUG | | AUGUGC | | 2409 | | | | | |
| | | GAACUG | | CCAA | | | | | | | |
| | | AGC | | | | | | | | | |
| SNCA_ | 748 | UCUGGG | 255 | UUCCAA | 675 | AT1288- | 42.67 | 1.51 | 82.36 | 1.71 | |
| 748 | | CACAUU | | UGUGCC | | 1289 | | | | | |
| | | GGAACU | | CAGA | | | | | | | |
| | | GAG | | | | | | | | | |
| SNCA_ | 749 | UACUGG | 256 | UCCAAU | 676 | AT2410- | 57.51 | 1.78 | 89.15 | 9.51 | |
| 749 | | GCACAU | | GUGCCC | | 2411 | | | | | |
| | | UGGAAC | | AGUA | | | | | | | |
| | | UGA | | | | | | | | | |
| SNCA_ | 750 | UGACUG | 257 | CCAAUG | 677 | AT1290- | 44.08 | 1.04 | 99.78 | 0.87 | |
| 750 | | GGCACA | | UGCCCA | | 1291 | | | | | |
| | | UUGGAA | | GUCA | | | | | | | |
| | | CUG | | | | | | | | | |
| SNCA_ | 751 | UUGACU | 258 | CAAUGU | 678 | AT1292- | 88.30 | 2.24 | 99.97 | 2.62 | |
| 751 | | GGGCAC | | GCCCAG | | 1293 | | | | | |
| | | AUUGGA | | UCAA | | | | | | | |
| | | ACU | | | | | | | | | |
| SNCA_ | 752 | UAUGAC | 259 | AAUGUG | 679 | AT1294- | 118.93 | 1.22 | 123.64 | 0.44 | |
| 752 | | UGGGCA | | CCCAGU | | 1295 | | | | | |
| | | CAUUGG | | CAUA | | | | | | | |
| | | AAC | | | | | | | | | |
| SNCA_ | 755 | UGUCAU | 260 | GUGCCC | 680 | AT1296- | 83.75 | 0.20 | 88.63 | 0.41 | |
| 755 | | GACUGG | | AGUCAU | | 1297 | | | | | |
| | | GCACAU | | GACA | | | | | | | |
| | | UGG | | | | | | | | | |
| SNCA_ | 758 | UAAUGU | 261 | CCCAGU | 681 | AT1298- | 89.23 | 0.27 | 96.17 | 0.19 | |
| 758 | | CAUGAC | | CAUGAC | | 1299 | | | | | |
| | | UGGGCA | | AUUA | | | | | | | |
| | | CAU | | | | | | | | | |
| SNCA_ | 759 | UAAAUG | 262 | CCAGUC | 682 | AT1300- | 67.00 | 1.05 | 113.12 | 0.17 | |
| 759 | | UCAUGA | | AUGACA | | 1301 | | | | | |
| | | CUGGGC | | UUUA | | | | | | | |
| | | ACA | | | | | | | | | |
| SNCA_ | 760 | UGAAAU | 263 | CAGUCA | 683 | AT1302- | 97.42 | 2.79 | 103.79 | 2.21 | |
| 760 | | GUCAUG | | UGACAU | | 1303 | | | | | |
| | | ACUGGG | | UUCA | | | | | | | |
| | | CAC | | | | | | | | | |
| SNCA_ | 763 | UUGAGA | 264 | UCAUGA | 684 | AT1304- | 322.29 | 2.83 | 129.19 | 4.10 | |
| 763 | | AAUGUC | | CAUUUC | | 1305 | | | | | |
| | | AUGACU | | UCAA | | | | | | | |
| | | GGG | | | | | | | | | |
| SNCA_ | 764 | UUUGAG | 265 | CAUGAC | 685 | AT1306- | 51.38 | 0.02 | 47.85 | 0.36 | |
| 764 | | AAAUGU | | AUUUCU | | 1307 | | | | | |
| | | CAUGAC | | CAAA | | | | | | | |
| | | UGG | | | | | | | | | |
| SNCA_ | 765 | UUUUGA | 266 | AUGACA | 686 | AT2412- | 27.57 | 0.38 | 48.03 | 6.40 | |
| 765 | | GAAAUG | | UUUCUC | | 2413 | | | | | |
| | | UCAUGA | | AAAA | | | | | | | |
| | | CUG | | | | | | | | | |
| SNCA_ | 766 | UCUUUG | 267 | UGACAU | 687 | AT1308- | 24.66 | 0.30 | 30.18 | 0.17 | 151 |
| 766 | | AGAAAU | | UUCUCA | | 1309 | | | | | |
| | | GUCAUG | | AAGA | | | | | | | |
| | | ACU | | | | | | | | | |
| SNCA_ | 767 | UACUUU | 268 | GACAUU | 688 | AT1310- | 72.62 | 0.33 | 58.66 | 1.32 | |
| 767 | | GAGAAA | | UCUCAA | | 1311 | | | | | |
| | | UGUCAU | | AGUA | | | | | | | |
| | | GAC | | | | | | | | | |
| SNCA_ | 787 | UCUUCG | 269 | CAGUGU | 689 | AT2414- | 41.76 | 0.71 | 64.88 | 10.93 | |
| 787 | | AGAUAC | | AUCUCG | | 2415 | | | | | |
| | | ACUGUA | | AAGA | | | | | | | |
| | | AAA | | | | | | | | | |
| SNCA_ | 788 | UACUUC | 270 | AGUGUA | 690 | AT2416- | 30.50 | 1.67 | 50.80 | 3.22 | |
| 788 | | GAGAUA | | UCUCGA | | 2417 | | | | | |
| | | CACUGU | | AGUA | | | | | | | |
| | | AAA | | | | | | | | | |
| SNCA_ | 789 | UGACUU | 271 | GUGUAU | 691 | AT1312- | 36.89 | 0.97 | 55.63 | 1.92 | |
| 789 | | CGAGAU | | CUCGAA | | 1313 | | | | | |
| | | ACACUG | | GUCA | | | | | | | |
| | | UAA | | | | | | | | | |
| SNCA_ | 790 | UAGACU | 272 | UGUAUC | 692 | AT1118- | 40.72 | 0.83 | | | |
| 790 | | UCGAGA | | UCGAAG | | 1119 | | | | | |
| | | UACACU | | UCUA | | | | | | | |
| | | GUA | | | | | | | | | |
| SNCA_ | 791 | UAAGAC | 273 | GUAUCU | 693 | AT2418- | 24.63 | 0.65 | 42.23 | 0.25 | 327 |
| 791 | | UUCGAG | | CGAAGU | | 2419 | | | | | |
| | | AUACAC | | CUUA | | | | | | | |
| | | UGU | | | | | | | | | |
| SNCA_ | 792 | UGAAGA | 274 | UAUCUC | 694 | AT1314- | 119.37 | 0.37 | 108.03 | 2.17 | |
| 792 | | CUUCGA | | GAAGUC | | 1315 | | | | | |
| | | GAUACA | | UUCA | | | | | | | |
| | | CUG | | | | | | | | | |
| SNCA_ | 796 | UGAUGG | 275 | UCGAAG | 695 | AT2420- | 47.19 | 0.74 | 63.35 | 3.93 | 616 |
| 796 | | AAGACU | | UCUUCC | | 2421 | | | | | |
| | | UCGAGA | | AUCA | | | | | | | |
| | | UAC | | | | | | | | | |
| SNCA_ | 797 | UUGAUG | 276 | CGAAGU | 696 | AT2422- | 54.87 | 1.38 | 68.42 | 4.62 | |
| 797 | | GAAGAC | | CUUCCA | | 2423 | | | | | |
| | | UUCGAG | | UCAA | | | | | | | |
| | | AUA | | | | | | | | | |
| SNCA_ | 798 | UCUGAU | 277 | GAAGUC | 697 | AT1316- | 105.24 | 1.89 | 96.39 | 1.85 | |
| 798 | | GGAAGA | | UUCCAU | | 1317 | | | | | |
| | | CUUCGA | | CAGA | | | | | | | |
| | | GAU | | | | | | | | | |
| SNCA_ | 799 | UGCUGA | 278 | AAGUCU | 698 | AT1318- | 38.66 | 1.23 | 57.82 | 0.96 | |
| 799 | | UGGAAG | | UCCAUC | | 1319 | | | | | |
| | | ACUUCG | | AGCA | | | | | | | |
| | | AGA | | | | | | | | | |
| SNCA_ | 008 | UUGCUG | 279 | AGUCUU | 699 | AT1616- | 129.88 | 1.72 | 87.01 | 2.05 | |
| 800 | | AUGGAA | | CCAUCA | | 1617 | | | | | |
| | | GACUUC | | GCAA | | | | | | | |
| | | GAG | | | | | | | | | |
| SNCA_ | 801 | UCUGCU | 280 | GUCUUC | 700 | AT1320- | 71.81 | 0.69 | 78.75 | 1.98 | |
| 801 | | GAUGGA | | CAUCAG | | 1321 | | | | | |
| | | AGACUU | | CAGA | | | | | | | |
| | | CGA | | | | | | | | | |
| SNCA_ | 802 | UACUGC | 281 | UCUUCC | 701 | AT1322- | 104.53 | 3.64 | 97.58 | 1.47 | |
| 802 | | UGAUGG | | AUCAGC | | 1323 | | | | | |
| | | AAGACU | | AGUA | | | | | | | |
| | | UCG | | | | | | | | | |
| SNCA_ | 803 | UCACUG | 282 | CUUCCA | 702 | AT2424- | 79.55 | 0.12 | 84.25 | 6.39 | |
| 803 | | CUGAUG | | UCAGCA | | 2425 | | | | | |
| | | GAAGAC | | GUGA | | | | | | | |
| | | UUC | | | | | | | | | |
| SNCA_ | 804 | UUCACU | 283 | UUCCAU | 703 | AT2426- | 74.67 | 4.48 | 79.98 | 3.02 | |
| 804 | | GCUGAU | | CAGCAG | | 2427 | | | | | |
| | | GGAAGA | | UGAA | | | | | | | |
| | | CUU | | | | | | | | | |
| SNCA_ | 805 | UAUCAC | 284 | UCCAUC | 704 | AT1324- | 87.39 | 0.20 | 91.28 | 0.66 | |
| 805 | | UGCUGA | | AGCAGU | | 1325 | | | | | |
| | | UGGAAG | | GAUA | | | | | | | |
| | | ACU | | | | | | | | | |
| SNCA_ | 806 | UAAUCA | 285 | CCAUCA | 705 | AT1326- | 81.50 | 1.45 | 92.99 | 1.52 | |
| 806 | | CUGCUG | | GCAGUG | | 1327 | | | | | |
| | | AUGGAA | | AUUA | | | | | | | |
| | | GAC | | | | | | | | | |
| SNCA_ | 807 | UCAAUC | 286 | CAUCAG | 706 | AT2428- | 33.69 | 3.40 | 49.66 | 0.32 | |
| 807 | | ACUGCU | | CAGUGA | | 2429 | | | | | |
| | | GAUGGA | | UUGA | | | | | | | |
| | | AGA | | | | | | | | | |
| SNCA_ | 808 | UUCAAU | 287 | AUCAGC | 707 | AT1328- | 119.29 | 1.41 | 105.24 | 1.02 | |
| 808 | | CACUGC | | AGUGAU | | 1329 | | | | | |
| | | UGAUGG | | UGAA | | | | | | | |
| | | AAG | | | | | | | | | |
| SNCA_ | 809 | UUUCAA | 288 | UCAGCA | 708 | AT1330- | 42.00 | 0.00 | 48.80 | 1.65 | |
| 809 | | UCACUG | | GUGAUU | | 1331 | | | | | |
| | | CUGAUG | | GAAA | | | | | | | |
| | | GAA | | | | | | | | | |
| SNCA_ | 810 | UCUUCA | 289 | CAGCAG | 709 | AT2430- | 39.99 | 0.25 | 59.24 | 1.92 | |
| 810 | | AUCACU | | UGAUUG | | 2431 | | | | | |
| | | GCUGAU | | AAGA | | | | | | | |
| | | GGA | | | | | | | | | |
| SNCA_ | 811 | UACUUC | 290 | AGCAGU | 710 | AT1332- | 59.66 | 2.17 | 74.75 | 0.99 | |
| 811 | | AAUCAC | | GAUUGA | | 1333 | | | | | |
| | | UGCUGA | | AGUA | | | | | | | |
| | | UGG | | | | | | | | | |
| SNCA_ | 812 | UUACUU | 291 | GCAGUG | 711 | AT2432- | 34.59 | 0.75 | 61.10 | 2.83 | |
| 812 | | CAAUCA | | AUUGAA | | 2433 | | | | | |
| | | CUGCUG | | GUAA | | | | | | | |
| | | AUG | | | | | | | | | |
| SNCA_ | 813 | UAUACU | 292 | CAGUGA | 712 | AT2434- | 26.73 | 1.39 | 43.03 | 5.13 | |
| 813 | | UCAAUC | | UUGAAG | | 2435 | | | | | |
| | | ACUGCU | | UAUA | | | | | | | |
| | | GAU | | | | | | | | | |
| SNCA_ | 820 | UGGUAC | 293 | UGAAGU | 713 | AT2436- | 57.48 | 7.43 | 81.70 | 1.40 | |
| 820 | | AGAUAC | | AUCUGU | | 2437 | | | | | |
| | | UUCAAU | | ACCA | | | | | | | |
| | | CAC | | | | | | | | | |
| SNCA_ | 821 | UAGGUA | 294 | GAAGUA | 714 | AT2438- | 40.15 | 2.94 | 71.31 | 4.03 | |
| 821 | | CAGAUA | | UCUGUA | | 2439 | | | | | |
| | | CUUCAA | | CCUA | | | | | | | |
| | | UCA | | | | | | | | | |
| SNCA_ | 822 | UCAGGU | 295 | AAGUAU | 715 | AT1668- | 25.79 | 0.44 | 57.75 | 0.07 | |
| 822 | | ACAGAU | | CUGUAC | | 1669 | | | | | |
| | | ACUUCA | | CUGA | | | | | | | |
| | | AUC | | | | | | | | | |
| SNCA_ | 823 | UGCAGG | 296 | AGUAUC | 716 | AT1334- | 78.38 | 0.90 | 97.50 | 2.27 | |
| 823 | | UACAGA | | UGUACC | | 1335 | | | | | |
| | | UACUUC | | UGCA | | | | | | | |
| | | AAU | | | | | | | | | |
| SNCA_ | 824 | UGGCAG | 297 | GUAUCU | 717 | AT2440- | 103.97 | 8.12 | 94.20 | 3.65 | |
| 824 | | GUACAG | | GUACCU | | 2441 | | | | | |
| | | AUACUU | | GCCA | | | | | | | |
| | | CAA | | | | | | | | | |
| SNCA_ | 825 | UGGGCA | 298 | UAUCUG | 718 | AT1336- | 95.03 | 0.77 | 96.62 | 0.71 | |
| 825 | | GGUACA | | UACCUG | | 1337 | | | | | |
| | | GAUACU | | CCCA | | | | | | | |
| | | UCA | | | | | | | | | |
| SNCA_ | 846 | UAAGCA | 299 | AGCAUU | 719 | AT1338- | 66.95 | 1.87 | 104.26 | 3.12 | |
| 846 | | CCGAAA | | UCGGUG | | 1339 | | | | | |
| | | UGCUGA | | CUUA | | | | | | | |
| | | GUG | | | | | | | | | |
| SNCA_ | 847 | UGAAGC | 300 | GCAUUU | 720 | AT1340- | 83.57 | 0.42 | 116.71 | 1.29 | |
| 847 | | ACCGAA | | CGGUGC | | 1341 | | | | | |
| | | AUGCUG | | UUCA | | | | | | | |
| | | AGU | | | | | | | | | |
| SNCA_ | 851 | UAAGGG | 301 | UUCGGU | 721 | AT1342- | 60.19 | 0.59 | 105.33 | 2.09 | |
| 851 | | AAGCAC | | GCUUCC | | 1343 | | | | | |
| | | CGAAAU | | CUUA | | | | | | | |
| | | GCU | | | | | | | | | |
| SNCA_ | 852 | UAAAGG | 302 | UCGGUG | 722 | AT1344- | 86.27 | 1.63 | 119.57 | 0.43 | |
| 852 | | GAAGCA | | CUUCCC | | 1345 | | | | | |
| | | CCGAAA | | UUUA | | | | | | | |
| | | UGC | | | | | | | | | |
| SNCA_ | 854 | UUGAAA | 303 | GGUGCU | 723 | AT1346- | 91.22 | 1.94 | 126.94 | 1.53 | |
| 854 | | GGGAAG | | UCCCUU | | 1347 | | | | | |
| | | CACCGA | | UCAA | | | | | | | |
| | | AAU | | | | | | | | | |
| SNCA_ | 855 | UGUGAA | 304 | GUGCUU | 724 | AT1348- | 89.48 | 2.34 | 105.49 | 1.18 | |
| 855 | | AGGGAA | | CCCUUU | | 1349 | | | | | |
| | | GCACCG | | CACA | | | | | | | |
| | | AAA | | | | | | | | | |
| SNCA_ | 856 | UAGUGA | 305 | UGCUUC | 725 | AT1120- | 81.59 | 0.08 | | | |
| 856 | | AAGGGA | | CCUUUC | | 1121 | | | | | |
| | | AGCACC | | ACUA | | | | | | | |
| | | GAA | | | | | | | | | |
| SNCA_ | 858 | UUCAGU | 306 | CUUCCC | 726 | AT1350- | 87.75 | 1.15 | 119.57 | 0.28 | |
| 858 | | GAAAGG | | UUUCAC | | 1351 | | | | | |
| | | GAAGCA | | UGAA | | | | | | | |
| | | CCG | | | | | | | | | |
| SNCA_ | 861 | UACUUC | 307 | CCCUUU | 727 | AT1352- | 74.26 | 0.16 | 109.87 | 0.84 | |
| 861 | | AGUGAA | | CACUGA | | 1353 | | | | | |
| | | AGGGAA | | AGUA | | | | | | | |
| | | GCA | | | | | | | | | |
| SNCA_ | 866 | UUAUUC | 308 | UCACUG | 728 | AT2442- | 81.32 | 1.85 | 69.80 | 2.04 | |
| 866 | | ACUUCA | | AAGUGA | | 2443 | | | | | |
| | | GUGAAA | | AUAA | | | | | | | |
| | | GGG | | | | | | | | | |
| SNCA_ | 867 | UGUAUU | 309 | CACUGA | 729 | AT1354- | 59.72 | 3.20 | 111.30 | 2.21 | |
| 867 | | CACUUC | | AGUGAA | | 1355 | | | | | |
| | | AGUGAA | | UACA | | | | | | | |
| | | AGG | | | | | | | | | |
| SNCA_ | 869 | UAUGUA | 310 | CUGAAG | 730 | AT2444- | 39.30 | 4.01 | 60.37 | 5.73 | |
| 869 | | UUCACU | | UGAAUA | | 2445 | | | | | |
| | | UCAGUG | | CAUA | | | | | | | |
| | | AAA | | | | | | | | | |
| SNCA_ | 870 | UCAUGU | 311 | UGAAGU | 731 | AT2446- | 35.87 | 1.93 | 61.59 | 0.03 | |
| 870 | | AUUCAC | | GAAUAC | | 2447 | | | | | |
| | | UUCAGU | | AUGA | | | | | | | |
| | | GAA | | | | | | | | | |
| SNCA_ | 871 | UCCAUG | 312 | GAAGUG | 732 | AT1356- | 28.58 | 0.24 | 75.21 | 0.14 | |
| 871 | | UAUUCA | | AAUACA | | 1357 | | | | | |
| | | CUUCAG | | UGGA | | | | | | | |
| | | UGA | | | | | | | | | |
| SNCA_ | 872 | UACCAU | 313 | AAGUGA | 733 | AT2448- | 38.42 | 0.63 | 59.34 | 1.27 | |
| 872 | | GUAUUC | | AUACAU | | 2449 | | | | | |
| | | ACUUCA | | GGUA | | | | | | | |
| | | GUG | | | | | | | | | |
| SNCA_ | 873 | UUACCA | 314 | AGUGAA | 734 | AT2450- | 37.95 | 0.33 | 61.84 | 1.11 | |
| 873 | | UGUAUU | | UACAUG | | 2451 | | | | | |
| | | CACUUC | | GUAA | | | | | | | |
| | | AGU | | | | | | | | | |
| SNCA_ | 880 | UACCCU | 315 | ACAUGG | 735 | AT1358- | 97.33 | 1.96 | 115.54 | 1.36 | |
| 880 | | GCUACC | | UAGCAG | | 1359 | | | | | |
| | | AUGUAU | | GGUA | | | | | | | |
| | | UCA | | | | | | | | | |
| SNCA_ | 888 | UACACA | 316 | GCAGGG | 736 | AT1360- | 78.77 | 2.42 | 122.58 | 5.97 | |
| 888 | | AAGACC | | UCUUUG | | 1361 | | | | | |
| | | CUGCUA | | UGUA | | | | | | | |
| | | CCA | | | | | | | | | |
| SNCA_ | 900 | UAAAAU | 317 | UGUGCU | 737 | AT1362- | 81.77 | 1.82 | 140.27 | 1.63 | |
| 900 | | CCACAG | | GUGGAU | | 1363 | | | | | |
| | | CACACA | | UUUA | | | | | | | |
| | | AAG | | | | | | | | | |
| SNCA_ | 902 | UACAAA | 318 | UGCUGU | 738 | AT1364- | 95.72 | 3.22 | 136.78 | 4.66 | |
| 902 | | AUCCAC | | GGAUUU | | 1365 | | | | | |
| | | AGCACA | | UGUA | | | | | | | |
| | | CAA | | | | | | | | | |
| SNCA_ | 915 | UGUAGA | 319 | GUGGCU | 739 | AT2452- | 31.78 | 0.81 | 61.78 | 3.03 | |
| 915 | | UUGAAG | | UCAAUC | | 2453 | | | | | |
| | | CCACAA | | UACA | | | | | | | |
| | | AAU | | | | | | | | | |
| SNCA_ | 916 | UCGUAG | 320 | UGGCUU | 740 | AT1366- | 73.27 | 0.27 | 113.24 | 2.17 | |
| 916 | | AUUGAA | | CAAUCU | | 1367 | | | | | |
| | | GCCACA | | ACGA | | | | | | | |
| | | AAA | | | | | | | | | |
| SNCA_ | 917 | UUCGUA | 321 | GGCUUC | 741 | AT1368- | 47.76 | 0.46 | 99.67 | 1.14 | |
| 917 | | GAUUGA | | AAUCUA | | 1369 | | | | | |
| | | AGCCAC | | CGAA | | | | | | | |
| | | AAA | | | | | | | | | |
| SNCA_ | 918 | UAUCGU | 322 | GCUUCA | 742 | AT1122- | 28.06 | 0.93 | | | |
| 918 | | AGAUUG | | AUCUAC | | 1123 | | | | | |
| | | AAGCCA | | GAUA | | | | | | | |
| | | CAA | | | | | | | | | |
| SNCA_ | 919 | UCAUCG | 323 | CUUCAA | 743 | AT2454- | 27.85 | 0.12 | 33.77 | 1.61 | |
| 919 | | UAGAUU | | UCUACG | | 2455 | | | | | |
| | | GAAGCC | | AUGA | | | | | | | |
| | | ACA | | | | | | | | | |
| SNCA_ | 920 | UACAUC | 324 | UUCAAU | 744 | AT1670- | 21.28 | 0.46 | 28.37 | 1.17 | 110 |
| 920 | | GUAGAU | | CUACGA | | 1671 | | | | | |
| | | UGAAGC | | UGUA | | | | | | | |
| | | CAC | | | | | | | | | |
| SNCA_ | 921 | UAACAU | 325 | UCAAUC | 745 | AT1370- | 44.82 | 0.64 | 95.05 | 0.79 | |
| 921 | | CGUAGA | | UACGAU | | 1371 | | | | | |
| | | UUGAAG | | GUUA | | | | | | | |
| | | CCA | | | | | | | | | |
| SNCA_ | 922 | UUAACA | 326 | CAAUCU | 746 | AT1124- | 33.29 | 0.46 | | | |
| 922 | | UCGUAG | | ACGAUG | | 1125 | | | | | |
| | | AUUGAA | | UUAA | | | | | | | |
| | | GCC | | | | | | | | | |
| SNCA_ | 923 | UUUAAC | 327 | AAUCUA | 747 | AT2456- | 29.92 | 0.91 | 35.17 | 0.65 | |
| 923 | | AUCGUA | | CGAUGU | | 2457 | | | | | |
| | | GAUUGA | | UAAA | | | | | | | |
| | | AGC | | | | | | | | | |
| SNCA_ | 926 | UGUUUU | 328 | CUACGA | 748 | AT1372- | 13.44 | 0.18 | 32.54 | 0.47 | 74.2 |
| 926 | | AACAUC | | UGUUAA | | 1373 | | | | | |
| | | GUAGAU | | AACA | | | | | | | |
| | | UGA | | | | | | | | | |
| SNCA_ | 952 | UGGUAG | 329 | ACCUAA | 749 | AT1618- | 33.58 | 0.91 | 70.83 | 3.23 | 579 |
| 952 | | UCACUU | | GUGACU | | 1619 | | | | | |
| | | AGGUGU | | ACCA | | | | | | | |
| | | UUU | | | | | | | | | |
| SNCA_ | 953 | UUGGUA | 330 | CCUAAG | 750 | AT1620- | 23.56 | 0.03 | 43.15 | 1.17 | 247 |
| 953 | | GUCACU | | UGACUA | | 1621 | | | | | |
| | | UAGGUG | | CCAA | | | | | | | |
| | | UUU | | | | | | | | | |
| SNCA_ | 954 | UGUGGU | 331 | CUAAGU | 751 | AT1622- | 20.93 | 0.37 | 34.84 | 0.91 | 141 |
| 954 | | AGUCAC | | GACUAC | | 1623 | | | | | |
| | | UUAGGU | | CACA | | | | | | | |
| | | GUU | | | | | | | | | |
| SNCA_ | 955 | UAGUGG | 332 | UAAGUG | 752 | AT1624- | 27.96 | 0.34 | 36.13 | 0.35 | 72.3 |
| 955 | | UAGUCA | | ACUACC | | 1625 | | | | | |
| | | CUUAGG | | ACUA | | | | | | | |
| | | UGU | | | | | | | | | |
| SNCA_ | 956 | UAAGUG | 333 | AAGUGA | 753 | AT2458- | 68.87 | 1.92 | 78.83 | 3.69 | |
| 956 | | GUAGUC | | CUACCA | | 2459 | | | | | |
| | | ACUUAG | | CUUA | | | | | | | |
| | | GUG | | | | | | | | | |
| SNCA_ | 957 | UUAAGU | 334 | AGUGAC | 754 | AT1626- | 93.33 | 2.05 | 102.73 | 2.09 | |
| 957 | | GGUAGU | | UACCAC | | 1627 | | | | | |
| | | CACUUA | | UUAA | | | | | | | |
| | | GGU | | | | | | | | | |
| SNCA_ | 958 | UAUAAG | 335 | GUGACU | 755 | AT1374- | 95.43 | 1.75 | 114.63 | 1.06 | |
| 958 | | UGGUAG | | ACCACU | | 1375 | | | | | |
| | | UCACUU | | UAUA | | | | | | | |
| | | AGG | | | | | | | | | |
| SNCA_ | 959 | UAAUAA | 336 | UGACUA | 756 | AT1376- | 96.25 | 1.41 | 111.52 | 0.07 | |
| 959 | | GUGGUA | | CCACUU | | 1377 | | | | | |
| | | GUCACU | | AUUA | | | | | | | |
| | | UAG | | | | | | | | | |
| SNCA_ | 960 | UAAAUA | 337 | GACUAC | 757 | AT1628- | 73.53 | 3.28 | 84.17 | 3.74 | |
| 960 | | AGUGGU | | CACUUA | | 1629 | | | | | |
| | | AGUCAC | | UUUA | | | | | | | |
| | | UUA | | | | | | | | | |
| SNCA_ | 961 | UGAAAU | 338 | ACUACC | 758 | AT1378- | 83.34 | 2.00 | 104.40 | 0.48 | |
| 961 | | AAGUGG | | ACUUAU | | 1379 | | | | | |
| | | UAGUCA | | UUCA | | | | | | | |
| | | CUU | | | | | | | | | |
| SNCA_ | 962 | UAGAAA | 339 | CUACCA | 759 | AT1380- | 72.93 | 2.89 | 114.57 | 3.37 | |
| 962 | | UAAGUG | | CUUAUU | | 1381 | | | | | |
| | | GUAGUC | | UCUA | | | | | | | |
| | | ACU | | | | | | | | | |
| SNCA_ | 963 | UUAGAA | 340 | UACCAC | 760 | AT2460- | 44.68 | 0.02 | 39.85 | 1.73 | 86.4 |
| 963 | | AUAAGU | | UUAUUU | | 2461 | | | | | |
| | | GGUAGU | | CUAA | | | | | | | |
| | | CAC | | | | | | | | | |
| SNCA_ | 964 | UUUAGA | 341 | ACCACU | 761 | AT1126- | 35.34 | 0.72 | | | |
| 964 | | AAUAAG | | UAUUUC | | 1127 | | | | | |
| | | UGGUAG | | UAAA | | | | | | | |
| | | UCA | | | | | | | | | |
| SNCA_ | 966 | UAUUUA | 342 | CACUUA | 762 | AT2462- | 47.56 | 0.96 | 49.14 | 5.00 | 101 |
| 966 | | GAAAUA | | UUUCUA | | 2463 | | | | | |
| | | AGUGGU | | AAUA | | | | | | | |
| | | AGU | | | | | | | | | |
| SNCA_ | 967 | UGAUUU | 343 | ACUUAU | 763 | AT1382- | 26.20 | 0.84 | 45.91 | 1.00 | |
| 967 | | AGAAAU | | UUCUAA | | 1383 | | | | | |
| | | AAGUGG | | AUCA | | | | | | | |
| | | UAG | | | | | | | | | |
| SNCA_ | 969 | UAGGAU | 344 | UUAUUU | 764 | AT1384- | 80.64 | 1.42 | 109.50 | 1.99 | |
| 969 | | UUAGAA | | CUAAAU | | 1385 | | | | | |
| | | AUAAGU | | CCUA | | | | | | | |
| | | GGU | | | | | | | | | |
| SNCA_ | 997 | UUCUGA | 345 | UUGCUG | 765 | AT1128- | 64.08 | 0.03 | | | |
| 997 | | ACAACA | | UUGUUC | | 1129 | | | | | |
| | | GCAACA | | AGAA | | | | | | | |
| | | AAA | | | | | | | | | |
| SNCA_ | 998 | UUUCUG | 346 | UGCUGU | 766 | AT1386- | 37.03 | 0.07 | 62.31 | 1.95 | |
| 998 | | AACAAC | | UGUUCA | | 1387 | | | | | |
| | | AGCAAC | | GAAA | | | | | | | |
| | | AAA | | | | | | | | | |
| SNCA_ | 999 | UCUUCU | 347 | GCUGUU | 767 | AT1388- | 29.00 | 0.23 | 63.19 | 0.67 | |
| 999 | | GAACAA | | GUUCAG | | 1389 | | | | | |
| | | CAGCAA | | AAGA | | | | | | | |
| | | CAA | | | | | | | | | |
| SNCA_ | 1000 | UACUUC | 348 | CUGUUG | 768 | AT2464- | 40.60 | 1.62 | 54.75 | 2.72 | |
| 1000 | | UGAACA | | UUCAGA | | 2465 | | | | | |
| | | ACAGCA | | AGUA | | | | | | | |
| | | ACA | | | | | | | | | |
| SNCA_ | 1001 | UAACUU | 349 | UGUUGU | 769 | AT1390- | 35.90 | 0.38 | 54.07 | 2.10 | |
| 1001 | | CUGAAC | | UCAGAA | | 1391 | | | | | |
| | | AACAGC | | GUUA | | | | | | | |
| | | AAC | | | | | | | | | |
| SNCA_ | 1002 | UCAACU | 350 | GUUGUU | 770 | AT1392- | 35.69 | 0.41 | 44.16 | 1.66 | |
| 1002 | | UCUGAA | | CAGAAG | | 1393 | | | | | |
| | | CAACAG | | UUGA | | | | | | | |
| | | CAA | | | | | | | | | |
| SNCA_ | 1003 | UACAAC | 351 | UUGUUC | 771 | AT1394- | 39.47 | 1.37 | 52.07 | 1.07 | |
| 1003 | | UUCUGA | | AGAAGU | | 1395 | | | | | |
| | | ACAACA | | UGUA | | | | | | | |
| | | GCA | | | | | | | | | |
| SNCA_ | 1004 | UAACAA | 352 | UGUUCA | 772 | AT1396- | 107.18 | 0.05 | 119.74 | 1.03 | |
| 1004 | | CUUCUG | | GAAGUU | | 1397 | | | | | |
| | | AACAAC | | GUUA | | | | | | | |
| | | AGC | | | | | | | | | |
| SNCA_ | 1005 | UUAACA | 353 | GUUCAG | 773 | AT1398- | 36.17 | 0.16 | 51.16 | 0.08 | |
| 1005 | | ACUUCU | | AAGUUG | | 1399 | | | | | |
| | | GAACAA | | UUAA | | | | | | | |
| | | CAG | | | | | | | | | |
| SNCA_ | 1006 | UCUAAC | 354 | UUCAGA | 774 | AT1400- | 41.80 | 0.30 | 63.67 | 0.99 | |
| 1006 | | AACUUC | | AGUUGU | | 1401 | | | | | |
| | | UGAACA | | UAGA | | | | | | | |
| | | ACA | | | | | | | | | |
| SNCA_ | 1007 | UACUAA | 355 | UCAGAA | 775 | AT2466- | 38.02 | 2.79 | 69.95 | 3.05 | |
| 1007 | | CAACUU | | GUUGUU | | 2467 | | | | | |
| | | CUGAAC | | AGUA | | | | | | | |
| | | AAC | | | | | | | | | |
| SNCA_ | 1008 | UCACUA | 356 | CAGAAG | 776 | AT2468- | 30.58 | 2.69 | 37.78 | 4.01 | |
| 1008 | | ACAACU | | UUGUUA | | 2469 | | | | | |
| | | UCUGAA | | GUGA | | | | | | | |
| | | CAA | | | | | | | | | |
| SNCA_ | 1009 | UUCACU | 357 | AGAAGU | 777 | AT1402- | 24.90 | 0.13 | 40.45 | 1.21 | 60.2 |
| 1009 | | AACAAC | | UGUUAG | | 1403 | | | | | |
| | | UUCUGA | | UGAA | | | | | | | |
| | | ACA | | | | | | | | | |
| SNCA_ | 1010 | UAUCAC | 358 | GAAGUU | 778 | AT2470- | 31.73 | 2.33 | 36.31 | 1.09 | |
| 1010 | | UAACAA | | GUUAGU | | 2471 | | | | | |
| | | CUUCUG | | GAUA | | | | | | | |
| | | AAC | | | | | | | | | |
| SNCA_ | 1011 | UAAUCA | 359 | AAGUUG | 779 | AT1404- | 26.83 | 0.14 | 58.18 | 0.51 | |
| 1011 | | CUAACA | | UUAGUG | | 1405 | | | | | |
| | | ACUUCU | | AUUA | | | | | | | |
| | | GAA | | | | | | | | | |
| SNCA_ | 1012 | UAAAUC | 360 | AGUUGU | 780 | AT1406- | 103.49 | 0.54 | 108.04 | 1.06 | |
| 1012 | | ACUAAC | | UAGUGA | | 1407 | | | | | |
| | | AACUUC | | UUUA | | | | | | | |
| | | UGA | | | | | | | | | |
| SNCA_ | 1013 | UCAAAU | 361 | GUUGUU | 781 | AT1408- | 29.85 | 0.81 | 47.56 | 1.26 | |
| 1013 | | CACUAA | | AGUGAU | | 1409 | | | | | |
| | | CAACUU | | UUGA | | | | | | | |
| | | CUG | | | | | | | | | |
| SNCA_ | 1014 | UGCAAA | 362 | UUGUUA | 782 | AT1410- | 35.44 | 1.06 | 56.37 | 3.08 | |
| 1014 | | UCACUA | | GUGAUU | | 1411 | | | | | |
| | | ACAACU | | UGCA | | | | | | | |
| | | UCU | | | | | | | | | |
| SNCA_ | 1015 | UAGCAA | 363 | UGUUAG | 783 | AT2472- | 32.24 | 3.11 | 36.17 | 1.38 | |
| 1015 | | AUCACU | | UGAUUU | | 2473 | | | | | |
| | | AACAAC | | GCUA | | | | | | | |
| | | UUC | | | | | | | | | |
| SNCA_ | 1016 | UUAGCA | 364 | GUUAGU | 784 | AT2474- | 34.74 | 1.68 | 42.34 | 0.19 | |
| 1016 | | AAUCAC | | GAUUUG | | 2475 | | | | | |
| | | UAACAA | | CUAA | | | | | | | |
| | | CUU | | | | | | | | | |
| SNCA_ | 1017 | UAUAGC | 365 | UUAGUG | 785 | AT1130- | 55.62 | 0.45 | | | |
| 1017 | | AAAUCA | | AUUUGC | | 1131 | | | | | |
| | | CUAACA | | UAUA | | | | | | | |
| | | ACU | | | | | | | | | |
| SNCA_ | 1018 | UGAUAG | 366 | UAGUGA | 786 | AT2476- | 42.69 | 1.00 | 51.40 | 0.10 | |
| 1018 | | CAAAUC | | UUUGCU | | 2477 | | | | | |
| | | ACUAAC | | AUCA | | | | | | | |
| | | AAC | | | | | | | | | |
| SNCA_ | 1019 | UUGAUA | 367 | AGUGAU | 787 | AT2478- | 42.74 | 3.67 | 58.63 | 2.36 | |
| 1019 | | GCAAAU | | UUGCUA | | 2479 | | | | | |
| | | CACUAA | | UCAA | | | | | | | |
| | | CAA | | | | | | | | | |
| SNCA_ | 1020 | UAUGAU | 368 | GUGAUU | 788 | AT1412- | 44.21 | 0.80 | 50.67 | 0.63 | |
| 1020 | | AGCAAA | | UGCUAU | | 1413 | | | | | |
| | | UCACUA | | CAUA | | | | | | | |
| | | ACA | | | | | | | | | |
| SNCA_ | 1054 | UUAUCA | 369 | UGUCUU | 789 | AT1414- | 41.41 | 1.36 | 70.73 | 2.44 | |
| 1054 | | UUAAAA | | UUAAUG | | 1415 | | | | | |
| | | GACACC | | AUAA | | | | | | | |
| | | UAA | | | | | | | | | |
| SNCA_ | 1055 | UGUAUC | 370 | GUCUUU | 790 | AT1416- | 47.30 | 0.27 | 68.37 | 0.66 | |
| 1055 | | AUUAAA | | UAAUGA | | 1417 | | | | | |
| | | AGACAC | | UACA | | | | | | | |
| | | CUA | | | | | | | | | |
| SNCA_ | 1056 | UAGUAU | 371 | UCUUUU | 791 | AT1418- | 37.09 | 0.11 | 53.92 | 0.26 | |
| 1056 | | CAUUAA | | AAUGAU | | 1419 | | | | | |
| | | AAGACA | | ACUA | | | | | | | |
| | | CCU | | | | | | | | | |
| SNCA_ | 1057 | UCAGUA | 372 | CUUUUA | 792 | AT1420- | 92.38 | 0.83 | 110.68 | 1.08 | |
| 1057 | | UCAUUA | | AUGAUA | | 1421 | | | | | |
| | | AAAGAC | | CUGA | | | | | | | |
| | | ACC | | | | | | | | | |
| SNCA_ | 1058 | UACAGU | 373 | UUUUAA | 793 | AT1422- | 46.20 | 0.05 | 60.28 | 2.14 | |
| 1058 | | AUCAUU | | UGAUAC | | 1423 | | | | | |
| | | AAAAGA | | UGUA | | | | | | | |
| | | CAC | | | | | | | | | |
| SNCA_ | 1059 | UGACAG | 374 | UUUAAU | 794 | AT2480- | 58.92 | 1.05 | 79.18 | 12.25 | |
| 1059 | | UAUCAU | | GAUACU | | 2481 | | | | | |
| | | UAAAAG | | GUCA | | | | | | | |
| | | ACA | | | | | | | | | |
| SNCA_ | 1069 | UAUUAU | 375 | CUGUCU | 795 | AT1424- | 92.94 | 1.46 | 96.26 | 2.16 | |
| 1069 | | UCUUAG | | AAGAAU | | 1425 | | | | | |
| | | ACAGUA | | AAUA | | | | | | | |
| | | UCA | | | | | | | | | |
| SNCA_ | 1071 | UUCAUU | 376 | GUCUAA | 796 | AT1426- | 26.53 | 1.87 | 50.90 | 5.02 | |
| 1071 | | AUUCUU | | GAAUAA | | 1427 | | | | | |
| | | AGACAG | | UGAA | | | | | | | |
| | | UAU | | | | | | | | | |
| SNCA_ | 1072 | UGUCAU | 377 | UCUAAG | 797 | AT1428- | 31.92 | 0.55 | 44.71 | 8.69 | |
| 1072 | | UAUUCU | | AAUAAU | | 1429 | | | | | |
| | | UAGACA | | GACA | | | | | | | |
| | | GUA | | | | | | | | | |
| SNCA_ | 1073 | UCGUCA | 378 | CUAAGA | 798 | AT2482- | 32.94 | 1.58 | 45.31 | 0.44 | |
| 1073 | | UUAUUC | | AUAAUG | | 2483 | | | | | |
| | | UUAGAC | | ACGA | | | | | | | |
| | | AGU | | | | | | | | | |
| SNCA_ | 1074 | UACGUC | 379 | UAAGAA | 799 | AT1430- | 32.58 | 0.30 | 37.05 | 2.37 | |
| 1074 | | AUUAUU | | UAAUGA | | 1431 | | | | | |
| | | CUUAGA | | CGUA | | | | | | | |
| | | CAG | | | | | | | | | |
| SNCA_ | 1075 | UUACGU | 380 | AAGAAU | 800 | AT1132- | 44.54 | 0.52 | | | |
| 1075 | | CAUUAU | | AAUGAC | | 1133 | | | | | |
| | | UCUUAG | | GUAA | | | | | | | |
| | | ACA | | | | | | | | | |
| SNCA_ | 1077 | UAAUAC | 381 | GAAUAA | 801 | AT1432- | 121.47 | 12.96 | 99.74 | 6.77 | |
| 1077 | | GUCAUU | | UGACGU | | 1433 | | | | | |
| | | AUUCUU | | AUUA | | | | | | | |
| | | AGA | | | | | | | | | |
| SNCA_ | 1079 | UACAAU | 382 | AUAAUG | 802 | AT1434- | 31.62 | 0.09 | 59.80 | 1.11 | |
| 1079 | | ACGUCA | | ACGUAU | | 1435 | | | | | |
| | | UUAUUC | | UGUA | | | | | | | |
| | | UUA | | | | | | | | | |
| SNCA_ | 1080 | UCACAA | 383 | UAAUGA | 803 | AT2484- | 32.80 | 3.53 | 35.19 | 0.03 | |
| 1080 | | UACGUC | | CGUAUU | | 2485 | | | | | |
| | | AUUAUU | | GUGA | | | | | | | |
| | | CUU | | | | | | | | | |
| SNCA_ | 1081 | UUCACA | 384 | AAUGAC | 804 | AT2486- | 34.91 | 0.71 | 42.38 | 0.04 | |
| 1081 | | AUACGU | | GUAUUG | | 2487 | | | | | |
| | | CAUUAU | | UGAA | | | | | | | |
| | | UCU | | | | | | | | | |
| SNCA_ | 1086 | UAAAUU | 385 | CGUAUU | 805 | AT1436- | 94.26 | 4.12 | 132.12 | 6.66 | |
| 1086 | | UCACAA | | GUGAAA | | 1437 | | | | | |
| | | UACGUC | | UUUA | | | | | | | |
| | | AUU | | | | | | | | | |
| SNCA_ | 1087 | UCAAAU | 386 | GUAUUG | 806 | AT1134- | 39.56 | 0.27 | | | |
| 1087 | | UUCACA | | UGAAAU | | 1135 | | | | | |
| | | AUACGU | | UUGA | | | | | | | |
| | | CAU | | | | | | | | | |
| SNCA_ | 1088 | UACAAA | 387 | UAUUGU | 807 | AT1136- | 40.04 | 0.27 | | | |
| 1088 | | UUUCAC | | GAAAUU | | 1137 | | | | | |
| | | AAUACG | | UGUA | | | | | | | |
| | | UCA | | | | | | | | | |
| SNCA_ | 1126 | UGUUUC | 388 | AUGUGA | 808 | AT2488- | 46.59 | 3.78 | 66.85 | 6.05 | |
| 1126 | | AUGCUC | | GCAUGA | | 2489 | | | | | |
| | | ACAUAU | | AACA | | | | | | | |
| | | UUU | | | | | | | | | |
| SNCA_ | 1127 | UAGUUU | 389 | UGUGAG | 809 | AT2490- | 38.29 | 0.00 | 60.84 | 0.66 | |
| 1127 | | CAUGCU | | CAUGAA | | 2491 | | | | | |
| | | CACAUA | | ACUA | | | | | | | |
| | | UUU | | | | | | | | | |
| SNCA_ | 1128 | UUAGUU | 390 | GUGAGC | 810 | AT1438- | 48.00 | 1.87 | 95.90 | 1.83 | 469 |
| 1128 | | UCAUGC | | AUGAAA | | 1439 | | | | | |
| | | UCACAU | | CUAA | | | | | | | |
| | | AUU | | | | | | | | | |
| SNCA_ | 1129 | UAUAGU | 391 | UGAGCA | 811 | AT1440- | 29.70 | 0.89 | 58.69 | 0.78 | |
| 1129 | | UUCAUG | | UGAAAC | | 1441 | | | | | |
| | | CUCACA | | UAUA | | | | | | | |
| | | UAU | | | | | | | | | |
| SNCA_ | 1130 | UCAUAG | 392 | GAGCAU | 812 | AT2492- | 64.05 | 7.10 | 81.28 | 7.32 | |
| 1130 | | UUUCAU | | GAAACU | | 2493 | | | | | |
| | | GCUCAC | | AUGA | | | | | | | |
| | | AUA | | | | | | | | | |
| SNCA_ | 1131 | UGCAUA | 393 | AGCAUG | 813 | AT2494- | 74.33 | 1.24 | 87.78 | 10.54 | |
| 1131 | | GUUUCA | | AAACUA | | 2495 | | | | | |
| | | UGCUCA | | UGCA | | | | | | | |
| | | CAU | | | | | | | | | |
| SNCA_ | 1132 | UUGCAU | 394 | GCAUGA | 814 | AT1138- | 39.83 | 0.68 | | | |
| 1132 | | AGUUUC | | AACUAU | | 1139 | | | | | |
| | | AUGCUC | | GCAA | | | | | | | |
| | | ACA | | | | | | | | | |
| SNCA_ | 1133 | UGUGCA | 395 | CAUGAA | 815 | AT1442- | 87.10 | 2.13 | 134.13 | 0.17 | |
| 1133 | | UAGUUU | | ACUAUG | | 1443 | | | | | |
| | | CAUGCU | | CACA | | | | | | | |
| | | CAC | | | | | | | | | |
| SNCA_ | 1134 | UGGUGC | 396 | AUGAAA | 816 | AT1444- | 33.57 | 1.20 | 74.13 | 1.02 | |
| 1134 | | AUAGUU | | CUAUGC | | 1445 | | | | | |
| | | UCAUGC | | ACCA | | | | | | | |
| | | UCA | | | | | | | | | |
| SNCA_ | 1135 | UAGGUG | 397 | UGAAAC | 817 | AT1446- | 31.14 | 0.11 | 60.86 | 0.58 | |
| 1135 | | CAUAGU | | UAUGCA | | 1447 | | | | | |
| | | UUCAUG | | CCUA | | | | | | | |
| | | CUC | | | | | | | | | |
| SNCA_ | 1136 | UUAGGU | 398 | GAAACU | 818 | AT1448- | 40.52 | 0.24 | 79.10 | 2.18 | |
| 1136 | | GCAUAG | | AUGCAC | | 1449 | | | | | |
| | | UUUCAU | | CUAA | | | | | | | |
| | | GCU | | | | | | | | | |
| SNCA_ | 1137 | UAUAGG | 399 | AAACUA | 819 | AT1450- | 101.53 | 3.24 | 125.31 | 3.05 | |
| 1137 | | UGCAUA | | UGCACC | | 1451 | | | | | |
| | | GUUUCA | | UAUA | | | | | | | |
| | | UGC | | | | | | | | | |
| SNCA_ | 1138 | UUAUAG | 400 | AACUAU | 820 | AT2496- | 76.10 | 5.24 | 81.07 | 3.00 | |
| 1138 | | GUGCAU | | GCACCU | | 2497 | | | | | |
| | | AGUUUC | | AUAA | | | | | | | |
| | | AUG | | | | | | | | | |
| SNCA_ | 1140 | UUUUAU | 401 | CUAUGC | 821 | AT1452- | 110.34 | 1.93 | 124.30 | 1.26 | |
| 1140 | | AGGUGC | | ACCUAU | | 1453 | | | | | |
| | | AUAGUU | | AAAA | | | | | | | |
| | | UCA | | | | | | | | | |
| SNCA_ | 1142 | UUAUUU | 402 | AUGCAC | 822 | AT2498- | 49.81 | 3.27 | 71.26 | 4.92 | |
| 1142 | | AUAGGU | | CUAUAA | | 2499 | | | | | |
| | | GCAUAG | | AUAA | | | | | | | |
| | | UUU | | | | | | | | | |
| SNCA_ | 1144 | UAGUAU | 403 | GCACCU | 823 | AT1454- | 89.46 | 0.68 | 110.34 | 1.33 | |
| 1144 | | UUAUAG | | AUAAAU | | 1455 | | | | | |
| | | GUGCAU | | ACUA | | | | | | | |
| | | AGU | | | | | | | | | |
| SNCA_ | 1168 | UGCAAA | 404 | AUUUUA | 824 | AT1456- | 57.72 | 1.34 | 85.91 | 4.51 | |
| 1168 | | AUGGUA | | CCAUUU | | 1457 | | | | | |
| | | AAAUUU | | UGCA | | | | | | | |
| | | CAU | | | | | | | | | |
| SNCA_ | 1172 | UCAUCG | 405 | UACCAU | 825 | AT1672- | 75.12 | 1.35 | 92.30 | 1.76 | |
| 1172 | | CAAAAU | | UUUGCG | | 1673 | | | | | |
| | | GGUAAA | | AUGA | | | | | | | |
| | | AUU | | | | | | | | | |
| SNCA_ | 1174 | UCACAU | 406 | CCAUUU | 826 | AT1140- | 68.16 | 2.50 | | | |
| 1174 | | CGCAAA | | UGCGAU | | 1141 | | | | | |
| | | AUGGUA | | GUGA | | | | | | | |
| | | AAA | | | | | | | | | |
| SNCA_ | 1175 | UACACA | 407 | CAUUUU | 827 | AT1458- | 103.81 | 0.71 | 140.57 | 1.75 | |
| 1175 | | UCGCAA | | GCGAUG | | 1459 | | | | | |
| | | AAUGGU | | UGUA | | | | | | | |
| | | AAA | | | | | | | | | |
| SNCA_ | 1176 | UAACAC | 408 | AUUUUG | 828 | AT1460- | 74.14 | 1.04 | 96.32 | 2.23 | |
| 1176 | | AUCGCA | | CGAUGU | | 1461 | | | | | |
| | | AAAUGG | | GUUA | | | | | | | |
| | | UAA | | | | | | | | | |
| SNCA_ | 1177 | UAAACA | 409 | UUUUGC | 829 | AT1462- | 70.60 | 0.63 | 88.29 | 0.20 | |
| 1177 | | CAUCGC | | GAUGUG | | 1463 | | | | | |
| | | AAAAUG | | UUUA | | | | | | | |
| | | GUA | | | | | | | | | |
| SNCA_ | 1178 | UAAAAC | 410 | UUUGCG | 830 | AT1142- | 75.63 | 1.22 | | | |
| 1178 | | ACAUCG | | AUGUGU | | 1143 | | | | | |
| | | CAAAAU | | UUUA | | | | | | | |
| | | GGU | | | | | | | | | |
| SNCA_ | 1179 | UUAAAA | 411 | UUGCGA | 831 | AT1464- | 110.49 | 0.02 | 121.81 | 0.49 | |
| 1179 | | CACAUC | | UGUGUU | | 1465 | | | | | |
| | | GCAAAA | | UUAA | | | | | | | |
| | | UGG | | | | | | | | | |
| SNCA_ | 1182 | UGAAUA | 412 | CGAUGU | 832 | AT1466- | 118.45 | 0.57 | 128.71 | 0.40 | |
| 1182 | | AAACAC | | GUUUUA | | 1467 | | | | | |
| | | AUCGCA | | UUCA | | | | | | | |
| | | AAA | | | | | | | | | |
| SNCA_ | 1183 | UUGAAU | 413 | GAUGUG | 833 | AT1468- | 72.48 | 1.26 | 84.16 | 1.21 | |
| 1183 | | AAAACA | | UUUUAU | | 1469 | | | | | |
| | | CAUCGC | | UCAA | | | | | | | |
| | | AAA | | | | | | | | | |
| SNCA_ | 1184 | UGUGAA | 414 | AUGUGU | 834 | AT1470- | 103.14 | 1.32 | 83.83 | 0.65 | |
| 1184 | | UAAAAC | | UUUAUU | | 1471 | | | | | |
| | | ACAUCG | | CACA | | | | | | | |
| | | CAA | | | | | | | | | |
| SNCA_ | 1186 | UAAGUG | 415 | GUGUUU | 835 | AT1472- | 100.14 | 0.34 | 104.22 | 0.04 | |
| 1186 | | AAUAAA | | UAUUCA | | 1473 | | | | | |
| | | ACACAU | | CUUA | | | | | | | |
| | | CGC | | | | | | | | | |
| SNCA_ | 1187 | UCAAGU | 416 | UGUUUU | 836 | AT1474- | 57.23 | 1.09 | 68.81 | 0.55 | |
| 1187 | | GAAUAA | | AUUCAC | | 1475 | | | | | |
| | | AACACA | | UUGA | | | | | | | |
| | | UCG | | | | | | | | | |
| SNCA_ | 1188 | UACAAG | 417 | GUUUUA | 837 | AT1476- | 64.08 | 0.34 | 88.53 | 3.23 | |
| 1188 | | UGAAUA | | UUCACU | | 1477 | | | | | |
| | | AAACAC | | UGUA | | | | | | | |
| | | AUC | | | | | | | | | |
| SNCA_ | 1189 | UCACAA | 418 | UUUUAU | 838 | AT1478- | 60.96 | 1.89 | 82.93 | 0.01 | |
| 1189 | | GUGAAU | | UCACUU | | 1479 | | | | | |
| | | AAAACA | | GUGA | | | | | | | |
| | | CAU | | | | | | | | | |
| SNCA_ | 1205 | UACCAU | 419 | UUGUAU | 839 | AT1144- | 97.07 | 1.76 | | | |
| 1205 | | UUAUAU | | AUAAAU | | 1145 | | | | | |
| | | ACAAAC | | GGUA | | | | | | | |
| | | ACA | | | | | | | | | |
| SNCA_ | 1207 | UUCACC | 420 | GUAUAU | 840 | AT1480- | 84.43 | 0.16 | 110.16 | 1.70 | |
| 1207 | | AUUUAU | | AAAUGG | | 1481 | | | | | |
| | | AUACAA | | UGAA | | | | | | | |
| | | ACA | | | | | | | | | |

### Example 2. In Vivo Screening

In vivo screening was performed using 12 week old hSNCA transgenic mice. Briefly, PBS or divalent siRNA was administered via bilateral stereotactic ICV injection at a dose of 1 nmol or 5 nmol in a total volume of 5 uL. One week after compound administration, animals were euthanized, perfused, and the brain was sliced into 1 mm thick cortical slices. Biopsy punches (1-2 mm diameter) were taken of the relevant brain regions (Striatum=Cpu, hippocampus=HP, temporal cortex=tCTx) and were snap frozen. Tissue punches were homogenized in Trizol (Invitrogen) using a Qiagen TissueLyser and total RNA was isolated the RNeasy kit (Qiagen). Quantitative reverse transcriptase polymerase change reaction (QRT-PCR) was performed to assess target (human SNCA) gene expression relative to housekeeping (mouse ATP5B) gene expression using TaqMan primer/probes and Fast Advanced master mix (Thermo). Data are presented as % SNCA target knockdown (N=8 animals, 4 male, 4 female, 2 technical replicates each) relative to PBS treated control animals, where 100% represents complete target knockdown and 0% represents no target knockdown.

TABLE 3
In Vivo Screening

| | | | In | % SNCA Knock-Down | | | | | |
|---|---|---|---|---|---|---|---|---|---|
| | 21 mer | 16 mer | vitro | Cpu | | HP | | tCTx | |
| | antisense | sense | duplex | 5 | 1 | 5 | 1 | 5 | 1 |
| Position | sequence | sequence | ID | nmol | nmol | nmol | nmol | nmol | nmol |
| SNCA_737 | UGGAACUGAGCACU | CAAGUGCUCAGUU | AT4511- | 60 | 30.9 | 67.2 | 33.1 | 65.8 | 42.8 |
| | UGUACAG | CCA | 4514 | | | | | | |
| | (SEQ ID NO: 244) | (SEQ ID NO: 664) | | | | | | | |
| SNCA_955 | UAGUGGUAGUCACU | UAAGUGACUACCA | AT4507- | 51.2 | 20.1 | 60.7 | 14.3 | 58.7 | 39.6 |
| | UAGGUGU | CUA | 4510 | | | | | | |
| | (SEQ ID NO: 332) | (SEQ ID NO: 752) | | | | | | | |
| SNCA_1009 | UUCACUAACAACUUC | AGAAGUUGUUAG | AT4518- | 33.5 | 18.9 | 14.2 | −9.5 | 20.1 | −9.5 |
| | UGAACA | UGAA | 4515 | | | | | | |
| | (SEQ ID NO: 357) | (SEQ ID NO: 777) | | | | | | | |
| SNCA_926 | UGUUUUAACAUCGU | CUACGAUGUUAA | AT4520- | 62.6 | 20.2 | 77.8 | −5.9 | 50.8 | −46.4 |
| | AGAUUGA | AACA | 4517 | | | | | | |
| | (SEQ ID NO: 328) | (SEQ ID NO: 748) | | | | | | | |
| SNCA_963 | UUAGAAAUAAGUGG | UACCACUUAUUUC | AT4512- | 77 | 32.9 | 61.7 | 47.2 | 60.8 | −13.8 |
| | UAGUCAC | UAA | 4509 | | | | | | |
| | (SEQ ID NO: 340) | (SEQ ID NO: 760) | | | | | | | |
| SNCA_739 | UUUGGAACUGAGCA | AGUGCUCAGUUCC | AT4516- | 47.1 | 24.5 | 63.3 | 54.6 | 41.4 | −7.7 |
| | CUUGUAC | AAA | 4513 | | | | | | |
| | (SEQ ID NO: 246) | (SEQ ID NO: 666) | | | | | | | |
| SNCA_421 | UGGUCUUCUCAGCCA | GUGGCUGAGAAG | AT4522- | −2 | 31.8 | 10.7 | 16.8 | −147.7 | −36.2 |
| | CUGUUG | ACCA | 4519 | | | | | | |
| | (SEQ ID NO: 131) | (SEQ ID NO: 551) | | | | | | | |
| SNCA_399 | UACACCAUGCACCAC | AGUGGUGCAUGG | AT4524- | 71.6 | 41.6 | 79.9 | 34.9 | 47.7 | −5.8 |
| | UCCCUC | UGUA | 4521 | | | | | | |
| | (SEQ ID NO: 115) | (SEQ ID NO: 535) | | | | | | | |
| SNCA_621 | UUCAUAAGCCUCAUU | CAAUGAGGCUUA | AT4547- | 48.5 | 48.5 | 50 | 41 | 35.8 | 17.7 |
| | GUCAGG | UGAA | 4546 | | | | | | |
| | (SEQ ID NO: 179) | (SEQ ID NO: 599) | | | | | | | |
| SNCA_966 | UAUUUAGAAAUAAG | CACUUAUUUCUAA | AT4563- | 54 | 29.7 | 58.6 | 37.6 | 47.1 | 7.5 |
| | UGGUAGU | AUA | 4562 | | | | | | |
| | (SEQ ID NO: 342) | (SEQ ID NO: 762) | | | | | | | |
| SNCA_744 | UGCACAUUGGAACU | UCAGUUCCAAUGU | AT4555- | 52.2 | 43.5 | 67.5 | 40.7 | 23.5 | −7.2 |
| | GAGCACU | GCA | 4554 | | | | | | |
| | (SEQ ID NO: 251) | (SEQ ID NO: 671) | | | | | | | |
| SNCA_270 | UGAAAGUCCUUUCA | CAUGAAAGGACU | AT4543- | 35.7 | 25.7 | 52.7 | 20.2 | 24.2 | 7.1 |
| | UGAAUAC | UUCA | 4542 | | | | | | |
| | (SEQ ID NO: 39) | (SEQ ID NO: 459) | | | | | | | |
| SNCA_740 | UAUUGGAACUGAGC | GUGCUCAGUUCCA | AT4553- | 22.8 | 26.6 | 39.5 | 26.5 | −6.6 | 4.3 |
| | ACUUGUA | AUA | 4552 | | | | | | |
| | (SEQ ID NO: 247) | (SEQ ID NO: 667) | | | | | | | |
| SNCA_953 | UUGGUAGUCACUUA | CCUAAGUGACUAC | AT4559- | 55 | 29.5 | 64.3 | 46.5 | 28.8 | −3 |
| | GGUGUUU | CAA | 4558 | | | | | | |
| | (SEQ ID NO: 330) | (SEQ ID NO: 750) | | | | | | | |
| SNCA_682 | UGAGCAAAGAUAUU | GAAAUAUCUUUG | AT4549- | 38.9 | 19.1 | 53.7 | 43.7 | 22.9 | 17.7 |
| | UCUUAGG | CUCA | 4548 | | | | | | |
| | (SEQ ID NO: 211) | (SEQ ID NO: 631) | | | | | | | |
| SNCA_736 | UGAACUGAGCACUU | ACAAGUGCUCAGU | AT4551- | 55 | 32.9 | 67.8 | 12.5 | 43.7 | −16.6 |
| | GUACAGG | UCA | 4550 | | | | | | |
| | (SEQ ID NO: 243) | (SEQ ID NO: 663) | | | | | | | |
| SNCA_954 | UGUGGUAGUCACUU | CUAAGUGACUACC | AT4561- | 60.4 | 20.7 | 61 | 31.5 | 21.7 | −33 |
| | AGGUGUU | ACA | 4560 | | | | | | |
| | (SEQ ID NO: 331) | (SEQ ID NO: 751) | | | | | | | |

### Example 3. In Vivo Inhibition of SNCA Gene Expression Using a Di-siRNA Sequence in Mouse

### Objective

This Example describes the results of a series of experiments undertaken to investigate the ability of an siRNA molecule complementary to a specific region within a human SNCA transcript to effectuate reduced expression of the SNCA gene.

In Vivo Administration of di-siRNA

An siRNA molecule of the disclosure was synthesized as a di-branched siRNA molecule having the structure of Formula XVII. The sense strand had the sequence of SEQ ID No.: 664 and had a pattern of chemical modifications having the broad structure of Formula III and the specific structure of Formula S1. The antisense strand had the sequence of SEQ ID No.: 244 and had a pattern of chemical modifications having the broad structure of Formula II and the specific structure of Formula A2. The antisense strand further included a 5′ vinyl phosphonate moiety of Formula XI.

In vivo durability was assessed in mouse using similar methods as described in Example 2. Briefly, PBS or the di-branched siRNA molecule described above was administered via bilateral stereotactic intracerebroventricular (ICV) injection at a dose of 30 nmol in a total volume of 10 μL.

One month, two months, three months, or four months after compound administration, animals were euthanized, perfused, and the brain was sliced into 1 mm thick cortical slices. Biopsy punches (1-2 mm diameter) were taken of the relevant brain regions (frontal cortex=fCTx, striatum=Cpu, hippocampus=HP, temporal cortex=tCTx, midbrain=Mb, pons=Pons, medulla=Med, cerebellum=CB, cervical spinal cord=SC-C, thoracic spinal cord=SC-T, and lumbar spinal cord=SC-L) and snap frozen.

### RNA Quantification Analysis

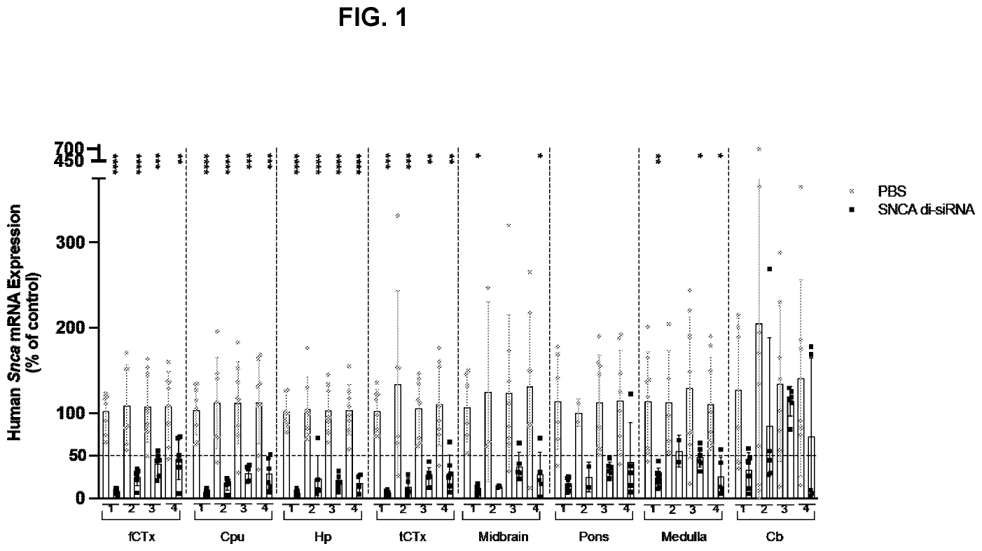

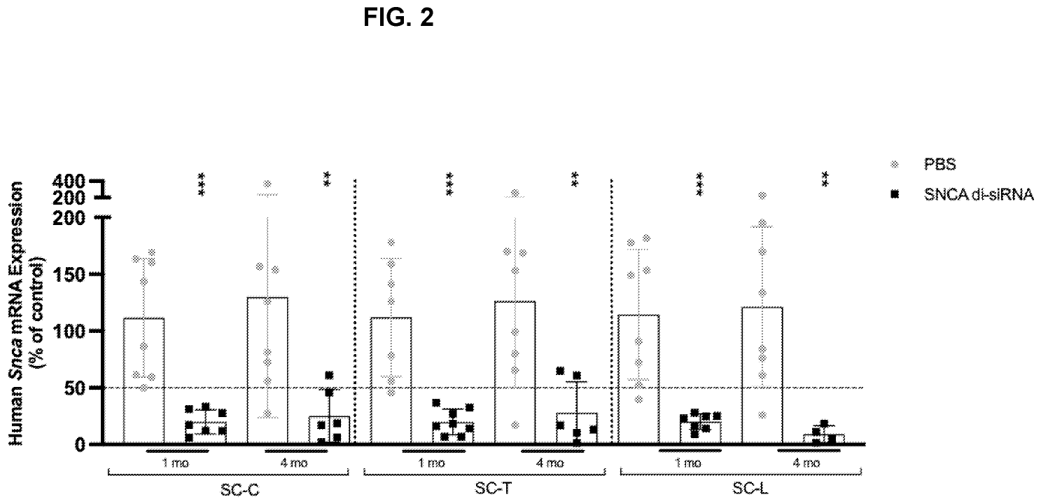

mRNA quantification was performed as described in Example 2. Comparative analysis was performed for relative SNCA mRNA quantitation against samples taken from time-matched PBS control mice. Results are shown in FIG. 1 and FIG. 2.

### Protein Expression Analysis

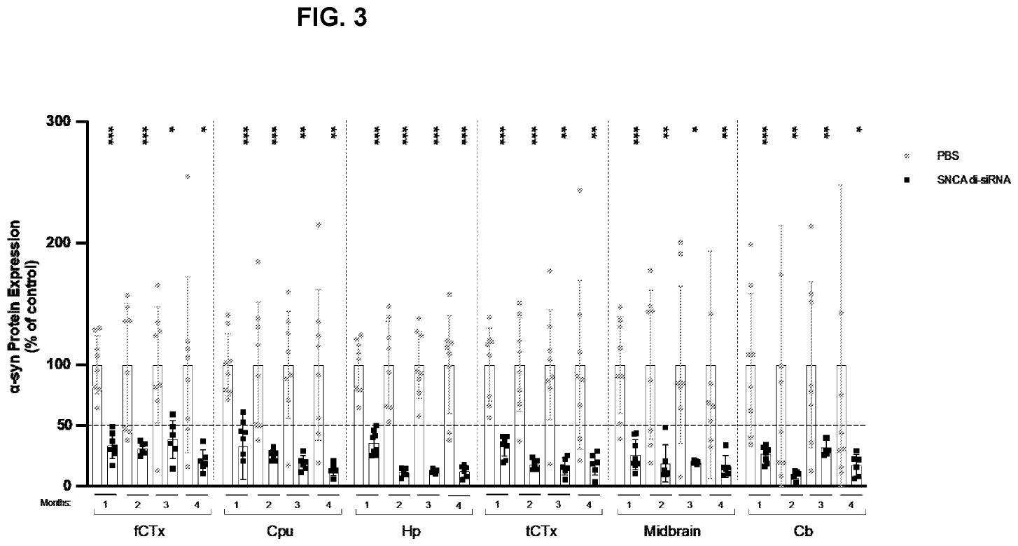

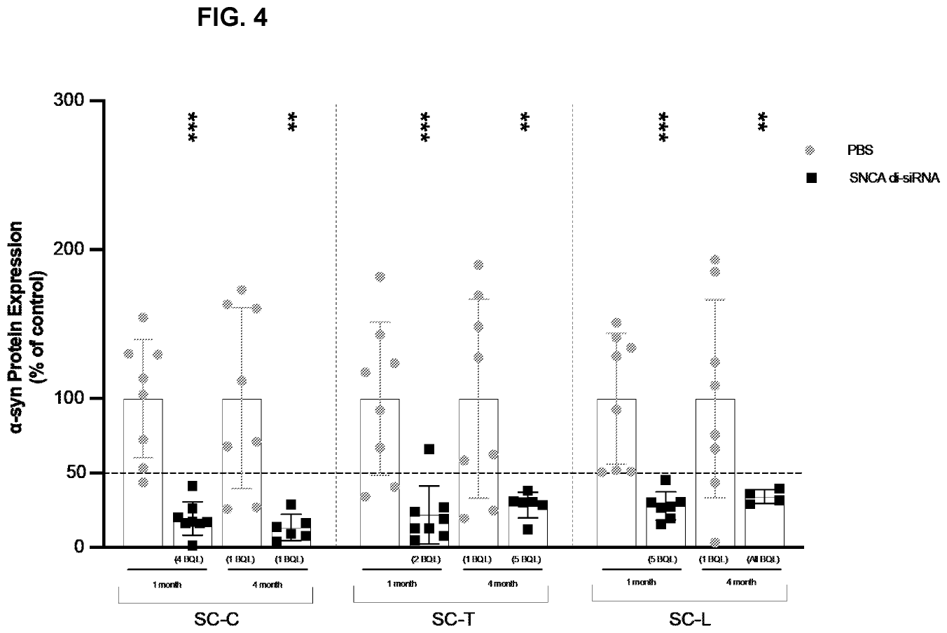

Human α-synuclein protein knockdown was quantified by colorimetric sandwich ELISA (Invitrogen KHB0061). Biological samples were lysed using tissue lysis buffer (CST Cell Lysis Buffer #9803) and bead-beater homogenization on a Qiagen Tissuelyser II. The ELISA was performed using the tissue lysates in accordance with the manufacturer's instructions. The ELISA utilizes a target-specific antibody-coated immunoassay plate to bind to the analyte which in turn binds to the capture antibody. An HRP conjugated secondary detection antibody is used in conjunction with a substrate to generate the measurable signal which is read on a colorimetric plate reader. Data are presented as % SNCA target knockdown (N=8 animals, 4 male, 4 female, 2 technical replicates each), with each siRNA treatment group normalized to time-matched PBS control animals. Results are shown in FIG. 3 and FIG. 4.

### Statistical Assessment

Statistical analysis was Mann-Whitney test, two-tailed. ****=p<0.0001, ***=p<0.001, **=p<0.01, *=p<0.05. BQL=values below the limit of quantification but above the limit of detection in the ELISA; values were included in the graph.

### Conclusion

The results in this example demonstrate that an siRNA molecule of the disclosure, with a sense strand having SEQ ID No.: 664 and an antisense strand having SEQ ID No.: 244, successfully reduced SNCA expression in vivo.

### Example 4: Dose Response of SNCA Knockdown In Vivo

### Objective

This Example describes the results of a series of experiments undertaken to investigate the in vivo dose response of an siRNA molecule complementary to a specific region within an SNCA transcript to effectuate reduced expression of the SNCA gene.

### Materials and Methods

In vivo dose response was assessed in mouse using similar methods as described in Example 3. Briefly, PBS or the di-branched siRNA molecule described in Example 3 was administered via bilateral stereotactic ICV injection at a dose of 2.5 nmol, 5 nmol, 10 nmol, 15 nmol, or 20 nmol in a total volume of 10 μL. Three months after compound administration, animals were euthanized and tissue harvest, mRNA quantification (human and mouse Snca), and protein quantitation (human SNCA) were performed as described in Example 2 and Example 3.

### Results

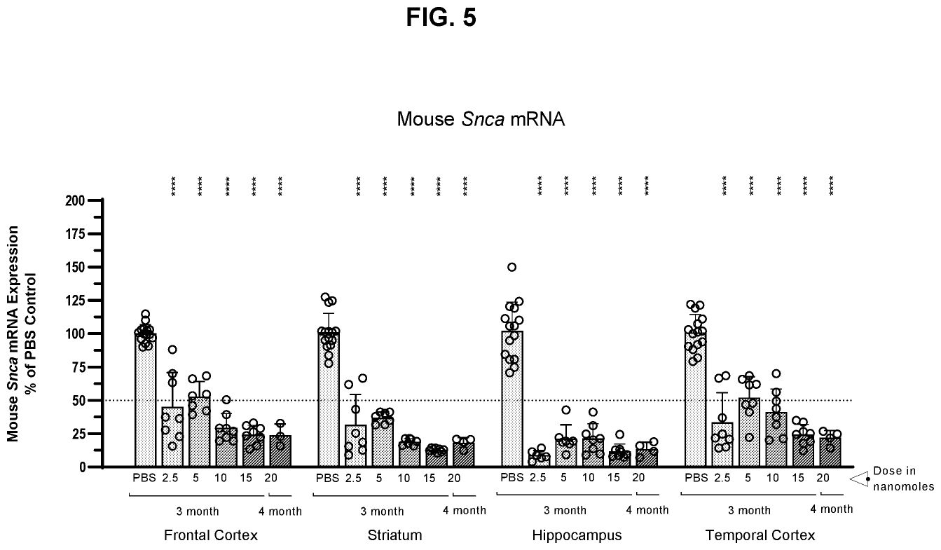

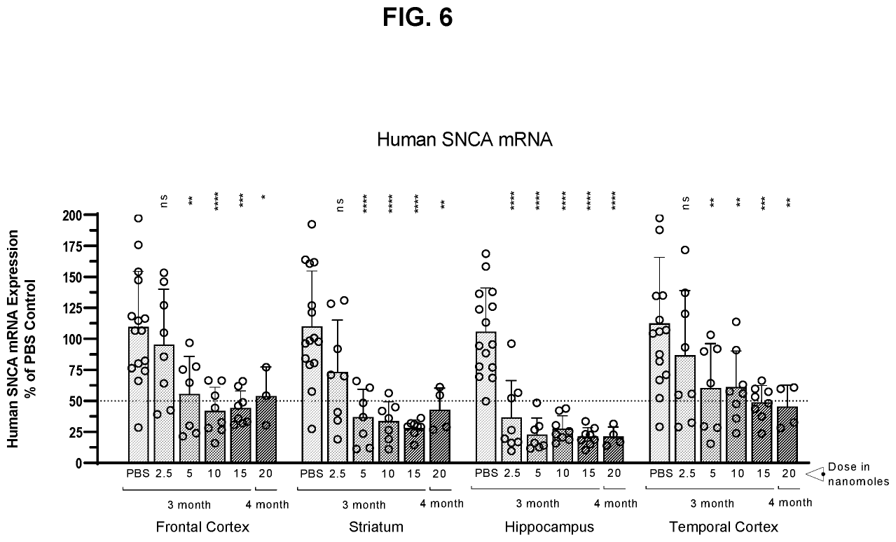

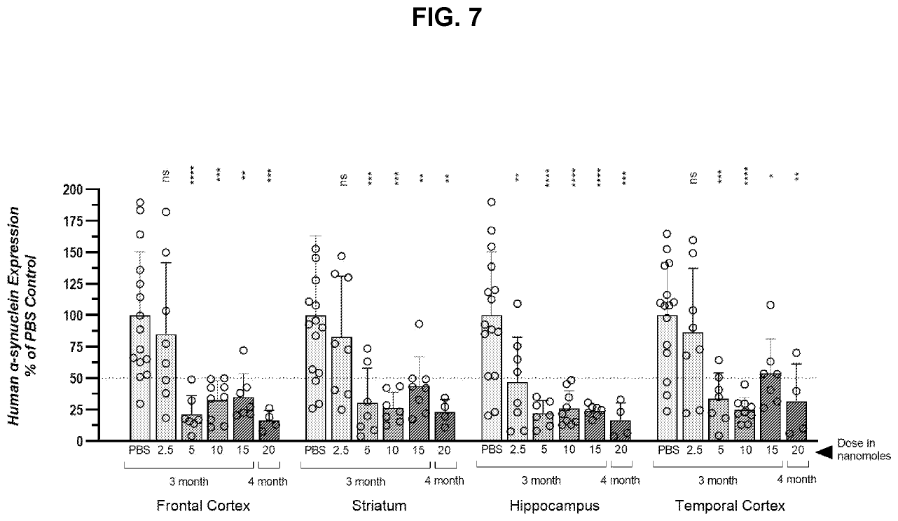

Statistical analysis was two-way ANOVA with Dunnett's multiple comparisons test. ****=p<0.0001, ***=p<0.001, **=p<0.01, *=p<0.05. Results are shown in FIGS. 5, 6, and 7.

### CONCLUSION

The results reported in this example demonstrate that an siRNA molecule of the disclosure successfully silences SNCA in a dose-dependent manner in various regions of the brain.

What is claimed is:

1. A small interfering RNA (siRNA) molecule comprising an antisense strand and sense strand having complementarity to the antisense strand, wherein the antisense strand is from 10 to 30 nucleotides in length and has complementarity sufficient to hybridize to a region within an alpha-synuclein (SNCA) mRNA transcript having the nucleic acid sequence of any one of SEQ ID NOs: 421-840, wherein the antisense strand has a nucleic acid sequence that is at least 95% identical to the nucleic acid sequence of SEQ ID NOs: 1-420.

2. The siRNA molecule of claim 1, wherein the antisense strand comprises at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or 30 contiguous nucleotides that are fully complementary to a contiguous polynucleotide segment of equal length within the region of the SNCA mRNA transcript having the nucleic acid sequence of any one of SEQ ID NOs: 421-840.

3. The siRNA molecule of claim 1 or 2, wherein the antisense strand comprises from 15 to 30 contiguous nucleotides that are fully complementary to a contiguous polynucleotide segment of equal length within the region of the SNCA mRNA transcript having the nucleic acid sequence of any one of SEQ ID NOs: 421-840, optionally wherein the antisense strand comprises 18, 19, 20, 21, 22, 23, 24, or 25 contiguous nucleotides that are fully complementary to a contiguous polynucleotide segment of equal length within the region of the SNCA mRNA transcript having the nucleic acid sequence of any one of SEQ ID NOs: 421-840.

4. The siRNA molecule of any one of claims 1-3, wherein the antisense strand comprises 3 or fewer nucleotide mismatches relative to a region of 21 contiguous nucleobases of the SNCA mRNA transcript having the nucleic acid sequence of any one of SEQ ID NOs: 421-840.

5. The siRNA molecule of any one of claims 1-4, wherein the region of the SNCA mRNA transcript has the nucleic acid sequence of any one of SEQ ID NOs: 459, 535, 551, 599, 631, 663, 664, 666, 667, 671, 748, 750, 751, 752, 760, 762, and 777.

6. The siRNA molecule of claim 5, wherein the region of the SNCA mRNA transcript has the nucleic acid sequence of any one of SEQ ID NOs: 535, 599, 664, 666, 671, 750, 752, 760, and 762.

7. The siRNA molecule of any one of claims 1-6, wherein the antisense strand has a nucleic acid sequence that is at least 96%, 97%, 98%, 99%, or 100% identical to the nucleic acid sequence of any one of SEQ ID NOs: 1-420.

8. The siRNA molecule of any one of claims 1-7, wherein the sense strand has a nucleic acid sequence that is at least 95% identical to the nucleic acid sequence of SEQ ID NOs: 421-840, optionally wherein the sense strand has a nucleic acid sequence that is at least 96%, 97%, 98%, 99%, or 100% identical to the nucleic acid sequence of any one of SEQ ID NOs: 421-840.

9. The siRNA molecule of any one of claims 1-8, wherein the antisense strand comprises:
(a) a structure represented by Formula I, wherein Formula I is, in the 5′-to-3′ direction: 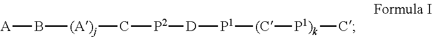
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7,
optionally wherein the antisense strand comprises a structure represented by Formula A1, wherein Formula A1 is, in the 5′-to-3′ direction: 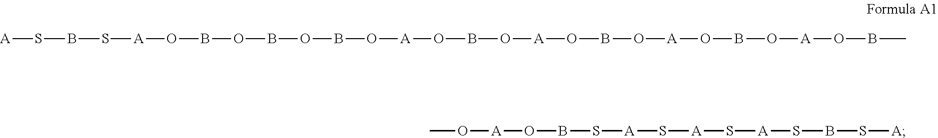
wherein A represents a 2′-O-Me ribonucleoside, B represents a 2′-F ribonucleoside, O represents a phosphodiester internucleoside linkage, and S represents a phosphorothioate internucleoside linkage;
(b) a structure represented by Formula II, wherein Formula II is, in the 5′-to-3′ direction: 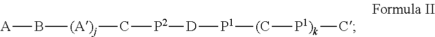
wherein A is represented by the formula C—P¹-D-P¹;
each A′ is represented by the formula C—P²-D-P²;
B is represented by the formula C—P²-D-P²-D-P²-D-P²;
each C is a 2′-O-methyl (2′-O-Me) ribonucleoside;
each C′, independently, is a 2′-O-Me ribonucleoside or a 2′-fluoro (2′-F) ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7,
optionally wherein the antisense strand comprises a structure represented by Formula A2, wherein Formula A2 is, in the 5′-to-3′ direction: 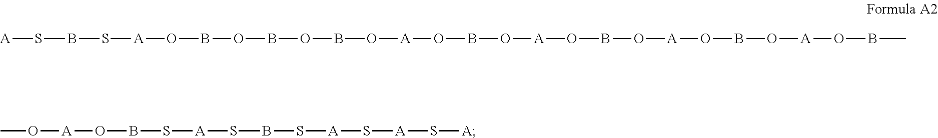
wherein A represents a 2′-O-Me ribonucleoside, B represents a 2′-F ribonucleoside, O represents a phosphodiester internucleoside linkage, and S represents a phosphorothioate internucleoside linkage;
(c) a structure represented by Formula IV, wherein Formula IV is, in the 5′-to-3′ direction: 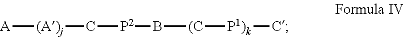
wherein A is represented by the formula C—P¹-D-P¹;
each A′ is represented by the formula C—P²-D-P²;
B is represented by the formula D-P¹—C—P¹-D-P¹;
each C is a 2′-O-Me ribonucleoside;
each C′, independently, is a 2′-O-Me ribonucleoside or a 2′-F ribonucleoside;
each D is a 2′-F ribonucleoside;
each P¹ is a phosphorothioate internucleoside linkage;
each P² is a phosphodiester internucleoside linkage;
j is an integer from 1 to 7; and
k is an integer from 1 to 7,
optionally wherein the antisense strand comprises a structure represented by Formula A3, wherein Formula A3 is, in the 5′-to-3′ direction: 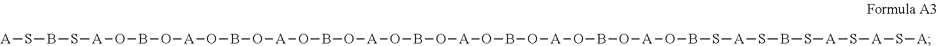
wherein A represents a 2′-O-Me ribonucleoside, B represents a 2′-F ribonucleoside, O represents a phosphodiester internucleoside linkage, and S represents a phosphorothioate internucleoside linkage; or
(d) a structure represented by Formula VI, wherein Formula VI is, in the 5′-to-3′ direction: 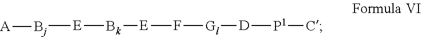
wherein A is represented by the formula C—P¹-D-P¹;
each B is represented by the formula C—P²;
each C is a 2′-O-Me ribonucleoside;
each C′, independently, is a 2′-O-Me ribonucleoside or a 2′-F ribonucleoside;
each D is a 2′-F ribonucleoside;
each E is represented by the formula D-P²—C—P²;
F is represented by the formula D-P¹—C—P¹;
each G is represented by the formula C—P¹;
each P¹ is a phosphorothioate internucleoside linkage;
each P² is a phosphodiester internucleoside linkage;
j is an integer from 1 to 7;
k is an integer from 1 to 7; and
l is an integer from 1 to 7,
optionally wherein the antisense strand comprises a structure represented by Formula A4, wherein Formula A4 is, in the 5′-to-3′ direction: 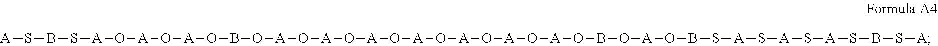
wherein A represents a 2′-O-Me ribonucleoside, B represents a 2′-F ribonucleoside, O represents a phosphodiester internucleoside linkage, and S represents a phosphorothioate internucleoside linkage.

10. The siRNA molecule of any one of claims 1-9, wherein the sense strand comprises:
(a) a structure represented by Formula III, wherein Formula III is, in the 5′-to-3′ direction: 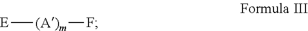
wherein E is represented by the formula (C—P¹)₂;
F is represented by the formula (C—P²)₃-D-P¹—C—P¹—C, (C—P²)₃-D-P²—C—P²—C, (C—P²)₃-D-P¹—C—P¹-D, or (C—P²)₃-D-P²—C—P²-D;
A′, C, D, P¹, and P² are as defined in Formula II; and
m is an integer from 1 to 7,
optionally wherein:
(i) the sense strand comprises a structure represented by Formula S1, wherein Formula S1 is, in the 5′-to-3′ direction: 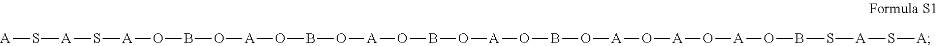
wherein A represents a 2′-O-Me ribonucleoside, B represents a 2′-F ribonucleoside, O represents a phosphodiester internucleoside linkage, and S represents a phosphorothioate internucleoside linkage;
(ii) the sense strand comprises a structure represented by Formula S2, wherein Formula S2 is, in the 5′-to-3′ direction: 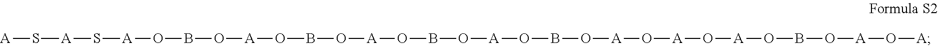
wherein A represents a 2′-O-Me ribonucleoside, B represents a 2′-F ribonucleoside, O represents a phosphodiester internucleoside linkage, and S represents a phosphorothioate internucleoside linkage;
(iii) the sense strand comprises a structure represented by Formula S3, wherein Formula S3 is, in the 5′-to-3′ direction: 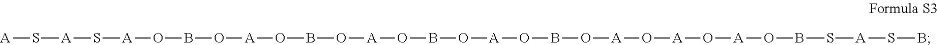
wherein A represents a 2′-O-Me ribonucleoside, B represents a 2′-F ribonucleoside, O represents a phosphodiester internucleoside linkage, and S represents a phosphorothioate internucleoside linkage; or
(iv) the sense strand comprises a structure represented by Formula S4, wherein Formula S4 is, in the 5′-to-3′ direction: 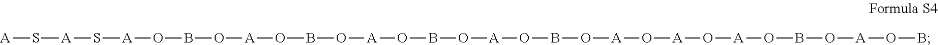
wherein A represents a 2′-O-Me ribonucleoside, B represents a 2′-F ribonucleoside, O represents a phosphodiester internucleoside linkage, and S represents a phosphorothioate internucleoside linkage;
(b) a structure represented by Formula V, wherein Formula V is, in the 5′-to-3′ direction: 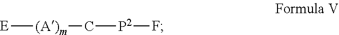
wherein E is represented by the formula (C—P¹)₂;
F is represented by the formula D-P¹—C—P¹—C, D-P²—C—P²—C, D-P¹—C—P¹-D, or D-P²—C—P²-D;
A′, C, D, P¹ and P² are as defined in Formula IV; and
m is an integer from 1 to 7,
optionally wherein:
(i) the sense strand comprises a structure represented by Formula S5, wherein Formula S5 is, in the 5′-to-3′ direction: 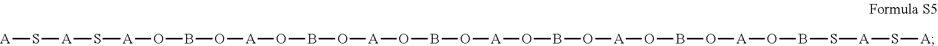
wherein A represents a 2′-O-Me ribonucleoside, B represents a 2′-F ribonucleoside, O represents a phosphodiester internucleoside linkage, and S represents a phosphorothioate internucleoside linkage;
(ii) the sense strand comprises a structure represented by Formula S6, wherein Formula S6 is, in the 5′-to-3′ direction: 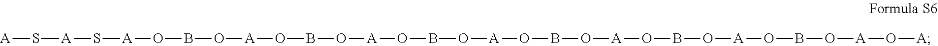
wherein A represents a 2′-O-Me ribonucleoside, B represents a 2′-F ribonucleoside, O represents a phosphodiester internucleoside linkage, and S represents a phosphorothioate internucleoside linkage;
(iii) the sense strand comprises a structure represented by Formula S7, wherein Formula S7 is, in the 5′-to-3′ direction: 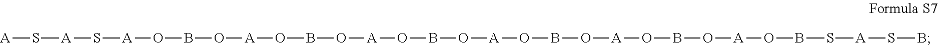
wherein A represents a 2′-O-Me ribonucleoside, B represents a 2′-F ribonucleoside, O represents a phosphodiester internucleoside linkage, and S represents a phosphorothioate internucleoside linkage; or
(iv) the sense strand comprises a structure represented by Formula S8, wherein Formula S8 is, in the 5′-to-3′ direction: 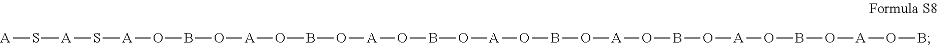
wherein A represents a 2′-O-Me ribonucleoside, B represents a 2′-F ribonucleoside, O represents a phosphodiester internucleoside linkage, and S represents a phosphorothioate internucleoside linkage; or
(c) a structure represented by Formula VII, wherein Formula VII is, in the 5′-to-3′ direction: 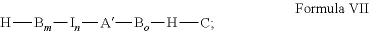
wherein A′ is represented by the formula C—P²-D-P²;
each H is represented by the formula (C—P¹)₂;
each I is represented by the formula (D-P²);
B, C, D, P¹ and P² are as defined in Formula VI;
m is an integer from 1 to 7;
n is an integer from 1 to 7; and
o is an integer from 1 to 7,
optionally wherein the sense strand comprises a structure represented by Formula S9, wherein Formula S9 is, in the 5′-to-3′ direction: 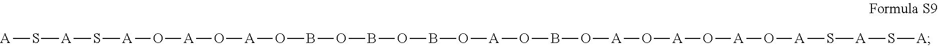
wherein A represents a 2′-O-Me ribonucleoside, B represents a 2′-F ribonucleoside, O represents a phosphodiester internucleoside linkage, and S represents a phosphorothioate internucleoside linkage.

11. The siRNA molecule of any one of claims 1-10, wherein the antisense strand further comprises a 5′ phosphorus stabilizing moiety at the 5′ end of the antisense strand and/or the sense strand further comprises a 5′ phosphorus stabilizing moiety at the 5′ end of the sense strand.

12. The siRNA molecule of claim 11, wherein each 5′ phosphorus stabilizing moiety is, independently, represented by any one of Formulas IX-XVI: 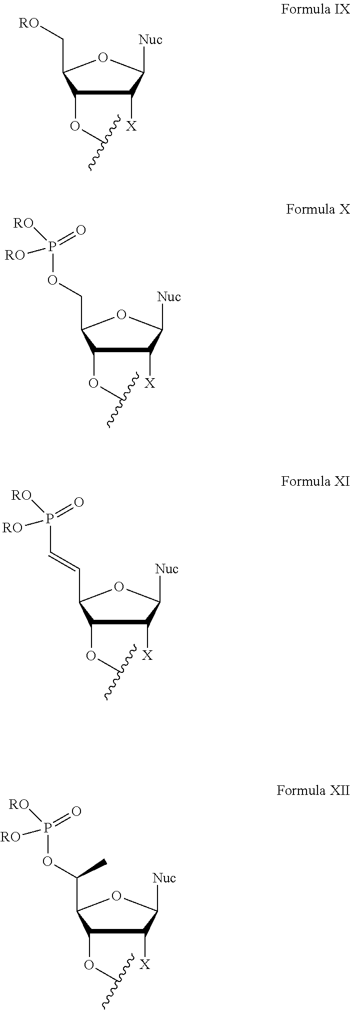 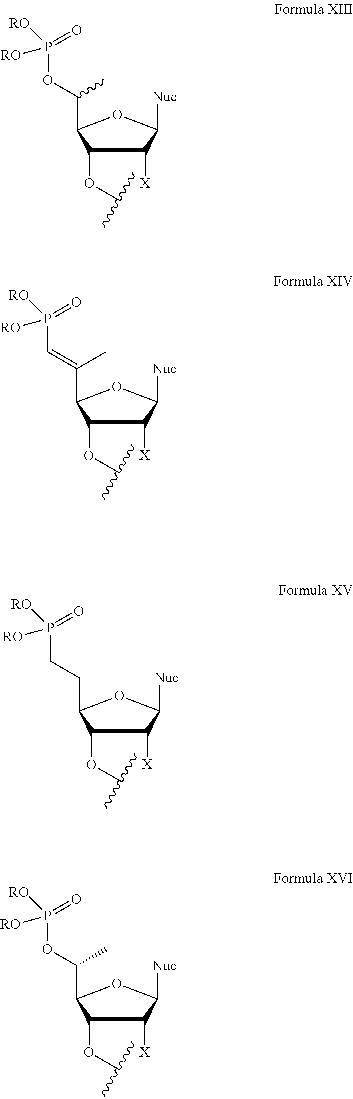
wherein Nuc represents a nucleobase selected from the group consisting of adenine, uracil, guanine, thymine, and cytosine, and R represents an optionally substituted alkyl, optionally substituted alkenyl, optionally substituted alkynyl, phenyl, benzyl, hydroxy, or hydrogen.

13. The siRNA molecule of any one of claims 1-12, wherein the siRNA molecule is a branched siRNA molecule.

14. The siRNA molecule of claim 13, wherein the branched siRNA molecule is a di-branched siRNA molecule, optionally wherein the di-branched siRNA molecule is represented by any one of Formulas XVII-XIX: 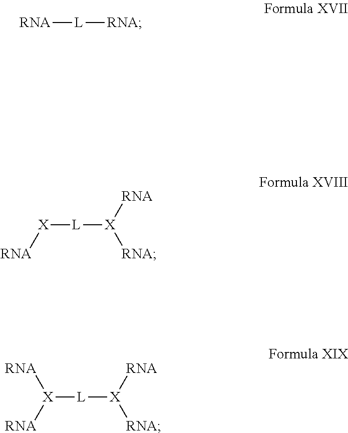
wherein each RNA is, independently, an siRNA molecule, L is a linker, and each X, independently, represents a branch point moiety.

15. An siRNA molecule comprising:
(I)
a) a sense strand comprising the sequence
(SEQ ID NO: 841)
(mU)#(mA)#(mC)(fC)(mA)(fC)(mU)(fU)(mA)
(fU)(mU)(mU)(mC)(fU)#(mA)#(mA)-DIO
and an antisense strand comprising the sequence
(SEQ ID NO: 842)
V(mU)#(fU)#(mA)(fG)(fA)(fA)(mA)(fU)(mA)
(fA)(mG)(fU)(mG)(fG)(mU)(fA)#(mG)#(mU)
#(mC)#(mA)#(mC);
b) a sense strand comprising the sequence
(SEQ ID NO: 843)
(mC)#(mA)#(mA)(fG)(mU)(fG)(mC)(fU)(mC)
(fA)(mG)(mU)(mU)(fC)#(mC)#(mA)-DIO
and an antisense strand comprising the sequence
(SEQ ID NO: 844)
V(mU)#(fG)#(mG)(fA)(fA)(fC)(mU)(fG)(mA)
(fG)(mC)(fA)(mC)(fU)(mU)(fG)#(mU)#(mA)
#(mC)#(mA)#(mG);
c) a sense strand comprising the sequence
(SEQ ID NO: 845)
(mA)#(mG)#(mU)(fG)(mG)(fU)(mG)(fC)(mA)
(fU)(mG)(mG)(mU)(fG)#(mU)#(mA)-DIO
and an antisense strand comprising the sequence
(SEQ ID NO: 846)
V(mU)#(fA)#(mC)(fA)(fC)(fC)(mA)(fU)(mG)
(fC)(mA)(fC)(mC)(fA)(mC)(fU)#(mC)#(mC)
#(mC)#(mU)#(mC).
d) a sense strand comprising the sequence
(SEQ ID NO: 847)
(mC)#(mA)#(mA)(fU)(mG)(fA)(mG)(fG)(mC)
(fU)(mU)(mA)(mU)(fG)#(mA)#(mA)-DIO
and an antisense strand comprising the sequence
(SEQ ID NO: 848)
V(mU)#(fU)#(mU)(fG)(fG)(fA)(mA)(fC)(mU)
(fG)(mA)(fG)(mC)(fA)(mC)(fU)#(mU)#(mG)
#(mU)#(mA)#(mC);
e) a sense strand comprising the sequence
(SEQ ID NO: 849)
(mU)#(mC)#(mA)(fG)(mU)(fU)(mC)(fC)(mA)
(fA)(mU)(mG)(mU)(fG)#(mC)#(mA)-DIO
and an antisense strand comprising the sequence
(SEQ ID NO: 850)
V(mU)#(fU)#(mU)(fG)(fG)(fA)(mA)(fC)(mU)
(fG)(mA)(fG)(mC)(fA)(mC)(fU)#(mU)#(mG)
#(mU)#(mA)#(mC).
f)
a sense strand comprising the sequence
(SEQ ID NO: 851)
(mC)#(mU)#(mA)(fC)(mG)(fA)(mU)(fG)(mU)
(fU)(mA)(mA)(mA)(fA)#(mC)#(mA)-DIO
and an antisense strand comprising the sequence
(SEQ ID NO: 852)
V(mU)#(fG)#(mC)(fA)(fC)(fA)(mU)(fU)(mG)
(fG)(mA)(fA)(mC)(fU)(mG)(fA)#(mG)#(mC)
#(mA)#(mC)#(mU);
or
g) a sense strand comprising the sequence
(SEQ ID NO: 853)
(mC)#(mU)#(mA)(fC)(mG)(fA)(mU)(fG)(mU)
(fU)(mA)(mA)(mA)(fA)#(mC)#(mA)-DIO
and an antisense strand comprising the sequence
(SEQ ID NO: 854)
V(mU)#(fG)#(mU)(fU)(fU)(fU)(mA)(fA)(mC)
(fA)(mU)(fC)(mG)(fU)(mA)(fG)#(mA)#(mU)
#(mU)#(mG)#(mA);
wherein m represents a 2′-O-Me ribonucleoside,
f represents a 2′-F ribonucleoside,
# represents a phosphorothioate
internucleoside linkage,
-DIO represents a divalent
oligonucleotide (DIO) linker; and
V represents a vinyl phosphonate;
or
(II)
a) a sense strand comprising the sequence
(SEQ ID NO: 841)
(mU)#(mA)#(mC)(fC)(mA)(fC)(mU)(fU)(mA)
(fU)(mU)(mU)(mC)(fU)#(mA)#(mA)-DIO
and an antisense strand comprising the sequence
(SEQ ID NO: 842)
V(mU)#(fU)#(mA)(fG)(fA)(fA)(mA)(fU)(mA)
(fA)(mG)(fU)(mG)(fG)(mU)(fA)#(mG)#
(mU)#(mC)#(mA)#(mC);
b) a sense strand comprising the sequence
(SEQ ID NO: 843)
(mC)#(mA)#(mA)(fG)(mU)(fG)(mC)(fU)(mC)
(fA)(mG)(mU)(mU)(fC)#(mC)#(mA)-DIO
and an antisense strand comprising the sequence
(SEQ ID NO: 844)
V(mU)#(fG)#(mG)(fA)(fA)(fC)(mU)(fG)(mA)
(fG)(mC)(fA)(mC)(fU)(mU)(fG)#(mU)#(mA)
#(mC)#(mA)#(mG);
c) a sense strand comprising the sequence
(SEQ ID NO: 845)
(mA)#(mG)#(mU)(fG)(mG)(fU)(mG)(fC)(mA)
(fU)(mG)(mG)(mU)(fG)#(mU)#(mA)-DIO
and an antisense strand comprising the sequence
(SEQ ID NO: 846)
V(mU)#(fA)#(mC)(fA)(fC)(fC)(mA)(fU)(mG)
(fC)(mA)(fC)(mC)(fA)(mC)(fU)#(mC)#(mC)
#(mC)#(mU)#(mC).
d) a sense strand comprising the sequence
(SEQ ID NO: 847)
(mC)#(mA)#(mA)(fU)(mG)(fA)(mG)(fG)(mC)
(fU)(mU)(mA)(mU)(fG)#(mA)#(mA)-DIO
and an antisense strand comprising the sequence
(SEQ ID NO: 855)
V(mU)#(fU)#(mC)(fA)(fU)(fA)(mA)(fG)(mC)
(fC)(mU)(fC)(mA)(fU)(mU)(fG)#(mU)#(mC)
#(mA)#(mG)#(mG)
e) a sense strand comprising the sequence
(SEQ ID NO: 856)
(mA)#(mG)#(mU)(fG)(mC)(fU)(mC)(fA)(mG)
(fU)(mU)(mC)(mC)(fA)#(mA)#(mA)-DIO
and an antisense strand comprising the sequence
(SEQ ID NO: 850)
V(mU)#(fU)#(mU)(fG)(fG)(fA)(mA)(fC)(mU)
(fG)(mA)(fG)(mC)(fA)(mC)(fU)#(mU)#(mG)
#(mU)#(mA)#(mC).
f) a sense strand comprising the sequence
(SEQ ID NO: 857)
(mU)#(mC)#(mA)(fG)(mU)(fU)(mC)(fC)(mA)
(fA)(mU)(mG)(mU)(fG)#(mC)#(mA)-DIO
and an antisense strand comprising
the sequence
(SEQ ID NO: 852)
V(mU)#(fG)#(mC)(fA)(fC)(fA)(mU)(fU)(mG)
(fG)(mA)(fA)(mC)(fU)(mG)(fA)#(mG)#(mC)
#(mA)#(mC)#(mU);
or
g) a sense strand comprising the sequence
(SEQ ID NO: 853)
(mC)#(mU)#(mA)(fC)(mG)(fA)(mU)(fG)(mU)
(fU)(mA)(mA)(mA)(fA)#(mC)#(mA)-DIO
and an antisense strand comprising the sequence
(SEQ ID NO: 854)
V(mU)#(fG)#(mU)(fU)(fU)(fU)(mA)(fA)(mC)
(fA)(mU)(fC)(mG)(fU)(mA)(fG)#(mA)#(mU)
#(mU)#(mG)#(mA);
wherein m represents a 2′-O-Me ribonucleoside,
f represents a 2′-F ribonucleoside,
# represents a phosphorothioate
internucleoside linkage,
-DIO represents a divalent
oligonucleotide (DIO) linker;
and V represents a vinyl phosphonate.

16. A pharmaceutical composition comprising the siRNA molecule of any one of claims 1-15 and a pharmaceutically acceptable excipient, carrier, or diluent.

17. A method of treating a synucleinopathy in a human subject in need thereof, the method comprising administering a therapeutically effective amount of the siRNA molecule of any one of claims 1-15 or the pharmaceutical composition of claim 16 to the subject.

18. The method of claim 17, wherein the synucleinopathy is Parkinson's disease, Alzheimer's disease, Lewy body dementia, or a multiple symptom atrophy.

19. The method of claim 17 or 18, wherein the siRNA molecule or the pharmaceutical composition is administered to the subject by way of intracerebroventricular, intrastriatal, intraparenchymal, or intrathecal injection.

20. A kit comprising the siRNA molecule of any one of claims 1-15, or the pharmaceutical composition of claim 16, and a package insert, wherein the package insert instructs a user of the kit to perform the method of any one of claims 17-19.


SEQUENCE LISTING (868 sequences)

SEQ ID NO: 1 - RNA, 21 nt, Synthetic construct
  source 1..21: mol_type = other RNA
taggcttgaaggcaaggcgtg

SEQ ID NO: 2 - RNA, 21 nt, Synthetic construct
  source 1..21: mol_type = other RNA
tgcagaaggcttgaaggcaag

SEQ ID NO: 3 - RNA, 21 nt, Synthetic construct
  source 1..21: mol_type = other RNA
taggcagaaggcttgaaggca

SEQ ID NO: 4 - RNA, 21 nt, Synthetic construct
  source 1..21: mol_type = other RNA
taaggcagaaggcttgaaggc

SEQ ID NO: 5 - RNA, 21 nt, Synthetic construct
  source 1..21: mol_type = other RNA
taaaggcagaaggcttgaagg

SEQ ID NO: 6 - RNA, 21 nt, Synthetic construct
  source 1..21: mol_type = other RNA
tgaaaggcagaaggcttgaag

SEQ ID NO: 7 - RNA, 21 nt, Synthetic construct
  source 1..21: mol_type = other RNA
ttggaaaggcagaaggcttga

SEQ ID NO: 8 - RNA, 21 nt, Synthetic construct
  source 1..21: mol_type = other RNA
tgtggaaaggcagaaggcttg

SEQ ID NO: 9 - RNA, 21 nt, Synthetic construct
  source 1..21: mol_type = other RNA
ttcacgagggtggaaaggcag

SEQ ID NO: 10 - RNA, 21 nt, Synthetic construct
  source 1..21: mol_type = other RNA
tgctcacgagggtggaaaggc

SEQ ID NO: 11 - RNA, 21 nt, Synthetic construct
  source 1..21: mol_type = other RNA
tgtcgtcgaatggccactccc

SEQ ID NO: 12 - RNA, 21 nt, Synthetic construct
  source 1..21: mol_type = other RNA
tttcctttacaccacactgtc

SEQ ID NO: 13 - RNA, 21 nt, Synthetic construct
  source 1..21: mol_type = other RNA
tattcctttacaccacactgt

SEQ ID NO: 14 - RNA, 21 nt, Synthetic construct
  source 1..21: mol_type = other RNA
taattcctttacaccacactg

SEQ ID NO: 15 - RNA, 21 nt, Synthetic construct
  source 1..21: mol_type = other RNA
tatgaattcctttacaccaca

SEQ ID NO: 16 - RNA, 21 nt, Synthetic construct
  source 1..21: mol_type = other RNA
taatgaattcctttacaccac

SEQ ID NO: 17 - RNA, 21 nt, Synthetic construct
  source 1..21: mol_type = other RNA
ttaatgaattcctttacacca

SEQ ID NO: 18 - RNA, 21 nt, Synthetic construct
  source 1..21: mol_type = other RNA
tctaatgaattcctttacacc

SEQ ID NO: 19 - RNA, 21 nt, Synthetic construct
  source 1..21: mol_type = other RNA
tgctaatgaattcctttacac

SEQ ID NO: 20 - RNA, 21 nt, Synthetic construct
  source 1..21: mol_type = other RNA
tggctaatgaattcctttaca

SEQ ID NO: 21 - RNA, 21 nt, Synthetic construct
  source 1..21: mol_type = other RNA
ttggctaatgaattcctttac

SEQ ID NO: 22 - RNA, 21 nt, Synthetic construct
  source 1..21: mol_type = other RNA
tatggctaatgaattccttta

SEQ ID NO: 23 - RNA, 21 nt, Synthetic construct
  source 1..21: mol_type = other RNA
ttccatggctaatgaattcct

SEQ ID NO: 24 - RNA, 21 nt, Synthetic construct
  source 1..21: mol_type = other RNA
tatccatggctaatgaattcc

SEQ ID NO: 25 - RNA, 21 nt, Synthetic construct
  source 1..21: mol_type = other RNA
ttacatccatggctaatgaat

SEQ ID NO: 26 - RNA, 21 nt, Synthetic construct
  source 1..21: mol_type = other RNA
tatacatccatggctaatgaa

SEQ ID NO: 27 - RNA, 21 nt, Synthetic construct
  source 1..21: mol_type = other RNA
taatacatccatggctaatga

SEQ ID NO: 28 - RNA, 21 nt, Synthetic construct
  source 1..21: mol_type = other RNA
tgaatacatccatggctaatg

SEQ ID NO: 29 - RNA, 21 nt, Synthetic construct
  source 1..21: mol_type = other RNA
ttgaatacatccatggctaat

SEQ ID NO: 30 - RNA, 21 nt, Synthetic construct
  source 1..21: mol_type = other RNA
tatgaatacatccatggctaa

SEQ ID NO: 31 - RNA, 21 nt, Synthetic construct
  source 1..21: mol_type = other RNA
tcatgaatacatccatggcta

SEQ ID NO: 32 - RNA, 21 nt, Synthetic construct
  source 1..21: mol_type = other RNA
ttcatgaatacatccatggct

SEQ ID NO: 33 - RNA, 21 nt, Synthetic construct
  source 1..21: mol_type = other RNA
tttcatgaatacatccatggc

SEQ ID NO: 34 - RNA, 21 nt, Synthetic construct
  source 1..21: mol_type = other RNA
ttttcatgaatacatccatgg

SEQ ID NO: 35 - RNA, 21 nt, Synthetic construct
  source 1..21: mol_type = other RNA
tgtcctttcatgaatacatcc

SEQ ID NO: 36 - RNA, 21 nt, Synthetic construct
  source 1..21: mol_type = other RNA
tagtcctttcatgaatacatc

SEQ ID NO: 37 - RNA, 21 nt, Synthetic construct
  source 1..21: mol_type = other RNA
taagtcctttcatgaatacat

SEQ ID NO: 38 - RNA, 21 nt, Synthetic construct
  source 1..21: mol_type = other RNA
taaagtcctttcatgaataca

SEQ ID NO: 39 - RNA, 21 nt, Synthetic construct
  source 1..21: mol_type = other RNA
tgaaagtcctttcatgaatac

SEQ ID NO: 40 - RNA, 21 nt, Synthetic construct
  source 1..21: mol_type = other RNA
ttttgaaagtcctttcatgaa

SEQ ID NO: 41 - RNA, 21 nt, Synthetic construct
  source 1..21: mol_type = other RNA
tctttgaaagtcctttcatga

SEQ ID NO: 42 - RNA, 21 nt, Synthetic construct
  source 1..21: mol_type = other RNA
tcctttgaaagtcctttcatg

SEQ ID NO: 43 - RNA, 21 nt, Synthetic construct
  source 1..21: mol_type = other RNA
tgcctttgaaagtcctttcat

SEQ ID NO: 44 - RNA, 21 nt, Synthetic construct
  source 1..21: mol_type = other RNA
tggcctttgaaagtcctttca

SEQ ID NO: 45 - RNA, 21 nt, Synthetic construct
  source 1..21: mol_type = other RNA
ttggcctttgaaagtcctttc

SEQ ID NO: 46 - RNA, 21 nt, Synthetic construct
  source 1..21: mol_type = other RNA
tttggcctttgaaagtccttt

SEQ ID NO: 47 - RNA, 21 nt, Synthetic construct
  source 1..21: mol_type = other RNA
tcttggcctttgaaagtcctt

SEQ ID NO: 48 - RNA, 21 nt, Synthetic construct
  source 1..21: mol_type = other RNA
tccttggcctttgaaagtcct

SEQ ID NO: 49 - RNA, 21 nt, Synthetic construct
  source 1..21: mol_type = other RNA
ttccttggcctttgaaagtcc

SEQ ID NO: 50 - RNA, 21 nt, Synthetic construct
  source 1..21: mol_type = other RNA
tctccttggcctttgaaagtc

SEQ ID NO: 51 - RNA, 21 nt, Synthetic construct
  source 1..21: mol_type = other RNA
tcctccttggcctttgaaagt

SEQ ID NO: 52 - RNA, 21 nt, Synthetic construct
  source 1..21: mol_type = other RNA
tccctccttggcctttgaaag

SEQ ID NO: 53 - RNA, 21 nt, Synthetic construct
  source 1..21: mol_type = other RNA
ttccctccttggcctttgaaa

SEQ ID NO: 54 - RNA, 21 nt, Synthetic construct
  source 1..21: mol_type = other RNA
tctccctccttggcctttgaa

SEQ ID NO: 55 - RNA, 21 nt, Synthetic construct
  source 1..21: mol_type = other RNA
tactccctccttggcctttga

SEQ ID NO: 56 - RNA, 21 nt, Synthetic construct
  source 1..21: mol_type = other RNA
taactccctccttggcctttg

SEQ ID NO: 57 - RNA, 21 nt, Synthetic construct
  source 1..21: mol_type = other RNA
tcaactccctccttggccttt

SEQ ID NO: 58 - RNA, 21 nt, Synthetic construct
  source 1..21: mol_type = other RNA
tacaactccctccttggcctt

SEQ ID NO: 59 - RNA, 21 nt, Synthetic construct
  source 1..21: mol_type = other RNA
tcacaactccctccttggcct

SEQ ID NO: 60 - RNA, 21 nt, Synthetic construct
  source 1..21: mol_type = other RNA
tccacaactccctccttggcc

SEQ ID NO: 61 - RNA, 21 nt, Synthetic construct
  source 1..21: mol_type = other RNA
tgccacaactccctccttggc

SEQ ID NO: 62 - RNA, 21 nt, Synthetic construct
  source 1..21: mol_type = other RNA
tagccacaactccctccttgg

SEQ ID NO: 63 - RNA, 21 nt, Synthetic construct
  source 1..21: mol_type = other RNA
tcagccacaactccctccttg

SEQ ID NO: 64 - RNA, 21 nt, Synthetic construct
  source 1..21: mol_type = other RNA
tgcagccacaactccctcctt

SEQ ID NO: 65 - RNA, 21 nt, Synthetic construct
  source 1..21: mol_type = other RNA
tagcagccacaactccctcct

SEQ ID NO: 66 - RNA, 21 nt, Synthetic construct
  source 1..21: mol_type = other RNA
tcagcagccacaactccctcc

SEQ ID NO: 67 - RNA, 21 nt, Synthetic construct
  source 1..21: mol_type = other RNA
tgcagcagccacaactccctc

SEQ ID NO: 68 - RNA, 21 nt, Synthetic construct
  source 1..21: mol_type = other RNA
tagcagcagccacaactccct

SEQ ID NO: 69 - RNA, 21 nt, Synthetic construct
  source 1..21: mol_type = other RNA
tcagcagcagccacaactccc

SEQ ID NO: 70 - RNA, 21 nt, Synthetic construct
  source 1..21: mol_type = other RNA
ttcagcagcagccacaactcc

SEQ ID NO: 71 - RNA, 21 nt, Synthetic construct
  source 1..21: mol_type = other RNA
tctcagcagcagccacaactc

SEQ ID NO: 72 - RNA, 21 nt, Synthetic construct
  source 1..21: mol_type = other RNA
ttctcagcagcagccacaact

SEQ ID NO: 73 - RNA, 21 nt, Synthetic construct
  source 1..21: mol_type = other RNA
tttctcagcagcagccacaac

SEQ ID NO: 74 - RNA, 21 nt, Synthetic construct
  source 1..21: mol_type = other RNA
ttttctcagcagcagccacaa

SEQ ID NO: 75 - RNA, 21 nt, Synthetic construct
  source 1..21: mol_type = other RNA
tgttttctcagcagcagccac

SEQ ID NO: 76 - RNA, 21 nt, Synthetic construct
  source 1..21: mol_type = other RNA
tggttttctcagcagcagcca

SEQ ID NO: 77 - RNA, 21 nt, Synthetic construct
  source 1..21: mol_type = other RNA
ttggttttctcagcagcagcc

SEQ ID NO: 78 - RNA, 21 nt, Synthetic construct
  source 1..21: mol_type = other RNA
tttggttttctcagcagcagc

SEQ ID NO: 79 - RNA, 21 nt, Synthetic construct
  source 1..21: mol_type = other RNA
ttttggttttctcagcagcag

SEQ ID NO: 80 - RNA, 21 nt, Synthetic construct
  source 1..21: mol_type = other RNA
tgtttggttttctcagcagca

SEQ ID NO: 81 - RNA, 21 nt, Synthetic construct
  source 1..21: mol_type = other RNA
ttgtttggttttctcagcagc

SEQ ID NO: 82 - RNA, 21 nt, Synthetic construct
  source 1..21: mol_type = other RNA
tctgtttggttttctcagcag

SEQ ID NO: 83 - RNA, 21 nt, Synthetic construct
  source 1..21: mol_type = other RNA
tcctgtttggttttctcagca

SEQ ID NO: 84 - RNA, 21 nt, Synthetic construct
  source 1..21: mol_type = other RNA
tccctgtttggttttctcagc

SEQ ID NO: 85 - RNA, 21 nt, Synthetic construct
  source 1..21: mol_type = other RNA
tgccacaccctgtttggtttt

SEQ ID NO: 86 - RNA, 21 nt, Synthetic construct
  source 1..21: mol_type = other RNA
ttgccacaccctgtttggttt

SEQ ID NO: 87 - RNA, 21 nt, Synthetic construct
  source 1..21: mol_type = other RNA
tctgccacaccctgtttggtt

SEQ ID NO: 88 - RNA, 21 nt, Synthetic construct
  source 1..21: mol_type = other RNA
ttctgccacaccctgtttggt

SEQ ID NO: 89 - RNA, 21 nt, Synthetic construct
  source 1..21: mol_type = other RNA
tttctgccacaccctgtttgg

SEQ ID NO: 90 - RNA, 21 nt, Synthetic construct
  source 1..21: mol_type = other RNA
ttctttcctgctgcttctgcc

SEQ ID NO: 91 - RNA, 21 nt, Synthetic construct
  source 1..21: mol_type = other RNA
tcttttgtctttcctgctgct

SEQ ID NO: 92 - RNA, 21 nt, Synthetic construct
  source 1..21: mol_type = other RNA
ttcttttgtctttcctgctgc

SEQ ID NO: 93 - RNA, 21 nt, Synthetic construct
  source 1..21: mol_type = other RNA
tctcttttgtctttcctgctg

SEQ ID NO: 94 - RNA, 21 nt, Synthetic construct
  source 1..21: mol_type = other RNA
tcctcttttgtctttcctgct

SEQ ID NO: 95 - RNA, 21 nt, Synthetic construct
  source 1..21: mol_type = other RNA
tccctcttttgtctttcctgc

SEQ ID NO: 96 - RNA, 21 nt, Synthetic construct
  source 1..21: mol_type = other RNA
taccctcttttgtctttcctg

SEQ ID NO: 97 - RNA, 21 nt, Synthetic construct
  source 1..21: mol_type = other RNA
tacaccctcttttgtctttcc

SEQ ID NO: 98 - RNA, 21 nt, Synthetic construct
  source 1..21: mol_type = other RNA
taacaccctcttttgtctttc

SEQ ID NO: 99 - RNA, 21 nt, Synthetic construct
  source 1..21: mol_type = other RNA
tgaacaccctcttttgtcttt

SEQ ID NO: 100 - RNA, 21 nt, Synthetic construct
  source 1..21: mol_type = other RNA
tagaacaccctcttttgtctt

SEQ ID NO: 101 - RNA, 21 nt, Synthetic construct
  source 1..21: mol_type = other RNA
tgagcctacatagagaacacc

SEQ ID NO: 102 - RNA, 21 nt, Synthetic construct
  source 1..21: mol_type = other RNA
tggagcctacatagagaacac

SEQ ID NO: 103 - RNA, 21 nt, Synthetic construct
  source 1..21: mol_type = other RNA
ttggagcctacatagagaaca

SEQ ID NO: 104 - RNA, 21 nt, Synthetic construct
  source 1..21: mol_type = other RNA
tttggagcctacatagagaac

SEQ ID NO: 105 - RNA, 21 nt, Synthetic construct
  source 1..21: mol_type = other RNA
ttttggagcctacatagagaa

SEQ ID NO: 106 - RNA, 21 nt, Synthetic construct
  source 1..21: mol_type = other RNA
tgttttggagcctacatagag

SEQ ID NO: 107 - RNA, 21 nt, Synthetic construct
  source 1..21: mol_type = other RNA
tccttggttttggagcctaca

SEQ ID NO: 108 - RNA, 21 nt, Synthetic construct
  source 1..21: mol_type = other RNA
ttccttggttttggagcctac

SEQ ID NO: 109 - RNA, 21 nt, Synthetic construct
  source 1..21: mol_type = other RNA
tctccttggttttggagccta

SEQ ID NO: 110 - RNA, 21 nt, Synthetic construct
  source 1..21: mol_type = other RNA
tcctccttggttttggagcct

SEQ ID NO: 111 - RNA, 21 nt, Synthetic construct
  source 1..21: mol_type = other RNA
tccctccttggttttggagcc

SEQ ID NO: 112 - RNA, 21 nt, Synthetic construct
  source 1..21: mol_type = other RNA
tccactccctccttggttttg

SEQ ID NO: 113 - RNA, 21 nt, Synthetic construct
  source 1..21: mol_type = other RNA
taccatgcaccactccctcct

SEQ ID NO: 114 - RNA, 21 nt, Synthetic construct
  source 1..21: mol_type = other RNA
tcaccatgcaccactccctcc

SEQ ID NO: 115 - RNA, 21 nt, Synthetic construct
  source 1..21: mol_type = other RNA
tacaccatgcaccactccctc

SEQ ID NO: 116 - RNA, 21 nt, Synthetic construct
  source 1..21: mol_type = other RNA
tcacaccatgcaccactccct

SEQ ID NO: 117 - RNA, 21 nt, Synthetic construct
  source 1..21: mol_type = other RNA
tccacaccatgcaccactccc

SEQ ID NO: 118 - RNA, 21 nt, Synthetic construct
  source 1..21: mol_type = other RNA
tgttgccacaccatgcaccac

SEQ ID NO: 119 - RNA, 21 nt, Synthetic construct
  source 1..21: mol_type = other RNA
ttgttgccacaccatgcacca

SEQ ID NO: 120 - RNA, 21 nt, Synthetic construct
  source 1..21: mol_type = other RNA
tctgttgccacaccatgcacc

SEQ ID NO: 121 - RNA, 21 nt, Synthetic construct
  source 1..21: mol_type = other RNA
tactgttgccacaccatgcac

SEQ ID NO: 122 - RNA, 21 nt, Synthetic construct
  source 1..21: mol_type = other RNA
tcactgttgccacaccatgca

SEQ ID NO: 123 - RNA, 21 nt, Synthetic construct
  source 1..21: mol_type = other RNA
tcagccactgttgccacacca

SEQ ID NO: 124 - RNA, 21 nt, Synthetic construct
  source 1..21: mol_type = other RNA
ttcagccactgttgccacacc

SEQ ID NO: 125 - RNA, 21 nt, Synthetic construct
  source 1..21: mol_type = other RNA
tctcagccactgttgccacac

SEQ ID NO: 126 - RNA, 21 nt, Synthetic construct
  source 1..21: mol_type = other RNA
ttctcagccactgttgccaca

SEQ ID NO: 127 - RNA, 21 nt, Synthetic construct
  source 1..21: mol_type = other RNA
tttctcagccactgttgccac

SEQ ID NO: 128 - RNA, 21 nt, Synthetic construct
  source 1..21: mol_type = other RNA
tcttctcagccactgttgcca

SEQ ID NO: 129 - RNA, 21 nt, Synthetic construct
  source 1..21: mol_type = other RNA
ttcttctcagccactgttgcc

SEQ ID NO: 130 - RNA, 21 nt, Synthetic construct
  source 1..21: mol_type = other RNA
tgtcttctcagccactgttgc

SEQ ID NO: 131 - RNA, 21 nt, Synthetic construct
  source 1..21: mol_type = other RNA
tggtcttctcagccactgttg

SEQ ID NO: 132 - RNA, 21 nt, Synthetic construct
  source 1..21: mol_type = other RNA
ttggtcttctcagccactgtt

SEQ ID NO: 133 - RNA, 21 nt, Synthetic construct
  source 1..21: mol_type = other RNA
tttggtcttctcagccactgt

SEQ ID NO: 134 - RNA, 21 nt, Synthetic construct
  source 1..21: mol_type = other RNA
ttttggtcttctcagccactg

SEQ ID NO: 135 - RNA, 21 nt, Synthetic construct
  source 1..21: mol_type = other RNA
tctttggtcttctcagccact

SEQ ID NO: 136 - RNA, 21 nt, Synthetic construct
  source 1..21: mol_type = other RNA
ttctttggtcttctcagccac

SEQ ID NO: 137 - RNA, 21 nt, Synthetic construct
  source 1..21: mol_type = other RNA
tctctttggtcttctcagcca

SEQ ID NO: 138 - RNA, 21 nt, Synthetic construct
  source 1..21: mol_type = other RNA
tgctctttggtcttctcagcc

SEQ ID NO: 139 - RNA, 21 nt, Synthetic construct
  source 1..21: mol_type = other RNA
tttgctctttggtcttctcag

SEQ ID NO: 140 - RNA, 21 nt, Synthetic construct
  source 1..21: mol_type = other RNA
tcttgctctttggtcttctca

SEQ ID NO: 141 - RNA, 21 nt, Synthetic construct
  source 1..21: mol_type = other RNA
tacttgctctttggtcttctc

SEQ ID NO: 142 - RNA, 21 nt, Synthetic construct
  source 1..21: mol_type = other RNA
tcacttgctctttggtcttct

SEQ ID NO: 143 - RNA, 21 nt, Synthetic construct
  source 1..21: mol_type = other RNA
ttcacttgctctttggtcttc

SEQ ID NO: 144 - RNA, 21 nt, Synthetic construct
  source 1..21: mol_type = other RNA
ttttgtcacttgctctttggt

SEQ ID NO: 145 - RNA, 21 nt, Synthetic construct
  source 1..21: mol_type = other RNA
tatttgtcacttgctctttgg

SEQ ID NO: 146 - RNA, 21 nt, Synthetic construct
  source 1..21: mol_type = other RNA
tcatttgtcacttgctctttg

SEQ ID NO: 147 - RNA, 21 nt, Synthetic construct
  source 1..21: mol_type = other RNA
tacatttgtcacttgctcttt

SEQ ID NO: 148 - RNA, 21 nt, Synthetic construct
  source 1..21: mol_type = other RNA
taacatttgtcacttgctctt

SEQ ID NO: 149 - RNA, 21 nt, Synthetic construct
  source 1..21: mol_type = other RNA
tcaacatttgtcacttgctct

SEQ ID NO: 150 - RNA, 21 nt, Synthetic construct
  source 1..21: mol_type = other RNA
tccaacatttgtcacttgctc

SEQ ID NO: 151 - RNA, 21 nt, Synthetic construct
  source 1..21: mol_type = other RNA
ttccaacatttgtcacttgct

SEQ ID NO: 152 - RNA, 21 nt, Synthetic construct
  source 1..21: mol_type = other RNA
tctccaacatttgtcacttgc

SEQ ID NO: 153 - RNA, 21 nt, Synthetic construct
  source 1..21: mol_type = other RNA
tcctccaacatttgtcacttg

SEQ ID NO: 154 - RNA, 21 nt, Synthetic construct
  source 1..21: mol_type = other RNA
tcccgtcaccactgctcctcc

SEQ ID NO: 155 - RNA, 21 nt, Synthetic construct
  source 1..21: mol_type = other RNA
tctgctgtcacacccgtcacc

SEQ ID NO: 156 - RNA, 21 nt, Synthetic construct
  source 1..21: mol_type = other RNA
tctactgctgtcacacccgtc

SEQ ID NO: 157 - RNA, 21 nt, Synthetic construct
  source 1..21: mol_type = other RNA
tttctgggctactgctgtcac

SEQ ID NO: 158 - RNA, 21 nt, Synthetic construct
  source 1..21: mol_type = other RNA
tcttctgggctactgctgtca

SEQ ID NO: 159 - RNA, 21 nt, Synthetic construct
  source 1..21: mol_type = other RNA
tcctccactgtcttctgggct

SEQ ID NO: 160 - RNA, 21 nt, Synthetic construct
  source 1..21: mol_type = other RNA
tccctccactgtcttctgggc

SEQ ID NO: 161 - RNA, 21 nt, Synthetic construct
  source 1..21: mol_type = other RNA
ttccctccactgtcttctggg

SEQ ID NO: 162 - RNA, 21 nt, Synthetic construct
  source 1..21: mol_type = other RNA
tctccctccactgtcttctgg

SEQ ID NO: 163 - RNA, 21 nt, Synthetic construct
  source 1..21: mol_type = other RNA
tgctccctccactgtcttctg

SEQ ID NO: 164 - RNA, 21 nt, Synthetic construct
  source 1..21: mol_type = other RNA
tatgctccctgctccctccac

SEQ ID NO: 165 - RNA, 21 nt, Synthetic construct
  source 1..21: mol_type = other RNA
tgcaatgctccctgctccctc

SEQ ID NO: 166 - RNA, 21 nt, Synthetic construct
  source 1..21: mol_type = other RNA
ttgcaatgctccctgctccct

SEQ ID NO: 167 - RNA, 21 nt, Synthetic construct
  source 1..21: mol_type = other RNA
tttcattcttgcccaactggt

SEQ ID NO: 168 - RNA, 21 nt, Synthetic construct
  source 1..21: mol_type = other RNA
tcttcattcttgcccaactgg

SEQ ID NO: 169 - RNA, 21 nt, Synthetic construct
  source 1..21: mol_type = other RNA
tttcttcattcttgcccaact

SEQ ID NO: 170 - RNA, 21 nt, Synthetic construct
  source 1..21: mol_type = other RNA
ttccagaattccttcctgtgg

SEQ ID NO: 171 - RNA, 21 nt, Synthetic construct
  source 1..21: mol_type = other RNA
tttccagaattccttcctgtg

SEQ ID NO: 172 - RNA, 21 nt, Synthetic construct
  source 1..21: mol_type = other RNA
tggcatatcttccagaattcc

SEQ ID NO: 173 - RNA, 21 nt, Synthetic construct
  source 1..21: mol_type = other RNA
tcacaggcatatcttccagaa

SEQ ID NO: 174 - RNA, 21 nt, Synthetic construct
  source 1..21: mol_type = other RNA
tcattgtcaggatccacaggc

SEQ ID NO: 175 - RNA, 21 nt, Synthetic construct
  source 1..21: mol_type = other RNA
tctcattgtcaggatccacag

SEQ ID NO: 176 - RNA, 21 nt, Synthetic construct
  source 1..21: mol_type = other RNA
tcctcattgtcaggatccaca

SEQ ID NO: 177 - RNA, 21 nt, Synthetic construct
  source 1..21: mol_type = other RNA
tataagcctcattgtcaggat

SEQ ID NO: 178 - RNA, 21 nt, Synthetic construct
  source 1..21: mol_type = other RNA
tcataagcctcattgtcagga

SEQ ID NO: 179 - RNA, 21 nt, Synthetic construct
  source 1..21: mol_type = other RNA
ttcataagcctcattgtcagg

SEQ ID NO: 180 - RNA, 21 nt, Synthetic construct
  source 1..21: mol_type = other RNA
tttcataagcctcattgtcag

SEQ ID NO: 181 - RNA, 21 nt, Synthetic construct
  source 1..21: mol_type = other RNA
ttttcataagcctcattgtca

SEQ ID NO: 182 - RNA, 21 nt, Synthetic construct
  source 1..21: mol_type = other RNA
tatttcataagcctcattgtc

SEQ ID NO: 183 - RNA, 21 nt, Synthetic construct
  source 1..21: mol_type = other RNA
tcatttcataagcctcattgt

SEQ ID NO: 184 - RNA, 21 nt, Synthetic construct
  source 1..21: mol_type = other RNA
tgcatttcataagcctcattg

SEQ ID NO: 185 - RNA, 21 nt, Synthetic construct
  source 1..21: mol_type = other RNA
tggcatttcataagcctcatt

SEQ ID NO: 186 - RNA, 21 nt, Synthetic construct
  source 1..21: mol_type = other RNA
taggcatttcataagcctcat

SEQ ID NO: 187 - RNA, 21 nt, Synthetic construct
  source 1..21: mol_type = other RNA
taaggcatttcataagcctca

SEQ ID NO: 188 - RNA, 21 nt, Synthetic construct
  source 1..21: mol_type = other RNA
tgaaggcatttcataagcctc

SEQ ID NO: 189 - RNA, 21 nt, Synthetic construct
  source 1..21: mol_type = other RNA
ttcagaaggcatttcataagc

SEQ ID NO: 190 - RNA, 21 nt, Synthetic construct
  source 1..21: mol_type = other RNA
tctcagaaggcatttcataag

SEQ ID NO: 191 - RNA, 21 nt, Synthetic construct
  source 1..21: mol_type = other RNA
tttcctcagaaggcatttcat

SEQ ID NO: 192 - RNA, 21 nt, Synthetic construct
  source 1..21: mol_type = other RNA
ttcttgatacccttcctcaga

SEQ ID NO: 193 - RNA, 21 nt, Synthetic construct
  source 1..21: mol_type = other RNA
tgtcttgatacccttcctcag

SEQ ID NO: 194 - RNA, 21 nt, Synthetic construct
  source 1..21: mol_type = other RNA
tcttcaggttcgtagtcttga

SEQ ID NO: 195 - RNA, 21 nt, Synthetic construct
  source 1..21: mol_type = other RNA
tgcttcaggttcgtagtcttg

SEQ ID NO: 196 - RNA, 21 nt, Synthetic construct
  source 1..21: mol_type = other RNA
tggcttcaggttcgtagtctt

SEQ ID NO: 197 - RNA, 21 nt, Synthetic construct
  source 1..21: mol_type = other RNA
tcttaggcttcaggttcgtag

SEQ ID NO: 198 - RNA, 21 nt, Synthetic construct
  source 1..21: mol_type = other RNA
ttcttaggcttcaggttcgta

SEQ ID NO: 199 - RNA, 21 nt, Synthetic construct
  source 1..21: mol_type = other RNA
ttcttaggcttcaggttcgta

SEQ ID NO: 200 - RNA, 21 nt, Synthetic construct
  source 1..21: mol_type = other RNA
tttcttaggcttcaggttcgt

SEQ ID NO: 201 - RNA, 21 nt, Synthetic construct
  source 1..21: mol_type = other RNA
ttttcttaggcttcaggttcg

SEQ ID NO: 202 - RNA, 21 nt, Synthetic construct
  source 1..21: mol_type = other RNA
tatttcttaggcttcaggttc

SEQ ID NO: 203 - RNA, 21 nt, Synthetic construct
  source 1..21: mol_type = other RNA
ttatttcttaggcttcaggtt

SEQ ID NO: 204 - RNA, 21 nt, Synthetic construct
  source 1..21: mol_type = other RNA
tgatatttcttaggcttcagg

SEQ ID NO: 205 - RNA, 21 nt, Synthetic construct
  source 1..21: mol_type = other RNA
tagatatttcttaggcttcag

SEQ ID NO: 206 - RNA, 21 nt, Synthetic construct
  source 1..21: mol_type = other RNA
taagatatttcttaggcttca

SEQ ID NO: 207 - RNA, 21 nt, Synthetic construct
  source 1..21: mol_type = other RNA
taaagatatttcttaggcttc

SEQ ID NO: 208 - RNA, 21 nt, Synthetic construct
  source 1..21: mol_type = other RNA
tcaaagatatttcttaggctt

SEQ ID NO: 209 - RNA, 21 nt, Synthetic construct
  source 1..21: mol_type = other RNA
tgcaaagatatttcttaggct

SEQ ID NO: 210 - RNA, 21 nt, Synthetic construct
  source 1..21: mol_type = other RNA
tagcaaagatatttcttaggc

SEQ ID NO: 211 - RNA, 21 nt, Synthetic construct
  source 1..21: mol_type = other RNA
tgagcaaagatatttcttagg

SEQ ID NO: 212 - RNA, 21 nt, Synthetic construct
  source 1..21: mol_type = other RNA
tggagcaaagatatttcttag

SEQ ID NO: 213 - RNA, 21 nt, Synthetic construct
  source 1..21: mol_type = other RNA
tgggagcaaagatatttctta

SEQ ID NO: 214 - RNA, 21 nt, Synthetic construct
  source 1..21: mol_type = other RNA
tcaagaaactgggagcaaaga

SEQ ID NO: 215 - RNA, 21 nt, Synthetic construct
  source 1..21: mol_type = other RNA
ttcaagaaactgggagcaaag

SEQ ID NO: 216 - RNA, 21 nt, Synthetic construct
  source 1..21: mol_type = other RNA
tctcaagaaactgggagcaaa

SEQ ID NO: 217 - RNA, 21 nt, Synthetic construct
  source 1..21: mol_type = other RNA
ttctcaagaaactgggagcaa

SEQ ID NO: 218 - RNA, 21 nt, Synthetic construct
  source 1..21: mol_type = other RNA
tatctcaagaaactgggagca

SEQ ID NO: 219 - RNA, 21 nt, Synthetic construct
  source 1..21: mol_type = other RNA
tgatctcaagaaactgggagc

SEQ ID NO: 220 - RNA, 21 nt, Synthetic construct
  source 1..21: mol_type = other RNA
tagatctcaagaaactgggag

SEQ ID NO: 221 - RNA, 21 nt, Synthetic construct
  source 1..21: mol_type = other RNA
tgcagatctcaagaaactggg

SEQ ID NO: 222 - RNA, 21 nt, Synthetic construct
  source 1..21: mol_type = other RNA
tgtcagcagatctcaagaaac

SEQ ID NO: 223 - RNA, 21 nt, Synthetic construct
  source 1..21: mol_type = other RNA
ttgtcagcagatctcaagaaa

SEQ ID NO: 224 - RNA, 21 nt, Synthetic construct
  source 1..21: mol_type = other RNA
tctgtcagcagatctcaagaa

SEQ ID NO: 225 - RNA, 21 nt, Synthetic construct
  source 1..21: mol_type = other RNA
ttctgtcagcagatctcaaga

SEQ ID NO: 226 - RNA, 21 nt, Synthetic construct
  source 1..21: mol_type = other RNA
tatctgtcagcagatctcaag

SEQ ID NO: 227 - RNA, 21 nt, Synthetic construct
  source 1..21: mol_type = other RNA
tcatctgtcagcagatctcaa

SEQ ID NO: 228 - RNA, 21 nt, Synthetic construct
  source 1..21: mol_type = other RNA
tacatctgtcagcagatctca

SEQ ID NO: 229 - RNA, 21 nt, Synthetic construct
  source 1..21: mol_type = other RNA
ttggaacatctgtcagcagat

SEQ ID NO: 230 - RNA, 21 nt, Synthetic construct
  source 1..21: mol_type = other RNA
tatggaacatctgtcagcaga

SEQ ID NO: 231 - RNA, 21 nt, Synthetic construct
  source 1..21: mol_type = other RNA
tgatggaacatctgtcagcag

SEQ ID NO: 232 - RNA, 21 nt, Synthetic construct
  source 1..21: mol_type = other RNA
tggatggaacatctgtcagca

SEQ ID NO: 233 - RNA, 21 nt, Synthetic construct
  source 1..21: mol_type = other RNA
taggatggaacatctgtcagc

SEQ ID NO: 234 - RNA, 21 nt, Synthetic construct
  source 1..21: mol_type = other RNA
tcaggatggaacatctgtcag

SEQ ID NO: 235 - RNA, 21 nt, Synthetic construct
  source 1..21: mol_type = other RNA
tacaggatggaacatctgtca

SEQ ID NO: 236 - RNA, 21 nt, Synthetic construct
  source 1..21: mol_type = other RNA
ttacaggatggaacatctgtc

SEQ ID NO: 237 - RNA, 21 nt, Synthetic construct
  source 1..21: mol_type = other RNA
tttgtacaggatggaacatct

SEQ ID NO: 238 - RNA, 21 nt, Synthetic construct
  source 1..21: mol_type = other RNA
tagcacttgtacaggatggaa

SEQ ID NO: 239 - RNA, 21 nt, Synthetic construct
  source 1..21: mol_type = other RNA
ttgagcacttgtacaggatgg

SEQ ID NO: 240 - RNA, 21 nt, Synthetic construct
  source 1..21: mol_type = other RNA
tctgagcacttgtacaggatg

SEQ ID NO: 241 - RNA, 21 nt, Synthetic construct
  source 1..21: mol_type = other RNA
tactgagcacttgtacaggat

SEQ ID NO: 242 - RNA, 21 nt, Synthetic construct
  source 1..21: mol_type = other RNA
taactgagcacttgtacagga

SEQ ID NO: 243 - RNA, 21 nt, Synthetic construct
  source 1..21: mol_type = other RNA
tgaactgagcacttgtacagg

SEQ ID NO: 244 - RNA, 21 nt, Synthetic construct
  source 1..21: mol_type = other RNA
tggaactgagcacttgtacag

SEQ ID NO: 245 - RNA, 21 nt, Synthetic construct
  source 1..21: mol_type = other RNA
ttggaactgagcacttgtaca

SEQ ID NO: 246 - RNA, 21 nt, Synthetic construct
  source 1..21: mol_type = other RNA
tttggaactgagcacttgtac

SEQ ID NO: 247 - RNA, 21 nt, Synthetic construct
  source 1..21: mol_type = other RNA
tattggaactgagcacttgta

SEQ ID NO: 248 - RNA, 21 nt, Synthetic construct
  source 1..21: mol_type = other RNA
tcattggaactgagcacttgt

SEQ ID NO: 249 - RNA, 21 nt, Synthetic construct
  source 1..21: mol_type = other RNA
tacattggaactgagcacttg

SEQ ID NO: 250 - RNA, 21 nt, Synthetic construct
  source 1..21: mol_type = other RNA
tcacattggaactgagcactt

SEQ ID NO: 251 - RNA, 21 nt, Synthetic construct
  source 1..21: mol_type = other RNA
tgcacattggaactgagcact

SEQ ID NO: 252 - RNA, 21 nt, Synthetic construct
  source 1..21: mol_type = other RNA
tggcacattggaactgagcac

SEQ ID NO: 253 - RNA, 21 nt, Synthetic construct
  source 1..21: mol_type = other RNA
tgggcacattggaactgagca

SEQ ID NO: 254 - RNA, 21 nt, Synthetic construct
  source 1..21: mol_type = other RNA
ttgggcacattggaactgagc

SEQ ID NO: 255 - RNA, 21 nt, Synthetic construct
  source 1..21: mol_type = other RNA
tctgggcacattggaactgag

SEQ ID NO: 256 - RNA, 21 nt, Synthetic construct
  source 1..21: mol_type = other RNA
tactgggcacattggaactga

SEQ ID NO: 257 - RNA, 21 nt, Synthetic construct
  source 1..21: mol_type = other RNA
tgactgggcacattggaactg

SEQ ID NO: 258 - RNA, 21 nt, Synthetic construct
  source 1..21: mol_type = other RNA
ttgactgggcacattggaact

SEQ ID NO: 259 - RNA, 21 nt, Synthetic construct
  source 1..21: mol_type = other RNA
tatgactgggcacattggaac

SEQ ID NO: 260 - RNA, 21 nt, Synthetic construct
  source 1..21: mol_type = other RNA
tgtcatgactgggcacattgg

SEQ ID NO: 261 - RNA, 21 nt, Synthetic construct
  source 1..21: mol_type = other RNA
taatgtcatgactgggcacat

SEQ ID NO: 262 - RNA, 21 nt, Synthetic construct
  source 1..21: mol_type = other RNA
taaatgtcatgactgggcaca

SEQ ID NO: 263 - RNA, 21 nt, Synthetic construct
  source 1..21: mol_type = other RNA
tgaaatgtcatgactgggcac

SEQ ID NO: 264 - RNA, 21 nt, Synthetic construct
  source 1..21: mol_type = other RNA
ttgagaaatgtcatgactggg

SEQ ID NO: 265 - RNA, 21 nt, Synthetic construct
  source 1..21: mol_type = other RNA
tttgagaaatgtcatgactgg

SEQ ID NO: 266 - RNA, 21 nt, Synthetic construct
  source 1..21: mol_type = other RNA
ttttgagaaatgtcatgactg

SEQ ID NO: 267 - RNA, 21 nt, Synthetic construct
  source 1..21: mol_type = other RNA
tctttgagaaatgtcatgact

SEQ ID NO: 268 - RNA, 21 nt, Synthetic construct
  source 1..21: mol_type = other RNA
tactttgagaaatgtcatgac

SEQ ID NO: 269 - RNA, 21 nt, Synthetic construct
  source 1..21: mol_type = other RNA
tcttcgagatacactgtaaaa

SEQ ID NO: 270 - RNA, 21 nt, Synthetic construct
  source 1..21: mol_type = other RNA
tacttcgagatacactgtaaa

SEQ ID NO: 271 - RNA, 21 nt, Synthetic construct
  source 1..21: mol_type = other RNA
tgacttcgagatacactgtaa

SEQ ID NO: 272 - RNA, 21 nt, Synthetic construct
  source 1..21: mol_type = other RNA
tagacttcgagatacactgta

SEQ ID NO: 273 - RNA, 21 nt, Synthetic construct
  source 1..21: mol_type = other RNA
taagacttcgagatacactgt

SEQ ID NO: 274 - RNA, 21 nt, Synthetic construct
  source 1..21: mol_type = other RNA
tgaagacttcgagatacactg

SEQ ID NO: 275 - RNA, 21 nt, Synthetic construct
  source 1..21: mol_type = other RNA
tgatggaagacttcgagatac

SEQ ID NO: 276 - RNA, 21 nt, Synthetic construct
  source 1..21: mol_type = other RNA
ttgatggaagacttcgagata

SEQ ID NO: 277 - RNA, 21 nt, Synthetic construct
  source 1..21: mol_type = other RNA
tctgatggaagacttcgagat

SEQ ID NO: 278 - RNA, 21 nt, Synthetic construct
  source 1..21: mol_type = other RNA
tgctgatggaagacttcgaga

SEQ ID NO: 279 - RNA, 21 nt, Synthetic construct
  source 1..21: mol_type = other RNA
ttgctgatggaagacttcgag

SEQ ID NO: 280 - RNA, 21 nt, Synthetic construct
  source 1..21: mol_type = other RNA
tctgctgatggaagacttcga

SEQ ID NO: 281 - RNA, 21 nt, Synthetic construct
  source 1..21: mol_type = other RNA
tactgctgatggaagacttcg

SEQ ID NO: 282 - RNA, 21 nt, Synthetic construct
  source 1..21: mol_type = other RNA
tcactgctgatggaagacttc

SEQ ID NO: 283 - RNA, 21 nt, Synthetic construct
  source 1..21: mol_type = other RNA
ttcactgctgatggaagactt

SEQ ID NO: 284 - RNA, 21 nt, Synthetic construct
  source 1..21: mol_type = other RNA
tatcactgctgatggaagact

SEQ ID NO: 285 - RNA, 21 nt, Synthetic construct
  source 1..21: mol_type = other RNA
taatcactgctgatggaagac

SEQ ID NO: 286 - RNA, 21 nt, Synthetic construct
  source 1..21: mol_type = other RNA
tcaatcactgctgatggaaga

SEQ ID NO: 287 - RNA, 21 nt, Synthetic construct
  source 1..21: mol_type = other RNA
ttcaatcactgctgatggaag

SEQ ID NO: 288 - RNA, 21 nt, Synthetic construct
  source 1..21: mol_type = other RNA
tttcaatcactgctgatggaa

SEQ ID NO: 289 - RNA, 21 nt, Synthetic construct
  source 1..21: mol_type = other RNA
tcttcaatcactgctgatgga

SEQ ID NO: 290 - RNA, 21 nt, Synthetic construct
  source 1..21: mol_type = other RNA
tacttcaatcactgctgatgg

SEQ ID NO: 291 - RNA, 21 nt, Synthetic construct
  source 1..21: mol_type = other RNA
ttacttcaatcactgctgatg

SEQ ID NO: 292 - RNA, 21 nt, Synthetic construct
  source 1..21: mol_type = other RNA
tatacttcaatcactgctgat

SEQ ID NO: 293 - RNA, 21 nt, Synthetic construct
  source 1..21: mol_type = other RNA
tggtacagatacttcaatcac

SEQ ID NO: 294 - RNA, 21 nt, Synthetic construct
  source 1..21: mol_type = other RNA
taggtacagatacttcaatca

SEQ ID NO: 295 - RNA, 21 nt, Synthetic construct
  source 1..21: mol_type = other RNA
tcaggtacagatacttcaatc

SEQ ID NO: 296 - RNA, 21 nt, Synthetic construct
  source 1..21: mol_type = other RNA
tgcaggtacagatacttcaat

SEQ ID NO: 297 - RNA, 21 nt, Synthetic construct
  source 1..21: mol_type = other RNA
tggcaggtacagatacttcaa

SEQ ID NO: 298 - RNA, 21 nt, Synthetic construct
  source 1..21: mol_type = other RNA
tgggcaggtacagatacttca

SEQ ID NO: 299 - RNA, 21 nt, Synthetic construct
  source 1..21: mol_type = other RNA
taagcaccgaaatgctgagtg

SEQ ID NO: 300 - RNA, 21 nt, Synthetic construct
  source 1..21: mol_type = other RNA
tgaagcaccgaaatgctgagt

SEQ ID NO: 301 - RNA, 21 nt, Synthetic construct
  source 1..21: mol_type = other RNA
taagggaagcaccgaaatgct

SEQ ID NO: 302 - RNA, 21 nt, Synthetic construct
  source 1..21: mol_type = other RNA
taaagggaagcaccgaaatgc

SEQ ID NO: 303 - RNA, 21 nt, Synthetic construct
  source 1..21: mol_type = other RNA
ttgaaagggaagcaccgaaat

SEQ ID NO: 304 - RNA, 21 nt, Synthetic construct
  source 1..21: mol_type = other RNA
tgtgaaagggaagcaccgaaa

SEQ ID NO: 305 - RNA, 21 nt, Synthetic construct
  source 1..21: mol_type = other RNA
tagtgaaagggaagcaccgaa

SEQ ID NO: 306 - RNA, 21 nt, Synthetic construct
  source 1..21: mol_type = other RNA
ttcagtgaaagggaagcaccg

SEQ ID NO: 307 - RNA, 21 nt, Synthetic construct
  source 1..21: mol_type = other RNA
tacttcagtgaaagggaagca

SEQ ID NO: 308 - RNA, 21 nt, Synthetic construct
  source 1..21: mol_type = other RNA
ttattcacttcagtgaaaggg

SEQ ID NO: 309 - RNA, 21 nt, Synthetic construct
  source 1..21: mol_type = other RNA
tgtattcacttcagtgaaagg

SEQ ID NO: 310 - RNA, 21 nt, Synthetic construct
  source 1..21: mol_type = other RNA
tatgtattcacttcagtgaaa

SEQ ID NO: 311 - RNA, 21 nt, Synthetic construct
  source 1..21: mol_type = other RNA
tcatgtattcacttcagtgaa

SEQ ID NO: 312 - RNA, 21 nt, Synthetic construct
  source 1..21: mol_type = other RNA
tccatgtattcacttcagtga

SEQ ID NO: 313 - RNA, 21 nt, Synthetic construct
  source 1..21: mol_type = other RNA
taccatgtattcacttcagtg

SEQ ID NO: 314 - RNA, 21 nt, Synthetic construct
  source 1..21: mol_type = other RNA
ttaccatgtattcacttcagt

SEQ ID NO: 315 - RNA, 21 nt, Synthetic construct
  source 1..21: mol_type = other RNA
taccctgctaccatgtattca

SEQ ID NO: 316 - RNA, 21 nt, Synthetic construct
  source 1..21: mol_type = other RNA
tacacaaagaccctgctacca

SEQ ID NO: 317 - RNA, 21 nt, Synthetic construct
  source 1..21: mol_type = other RNA
taaaatccacagcacacaaag

SEQ ID NO: 318 - RNA, 21 nt, Synthetic construct
  source 1..21: mol_type = other RNA
tacaaaatccacagcacacaa

SEQ ID NO: 319 - RNA, 21 nt, Synthetic construct
  source 1..21: mol_type = other RNA
tgtagattgaagccacaaaat

SEQ ID NO: 320 - RNA, 21 nt, Synthetic construct
  source 1..21: mol_type = other RNA
tcgtagattgaagccacaaaa

SEQ ID NO: 321 - RNA, 21 nt, Synthetic construct
  source 1..21: mol_type = other RNA
ttcgtagattgaagccacaaa

SEQ ID NO: 322 - RNA, 21 nt, Synthetic construct
  source 1..21: mol_type = other RNA
tatcgtagattgaagccacaa

SEQ ID NO: 323 - RNA, 21 nt, Synthetic construct
  source 1..21: mol_type = other RNA
tcatcgtagattgaagccaca

SEQ ID NO: 324 - RNA, 21 nt, Synthetic construct
  source 1..21: mol_type = other RNA
tacatcgtagattgaagccac

SEQ ID NO: 325 - RNA, 21 nt, Synthetic construct
  source 1..21: mol_type = other RNA
taacatcgtagattgaagcca

SEQ ID NO: 326 - RNA, 21 nt, Synthetic construct
  source 1..21: mol_type = other RNA
ttaacatcgtagattgaagcc

SEQ ID NO: 327 - RNA, 21 nt, Synthetic construct
  source 1..21: mol_type = other RNA
tttaacatcgtagattgaagc

SEQ ID NO: 328 - RNA, 21 nt, Synthetic construct
  source 1..21: mol_type = other RNA
tgttttaacatcgtagattga

SEQ ID NO: 329 - RNA, 21 nt, Synthetic construct
  source 1..21: mol_type = other RNA
tggtagtcacttaggtgtttt

SEQ ID NO: 330 - RNA, 21 nt, Synthetic construct
  source 1..21: mol_type = other RNA
ttggtagtcacttaggtgttt

SEQ ID NO: 331 - RNA, 21 nt, Synthetic construct
  source 1..21: mol_type = other RNA
tgtggtagtcacttaggtgtt

SEQ ID NO: 332 - RNA, 21 nt, Synthetic construct
  source 1..21: mol_type = other RNA
tagtggtagtcacttaggtgt

SEQ ID NO: 333 - RNA, 21 nt, Synthetic construct
  source 1..21: mol_type = other RNA
taagtggtagtcacttaggtg

SEQ ID NO: 334 - RNA, 21 nt, Synthetic construct
  source 1..21: mol_type = other RNA
ttaagtggtagtcacttaggt

SEQ ID NO: 335 - RNA, 21 nt, Synthetic construct
  source 1..21: mol_type = other RNA
tataagtggtagtcacttagg

SEQ ID NO: 336 - RNA, 21 nt, Synthetic construct
  source 1..21: mol_type = other RNA
taataagtggtagtcacttag

SEQ ID NO: 337 - RNA, 21 nt, Synthetic construct
  source 1..21: mol_type = other RNA
taaataagtggtagtcactta

SEQ ID NO: 338 - RNA, 21 nt, Synthetic construct
  source 1..21: mol_type = other RNA
tgaaataagtggtagtcactt

SEQ ID NO: 339 - RNA, 21 nt, Synthetic construct
  source 1..21: mol_type = other RNA
tagaaataagtggtagtcact

SEQ ID NO: 340 - RNA, 21 nt, Synthetic construct
  source 1..21: mol_type = other RNA
ttagaaataagtggtagtcac

SEQ ID NO: 341 - RNA, 21 nt, Synthetic construct
  source 1..21: mol_type = other RNA
tttagaaataagtggtagtca

SEQ ID NO: 342 - RNA, 21 nt, Synthetic construct
  source 1..21: mol_type = other RNA
tatttagaaataagtggtagt

SEQ ID NO: 343 - RNA, 21 nt, Synthetic construct
  source 1..21: mol_type = other RNA
tgatttagaaataagtggtag

SEQ ID NO: 344 - RNA, 21 nt, Synthetic construct
  source 1..21: mol_type = other RNA
taggatttagaaataagtggt

SEQ ID NO: 345 - RNA, 21 nt, Synthetic construct
  source 1..21: mol_type = other RNA
ttctgaacaacagcaacaaaa

SEQ ID NO: 346 - RNA, 21 nt, Synthetic construct
  source 1..21: mol_type = other RNA
tttctgaacaacagcaacaaa

SEQ ID NO: 347 - RNA, 21 nt, Synthetic construct
  source 1..21: mol_type = other RNA
tcttctgaacaacagcaacaa

SEQ ID NO: 348 - RNA, 21 nt, Synthetic construct
  source 1..21: mol_type = other RNA
tacttctgaacaacagcaaca

SEQ ID NO: 349 - RNA, 21 nt, Synthetic construct
  source 1..21: mol_type = other RNA
taacttctgaacaacagcaac

SEQ ID NO: 350 - RNA, 21 nt, Synthetic construct
  source 1..21: mol_type = other RNA
tcaacttctgaacaacagcaa

SEQ ID NO: 351 - RNA, 21 nt, Synthetic construct
  source 1..21: mol_type = other RNA
tacaacttctgaacaacagca

SEQ ID NO: 352 - RNA, 21 nt, Synthetic construct
  source 1..21: mol_type = other RNA
taacaacttctgaacaacagc

SEQ ID NO: 353 - RNA, 21 nt, Synthetic construct
  source 1..21: mol_type = other RNA
ttaacaacttctgaacaacag

SEQ ID NO: 354 - RNA, 21 nt, Synthetic construct
  source 1..21: mol_type = other RNA
tctaacaacttctgaacaaca

SEQ ID NO: 355 - RNA, 21 nt, Synthetic construct
  source 1..21: mol_type = other RNA
tactaacaacttctgaacaac

SEQ ID NO: 356 - RNA, 21 nt, Synthetic construct
  source 1..21: mol_type = other RNA
tcactaacaacttctgaacaa

SEQ ID NO: 357 - RNA, 21 nt, Synthetic construct
  source 1..21: mol_type = other RNA
ttcactaacaacttctgaaca

SEQ ID NO: 358 - RNA, 21 nt, Synthetic construct
  source 1..21: mol_type = other RNA
tatcactaacaacttctgaac

SEQ ID NO: 359 - RNA, 21 nt, Synthetic construct
  source 1..21: mol_type = other RNA
taatcactaacaacttctgaa

SEQ ID NO: 360 - RNA, 21 nt, Synthetic construct
  source 1..21: mol_type = other RNA
taaatcactaacaacttctga

SEQ ID NO: 361 - RNA, 21 nt, Synthetic construct
  source 1..21: mol_type = other RNA
tcaaatcactaacaacttctg

SEQ ID NO: 362 - RNA, 21 nt, Synthetic construct
  source 1..21: mol_type = other RNA
tgcaaatcactaacaacttct

SEQ ID NO: 363 - RNA, 21 nt, Synthetic construct
  source 1..21: mol_type = other RNA
tagcaaatcactaacaacttc

SEQ ID NO: 364 - RNA, 21 nt, Synthetic construct
  source 1..21: mol_type = other RNA
ttagcaaatcactaacaactt

SEQ ID NO: 365 - RNA, 21 nt, Synthetic construct
  source 1..21: mol_type = other RNA
tatagcaaatcactaacaact

SEQ ID NO: 366 - RNA, 21 nt, Synthetic construct
  source 1..21: mol_type = other RNA
tgatagcaaatcactaacaac

SEQ ID NO: 367 - RNA, 21 nt, Synthetic construct
  source 1..21: mol_type = other RNA
ttgatagcaaatcactaacaa

SEQ ID NO: 368 - RNA, 21 nt, Synthetic construct
  source 1..21: mol_type = other RNA
tatgatagcaaatcactaaca

SEQ ID NO: 369 - RNA, 21 nt, Synthetic construct
  source 1..21: mol_type = other RNA
ttatcattaaaagacacctaa

SEQ ID NO: 370 - RNA, 21 nt, Synthetic construct
  source 1..21: mol_type = other RNA
tgtatcattaaaagacaccta

SEQ ID NO: 371 - RNA, 21 nt, Synthetic construct
  source 1..21: mol_type = other RNA
tagtatcattaaaagacacct

SEQ ID NO: 372 - RNA, 21 nt, Synthetic construct
  source 1..21: mol_type = other RNA
tcagtatcattaaaagacacc

SEQ ID NO: 373 - RNA, 21 nt, Synthetic construct
  source 1..21: mol_type = other RNA
tacagtatcattaaaagacac

SEQ ID NO: 374 - RNA, 21 nt, Synthetic construct
  source 1..21: mol_type = other RNA
tgacagtatcattaaaagaca

SEQ ID NO: 375 - RNA, 21 nt, Synthetic construct
  source 1..21: mol_type = other RNA
tattattcttagacagtatca

SEQ ID NO: 376 - RNA, 21 nt, Synthetic construct
  source 1..21: mol_type = other RNA
ttcattattcttagacagtat

SEQ ID NO: 377 - RNA, 21 nt, Synthetic construct
  source 1..21: mol_type = other RNA
tgtcattattcttagacagta

SEQ ID NO: 378 - RNA, 21 nt, Synthetic construct
  source 1..21: mol_type = other RNA
tcgtcattattcttagacagt

SEQ ID NO: 379 - RNA, 21 nt, Synthetic construct
  source 1..21: mol_type = other RNA
tacgtcattattcttagacag

SEQ ID NO: 380 - RNA, 21 nt, Synthetic construct
  source 1..21: mol_type = other RNA
ttacgtcattattcttagaca

SEQ ID NO: 381 - RNA, 21 nt, Synthetic construct
  source 1..21: mol_type = other RNA
taatacgtcattattcttaga

SEQ ID NO: 382 - RNA, 21 nt, Synthetic construct
  source 1..21: mol_type = other RNA
tacaatacgtcattattctta

SEQ ID NO: 383 - RNA, 21 nt, Synthetic construct
  source 1..21: mol_type = other RNA
tcacaatacgtcattattctt

SEQ ID NO: 384 - RNA, 21 nt, Synthetic construct
  source 1..21: mol_type = other RNA
ttcacaatacgtcattattct

SEQ ID NO: 385 - RNA, 21 nt, Synthetic construct
  source 1..21: mol_type = other RNA
taaatttcacaatacgtcatt

SEQ ID NO: 386 - RNA, 21 nt, Synthetic construct
  source 1..21: mol_type = other RNA
tcaaatttcacaatacgtcat

SEQ ID NO: 387 - RNA, 21 nt, Synthetic construct
  source 1..21: mol_type = other RNA
tacaaatttcacaatacgtca

SEQ ID NO: 388 - RNA, 21 nt, Synthetic construct
  source 1..21: mol_type = other RNA
tgtttcatgctcacatatttt

SEQ ID NO: 389 - RNA, 21 nt, Synthetic construct
  source 1..21: mol_type = other RNA
tagtttcatgctcacatattt

SEQ ID NO: 390 - RNA, 21 nt, Synthetic construct
  source 1..21: mol_type = other RNA
ttagtttcatgctcacatatt

SEQ ID NO: 391 - RNA, 21 nt, Synthetic construct
  source 1..21: mol_type = other RNA
tatagtttcatgctcacatat

SEQ ID NO: 392 - RNA, 21 nt, Synthetic construct
  source 1..21: mol_type = other RNA
tcatagtttcatgctcacata

SEQ ID NO: 393 - RNA, 21 nt, Synthetic construct
  source 1..21: mol_type = other RNA
tgcatagtttcatgctcacat

SEQ ID NO: 394 - RNA, 21 nt, Synthetic construct
  source 1..21: mol_type = other RNA
ttgcatagtttcatgctcaca

SEQ ID NO: 395 - RNA, 21 nt, Synthetic construct
  source 1..21: mol_type = other RNA
tgtgcatagtttcatgctcac

SEQ ID NO: 396 - RNA, 21 nt, Synthetic construct
  source 1..21: mol_type = other RNA
tggtgcatagtttcatgctca

SEQ ID NO: 397 - RNA, 21 nt, Synthetic construct
  source 1..21: mol_type = other RNA
taggtgcatagtttcatgctc

SEQ ID NO: 398 - RNA, 21 nt, Synthetic construct
  source 1..21: mol_type = other RNA
ttaggtgcatagtttcatgct

SEQ ID NO: 399 - RNA, 21 nt, Synthetic construct
  source 1..21: mol_type = other RNA
tataggtgcatagtttcatgc

SEQ ID NO: 400 - RNA, 21 nt, Synthetic construct
  source 1..21: mol_type = other RNA
ttataggtgcatagtttcatg

SEQ ID NO: 401 - RNA, 21 nt, Synthetic construct
  source 1..21: mol_type = other RNA
ttttataggtgcatagtttca

SEQ ID NO: 402 - RNA, 21 nt, Synthetic construct
  source 1..21: mol_type = other RNA
ttatttataggtgcatagttt

SEQ ID NO: 403 - RNA, 21 nt, Synthetic construct
  source 1..21: mol_type = other RNA
tagtatttataggtgcatagt

SEQ ID NO: 404 - RNA, 21 nt, Synthetic construct
  source 1..21: mol_type = other RNA
tgcaaaatggtaaaatttcat

SEQ ID NO: 405 - RNA, 21 nt, Synthetic construct
  source 1..21: mol_type = other RNA
tcatcgcaaaatggtaaaatt

SEQ ID NO: 406 - RNA, 21 nt, Synthetic construct
  source 1..21: mol_type = other RNA
tcacatcgcaaaatggtaaaa

SEQ ID NO: 407 - RNA, 21 nt, Synthetic construct
  source 1..21: mol_type = other RNA
tacacatcgcaaaatggtaaa

SEQ ID NO: 408 - RNA, 21 nt, Synthetic construct
  source 1..21: mol_type = other RNA
taacacatcgcaaaatggtaa

SEQ ID NO: 409 - RNA, 21 nt, Synthetic construct
  source 1..21: mol_type = other RNA
taaacacatcgcaaaatggta

SEQ ID NO: 410 - RNA, 21 nt, Synthetic construct
  source 1..21: mol_type = other RNA
taaaacacatcgcaaaatggt

SEQ ID NO: 411 - RNA, 21 nt, Synthetic construct
  source 1..21: mol_type = other RNA
ttaaaacacatcgcaaaatgg

SEQ ID NO: 412 - RNA, 21 nt, Synthetic construct
  source 1..21: mol_type = other RNA
tgaataaaacacatcgcaaaa

SEQ ID NO: 413 - RNA, 21 nt, Synthetic construct
  source 1..21: mol_type = other RNA
ttgaataaaacacatcgcaaa

SEQ ID NO: 414 - RNA, 21 nt, Synthetic construct
  source 1..21: mol_type = other RNA
tgtgaataaaacacatcgcaa

SEQ ID NO: 415 - RNA, 21 nt, Synthetic construct
  source 1..21: mol_type = other RNA
taagtgaataaaacacatcgc

SEQ ID NO: 416 - RNA, 21 nt, Synthetic construct
  source 1..21: mol_type = other RNA
tcaagtgaataaaacacatcg

SEQ ID NO: 417 - RNA, 21 nt, Synthetic construct
  source 1..21: mol_type = other RNA
tacaagtgaataaaacacatc

SEQ ID NO: 418 - RNA, 21 nt, Synthetic construct
  source 1..21: mol_type = other RNA
tcacaagtgaataaaacacat

SEQ ID NO: 419 - RNA, 21 nt, Synthetic construct
  source 1..21: mol_type = other RNA
taccatttatatacaaacaca

SEQ ID NO: 420 - RNA, 21 nt, Synthetic construct
  source 1..21: mol_type = other RNA
ttcaccatttatatacaaaca

SEQ ID NO: 421 - RNA, 16 nt, Synthetic construct
  source 1..16: mol_type = other RNA
cttgccttcaagccta

SEQ ID NO: 422 - RNA, 16 nt, Synthetic construct
  source 1..16: mol_type = other RNA
cttcaagccttctgca

SEQ ID NO: 423 - RNA, 16 nt, Synthetic construct
  source 1..16: mol_type = other RNA
tcaagccttctgccta

SEQ ID NO: 424 - RNA, 16 nt, Synthetic construct
  source 1..16: mol_type = other RNA
caagccttctgcctta

SEQ ID NO: 425 - RNA, 16 nt, Synthetic construct
  source 1..16: mol_type = other RNA
aagccttctgccttta

SEQ ID NO: 426 - RNA, 16 nt, Synthetic construct
  source 1..16: mol_type = other RNA
agccttctgcctttca

SEQ ID NO: 427 - RNA, 16 nt, Synthetic construct
  source 1..16: mol_type = other RNA
ccttctgcctttccaa

SEQ ID NO: 428 - RNA, 16 nt, Synthetic construct
  source 1..16: mol_type = other RNA
cttctgcctttccaca

SEQ ID NO: 429 - RNA, 16 nt, Synthetic construct
  source 1..16: mol_type = other RNA
tttccaccctcgtgaa

SEQ ID NO: 430 - RNA, 16 nt, Synthetic construct
  source 1..16: mol_type = other RNA
tccaccctcgtgagca

SEQ ID NO: 431 - RNA, 16 nt, Synthetic construct
  source 1..16: mol_type = other RNA
tggccattcgacgaca

SEQ ID NO: 432 - RNA, 16 nt, Synthetic construct
  source 1..16: mol_type = other RNA
tgtggtgtaaaggaaa

SEQ ID NO: 433 - RNA, 16 nt, Synthetic construct
  source 1..16: mol_type = other RNA
gtggtgtaaaggaata

SEQ ID NO: 434 - RNA, 16 nt, Synthetic construct
  source 1..16: mol_type = other RNA
tggtgtaaaggaatta

SEQ ID NO: 435 - RNA, 16 nt, Synthetic construct
  source 1..16: mol_type = other RNA
tgtaaaggaattcata

SEQ ID NO: 436 - RNA, 16 nt, Synthetic construct
  source 1..16: mol_type = other RNA
gtaaaggaattcatta

SEQ ID NO: 437 - RNA, 16 nt, Synthetic construct
  source 1..16: mol_type = other RNA
taaaggaattcattaa

SEQ ID NO: 438 - RNA, 16 nt, Synthetic construct
  source 1..16: mol_type = other RNA
aaaggaattcattaga

SEQ ID NO: 439 - RNA, 16 nt, Synthetic construct
  source 1..16: mol_type = other RNA
aaggaattcattagca

SEQ ID NO: 440 - RNA, 16 nt, Synthetic construct
  source 1..16: mol_type = other RNA
aggaattcattagcca

SEQ ID NO: 441 - RNA, 16 nt, Synthetic construct
  source 1..16: mol_type = other RNA
ggaattcattagccaa

SEQ ID NO: 442 - RNA, 16 nt, Synthetic construct
  source 1..16: mol_type = other RNA
gaattcattagccata

SEQ ID NO: 443 - RNA, 16 nt, Synthetic construct
  source 1..16: mol_type = other RNA
ttcattagccatggaa

SEQ ID NO: 444 - RNA, 16 nt, Synthetic construct
  source 1..16: mol_type = other RNA
tcattagccatggata

SEQ ID NO: 445 - RNA, 16 nt, Synthetic construct
  source 1..16: mol_type = other RNA
ttagccatggatgtaa

SEQ ID NO: 446 - RNA, 16 nt, Synthetic construct
  source 1..16: mol_type = other RNA
tagccatggatgtata

SEQ ID NO: 447 - RNA, 16 nt, Synthetic construct
  source 1..16: mol_type = other RNA
agccatggatgtatta

SEQ ID NO: 448 - RNA, 16 nt, Synthetic construct
  source 1..16: mol_type = other RNA
gccatggatgtattca

SEQ ID NO: 449 - RNA, 16 nt, Synthetic construct
  source 1..16: mol_type = other RNA
ccatggatgtattcaa

SEQ ID NO: 450 - RNA, 16 nt, Synthetic construct
  source 1..16: mol_type = other RNA
catggatgtattcata

SEQ ID NO: 451 - RNA, 16 nt, Synthetic construct
  source 1..16: mol_type = other RNA
atggatgtattcatga

SEQ ID NO: 452 - RNA, 16 nt, Synthetic construct
  source 1..16: mol_type = other RNA
tggatgtattcatgaa

SEQ ID NO: 453 - RNA, 16 nt, Synthetic construct
  source 1..16: mol_type = other RNA
ggatgtattcatgaaa

SEQ ID NO: 454 - RNA, 16 nt, Synthetic construct
  source 1..16: mol_type = other RNA
gatgtattcatgaaaa

SEQ ID NO: 455 - RNA, 16 nt, Synthetic construct
  source 1..16: mol_type = other RNA
tattcatgaaaggaca

SEQ ID NO: 456 - RNA, 16 nt, Synthetic construct
  source 1..16: mol_type = other RNA
attcatgaaaggacta

SEQ ID NO: 457 - RNA, 16 nt, Synthetic construct
  source 1..16: mol_type = other RNA
ttcatgaaaggactta

SEQ ID NO: 458 - RNA, 16 nt, Synthetic construct
  source 1..16: mol_type = other RNA
tcatgaaaggacttta

SEQ ID NO: 459 - RNA, 16 nt, Synthetic construct
  source 1..16: mol_type = other RNA
catgaaaggactttca

SEQ ID NO: 460 - RNA, 16 nt, Synthetic construct
  source 1..16: mol_type = other RNA
gaaaggactttcaaaa

SEQ ID NO: 461 - RNA, 16 nt, Synthetic construct
  source 1..16: mol_type = other RNA
aaaggactttcaaaga

SEQ ID NO: 462 - RNA, 16 nt, Synthetic construct
  source 1..16: mol_type = other RNA
aaggactttcaaagga

SEQ ID NO: 463 - RNA, 16 nt, Synthetic construct
  source 1..16: mol_type = other RNA
aggactttcaaaggca

SEQ ID NO: 464 - RNA, 16 nt, Synthetic construct
  source 1..16: mol_type = other RNA
ggactttcaaaggcca

SEQ ID NO: 465 - RNA, 16 nt, Synthetic construct
  source 1..16: mol_type = other RNA
gactttcaaaggccaa

SEQ ID NO: 466 - RNA, 16 nt, Synthetic construct
  source 1..16: mol_type = other RNA
actttcaaaggccaaa

SEQ ID NO: 467 - RNA, 16 nt, Synthetic construct
  source 1..16: mol_type = other RNA
ctttcaaaggccaaga

SEQ ID NO: 468 - RNA, 16 nt, Synthetic construct
  source 1..16: mol_type = other RNA
tttcaaaggccaagga

SEQ ID NO: 469 - RNA, 16 nt, Synthetic construct
  source 1..16: mol_type = other RNA
ttcaaaggccaaggaa

SEQ ID NO: 470 - RNA, 16 nt, Synthetic construct
  source 1..16: mol_type = other RNA
tcaaaggccaaggaga

SEQ ID NO: 471 - RNA, 16 nt, Synthetic construct
  source 1..16: mol_type = other RNA
caaaggccaaggagga

SEQ ID NO: 472 - RNA, 16 nt, Synthetic construct
  source 1..16: mol_type = other RNA
aaaggccaaggaggga

SEQ ID NO: 473 - RNA, 16 nt, Synthetic construct
  source 1..16: mol_type = other RNA
aaggccaaggagggaa

SEQ ID NO: 474 - RNA, 16 nt, Synthetic construct
  source 1..16: mol_type = other RNA
aggccaaggagggaga

SEQ ID NO: 475 - RNA, 16 nt, Synthetic construct
  source 1..16: mol_type = other RNA
ggccaaggagggagta

SEQ ID NO: 476 - RNA, 16 nt, Synthetic construct
  source 1..16: mol_type = other RNA
gccaaggagggagtta

SEQ ID NO: 477 - RNA, 16 nt, Synthetic construct
  source 1..16: mol_type = other RNA
ccaaggagggagttga

SEQ ID NO: 478 - RNA, 16 nt, Synthetic construct
  source 1..16: mol_type = other RNA
caaggagggagttgta

SEQ ID NO: 479 - RNA, 16 nt, Synthetic construct
  source 1..16: mol_type = other RNA
aaggagggagttgtga

SEQ ID NO: 480 - RNA, 16 nt, Synthetic construct
  source 1..16: mol_type = other RNA
aggagggagttgtgga

SEQ ID NO: 481 - RNA, 16 nt, Synthetic construct
  source 1..16: mol_type = other RNA
ggagggagttgtggca

SEQ ID NO: 482 - RNA, 16 nt, Synthetic construct
  source 1..16: mol_type = other RNA
gagggagttgtggcta

SEQ ID NO: 483 - RNA, 16 nt, Synthetic construct
  source 1..16: mol_type = other RNA
agggagttgtggctga

SEQ ID NO: 484 - RNA, 16 nt, Synthetic construct
  source 1..16: mol_type = other RNA
gggagttgtggctgca

SEQ ID NO: 485 - RNA, 16 nt, Synthetic construct
  source 1..16: mol_type = other RNA
ggagttgtggctgcta

SEQ ID NO: 486 - RNA, 16 nt, Synthetic construct
  source 1..16: mol_type = other RNA
gagttgtggctgctga

SEQ ID NO: 487 - RNA, 16 nt, Synthetic construct
  source 1..16: mol_type = other RNA
agttgtggctgctgca

SEQ ID NO: 488 - RNA, 16 nt, Synthetic construct
  source 1..16: mol_type = other RNA
gttgtggctgctgcta

SEQ ID NO: 489 - RNA, 16 nt, Synthetic construct
  source 1..16: mol_type = other RNA
ttgtggctgctgctga

SEQ ID NO: 490 - RNA, 16 nt, Synthetic construct
  source 1..16: mol_type = other RNA
tgtggctgctgctgaa

SEQ ID NO: 491 - RNA, 16 nt, Synthetic construct
  source 1..16: mol_type = other RNA
gtggctgctgctgaga

SEQ ID NO: 492 - RNA, 16 nt, Synthetic construct
  source 1..16: mol_type = other RNA
tggctgctgctgagaa

SEQ ID NO: 493 - RNA, 16 nt, Synthetic construct
  source 1..16: mol_type = other RNA
ggctgctgctgagaaa

SEQ ID NO: 494 - RNA, 16 nt, Synthetic construct
  source 1..16: mol_type = other RNA
gctgctgctgagaaaa

SEQ ID NO: 495 - RNA, 16 nt, Synthetic construct
  source 1..16: mol_type = other RNA
tgctgctgagaaaaca

SEQ ID NO: 496 - RNA, 16 nt, Synthetic construct
  source 1..16: mol_type = other RNA
gctgctgagaaaacca

SEQ ID NO: 497 - RNA, 16 nt, Synthetic construct
  source 1..16: mol_type = other RNA
ctgctgagaaaaccaa

SEQ ID NO: 498 - RNA, 16 nt, Synthetic construct
  source 1..16: mol_type = other RNA
tgctgagaaaaccaaa

SEQ ID NO: 499 - RNA, 16 nt, Synthetic construct
  source 1..16: mol_type = other RNA
gctgagaaaaccaaaa

SEQ ID NO: 500 - RNA, 16 nt, Synthetic construct
  source 1..16: mol_type = other RNA
ctgagaaaaccaaaca

SEQ ID NO: 501 - RNA, 16 nt, Synthetic construct
  source 1..16: mol_type = other RNA
tgagaaaaccaaacaa

SEQ ID NO: 502 - RNA, 16 nt, Synthetic construct
  source 1..16: mol_type = other RNA
gagaaaaccaaacaga

SEQ ID NO: 503 - RNA, 16 nt, Synthetic construct
  source 1..16: mol_type = other RNA
agaaaaccaaacagga

SEQ ID NO: 504 - RNA, 16 nt, Synthetic construct
  source 1..16: mol_type = other RNA
gaaaaccaaacaggga

SEQ ID NO: 505 - RNA, 16 nt, Synthetic construct
  source 1..16: mol_type = other RNA
caaacagggtgtggca

SEQ ID NO: 506 - RNA, 16 nt, Synthetic construct
  source 1..16: mol_type = other RNA
aaacagggtgtggcaa

SEQ ID NO: 507 - RNA, 16 nt, Synthetic construct
  source 1..16: mol_type = other RNA
aacagggtgtggcaga

SEQ ID NO: 508 - RNA, 16 nt, Synthetic construct
  source 1..16: mol_type = other RNA
acagggtgtggcagaa

SEQ ID NO: 509 - RNA, 16 nt, Synthetic construct
  source 1..16: mol_type = other RNA
cagggtgtggcagaaa

SEQ ID NO: 510 - RNA, 16 nt, Synthetic construct
  source 1..16: mol_type = other RNA
aagcagcaggaaagaa

SEQ ID NO: 511 - RNA, 16 nt, Synthetic construct
  source 1..16: mol_type = other RNA
caggaaagacaaaaga

SEQ ID NO: 512 - RNA, 16 nt, Synthetic construct
  source 1..16: mol_type = other RNA
aggaaagacaaaagaa

SEQ ID NO: 513 - RNA, 16 nt, Synthetic construct
  source 1..16: mol_type = other RNA
ggaaagacaaaagaga

SEQ ID NO: 514 - RNA, 16 nt, Synthetic construct
  source 1..16: mol_type = other RNA
gaaagacaaaagagga

SEQ ID NO: 515 - RNA, 16 nt, Synthetic construct
  source 1..16: mol_type = other RNA
aaagacaaaagaggga

SEQ ID NO: 516 - RNA, 16 nt, Synthetic construct
  source 1..16: mol_type = other RNA
aagacaaaagagggta

SEQ ID NO: 517 - RNA, 16 nt, Synthetic construct
  source 1..16: mol_type = other RNA
gacaaaagagggtgta

SEQ ID NO: 518 - RNA, 16 nt, Synthetic construct
  source 1..16: mol_type = other RNA
acaaaagagggtgtta

SEQ ID NO: 519 - RNA, 16 nt, Synthetic construct
  source 1..16: mol_type = other RNA
caaaagagggtgttca

SEQ ID NO: 520 - RNA, 16 nt, Synthetic construct
  source 1..16: mol_type = other RNA
aaaagagggtgttcta

SEQ ID NO: 521 - RNA, 16 nt, Synthetic construct
  source 1..16: mol_type = other RNA
tctctatgtaggctca

SEQ ID NO: 522 - RNA, 16 nt, Synthetic construct
  source 1..16: mol_type = other RNA
ctctatgtaggctcca

SEQ ID NO: 523 - RNA, 16 nt, Synthetic construct
  source 1..16: mol_type = other RNA
tctatgtaggctccaa

SEQ ID NO: 524 - RNA, 16 nt, Synthetic construct
  source 1..16: mol_type = other RNA
ctatgtaggctccaaa

SEQ ID NO: 525 - RNA, 16 nt, Synthetic construct
  source 1..16: mol_type = other RNA
tatgtaggctccaaaa

SEQ ID NO: 526 - RNA, 16 nt, Synthetic construct
  source 1..16: mol_type = other RNA
tgtaggctccaaaaca

SEQ ID NO: 527 - RNA, 16 nt, Synthetic construct
  source 1..16: mol_type = other RNA
gctccaaaaccaagga

SEQ ID NO: 528 - RNA, 16 nt, Synthetic construct
  source 1..16: mol_type = other RNA
ctccaaaaccaaggaa

SEQ ID NO: 529 - RNA, 16 nt, Synthetic construct
  source 1..16: mol_type = other RNA
tccaaaaccaaggaga

SEQ ID NO: 530 - RNA, 16 nt, Synthetic construct
  source 1..16: mol_type = other RNA
ccaaaaccaaggagga

SEQ ID NO: 531 - RNA, 16 nt, Synthetic construct
  source 1..16: mol_type = other RNA
caaaaccaaggaggga

SEQ ID NO: 532 - RNA, 16 nt, Synthetic construct
  source 1..16: mol_type = other RNA
ccaaggagggagtgga

SEQ ID NO: 533 - RNA, 16 nt, Synthetic construct
  source 1..16: mol_type = other RNA
ggagtggtgcatggta

SEQ ID NO: 534 - RNA, 16 nt, Synthetic construct
  source 1..16: mol_type = other RNA
gagtggtgcatggtga

SEQ ID NO: 535 - RNA, 16 nt, Synthetic construct
  source 1..16: mol_type = other RNA
agtggtgcatggtgta

SEQ ID NO: 536 - RNA, 16 nt, Synthetic construct
  source 1..16: mol_type = other RNA
gtggtgcatggtgtga

SEQ ID NO: 537 - RNA, 16 nt, Synthetic construct
  source 1..16: mol_type = other RNA
tggtgcatggtgtgga

SEQ ID NO: 538 - RNA, 16 nt, Synthetic construct
  source 1..16: mol_type = other RNA
gcatggtgtggcaaca

SEQ ID NO: 539 - RNA, 16 nt, Synthetic construct
  source 1..16: mol_type = other RNA
catggtgtggcaacaa

SEQ ID NO: 540 - RNA, 16 nt, Synthetic construct
  source 1..16: mol_type = other RNA
atggtgtggcaacaga

SEQ ID NO: 541 - RNA, 16 nt, Synthetic construct
  source 1..16: mol_type = other RNA
tggtgtggcaacagta

SEQ ID NO: 542 - RNA, 16 nt, Synthetic construct
  source 1..16: mol_type = other RNA
ggtgtggcaacagtga

SEQ ID NO: 543 - RNA, 16 nt, Synthetic construct
  source 1..16: mol_type = other RNA
tggcaacagtggctga

SEQ ID NO: 544 - RNA, 16 nt, Synthetic construct
  source 1..16: mol_type = other RNA
ggcaacagtggctgaa

SEQ ID NO: 545 - RNA, 16 nt, Synthetic construct
  source 1..16: mol_type = other RNA
gcaacagtggctgaga

SEQ ID NO: 546 - RNA, 16 nt, Synthetic construct
  source 1..16: mol_type = other RNA
caacagtggctgagaa

SEQ ID NO: 547 - RNA, 16 nt, Synthetic construct
  source 1..16: mol_type = other RNA
aacagtggctgagaaa

SEQ ID NO: 548 - RNA, 16 nt, Synthetic construct
  source 1..16: mol_type = other RNA
acagtggctgagaaga

SEQ ID NO: 549 - RNA, 16 nt, Synthetic construct
  source 1..16: mol_type = other RNA
cagtggctgagaagaa

SEQ ID NO: 550 - RNA, 16 nt, Synthetic construct
  source 1..16: mol_type = other RNA
agtggctgagaagaca

SEQ ID NO: 551 - RNA, 16 nt, Synthetic construct
  source 1..16: mol_type = other RNA
gtggctgagaagacca

SEQ ID NO: 552 - RNA, 16 nt, Synthetic construct
  source 1..16: mol_type = other RNA
tggctgagaagaccaa

SEQ ID NO: 553 - RNA, 16 nt, Synthetic construct
  source 1..16: mol_type = other RNA
ggctgagaagaccaaa

SEQ ID NO: 554 - RNA, 16 nt, Synthetic construct
  source 1..16: mol_type = other RNA
gctgagaagaccaaaa

SEQ ID NO: 555 - RNA, 16 nt, Synthetic construct
  source 1..16: mol_type = other RNA
ctgagaagaccaaaga

SEQ ID NO: 556 - RNA, 16 nt, Synthetic construct
  source 1..16: mol_type = other RNA
tgagaagaccaaagaa

SEQ ID NO: 557 - RNA, 16 nt, Synthetic construct
  source 1..16: mol_type = other RNA
gagaagaccaaagaga

SEQ ID NO: 558 - RNA, 16 nt, Synthetic construct
  source 1..16: mol_type = other RNA
agaagaccaaagagca

SEQ ID NO: 559 - RNA, 16 nt, Synthetic construct
  source 1..16: mol_type = other RNA
aagaccaaagagcaaa

SEQ ID NO: 560 - RNA, 16 nt, Synthetic construct
  source 1..16: mol_type = other RNA
agaccaaagagcaaga

SEQ ID NO: 561 - RNA, 16 nt, Synthetic construct
  source 1..16: mol_type = other RNA
gaccaaagagcaagta

SEQ ID NO: 562 - RNA, 16 nt, Synthetic construct
  source 1..16: mol_type = other RNA
accaaagagcaagtga

SEQ ID NO: 563 - RNA, 16 nt, Synthetic construct
  source 1..16: mol_type = other RNA
ccaaagagcaagtgaa

SEQ ID NO: 564 - RNA, 16 nt, Synthetic construct
  source 1..16: mol_type = other RNA
agagcaagtgacaaaa

SEQ ID NO: 565 - RNA, 16 nt, Synthetic construct
  source 1..16: mol_type = other RNA
gagcaagtgacaaata

SEQ ID NO: 566 - RNA, 16 nt, Synthetic construct
  source 1..16: mol_type = other RNA
agcaagtgacaaatga

SEQ ID NO: 567 - RNA, 16 nt, Synthetic construct
  source 1..16: mol_type = other RNA
gcaagtgacaaatgta

SEQ ID NO: 568 - RNA, 16 nt, Synthetic construct
  source 1..16: mol_type = other RNA
caagtgacaaatgtta

SEQ ID NO: 569 - RNA, 16 nt, Synthetic construct
  source 1..16: mol_type = other RNA
aagtgacaaatgttga

SEQ ID NO: 570 - RNA, 16 nt, Synthetic construct
  source 1..16: mol_type = other RNA
agtgacaaatgttgga

SEQ ID NO: 571 - RNA, 16 nt, Synthetic construct
  source 1..16: mol_type = other RNA
gtgacaaatgttggaa

SEQ ID NO: 572 - RNA, 16 nt, Synthetic construct
  source 1..16: mol_type = other RNA
tgacaaatgttggaga

SEQ ID NO: 573 - RNA, 16 nt, Synthetic construct
  source 1..16: mol_type = other RNA
gacaaatgttggagga

SEQ ID NO: 574 - RNA, 16 nt, Synthetic construct
  source 1..16: mol_type = other RNA
agcagtggtgacggga

SEQ ID NO: 575 - RNA, 16 nt, Synthetic construct
  source 1..16: mol_type = other RNA
cgggtgtgacagcaga

SEQ ID NO: 576 - RNA, 16 nt, Synthetic construct
  source 1..16: mol_type = other RNA
gtgtgacagcagtaga

SEQ ID NO: 577 - RNA, 16 nt, Synthetic construct
  source 1..16: mol_type = other RNA
agcagtagcccagaaa

SEQ ID NO: 578 - RNA, 16 nt, Synthetic construct
  source 1..16: mol_type = other RNA
gcagtagcccagaaga

SEQ ID NO: 579 - RNA, 16 nt, Synthetic construct
  source 1..16: mol_type = other RNA
agaagacagtggagga

SEQ ID NO: 580 - RNA, 16 nt, Synthetic construct
  source 1..16: mol_type = other RNA
gaagacagtggaggga

SEQ ID NO: 581 - RNA, 16 nt, Synthetic construct
  source 1..16: mol_type = other RNA
aagacagtggagggaa

SEQ ID NO: 582 - RNA, 16 nt, Synthetic construct
  source 1..16: mol_type = other RNA
agacagtggagggaga

SEQ ID NO: 583 - RNA, 16 nt, Synthetic construct
  source 1..16: mol_type = other RNA
gacagtggagggagca

SEQ ID NO: 584 - RNA, 16 nt, Synthetic construct
  source 1..16: mol_type = other RNA
gggagcagggagcata

SEQ ID NO: 585 - RNA, 16 nt, Synthetic construct
  source 1..16: mol_type = other RNA
agcagggagcattgca

SEQ ID NO: 586 - RNA, 16 nt, Synthetic construct
  source 1..16: mol_type = other RNA
gcagggagcattgcaa

SEQ ID NO: 587 - RNA, 16 nt, Synthetic construct
  source 1..16: mol_type = other RNA
ttgggcaagaatgaaa

SEQ ID NO: 588 - RNA, 16 nt, Synthetic construct
  source 1..16: mol_type = other RNA
tgggcaagaatgaaga

SEQ ID NO: 589 - RNA, 16 nt, Synthetic construct
  source 1..16: mol_type = other RNA
ggcaagaatgaagaaa

SEQ ID NO: 590 - RNA, 16 nt, Synthetic construct
  source 1..16: mol_type = other RNA
ggaaggaattctggaa

SEQ ID NO: 591 - RNA, 16 nt, Synthetic construct
  source 1..16: mol_type = other RNA
gaaggaattctggaaa

SEQ ID NO: 592 - RNA, 16 nt, Synthetic construct
  source 1..16: mol_type = other RNA
tctggaagatatgcca

SEQ ID NO: 593 - RNA, 16 nt, Synthetic construct
  source 1..16: mol_type = other RNA
gaagatatgcctgtga

SEQ ID NO: 594 - RNA, 16 nt, Synthetic construct
  source 1..16: mol_type = other RNA
tggatcctgacaatga

SEQ ID NO: 595 - RNA, 16 nt, Synthetic construct
  source 1..16: mol_type = other RNA
gatcctgacaatgaga

SEQ ID NO: 596 - RNA, 16 nt, Synthetic construct
  source 1..16: mol_type = other RNA
atcctgacaatgagga

SEQ ID NO: 597 - RNA, 16 nt, Synthetic construct
  source 1..16: mol_type = other RNA
gacaatgaggcttata

SEQ ID NO: 598 - RNA, 16 nt, Synthetic construct
  source 1..16: mol_type = other RNA
acaatgaggcttatga

SEQ ID NO: 599 - RNA, 16 nt, Synthetic construct
  source 1..16: mol_type = other RNA
caatgaggcttatgaa

SEQ ID NO: 600 - RNA, 16 nt, Synthetic construct
  source 1..16: mol_type = other RNA
aatgaggcttatgaaa

SEQ ID NO: 601 - RNA, 16 nt, Synthetic construct
  source 1..16: mol_type = other RNA
atgaggcttatgaaaa

SEQ ID NO: 602 - RNA, 16 nt, Synthetic construct
  source 1..16: mol_type = other RNA
tgaggcttatgaaata

SEQ ID NO: 603 - RNA, 16 nt, Synthetic construct
  source 1..16: mol_type = other RNA
gaggcttatgaaatga

SEQ ID NO: 604 - RNA, 16 nt, Synthetic construct
  source 1..16: mol_type = other RNA
aggcttatgaaatgca

SEQ ID NO: 605 - RNA, 16 nt, Synthetic construct
  source 1..16: mol_type = other RNA
ggcttatgaaatgcca

SEQ ID NO: 606 - RNA, 16 nt, Synthetic construct
  source 1..16: mol_type = other RNA
gcttatgaaatgccta

SEQ ID NO: 607 - RNA, 16 nt, Synthetic construct
  source 1..16: mol_type = other RNA
cttatgaaatgcctta

SEQ ID NO: 608 - RNA, 16 nt, Synthetic construct
  source 1..16: mol_type = other RNA
ttatgaaatgccttca

SEQ ID NO: 609 - RNA, 16 nt, Synthetic construct
  source 1..16: mol_type = other RNA
tgaaatgccttctgaa

SEQ ID NO: 610 - RNA, 16 nt, Synthetic construct
  source 1..16: mol_type = other RNA
gaaatgccttctgaga

SEQ ID NO: 611 - RNA, 16 nt, Synthetic construct
  source 1..16: mol_type = other RNA
atgccttctgaggaaa

SEQ ID NO: 612 - RNA, 16 nt, Synthetic construct
  source 1..16: mol_type = other RNA
ggaagggtatcaagaa

SEQ ID NO: 613 - RNA, 16 nt, Synthetic construct
  source 1..16: mol_type = other RNA
gaagggtatcaagaca

SEQ ID NO: 614 - RNA, 16 nt, Synthetic construct
  source 1..16: mol_type = other RNA
actacgaacctgaaga

SEQ ID NO: 615 - RNA, 16 nt, Synthetic construct
  source 1..16: mol_type = other RNA
ctacgaacctgaagca

SEQ ID NO: 616 - RNA, 16 nt, Synthetic construct
  source 1..16: mol_type = other RNA
tacgaacctgaagcca

SEQ ID NO: 617 - RNA, 16 nt, Synthetic construct
  source 1..16: mol_type = other RNA
aacctgaagcctaaga

SEQ ID NO: 618 - RNA, 16 nt, Synthetic construct
  source 1..16: mol_type = other RNA
acctgaagcctaagaa

SEQ ID NO: 619 - RNA, 16 nt, Synthetic construct
  source 1..16: mol_type = other RNA
acctgaagcctaagaa

SEQ ID NO: 620 - RNA, 16 nt, Synthetic construct
  source 1..16: mol_type = other RNA
cctgaagcctaagaaa

SEQ ID NO: 621 - RNA, 16 nt, Synthetic construct
  source 1..16: mol_type = other RNA
ctgaagcctaagaaaa

SEQ ID NO: 622 - RNA, 16 nt, Synthetic construct
  source 1..16: mol_type = other RNA
tgaagcctaagaaata

SEQ ID NO: 623 - RNA, 16 nt, Synthetic construct
  source 1..16: mol_type = other RNA
gaagcctaagaaataa

SEQ ID NO: 624 - RNA, 16 nt, Synthetic construct
  source 1..16: mol_type = other RNA
agcctaagaaatatca

SEQ ID NO: 625 - RNA, 16 nt, Synthetic construct
  source 1..16: mol_type = other RNA
gcctaagaaatatcta

SEQ ID NO: 626 - RNA, 16 nt, Synthetic construct
  source 1..16: mol_type = other RNA
cctaagaaatatctta

SEQ ID NO: 627 - RNA, 16 nt, Synthetic construct
  source 1..16: mol_type = other RNA
ctaagaaatatcttta

SEQ ID NO: 628 - RNA, 16 nt, Synthetic construct
  source 1..16: mol_type = other RNA
taagaaatatctttga

SEQ ID NO: 629 - RNA, 16 nt, Synthetic construct
  source 1..16: mol_type = other RNA
aagaaatatctttgca

SEQ ID NO: 630 - RNA, 16 nt, Synthetic construct
  source 1..16: mol_type = other RNA
agaaatatctttgcta

SEQ ID NO: 631 - RNA, 16 nt, Synthetic construct
  source 1..16: mol_type = other RNA
gaaatatctttgctca

SEQ ID NO: 632 - RNA, 16 nt, Synthetic construct
  source 1..16: mol_type = other RNA
aaatatctttgctcca

SEQ ID NO: 633 - RNA, 16 nt, Synthetic construct
  source 1..16: mol_type = other RNA
aatatctttgctccca

SEQ ID NO: 634 - RNA, 16 nt, Synthetic construct
  source 1..16: mol_type = other RNA
gctcccagtttcttga

SEQ ID NO: 635 - RNA, 16 nt, Synthetic construct
  source 1..16: mol_type = other RNA
ctcccagtttcttgaa

SEQ ID NO: 636 - RNA, 16 nt, Synthetic construct
  source 1..16: mol_type = other RNA
tcccagtttcttgaga

SEQ ID NO: 637 - RNA, 16 nt, Synthetic construct
  source 1..16: mol_type = other RNA
cccagtttcttgagaa

SEQ ID NO: 638 - RNA, 16 nt, Synthetic construct
  source 1..16: mol_type = other RNA
ccagtttcttgagata

SEQ ID NO: 639 - RNA, 16 nt, Synthetic construct
  source 1..16: mol_type = other RNA
cagtttcttgagatca

SEQ ID NO: 640 - RNA, 16 nt, Synthetic construct
  source 1..16: mol_type = other RNA
agtttcttgagatcta

SEQ ID NO: 641 - RNA, 16 nt, Synthetic construct
  source 1..16: mol_type = other RNA
tttcttgagatctgca

SEQ ID NO: 642 - RNA, 16 nt, Synthetic construct
  source 1..16: mol_type = other RNA
ttgagatctgctgaca

SEQ ID NO: 643 - RNA, 16 nt, Synthetic construct
  source 1..16: mol_type = other RNA
tgagatctgctgacaa

SEQ ID NO: 644 - RNA, 16 nt, Synthetic construct
  source 1..16: mol_type = other RNA
gagatctgctgacaga

SEQ ID NO: 645 - RNA, 16 nt, Synthetic construct
  source 1..16: mol_type = other RNA
agatctgctgacagaa

SEQ ID NO: 646 - RNA, 16 nt, Synthetic construct
  source 1..16: mol_type = other RNA
gatctgctgacagata

SEQ ID NO: 647 - RNA, 16 nt, Synthetic construct
  source 1..16: mol_type = other RNA
atctgctgacagatga

SEQ ID NO: 648 - RNA, 16 nt, Synthetic construct
  source 1..16: mol_type = other RNA
tctgctgacagatgta

SEQ ID NO: 649 - RNA, 16 nt, Synthetic construct
  source 1..16: mol_type = other RNA
ctgacagatgttccaa

SEQ ID NO: 650 - RNA, 16 nt, Synthetic construct
  source 1..16: mol_type = other RNA
tgacagatgttccata

SEQ ID NO: 651 - RNA, 16 nt, Synthetic construct
  source 1..16: mol_type = other RNA
gacagatgttccatca

SEQ ID NO: 652 - RNA, 16 nt, Synthetic construct
  source 1..16: mol_type = other RNA
acagatgttccatcca

SEQ ID NO: 653 - RNA, 16 nt, Synthetic construct
  source 1..16: mol_type = other RNA
cagatgttccatccta

SEQ ID NO: 654 - RNA, 16 nt, Synthetic construct
  source 1..16: mol_type = other RNA
agatgttccatcctga

SEQ ID NO: 655 - RNA, 16 nt, Synthetic construct
  source 1..16: mol_type = other RNA
gatgttccatcctgta

SEQ ID NO: 656 - RNA, 16 nt, Synthetic construct
  source 1..16: mol_type = other RNA
atgttccatcctgtaa

SEQ ID NO: 657 - RNA, 16 nt, Synthetic construct
  source 1..16: mol_type = other RNA
ttccatcctgtacaaa

SEQ ID NO: 658 - RNA, 16 nt, Synthetic construct
  source 1..16: mol_type = other RNA
tcctgtacaagtgcta

SEQ ID NO: 659 - RNA, 16 nt, Synthetic construct
  source 1..16: mol_type = other RNA
ctgtacaagtgctcaa

SEQ ID NO: 660 - RNA, 16 nt, Synthetic construct
  source 1..16: mol_type = other RNA
tgtacaagtgctcaga

SEQ ID NO: 661 - RNA, 16 nt, Synthetic construct
  source 1..16: mol_type = other RNA
gtacaagtgctcagta

SEQ ID NO: 662 - RNA, 16 nt, Synthetic construct
  source 1..16: mol_type = other RNA
tacaagtgctcagtta

SEQ ID NO: 663 - RNA, 16 nt, Synthetic construct
  source 1..16: mol_type = other RNA
acaagtgctcagttca

SEQ ID NO: 664 - RNA, 16 nt, Synthetic construct
  source 1..16: mol_type = other RNA
caagtgctcagttcca

SEQ ID NO: 665 - RNA, 16 nt, Synthetic construct
  source 1..16: mol_type = other RNA
aagtgctcagttccaa

SEQ ID NO: 666 - RNA, 16 nt, Synthetic construct
  source 1..16: mol_type = other RNA
agtgctcagttccaaa

SEQ ID NO: 667 - RNA, 16 nt, Synthetic construct
  source 1..16: mol_type = other RNA
gtgctcagttccaata

SEQ ID NO: 668 - RNA, 16 nt, Synthetic construct
  source 1..16: mol_type = other RNA
tgctcagttccaatga

SEQ ID NO: 669 - RNA, 16 nt, Synthetic construct
  source 1..16: mol_type = other RNA
gctcagttccaatgta

SEQ ID NO: 670 - RNA, 16 nt, Synthetic construct
  source 1..16: mol_type = other RNA
ctcagttccaatgtga

SEQ ID NO: 671 - RNA, 16 nt, Synthetic construct
  source 1..16: mol_type = other RNA
tcagttccaatgtgca

SEQ ID NO: 672 - RNA, 16 nt, Synthetic construct
  source 1..16: mol_type = other RNA
cagttccaatgtgcca

SEQ ID NO: 673 - RNA, 16 nt, Synthetic construct
  source 1..16: mol_type = other RNA
agttccaatgtgccca

SEQ ID NO: 674 - RNA, 16 nt, Synthetic construct
  source 1..16: mol_type = other RNA
gttccaatgtgcccaa

SEQ ID NO: 675 - RNA, 16 nt, Synthetic construct
  source 1..16: mol_type = other RNA
ttccaatgtgcccaga

SEQ ID NO: 676 - RNA, 16 nt, Synthetic construct
  source 1..16: mol_type = other RNA
tccaatgtgcccagta

SEQ ID NO: 677 - RNA, 16 nt, Synthetic construct
  source 1..16: mol_type = other RNA
ccaatgtgcccagtca

SEQ ID NO: 678 - RNA, 16 nt, Synthetic construct
  source 1..16: mol_type = other RNA
caatgtgcccagtcaa

SEQ ID NO: 679 - RNA, 16 nt, Synthetic construct
  source 1..16: mol_type = other RNA
aatgtgcccagtcata

SEQ ID NO: 680 - RNA, 16 nt, Synthetic construct
  source 1..16: mol_type = other RNA
gtgcccagtcatgaca

SEQ ID NO: 681 - RNA, 16 nt, Synthetic construct
  source 1..16: mol_type = other RNA
cccagtcatgacatta

SEQ ID NO: 682 - RNA, 16 nt, Synthetic construct
  source 1..16: mol_type = other RNA
ccagtcatgacattta

SEQ ID NO: 683 - RNA, 16 nt, Synthetic construct
  source 1..16: mol_type = other RNA
cagtcatgacatttca

SEQ ID NO: 684 - RNA, 16 nt, Synthetic construct
  source 1..16: mol_type = other RNA
tcatgacatttctcaa

SEQ ID NO: 685 - RNA, 16 nt, Synthetic construct
  source 1..16: mol_type = other RNA
catgacatttctcaaa

SEQ ID NO: 686 - RNA, 16 nt, Synthetic construct
  source 1..16: mol_type = other RNA
atgacatttctcaaaa

SEQ ID NO: 687 - RNA, 16 nt, Synthetic construct
  source 1..16: mol_type = other RNA
tgacatttctcaaaga

SEQ ID NO: 688 - RNA, 16 nt, Synthetic construct
  source 1..16: mol_type = other RNA
gacatttctcaaagta

SEQ ID NO: 689 - RNA, 16 nt, Synthetic construct
  source 1..16: mol_type = other RNA
cagtgtatctcgaaga

SEQ ID NO: 690 - RNA, 16 nt, Synthetic construct
  source 1..16: mol_type = other RNA
agtgtatctcgaagta

SEQ ID NO: 691 - RNA, 16 nt, Synthetic construct
  source 1..16: mol_type = other RNA
gtgtatctcgaagtca

SEQ ID NO: 692 - RNA, 16 nt, Synthetic construct
  source 1..16: mol_type = other RNA
tgtatctcgaagtcta

SEQ ID NO: 693 - RNA, 16 nt, Synthetic construct
  source 1..16: mol_type = other RNA
gtatctcgaagtctta

SEQ ID NO: 694 - RNA, 16 nt, Synthetic construct
  source 1..16: mol_type = other RNA
tatctcgaagtcttca

SEQ ID NO: 695 - RNA, 16 nt, Synthetic construct
  source 1..16: mol_type = other RNA
tcgaagtcttccatca

SEQ ID NO: 696 - RNA, 16 nt, Synthetic construct
  source 1..16: mol_type = other RNA
cgaagtcttccatcaa

SEQ ID NO: 697 - RNA, 16 nt, Synthetic construct
  source 1..16: mol_type = other RNA
gaagtcttccatcaga

SEQ ID NO: 698 - RNA, 16 nt, Synthetic construct
  source 1..16: mol_type = other RNA
aagtcttccatcagca

SEQ ID NO: 699 - RNA, 16 nt, Synthetic construct
  source 1..16: mol_type = other RNA
agtcttccatcagcaa

SEQ ID NO: 700 - RNA, 16 nt, Synthetic construct
  source 1..16: mol_type = other RNA
gtcttccatcagcaga

SEQ ID NO: 701 - RNA, 16 nt, Synthetic construct
  source 1..16: mol_type = other RNA
tcttccatcagcagta

SEQ ID NO: 702 - RNA, 16 nt, Synthetic construct
  source 1..16: mol_type = other RNA
cttccatcagcagtga

SEQ ID NO: 703 - RNA, 16 nt, Synthetic construct
  source 1..16: mol_type = other RNA
ttccatcagcagtgaa

SEQ ID NO: 704 - RNA, 16 nt, Synthetic construct
  source 1..16: mol_type = other RNA
tccatcagcagtgata

SEQ ID NO: 705 - RNA, 16 nt, Synthetic construct
  source 1..16: mol_type = other RNA
ccatcagcagtgatta

SEQ ID NO: 706 - RNA, 16 nt, Synthetic construct
  source 1..16: mol_type = other RNA
catcagcagtgattga

SEQ ID NO: 707 - RNA, 16 nt, Synthetic construct
  source 1..16: mol_type = other RNA
atcagcagtgattgaa

SEQ ID NO: 708 - RNA, 16 nt, Synthetic construct
  source 1..16: mol_type = other RNA
tcagcagtgattgaaa

SEQ ID NO: 709 - RNA, 16 nt, Synthetic construct
  source 1..16: mol_type = other RNA
cagcagtgattgaaga

SEQ ID NO: 710 - RNA, 16 nt, Synthetic construct
  source 1..16: mol_type = other RNA
agcagtgattgaagta

SEQ ID NO: 711 - RNA, 16 nt, Synthetic construct
  source 1..16: mol_type = other RNA
gcagtgattgaagtaa

SEQ ID NO: 712 - RNA, 16 nt, Synthetic construct
  source 1..16: mol_type = other RNA
cagtgattgaagtata

SEQ ID NO: 713 - RNA, 16 nt, Synthetic construct
  source 1..16: mol_type = other RNA
tgaagtatctgtacca

SEQ ID NO: 714 - RNA, 16 nt, Synthetic construct
  source 1..16: mol_type = other RNA
gaagtatctgtaccta

SEQ ID NO: 715 - RNA, 16 nt, Synthetic construct
  source 1..16: mol_type = other RNA
aagtatctgtacctga

SEQ ID NO: 716 - RNA, 16 nt, Synthetic construct
  source 1..16: mol_type = other RNA
agtatctgtacctgca

SEQ ID NO: 717 - RNA, 16 nt, Synthetic construct
  source 1..16: mol_type = other RNA
gtatctgtacctgcca

SEQ ID NO: 718 - RNA, 16 nt, Synthetic construct
  source 1..16: mol_type = other RNA
tatctgtacctgccca

SEQ ID NO: 719 - RNA, 16 nt, Synthetic construct
  source 1..16: mol_type = other RNA
agcatttcggtgctta

SEQ ID NO: 720 - RNA, 16 nt, Synthetic construct
  source 1..16: mol_type = other RNA
gcatttcggtgcttca

SEQ ID NO: 721 - RNA, 16 nt, Synthetic construct
  source 1..16: mol_type = other RNA
ttcggtgcttccctta

SEQ ID NO: 722 - RNA, 16 nt, Synthetic construct
  source 1..16: mol_type = other RNA
tcggtgcttcccttta

SEQ ID NO: 723 - RNA, 16 nt, Synthetic construct
  source 1..16: mol_type = other RNA
ggtgcttccctttcaa

SEQ ID NO: 724 - RNA, 16 nt, Synthetic construct
  source 1..16: mol_type = other RNA
gtgcttccctttcaca

SEQ ID NO: 725 - RNA, 16 nt, Synthetic construct
  source 1..16: mol_type = other RNA
tgcttccctttcacta

SEQ ID NO: 726 - RNA, 16 nt, Synthetic construct
  source 1..16: mol_type = other RNA
cttccctttcactgaa

SEQ ID NO: 727 - RNA, 16 nt, Synthetic construct
  source 1..16: mol_type = other RNA
ccctttcactgaagta

SEQ ID NO: 728 - RNA, 16 nt, Synthetic construct
  source 1..16: mol_type = other RNA
tcactgaagtgaataa

SEQ ID NO: 729 - RNA, 16 nt, Synthetic construct
  source 1..16: mol_type = other RNA
cactgaagtgaataca

SEQ ID NO: 730 - RNA, 16 nt, Synthetic construct
  source 1..16: mol_type = other RNA
ctgaagtgaatacata

SEQ ID NO: 731 - RNA, 16 nt, Synthetic construct
  source 1..16: mol_type = other RNA
tgaagtgaatacatga

SEQ ID NO: 732 - RNA, 16 nt, Synthetic construct
  source 1..16: mol_type = other RNA
gaagtgaatacatgga

SEQ ID NO: 733 - RNA, 16 nt, Synthetic construct
  source 1..16: mol_type = other RNA
aagtgaatacatggta

SEQ ID NO: 734 - RNA, 16 nt, Synthetic construct
  source 1..16: mol_type = other RNA
agtgaatacatggtaa

SEQ ID NO: 735 - RNA, 16 nt, Synthetic construct
  source 1..16: mol_type = other RNA
acatggtagcagggta

SEQ ID NO: 736 - RNA, 16 nt, Synthetic construct
  source 1..16: mol_type = other RNA
gcagggtctttgtgta

SEQ ID NO: 737 - RNA, 16 nt, Synthetic construct
  source 1..16: mol_type = other RNA
tgtgctgtggatttta

SEQ ID NO: 738 - RNA, 16 nt, Synthetic construct
  source 1..16: mol_type = other RNA
tgctgtggattttgta

SEQ ID NO: 739 - RNA, 16 nt, Synthetic construct
  source 1..16: mol_type = other RNA
gtggcttcaatctaca

SEQ ID NO: 740 - RNA, 16 nt, Synthetic construct
  source 1..16: mol_type = other RNA
tggcttcaatctacga

SEQ ID NO: 741 - RNA, 16 nt, Synthetic construct
  source 1..16: mol_type = other RNA
ggcttcaatctacgaa

SEQ ID NO: 742 - RNA, 16 nt, Synthetic construct
  source 1..16: mol_type = other RNA
gcttcaatctacgata

SEQ ID NO: 743 - RNA, 16 nt, Synthetic construct
  source 1..16: mol_type = other RNA
cttcaatctacgatga

SEQ ID NO: 744 - RNA, 16 nt, Synthetic construct
  source 1..16: mol_type = other RNA
ttcaatctacgatgta

SEQ ID NO: 745 - RNA, 16 nt, Synthetic construct
  source 1..16: mol_type = other RNA
tcaatctacgatgtta

SEQ ID NO: 746 - RNA, 16 nt, Synthetic construct
  source 1..16: mol_type = other RNA
caatctacgatgttaa

SEQ ID NO: 747 - RNA, 16 nt, Synthetic construct
  source 1..16: mol_type = other RNA
aatctacgatgttaaa

SEQ ID NO: 748 - RNA, 16 nt, Synthetic construct
  source 1..16: mol_type = other RNA
ctacgatgttaaaaca

SEQ ID NO: 749 - RNA, 16 nt, Synthetic construct
  source 1..16: mol_type = other RNA
acctaagtgactacca

SEQ ID NO: 750 - RNA, 16 nt, Synthetic construct
  source 1..16: mol_type = other RNA
cctaagtgactaccaa

SEQ ID NO: 751 - RNA, 16 nt, Synthetic construct
  source 1..16: mol_type = other RNA
ctaagtgactaccaca

SEQ ID NO: 752 - RNA, 16 nt, Synthetic construct
  source 1..16: mol_type = other RNA
taagtgactaccacta

SEQ ID NO: 753 - RNA, 16 nt, Synthetic construct
  source 1..16: mol_type = other RNA
aagtgactaccactta

SEQ ID NO: 754 - RNA, 16 nt, Synthetic construct
  source 1..16: mol_type = other RNA
agtgactaccacttaa

SEQ ID NO: 755 - RNA, 16 nt, Synthetic construct
  source 1..16: mol_type = other RNA
gtgactaccacttata

SEQ ID NO: 756 - RNA, 16 nt, Synthetic construct
  source 1..16: mol_type = other RNA
tgactaccacttatta

SEQ ID NO: 757 - RNA, 16 nt, Synthetic construct
  source 1..16: mol_type = other RNA
gactaccacttattta

SEQ ID NO: 758 - RNA, 16 nt, Synthetic construct
  source 1..16: mol_type = other RNA
actaccacttatttca

SEQ ID NO: 759 - RNA, 16 nt, Synthetic construct
  source 1..16: mol_type = other RNA
ctaccacttatttcta

SEQ ID NO: 760 - RNA, 16 nt, Synthetic construct
  source 1..16: mol_type = other RNA
taccacttatttctaa

SEQ ID NO: 761 - RNA, 16 nt, Synthetic construct
  source 1..16: mol_type = other RNA
accacttatttctaaa

SEQ ID NO: 762 - RNA, 16 nt, Synthetic construct
  source 1..16: mol_type = other RNA
cacttatttctaaata

SEQ ID NO: 763 - RNA, 16 nt, Synthetic construct
  source 1..16: mol_type = other RNA
acttatttctaaatca

SEQ ID NO: 764 - RNA, 16 nt, Synthetic construct
  source 1..16: mol_type = other RNA
ttatttctaaatccta

SEQ ID NO: 765 - RNA, 16 nt, Synthetic construct
  source 1..16: mol_type = other RNA
ttgctgttgttcagaa

SEQ ID NO: 766 - RNA, 16 nt, Synthetic construct
  source 1..16: mol_type = other RNA
tgctgttgttcagaaa

SEQ ID NO: 767 - RNA, 16 nt, Synthetic construct
  source 1..16: mol_type = other RNA
gctgttgttcagaaga

SEQ ID NO: 768 - RNA, 16 nt, Synthetic construct
  source 1..16: mol_type = other RNA
ctgttgttcagaagta

SEQ ID NO: 769 - RNA, 16 nt, Synthetic construct
  source 1..16: mol_type = other RNA
tgttgttcagaagtta

SEQ ID NO: 770 - RNA, 16 nt, Synthetic construct
  source 1..16: mol_type = other RNA
gttgttcagaagttga

SEQ ID NO: 771 - RNA, 16 nt, Synthetic construct
  source 1..16: mol_type = other RNA
ttgttcagaagttgta

SEQ ID NO: 772 - RNA, 16 nt, Synthetic construct
  source 1..16: mol_type = other RNA
tgttcagaagttgtta

SEQ ID NO: 773 - RNA, 16 nt, Synthetic construct
  source 1..16: mol_type = other RNA
gttcagaagttgttaa

SEQ ID NO: 774 - RNA, 16 nt, Synthetic construct
  source 1..16: mol_type = other RNA
ttcagaagttgttaga

SEQ ID NO: 775 - RNA, 16 nt, Synthetic construct
  source 1..16: mol_type = other RNA
tcagaagttgttagta

SEQ ID NO: 776 - RNA, 16 nt, Synthetic construct
  source 1..16: mol_type = other RNA
cagaagttgttagtga

SEQ ID NO: 777 - RNA, 16 nt, Synthetic construct
  source 1..16: mol_type = other RNA
agaagttgttagtgaa

SEQ ID NO: 778 - RNA, 16 nt, Synthetic construct
  source 1..16: mol_type = other RNA
gaagttgttagtgata

SEQ ID NO: 779 - RNA, 16 nt, Synthetic construct
  source 1..16: mol_type = other RNA
aagttgttagtgatta

SEQ ID NO: 780 - RNA, 16 nt, Synthetic construct
  source 1..16: mol_type = other RNA
agttgttagtgattta

SEQ ID NO: 781 - RNA, 16 nt, Synthetic construct
  source 1..16: mol_type = other RNA
gttgttagtgatttga

SEQ ID NO: 782 - RNA, 16 nt, Synthetic construct
  source 1..16: mol_type = other RNA
ttgttagtgatttgca

SEQ ID NO: 783 - RNA, 16 nt, Synthetic construct
  source 1..16: mol_type = other RNA
tgttagtgatttgcta

SEQ ID NO: 784 - RNA, 16 nt, Synthetic construct
  source 1..16: mol_type = other RNA
gttagtgatttgctaa

SEQ ID NO: 785 - RNA, 16 nt, Synthetic construct
  source 1..16: mol_type = other RNA
ttagtgatttgctata

SEQ ID NO: 786 - RNA, 16 nt, Synthetic construct
  source 1..16: mol_type = other RNA
tagtgatttgctatca

SEQ ID NO: 787 - RNA, 16 nt, Synthetic construct
  source 1..16: mol_type = other RNA
agtgatttgctatcaa

SEQ ID NO: 788 - RNA, 16 nt, Synthetic construct
  source 1..16: mol_type = other RNA
gtgatttgctatcata

SEQ ID NO: 789 - RNA, 16 nt, Synthetic construct
  source 1..16: mol_type = other RNA
tgtcttttaatgataa

SEQ ID NO: 790 - RNA, 16 nt, Synthetic construct
  source 1..16: mol_type = other RNA
gtcttttaatgataca

SEQ ID NO: 791 - RNA, 16 nt, Synthetic construct
  source 1..16: mol_type = other RNA
tcttttaatgatacta

SEQ ID NO: 792 - RNA, 16 nt, Synthetic construct
  source 1..16: mol_type = other RNA
cttttaatgatactga

SEQ ID NO: 793 - RNA, 16 nt, Synthetic construct
  source 1..16: mol_type = other RNA
ttttaatgatactgta

SEQ ID NO: 794 - RNA, 16 nt, Synthetic construct
  source 1..16: mol_type = other RNA
tttaatgatactgtca

SEQ ID NO: 795 - RNA, 16 nt, Synthetic construct
  source 1..16: mol_type = other RNA
ctgtctaagaataata

SEQ ID NO: 796 - RNA, 16 nt, Synthetic construct
  source 1..16: mol_type = other RNA
gtctaagaataatgaa

SEQ ID NO: 797 - RNA, 16 nt, Synthetic construct
  source 1..16: mol_type = other RNA
tctaagaataatgaca

SEQ ID NO: 798 - RNA, 16 nt, Synthetic construct
  source 1..16: mol_type = other RNA
ctaagaataatgacga

SEQ ID NO: 799 - RNA, 16 nt, Synthetic construct
  source 1..16: mol_type = other RNA
taagaataatgacgta

SEQ ID NO: 800 - RNA, 16 nt, Synthetic construct
  source 1..16: mol_type = other RNA
aagaataatgacgtaa

SEQ ID NO: 801 - RNA, 16 nt, Synthetic construct
  source 1..16: mol_type = other RNA
gaataatgacgtatta

SEQ ID NO: 802 - RNA, 16 nt, Synthetic construct
  source 1..16: mol_type = other RNA
ataatgacgtattgta

SEQ ID NO: 803 - RNA, 16 nt, Synthetic construct
  source 1..16: mol_type = other RNA
taatgacgtattgtga

SEQ ID NO: 804 - RNA, 16 nt, Synthetic construct
  source 1..16: mol_type = other RNA
aatgacgtattgtgaa

SEQ ID NO: 805 - RNA, 16 nt, Synthetic construct
  source 1..16: mol_type = other RNA
cgtattgtgaaattta

SEQ ID NO: 806 - RNA, 16 nt, Synthetic construct
  source 1..16: mol_type = other RNA
gtattgtgaaatttga

SEQ ID NO: 807 - RNA, 16 nt, Synthetic construct
  source 1..16: mol_type = other RNA
tattgtgaaatttgta

SEQ ID NO: 808 - RNA, 16 nt, Synthetic construct
  source 1..16: mol_type = other RNA
atgtgagcatgaaaca

SEQ ID NO: 809 - RNA, 16 nt, Synthetic construct
  source 1..16: mol_type = other RNA
tgtgagcatgaaacta

SEQ ID NO: 810 - RNA, 16 nt, Synthetic construct
  source 1..16: mol_type = other RNA
gtgagcatgaaactaa

SEQ ID NO: 811 - RNA, 16 nt, Synthetic construct
  source 1..16: mol_type = other RNA
tgagcatgaaactata

SEQ ID NO: 812 - RNA, 16 nt, Synthetic construct
  source 1..16: mol_type = other RNA
gagcatgaaactatga

SEQ ID NO: 813 - RNA, 16 nt, Synthetic construct
  source 1..16: mol_type = other RNA
agcatgaaactatgca

SEQ ID NO: 814 - RNA, 16 nt, Synthetic construct
  source 1..16: mol_type = other RNA
gcatgaaactatgcaa

SEQ ID NO: 815 - RNA, 16 nt, Synthetic construct
  source 1..16: mol_type = other RNA
catgaaactatgcaca

SEQ ID NO: 816 - RNA, 16 nt, Synthetic construct
  source 1..16: mol_type = other RNA
atgaaactatgcacca

SEQ ID NO: 817 - RNA, 16 nt, Synthetic construct
  source 1..16: mol_type = other RNA
tgaaactatgcaccta

SEQ ID NO: 818 - RNA, 16 nt, Synthetic construct
  source 1..16: mol_type = other RNA
gaaactatgcacctaa

SEQ ID NO: 819 - RNA, 16 nt, Synthetic construct
  source 1..16: mol_type = other RNA
aaactatgcacctata

SEQ ID NO: 820 - RNA, 16 nt, Synthetic construct
  source 1..16: mol_type = other RNA
aactatgcacctataa

SEQ ID NO: 821 - RNA, 16 nt, Synthetic construct
  source 1..16: mol_type = other RNA
ctatgcacctataaaa

SEQ ID NO: 822 - RNA, 16 nt, Synthetic construct
  source 1..16: mol_type = other RNA
atgcacctataaataa

SEQ ID NO: 823 - RNA, 16 nt, Synthetic construct
  source 1..16: mol_type = other RNA
gcacctataaatacta

SEQ ID NO: 824 - RNA, 16 nt, Synthetic construct
  source 1..16: mol_type = other RNA
attttaccattttgca

SEQ ID NO: 825 - RNA, 16 nt, Synthetic construct
  source 1..16: mol_type = other RNA
taccattttgcgatga

SEQ ID NO: 826 - RNA, 16 nt, Synthetic construct
  source 1..16: mol_type = other RNA
ccattttgcgatgtga

SEQ ID NO: 827 - RNA, 16 nt, Synthetic construct
  source 1..16: mol_type = other RNA
cattttgcgatgtgta

SEQ ID NO: 828 - RNA, 16 nt, Synthetic construct
  source 1..16: mol_type = other RNA
attttgcgatgtgtta

SEQ ID NO: 829 - RNA, 16 nt, Synthetic construct
  source 1..16: mol_type = other RNA
ttttgcgatgtgttta

SEQ ID NO: 830 - RNA, 16 nt, Synthetic construct
  source 1..16: mol_type = other RNA
tttgcgatgtgtttta

SEQ ID NO: 831 - RNA, 16 nt, Synthetic construct
  source 1..16: mol_type = other RNA
ttgcgatgtgttttaa

SEQ ID NO: 832 - RNA, 16 nt, Synthetic construct
  source 1..16: mol_type = other RNA
cgatgtgttttattca

SEQ ID NO: 833 - RNA, 16 nt, Synthetic construct
  source 1..16: mol_type = other RNA
gatgtgttttattcaa

SEQ ID NO: 834 - RNA, 16 nt, Synthetic construct
  source 1..16: mol_type = other RNA
atgtgttttattcaca

SEQ ID NO: 835 - RNA, 16 nt, Synthetic construct
  source 1..16: mol_type = other RNA
gtgttttattcactta

SEQ ID NO: 836 - RNA, 16 nt, Synthetic construct
  source 1..16: mol_type = other RNA
tgttttattcacttga

SEQ ID NO: 837 - RNA, 16 nt, Synthetic construct
  source 1..16: mol_type = other RNA
gttttattcacttgta

SEQ ID NO: 838 - RNA, 16 nt, Synthetic construct
  source 1..16: mol_type = other RNA
ttttattcacttgtga

SEQ ID NO: 839 - RNA, 16 nt, Synthetic construct
  source 1..16: mol_type = other RNA
ttgtatataaatggta

SEQ ID NO: 840 - RNA, 16 nt, Synthetic construct
  source 1..16: mol_type = other RNA
gtatataaatggtgaa

SEQ ID NO: 841 - RNA, 16 nt, Synthetic construct
  source 1..16: mol_type = other RNA
  modified_base order(1..3,5,7,9,11..13,15,16): mod_base = OTHER; note = 2'-O-methyl
  modified_base order(4,6,8,10,14): mod_base = OTHER; note = 2'-fluoro-2'-deoxy
  modified_base 1..3: mod_base = OTHER; note = phosphorothioate internucleoside linkage
  modified_base 14..16: mod_base = OTHER; note = phosphorothioate internucleoside linkage
taccacttatttctaa

SEQ ID NO: 842 - RNA, 21 nt, Synthetic construct
  source 1..21: mol_type = other RNA
  modified_base 1: mod_base = OTHER; note = vinyl phosphonate
  modified_base order(1,3,7,9,11,13,15,17..21): mod_base = OTHER; note = 2'-O-methyl
  modified_base order(2,4..6,8,10,12,14,16): mod_base = OTHER; note = 2'-fluoro-2'-deoxy
  modified_base 1..3: mod_base = OTHER; note = phosphorothioate internucleoside linkage
  modified_base 16..21: mod_base = OTHER; note = phosphorothioate internucleoside linkage
ttagaaataagtggtagtcac

SEQ ID NO: 843 - RNA, 16 nt, Synthetic construct
  source 1..16: mol_type = other RNA
  modified_base order(1..3,5,7,9,11..13,15,16): mod_base = OTHER; note = 2'-O-methyl
  modified_base order(4,6,8,10,14): mod_base = OTHER; note = 2'-fluoro-2'-deoxy
  modified_base 1..3: mod_base = OTHER; note = phosphorothioate internucleoside linkage
  modified_base 14..16: mod_base = OTHER; note = phosphorothioate internucleoside linkage
caagtgctcagttcca

SEQ ID NO: 844 - RNA, 21 nt, Synthetic construct
  source 1..21: mol_type = other RNA
  modified_base 1: mod_base = OTHER; note = vinyl phosphonate
  modified_base order(1,3,7,9,11,13,15,17..21): mod_base = OTHER; note = 2'-O-methyl
  modified_base order(2,4..6,8,10,12,14,16): mod_base = OTHER; note = 2'-fluoro-2'-deoxy
  modified_base 1..3: mod_base = OTHER; note = phosphorothioate internucleoside linkage
  modified_base 16..21: mod_base = OTHER; note = phosphorothioate internucleoside linkage
tggaactgagcacttgtacag

SEQ ID NO: 845 - RNA, 16 nt, Synthetic construct
  source 1..16: mol_type = other RNA
  modified_base order(1..3,5,7,9,11..13,15,16): mod_base = OTHER; note = 2'-O-methyl
  modified_base order(4,6,8,10,14): mod_base = OTHER; note = 2'-fluoro-2'-deoxy
  modified_base 1..3: mod_base = OTHER; note = phosphorothioate internucleoside linkage
  modified_base 14..16: mod_base = OTHER; note = phosphorothioate internucleoside linkage
agtggtgcatggtgta

SEQ ID NO: 846 - RNA, 21 nt, Synthetic construct
  source 1..21: mol_type = other RNA
  modified_base 1: mod_base = OTHER; note = vinyl phosphonate
  modified_base order(1,3,7,9,11,13,15,17..21): mod_base = OTHER; note = 2'-O-methyl
  modified_base order(2,4..6,8,10,12,14,16): mod_base = OTHER; note = 2'-fluoro-2'-deoxy
  modified_base 1..3: mod_base = OTHER; note = phosphorothioate internucleoside linkage
  modified_base 16..21: mod_base = OTHER; note = phosphorothioate internucleoside linkage
tacaccatgcaccactccctc

SEQ ID NO: 847 - RNA, 16 nt, Synthetic construct
  source 1..16: mol_type = other RNA
  modified_base order(1..3,5,7,9,11..13,15,16): mod_base = OTHER; note = 2'-O-methyl
  modified_base order(4,6,8,10,14): mod_base = OTHER; note = 2'-fluoro-2'-deoxy
  modified_base 1..3: mod_base = OTHER; note = phosphorothioate internucleoside linkage
  modified_base 14..16: mod_base = OTHER; note = phosphorothioate internucleoside linkage
caatgaggcttatgaa

SEQ ID NO: 848 - RNA, 21 nt, Synthetic construct
  source 1..21: mol_type = other RNA
  modified_base 1: mod_base = OTHER; note = vinyl phosphonate
  modified_base order(1,3,7,9,11,13,15,17..21): mod_base = OTHER; note = 2'-O-methyl
  modified_base order(2,4..6,8,10,12,14,16): mod_base = OTHER; note = 2'-fluoro-2'-deoxy
  modified_base 1..3: mod_base = OTHER; note = phosphorothioate internucleoside linkage
  modified_base 16..21: mod_base = OTHER; note = phosphorothioate internucleoside linkage
tttggaactgagcacttgtac

SEQ ID NO: 849 - RNA, 16 nt, Synthetic construct
  source 1..16: mol_type = other RNA
  modified_base order(1..3,5,7,9,11..13,15,16): mod_base = OTHER; note = 2'-O-methyl
  modified_base order(4,6,8,10,14): mod_base = OTHER; note = 2'-fluoro-2'-deoxy
  modified_base 1..3: mod_base = OTHER; note = phosphorothioate internucleoside linkage
  modified_base 14..16: mod_base = OTHER; note = phosphorothioate internucleoside linkage
tcagttccaatgtgca

SEQ ID NO: 850 - RNA, 21 nt, Synthetic construct
  source 1..21: mol_type = other RNA
  modified_base 1: mod_base = OTHER; note = vinyl phosphonate
  modified_base order(1,3,7,9,11,13,15,17..21): mod_base = OTHER; note = 2'-O-methyl
  modified_base order(2,4..6,8,10,12,14,16): mod_base = OTHER; note = 2'-fluoro-2'-deoxy
  modified_base 1..3: mod_base = OTHER; note = phosphorothioate internucleoside linkage
  modified_base 16..21: mod_base = OTHER; note = phosphorothioate internucleoside linkage
tttggaactgagcacttgtac

SEQ ID NO: 851 - RNA, 16 nt, Synthetic construct
  source 1..16: mol_type = other RNA
  modified_base order(1..3,5,7,9,11..13,15,16): mod_base = OTHER; note = 2'-O-methyl
  modified_base order(4,6,8,10,14): mod_base = OTHER; note = 2'-fluoro-2'-deoxy
  modified_base 1..3: mod_base = OTHER; note = phosphorothioate internucleoside linkage
  modified_base 14..16: mod_base = OTHER; note = phosphorothioate internucleoside linkage
ctacgatgttaaaaca

SEQ ID NO: 852 - RNA, 21 nt, Synthetic construct
  source 1..21: mol_type = other RNA
  modified_base 1: mod_base = OTHER; note = vinyl phosphonate
  modified_base order(1,3,7,9,11,13,15,17..21): mod_base = OTHER; note = 2'-O-methyl
  modified_base order(2,4..6,8,10,12,14,16): mod_base = OTHER; note = 2'-fluoro-2'-deoxy
  modified_base 1..3: mod_base = OTHER; note = phosphorothioate internucleoside linkage
  modified_base 16..21: mod_base = OTHER; note = phosphorothioate internucleoside linkage
tgcacattggaactgagcact

SEQ ID NO: 853 - RNA, 16 nt, Synthetic construct
  source 1..16: mol_type = other RNA
  modified_base order(1..3,5,7,9,11..13,15,16): mod_base = OTHER; note = 2'-O-methyl
  modified_base order(4,6,8,10,14): mod_base = OTHER; note = 2'-fluoro-2'-deoxy
  modified_base 1..3: mod_base = OTHER; note = phosphorothioate internucleoside linkage
  modified_base 14..16: mod_base = OTHER; note = phosphorothioate internucleoside linkage
ctacgatgttaaaaca

SEQ ID NO: 854 - RNA, 21 nt, Synthetic construct
  source 1..21: mol_type = other RNA
  modified_base 1: mod_base = OTHER; note = vinyl phosphonate
  modified_base order(1,3,7,9,11,13,15,17..21): mod_base = OTHER; note = 2'-O-methyl
  modified_base order(2,4..6,8,10,12,14,16): mod_base = OTHER; note = 2'-fluoro-2'-deoxy
  modified_base 1..3: mod_base = OTHER; note = phosphorothioate internucleoside linkage
  modified_base 16..21: mod_base = OTHER; note = phosphorothioate internucleoside linkage
tgttttaacatcgtagattga

SEQ ID NO: 855 - RNA, 21 nt, Synthetic construct
  source 1..21: mol_type = other RNA
  modified_base 1: mod_base = OTHER; note = vinyl phosphonate
  modified_base order(1,3,7,9,11,13,15,17..21): mod_base = OTHER; note = 2'-O-methyl
  modified_base order(2,4..6,8,10,12,14,16): mod_base = OTHER; note = 2'-fluoro-2'-deoxy
  modified_base 1..3: mod_base = OTHER; note = phosphorothioate internucleoside linkage
  modified_base 16..21: mod_base = OTHER; note = phosphorothioate internucleoside linkage
ttcataagcctcattgtcagg

SEQ ID NO: 856 - RNA, 16 nt, Synthetic construct
  source 1..16: mol_type = other RNA
  modified_base order(1..3,5,7,9,11..13,15,16): mod_base = OTHER; note = 2'-O-methyl
  modified_base order(4,6,8,10,14): mod_base = OTHER; note = 2'-fluoro-2'-deoxy
  modified_base 1..3: mod_base = OTHER; note = phosphorothioate internucleoside linkage
  modified_base 14..16: mod_base = OTHER; note = phosphorothioate internucleoside linkage
agtgctcagttccaaa

SEQ ID NO: 857 - RNA, 16 nt, Synthetic construct
  source 1..16: mol_type = other RNA
  modified_base order(1..3,5,7,9,11..13,15,16): mod_base = OTHER; note = 2'-O-methyl
  modified_base order(4,6,8,10,14): mod_base = OTHER; note = 2'-fluoro-2'-deoxy
  modified_base 1..3: mod_base = OTHER; note = phosphorothioate internucleoside linkage
  modified_base 14..16: mod_base = OTHER; note = phosphorothioate internucleoside linkage
tcagttccaatgtgca

SEQ ID NO: 858 - RNA, 3215 nt, Homo sapiens
  source 1..3215: mol_type = mRNA
aggagaaggagaaggaggaggactaggaggaggaggacggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 859 - RNA, 3211 nt, Homo sapiens
  source 1..3211: mol_type = mRNA
gccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaagagagaggcggggaggagtcggagttgtggagaagcagagggactcagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 860 - RNA, 3022 nt, Homo sapiens
  source 1..3022: mol_type = mRNA
attctggtgtgatccaggaacagctgtcttccagctctgaaagagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 861 - RNA, 3127 nt, Homo sapiens
  source 1..3127: mol_type = mRNA
gccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaagagagaggcggggaggagtcggagttgtggagaagcagagggactcagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 862 - RNA, 21 nt, Homo sapiens
  source 1..21: mol_type = mRNA
gtgactaccacttatttctaa

SEQ ID NO: 863 - RNA, 21 nt, Homo sapiens
  source 1..21: mol_type = mRNA
ctgtacaagtgctcagttcca

SEQ ID NO: 864 - RNA, 21 nt, Homo sapiens
  source 1..21: mol_type = mRNA
gagggagtggtgcatggtgtg

SEQ ID NO: 865 - RNA, 21 nt, Homo sapiens
  source 1..21: mol_type = mRNA
cctgacaatgaggcttatgaa

SEQ ID NO: 866 - RNA, 21 nt, Homo sapiens
  source 1..21: mol_type = mRNA
gtacaagtgctcagttccaat

SEQ ID NO: 867 - RNA, 21 nt, Homo sapiens
  source 1..21: mol_type = mRNA
agtgctcagttccaatgtgcc

SEQ ID NO: 868 - RNA, 21 nt, Homo sapiens
  source 1..21: mol_type = mRNA
tcaatctacgatgttaaaaca